(19) United States
(12) Patent Application Publication
(10) Pub. No.: US 2025/0108038 A1
(43) Pub. Date: Apr. 3, 2025

(54) SUBSTITUTED DERIVATIVES OF ISOINDOLES AND USES THEREOF

(71) Applicant: Supernus Pharmaceuticals, Inc., Rockville, MD (US)

(72) Inventors: Gary L. Olson, Mountainside, NJ (US); Bhaskara Rao Nallaganchu, Hillsborough, NJ (US); Yanjun Sun, Kendall Park, NJ (US); Chungping YU, Gaithersburg, MD (US)

(73) Assignee: Supernus Pharmaceuticals, Inc., Rockville, MD (US)

(21) Appl. No.: 18/899,299

(22) Filed: Sep. 27, 2024

Related U.S. Application Data
(60) Provisional application No. 63/541,465, filed on Sep. 29, 2023.
(60) Provisional application No. 63/683,074, filed on Aug. 14, 2024.

(51) Int. Cl. A61K 31/4188; A61K 31/4164; A61K 31/4178; A61K 31/496; A61P 3/04

(52) U.S. Cl. CPC A61K 31/4188; A61K 31/4164; A61K 31/4178; A61K 31/496; A61P 3/04

(57) ABSTRACT

Substituted derivatives of 5-(4-chlorophenyl)-2,3-dihydroimidazo[1,2-b]isoindol-5-ol and their uses in pharmaceutical compositions for the treatment of central nervous system diseases or disorders are provided.

### RELATED APPLICATIONS

The present application claims priority to U.S. provisional applications Nos. 63/541,465 filed Sep. 29, 2023 and 63/683,074 filed Aug. 14, 2024, each of which is incorporated herein by reference in its entirety.

### FIELD

The present technology generally relates to substituted derivatives of isoindoles, analogs of isoindoles, prodrugs of isoindoles, and their uses in pharmaceutical compositions for the treatment of diseases or disorders including the treatment of central nervous system (“CNS”) disorders.

### BACKGROUND

The compound 5-(4-chlorophenyl)-2,3-dihydroimidazo[1,2-b]isoindol-5-ol (Compound A) is a sympathomimetic amine that stimulates the central nervous system (“CNS”), increasing heart rate, blood pressure, and decreasing appetite. Compound A acts as a reuptake inhibitor of norepinephrine, dopamine, and serotonin. However, treatment with Compound A has been associated with numerous side effects including hypertension, anorexia, gastrointestinal discomfort, nervousness, nausea, constipation, urinary retention, angioneurotic edema, vomiting, tremor, xerostomia, insomnia or difficulty sleeping, and EKG and EEG abnormalities.

Indoline derivatives have previously been disclosed in the art. A prior disclosure indicated that isoindoles can be used for the treatment or prevention of neurobehavioral disorders (see U.S. 2009/0318520). Previously disclosed syntheses of these Compound A analogs, prodrugs, and derivatives suffer from a number of deficiencies, such as low reaction yield, reaction byproducts, difficult isolation of the product and impurities in the resulting product. Effective elimination or removal of impurities, especially those impurities possessing genotoxicity or other toxicities, is critical to render safe pharmaceutical products. Disclosed herein are solutions to these and other related problems.

In order to minimize side effects associated with Compound A, the present inventors have synthesized derivatives, prodrugs, and analogs that retain the pharmacological properties of Compound A. Prodrugs are a class of derivatives that in many instances have little or no pharmacological activity, which are converted in vivo to therapeutically active compounds. In some instances, the prodrug itself may possess biological activity. Prodrug activation may occur by enzymatic or non-enzymatic cleavage of the temporary bond between the carrier and the drug molecule, or a sequential or simultaneous combination of both.

Prodrugs may provide compounds with superior physicochemical and/or pharmaceutical properties as compared to the parent molecule, which may overcome barriers for solubilization, absorption, distribution, metabolism, excretion, and toxicity (ADMET). These prodrugs may show improved absorption, solubility, permeability, stability, and pharmacokinetic performance. The prodrug may exhibit a longer half-life as compared to the parent molecule. Prodrugs may be prepared by coupling of the parent drug at reactive sites with prodrug moieties that modify the parent drug and are convertible from the prodrug to the parent drug by enzymatic or non-enzymatic processes. The reactive sites on the drug may include, but are not limited to, hydroxyl, carboxyl, amino, heteroamino, thiol, amide, and related reactive groups. These are coupled to form prodrugs with alkyl, aralkyl, acyl, carbamoyl, acyloxy, and moieties that have combined groups such as diacylacetals or acylhydroxyalkyl, groups. Other examples are described in the literature (see Yang, Liu, et al., Acta Pharmaceutica Sinica B 2011: 1(3), 143-159 and references described therein). Additional methods for synthesizing prodrugs of Compound A would be beneficial.

Drug analogs are a class of compounds sharing chemical and/or therapeutic similarities with an existing pharmaceutical. Drug analogs generally present in three categories: (1) analogs with chemical and pharmacological similarity; (2) analogs with only chemical similarity; and (3) analogs with similar pharmacological properties but different chemical structures. Although the analog may have similar physical properties, the analog may have distinct chemical and biological properties. Alternatively, the analog may share chemical and therapeutic similarities with the existing drug. Analogs of a compound may provide additional pharmacological agents for treatment of disorders with varying properties that may translate to additional efficacy, reduced toxicity, or increased tolerance.

The present inventors synthesized novel derivatives of isoindoles with the derivatization occurring at several locations. In one approach, the derivatization is of the hydroxy group on the Compound A. In other approaches, the derivatization occurs at the amine group of the imidazoline ring of the keto tautomer. The newly synthesized Compound A derivatives can be used in pharmaceutical compositions and for the treatment of diseases or disorders including the treatment of CNS disorders.

### SUMMARY OF THE INVENTION

In one aspect, the treatment of disease or disorders, including the treatment of central nervous system (“CNS”) disorders, is provided by administering to a subject in need thereof a therapeutically effective amount of a compound of Formula I, II, or III, a stereoisomer thereof, or a pharmaceutically acceptable salt thereof:

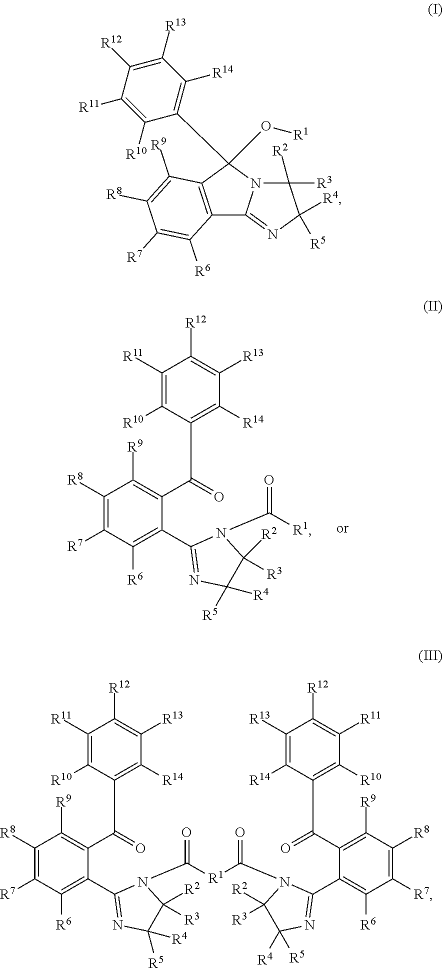

where: for Formula I, R¹ is an alkyl group, an alkenyl group, an aralkyl group, an alkoxyalkyl group, a carboxyalkyl ester group, a cinnamyl group, a heteroalkyl group or a heterocycloalkyl group; R²-R⁵ are each independently H or alkyl; and R⁶-R¹⁴ are each independently H, alkyl, alkoxy, F, Cl, Br, CF₃ or I; for Formula II, R¹ is a piperazinyl group, an alkyl group, an alkenyl group, a benzyl group, a phenyl group, or an amino acid residue derivative; and R²-R¹⁴ are each independently H, F, Cl, Br, or I; and for Formula III, R¹ is an alkyl group, an alkenyl group, an aryl group, or a heteroaryl group; and R²-R¹⁴ are each independently H, F, Cl, Br, or I.

### BRIEF DESCRIPTION OF THE DRAWINGS

FIG. 1A. Stability of Compound I-19 in 3 different fluid matrices modeling 3 different body fluids: SGF (gastric, pH 2.0), SIF (upper intestinal, pH 6.0), and PBS (systemic fluid, pH 7.4). A stock solution of 10 mM was prepared and a 10 μL aliquot was added to each fluid matrix at 0 min. (final concentration 0.1 mM) and the mixture was incubated at 37° C. for 120 min. total. Samples were taken at 0, 15, 30, 60, and 120 min. and were analyzed by LCMSMS for % parent compound remaining. Values represent mean+SEM of n=3 separate experiments.

FIG. 1B. Stability of Compound I-4 in 3 different fluid matrices modeling 3 different body fluids: SGF (gastric, pH 2.0), SIF (upper intestinal, pH 6.0), and PBS (systemic fluid, pH 7.4). A stock solution of 10 mM was prepared and a 10 μL aliquot was added to each fluid matrix at 0 min. (final concentration 0.1 mM) and the mixture was incubated at 37° C. for 120 min. total. Samples were taken at 0, 15, 30, 60, and 120 min. and were analyzed by LCMSMS for % parent compound remaining. Values represent mean+SEM of n=3 separate experiments.

FIG. 2A. Intestinal permeability of Compound I-19. Compound I-19 was determined in the human 3D intestinal model (EpiIntestinal) from MatTek Corp. Tissues were dosed apically with 100 μl of 10 mM stock solution (4.25 μg) at 0 min. and the tissues were incubated with the test articles at 37° C. for a total of 120 min. Samples were taken from the basolateral compartment at 0, 15, 30, 60, and 120 min. and were analyzed by LCMSMS for presence of parent compound. Values represent mean+SEM of n=3 separate experiments.

FIG. 2B. Intestinal permeability of Compound I-4 was determined in the human 3D intestinal model (Epilntestinal) from MatTek Corp. Tissues were dosed apically with 100 μL of 10 mM stock solution (4.25 μg) at 0 min. and the tissues were incubated with the test articles at 37° C. for a total of 120 min. Samples were taken from the basolateral compartment at 0, 15, 30, 60, and 120 min. and were analyzed by LCMSMS for presence of parent compound. Values represent mean+SEM of n=3 separate experiments.

FIG. 3A. Stability of Compound I-19. Compound I-19 in components of human blood. Red blood cells and plasma were isolated from whole blood. A stock solution of 10 mM was prepared and a 50 μL aliquot was added to each blood matrix at 0 min. (final concentration 0.5 mM) and the mixtures were incubated with the test articles at 37° C. fora total of 120 min. Samples were taken at 0, 15, 30, 60, and 120 min. and were analyzed by LCMSMS for % parent compound remaining. Values represent mean+SEM of n=3 separate experiments.

FIG. 3B. Stability of Compound I-4 in components of human blood. Red blood cells and plasma were isolated from whole blood. A stock solution of 10 mM was prepared and a 50 μl aliquot was added to each blood matrix at 0 min. (final concentration 0.5 mM) and the mixtures were incubated with the test articles at 37° C. for a total of 120 min. Samples were taken at 0, 15, 30, 60, and 120 min. and were analyzed by LCMSMS for % parent compound remaining. Values represent mean+SEM of n=3 separate experiments.

FIG. 4A. Hemolytic Potential of Compound I-19. Compound I-19 in human blood. Red blood cells were isolated from whole blood and resuspended in PBS. 190 μl of diluted RBC suspension and 10 L of Compound I-19. Compound I-19 solutions were mixed at 0 min. (final concentrations 25, 50, and 100 μM) and the mixtures were incubated at 37° C. for 60 min. After the incubation, the samples were centrifuged and the absorbance of 100 μL of supernatant was read at 410 nm. % RBC lysis is expressed relative to 100% lysis control. Values represent mean+SEM of n=3 separate experiments.

FIG. 4B. Hemolytic Potential of Compound I-4 in human blood. Red blood cells were isolated from whole blood and resuspended in PBS. 190 μL of diluted RBC suspension and 10 μL of Compound I-4 solutions were mixed at 0 min. (final concentrations 25, 50, and 100 μM) and the mixtures were incubated at 37° C. for 60 min. After the incubation, the samples were centrifuged and the absorbance of 100 μL of supernatant was read at 410 nm. % RBC lysis is expressed relative to 100% lysis control. Values represent mean+SEM of n=3 separate experiments.

FIG. 4C. Hemolytic Potential of Amphotericin B in human blood (positive assay control). Red blood cells were isolated from whole blood and resuspended in PBS. 190 μL of diluted RBC suspension and 10 μl of amphotericin B solutions were mixed at 0 min. (final concentrations 1, 10, 30, and 100 μM) and the mixtures were incubated at 37° C. for 60 min. After the incubation, the samples were centrifuged and the absorbance of 100 μL of supernatant was read at 410 nm. % RBC lysis is expressed relative to 100% lysis control. Values represent mean+SEM of n=3 separate experiments.

FIG. 5A. Stability of Compound I-19. Compound I-19 in components of rat blood. Red blood cells and plasma were isolated from whole blood. A stock solution of 10 mM was prepared and a 50 μL aliquot was added to each blood matrix at O min. (final concentration 0.5 mM) and the mixtures were incubated with the test articles at 37° C. for a total of 120 min. Samples were taken at 0, 15, 30, 60, and 120 min. and were analyzed by LCMSMS for % parent compound remaining. Values represent mean+SEM of n=3 separate experiments.

FIG. 5B. Stability of Compound I-4 in components of rat blood. Red blood cells and plasma were isolated from whole blood. A stock solution of 10 mM was prepared and a 50 μL aliquot was added to each blood matrix at 0 min. (final concentration 0.5 mM) and the mixtures were incubated with the test articles at 37° C. for a total of 120 min. Samples were taken at 0, 15, 30, 60, and 120 min. and were analyzed by LCMSMS for % parent compound remaining. Values represent mean+SEM of n=3 separate experiments.

FIG. 6A. Hemolytic Potential of Compound I-19. Compound I-19 in rat blood. Red blood cells were isolated from whole blood and resuspended in PBS. 190 μL of diluted RBC suspension and 10 μL of Compound I-19. Compound I-19 solutions were mixed at 0 min. (final concentrations 25, 50, and 100 μM) and the mixtures were incubated at 37° C. for 60 min. After the incubation, the samples were centrifuged and the absorbance of 100 μL of supernatant was read at 410 nm. % RBC lysis is expressed relative to 100% lysis control. Values represent mean+SEM of n=3 separate experiments.

FIG. 6B. Hemolytic Potential of Compound I-19. Compound I-19 in rat blood. Red blood cells were isolated from whole blood and resuspended in PBS. 190 UL of diluted RBC suspension and 10 μL of Compound I-19. Compound I-19 solutions were mixed at 0 min. (final concentrations 25, 50, and 100 μM) and the mixtures were incubated at 37° C. for 60 min. After the incubation, the samples were centrifuged and the absorbance of 100 μl of supernatant was read at 410 nm. % RBC lysis is expressed relative to 100% lysis control. Values represent mean+SEM of n=3 separate experiments.

FIG. 6C. Hemolytic Potential of Amphotericin B in rat blood (positive assay control). Red blood cells were isolated from whole blood and resuspended in PBS. 190 μL of diluted RBC suspension and 10 μL of amphotericin B solutions were mixed at 0 min. (final concentrations 1, 10, 30, and 100 μM) and the mixtures were incubated at 37° C. for 60 min. After the incubation, the samples were centrifuged and the absorbance of 100 μL of supernatant was read at 410 nm. % RBC lysis is expressed relative to 100% lysis control. Values represent mean+SEM of n=3 separate experiments.

FIG. 7A. Stability of Compound I-19 in human and rat fecal matter. Gut microbiota from rat and human sources were extracted and purified in PBS at pH 6.0 or 7.4. A stock solution of 10 mM was prepared and a 10 μL aliquot was added to extracted microorganisms at 0 min. (final concentration 0.5 mM) and the mixtures were incubated at 37° C. for a total of 120 min. Samples were taken at 0, 15, 30, 60, and 120 min. and analyzed by LCMSMS for % parent compound remaining. Values represent mean+SEM of n=3 separate experiments. It should be noted that the 0 min. time point for rat, PBS pH 6.0 was showing issues and the rat PBS pH 7.4 0 min. time point was used as a comparison for this sample.

FIG. 7B. Stability of Compound I-4 in human and rat fecal matter. Gut microbiota from rat and human sources were extracted and purified in PBS at pH 6.0 or 7.4. A stock solution of 10 mM was prepared and a 10 μL aliquot was added to extracted microorganisms at 0 min. (final concentration 0.5 mM) and the mixtures were incubated at 37° C. for a total of 120 min. Samples were taken at 0, 15, 30, 60, and 120 min. and analyzed by LC/MS/MS for % parent compound remaining. Values represent mean+SEM of n=3 separate experiments. It should be noted that the 0 min. time point for rat, PBS pH 6.0 was showing issues and the rat PBS pH 7.4 0 min. time point was used as a comparison for this sample.

FIG. 8A shows the percent remaining of Compound I-4 in SD Rat Plasma. FIG. 8B shows the percent remaining of Compound I-4 in Dog Plasma. FIG. 8C shows the percent remaining of Compound I-4 in Human Plasma. FIG. 8D shows the percent remaining of Compound I-19 in SD Rat Plasma. FIG. 8E shows the percent remaining of Compound I-19 in Human Plasma.

FIG. 9 shows plasma concentration-time profiles of Compound I-17 following single PO administration to male and female beagle dogs at 10 mg/kg.

FIG. 10 shows plasma concentration-time profiles of Compound A following single PO administration of Compound I-17 to male and female beagle dogs at 10 mg/kg.

FIG. 11 shows plasma concentration-time profiles of Compound I-19 following single PO administration to male and female beagle dogs at 10 mg/kg.

FIG. 12 shows plasma concentration-time profiles of Compound A following single PO administration of Compound I-19 to male and female beagle dogs at 10 mg/kg.

FIG. 13 shows individual and mean plasma concentration-time profiles of Compound A following single PO administration to male CD-1 mice at 5.102 mg/kg.

FIG. 14 shows individual and mean plasma concentration-time profiles of Compound I-19 following single PO administration to male CD-1 mice at 6.645 mg/kg.

FIG. 15 shows individual and mean plasma concentration-time profiles of Compound A following single PO administration of Compound I-19 to male CD-1 mice at 6.645 mg/kg.

FIG. 16 shows individual and mean plasma concentration-time profiles of Compound I-19 following single IP administration to male CD-1 mice at 4.061 mg/kg.

FIG. 17 shows individual and mean plasma concentration-time profiles of Compound A following single IP administration of Compound I-19 to male CD-1 mice at 4.061 mg/kg.

FIG. 18 shows individual and mean plasma concentration-time profiles of Compound B following single IP administration of Compound I-19 to male CD-1 mice at 4.061 mg/kg.

FIG. 19 shows individual and mean plasma concentration-time profiles of Compound I-4 following single PO administration to male Wister Han rats at 3.708 mg/kg.

FIG. 20 shows individual and mean plasma concentration-time profiles of Compound I-4 following single IP administration to male Wister Han rats at 3.708 mg/kg.

FIG. 21 shows individual and mean plasma concentration-time profiles of Compound I-19 following single PO administration to male Wister Han rats at 4.061 mg/kg.

FIG. 22 shows individual and mean plasma concentration-time profiles of Compound I-19 following single IP administration to male Wister Han rats at 4.061 mg/kg.

FIG. 23 shows individual and mean plasma concentration-time profiles of Compound A following single PO administration of Compound I-4 to male Wistar Han rats at 3.708 mg/kg.

FIG. 24 shows individual and mean plasma concentration-time profiles of Compound A following single IP administration of Compound I-4 to male Wistar Han rats at 3.708 mg/kg.

FIG. 25 shows individual and mean plasma concentration-time profiles of Compound A following single PO administration of Compound I-19 to male Wistar Han rats at 4.061 mg/kg.

FIG. 26 shows individual and mean plasma concentration-time profiles of Compound A following single IP administration of Compound I-19 to male Wistar Han rats at 4.061 mg/kg.

FIG. 27 shows individual and mean plasma concentration-time profiles of Compound B following single PO administration of Compound I-4 to male Wister Han rats at 3.708 mg/kg.

FIG. 28 shows individual and mean plasma concentration-time profiles of compound B following single IP administration of Compound I-4 to male Wister Han rats at 3.708 mg/kg.

FIG. 29 shows individual and mean plasma concentration-time profiles of Compound B following single PO administration of Compound I-19 to male Wister Han rats at 4.061 mg/kg.

FIG. 30 shows individual and mean plasma concentration-time profiles of Compound B following single IP administration of Compound I-19 to male Wister Han rats at 4.061 mg/kg.

FIG. 31 shows in vivo PK parameters in mice for Compound I-19 and Compound A following 3 mg/kg IP administration as plasma and brain concentration versus time profiles.

FIG. 32 shows stability of Compound I-19 in DPBS, plasma and brain homogenate.

FIG. 33 shows changes in % unbound and bound of Compound I-19 over 6 hours for DPBS, plasma and brain homogenate.

### DETAILED DESCRIPTION

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein merely intended to serve as shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³², and S³⁵ are thus within the scope of the present technology. Procedures for inserting such labels into the compound of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include, but are not limited to: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SFs), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

As used herein, “alkyl” groups include straight chain and branched alkyl groups having from 1 to about 20 carbon atoms, and typically from 1 to 12 carbons or, in some embodiments, from 1 to 8 carbon atoms. As employed herein, “alkyl groups” include cycloalkyl groups as defined below. Alkyl groups may be substituted or unsubstituted. Examples of straight chain alkyl groups include, but are not limited to, methyl, ethyl, n-propyl, n-butyl, n-hexyl, n-heptyl, and n-octyl groups. Examples of branched alkyl groups include, but are not limited to, isopropyl, sec-butyl, t-butyl, neopentyl, and isopentyl groups. Representative substituted alkyl groups may be substituted one or more times with, for example, amino, thio, hydroxy, cyano, alkoxy, and/or halo groups such as F, Cl, Br, and I groups. As used herein, the term haloalkyl is an alkyl group having one or more halo groups. In some embodiments, haloalkyl refers to a per-haloalkyl group.

Cycloalkyl groups are cyclic alkyl groups such as, without limitation, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 6, or 7. Cycloalkyl groups may be substituted or unsubstituted. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups may be mono-substituted or substituted more than once, such as, without limitation: 2,2-; 2,3-; 2,4-; 2,5-; or 2,6-disubstituted cyclohexyl groups or mono-, di-, or tri-substituted norbornyl or cycloheptyl groups, which may be substituted with, for example, alkyl, alkoxy, amino, thio, hydroxy, cyano, and/or halo groups.

Alkenyl groups are straight chain, branched or cyclic alkyl groups having 2 to about 20 carbon atoms, and further including at least one double bond. In some embodiments alkenyl groups have from 1 to 12 carbons, or, typically, from 1 to 8 carbon atoms. Alkenyl groups may be substituted or unsubstituted. Alkenyl groups include, for instance, vinyl, propenyl, 2-butenyl, 3-butenyl, isobutenyl, cyclohexenyl, cyclopentenyl, cyclohexadienyl, butadienyl, pentadienyl, and hexadienyl groups among others. Alkenyl groups may be substituted similarly to alkyl groups. Divalent alkenyl groups, i.e., alkenyl groups with two points of attachment, include, but are not limited to, CH—CH═CH₂, C═CH₂, or C═CHCH₃.

Alkynyl groups are hydrocarbon moieties having 2 to about 20 carbon atoms, and further including at least one triple bond. In some embodiments alkynyl groups have from 1 to 12 carbons, or, typically, from 1 to 8 carbon atoms. Alkynyl groups may be substituted or unsubstituted. Alkynyl groups include, for instance, ethyne, propyne, butyne, pentyne, hexyne, heptyne, octyne, nonyne, among others.

As used herein, “aryl”, or “aromatic,” groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. The phrase “aryl groups” includes groups containing fused rings, such as fused aromatic-aliphatic ring systems (e.g., indanyl, tetrahydronaphthyl, and the like). Aryl groups may be substituted or unsubstituted.

As used herein, “aralkyl” refers to a moiety of the formula —R^{b}R^{c} where R^{b} is an alkylene group and R^{c} is an aryl group as defined herein. Exemplary aralkyl groups include, but are not limited to, benzyl, phenylethyl, 3-(3-chlorophenyl)-2-methylpentyl, and the like. The term “optionally substituted aralkyl” means the aryl group is optionally substituted with one or more, typically, one to three, and often one or two, substituents. Exemplary substituents for the aryl group include, but are not limited to, alkyl, haloalkyl, thioalkyl, heteroalkyl, halo, nitro, cyano, cycloalkyl, aryl, heteroaryl, heterocyclyl, haloalkoxy, aryloxy, heteroaryloxy, etc.

As used herein, “heteroaryl” refers to a cyclic aromatic compound that contains one or more heteroatoms selected from the group consisting of nitrogen, oxygen, and sulfur in the ring. The “heteroaryl” group can be made up of two or more fused rings (rings that share two adjacent atoms). When the heteroaryl is a fused ring system, then the ring that is connected to the rest of the molecule has a fully delocalized pi-electron system. The other ring(s) in the fused ring system may or may not have a fully delocalized pi-electron system. Examples of heteroaryl rings include, without limitation, furan, thiophene, phthalazinone, pyrrole, oxazole, thiazole, imidazole, pyrazole, isoxazole, isothiazole, triazole, thiadiazole, pyridine, pyridazine, pyrimidine, pyrazine and triazine.

Wherever “hetero” is used it is intended to mean a group as specified, such as an alkyl or an aryl group, where at least one carbon atom has been replaced with a heteroatom selected from the group consisting of nitrogen, oxygen, and sulfur.

As used herein, “heterocycloalkyl,” refers to a ring having in the ring system one or more heteroatoms independently selected from nitrogen, oxygen and sulfur. The ring may also contain one or more double bonds provided that they do not form a fully delocalized pi-electron system in the rings. The ring defined herein can be a stable 3- to 18-membered ring that consists of carbon atoms and from one to five heteroatoms selected from the group consisting of nitrogen, oxygen, and sulfur. Heterocycloalkyl groups of the presently disclosed compounds may be unsubstituted or substituted. When substituted, the substituent(s) may be one or more groups independently selected from the group consisting of halogen, hydroxy, protected hydroxy, cyano, nitro, alkyl, alkoxy, acyl, acyloxy, carboxy, protected carboxy, amino, protected amino, carboxamide, protected carboxamide, alkylsulfonamido and trifluoromethane-sulfonamido. The “heterocycloalkyl” group can be made up of two or more fused rings (rings that share two adjacent carbon atoms). When the heterocycloalkyl is a fused ring system, then the ring that is connected to the rest of the molecule is a heterocycloalkyl as defined above. The other ring(s) in the fused ring system may be a cycloalkyl, a cycloalkenyl, an aryl, a heteroaryl, or a heterocycloalkyl.

As used herein, “alkoxy” refers to an alkyl group attached to an oxygen (—O— alkyl-). “Alkoxy” groups also include an alkenyl group attached to an oxygen (“alkenyloxy”) or an alkynyl group attached to an oxygen (“alkynyloxy”) groups. Exemplary alkoxy groups include, but are not limited to, groups with an alkyl, alkenyl or alkynyl group of 1-22, 1-8, or 1-6 carbon atoms, referred to herein as (C₁-C₂₂)alkoxy, (C₁-C₈)alkoxy, and (C₁-C₆)alkoxy, respectively. Exemplary alkoxy groups include, but are not limited to methoxy, ethoxy, etc.

As used herein, “ether” refers to the structure —R₁—O—R₂—, where R₁ and R₂ are independently alkyl, alkenyl, aryl, cycloalkyl, heterocyclyl, heteroaryl, or heterocycloalkyl. Exemplary ethers include, but are not limited to, alkoxyalkyl and alkoxyaryl groups. Ethers also includes polyethers, e.g., where one or both of R₁ and R₂ are ethers.

The term “carboxylate” as used herein refers to the conjugate base of a carboxylic acid with the chemical formula —COO⁻.

The term “ester” as used herein refers to —COOR^{b}— and C(O)O-G groups. R^{b} is a substituted or unsubstituted alkyl, cycloalkyl, alkenyl, alkynyl, aryl, aralkyl, hetetocyclyalkyl or heterocyclyl group as defined herein. G is a carboxylate protecting group. Carboxylated protecting groups are well known to one of ordinary skill in the art. An extensive list of carboxylate protecting groups may be found in Protective Groups in Organic Synthesis, Greene, T. W.; Wuts, P.G.M., John Wiley & Sons, New York, NY (3^{rd} Edition, 1999) which can be added or removed using the procedures set forth therein and which is hereby incorporated by reference in its entirety for any and all purposes as set form herein.

The term “amide” (or “amido”) includes C- and N-amide groups, i.e., C(O)NR²R³, and —NR²C(O)—R³ groups, respectively. R² and R³ are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR²C(O)—(C₁₋₅ alkyl) and the group is termed “carbonylamino,” and in others the amide is —NHC(O)-alkyl and the group is termed “alkanoylamino.”

The term “amine” (or “amino”) as used herein refers to —NR^{e}R^{f} groups, wherein R^{e} and R^{f} are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “halogen” or “halo” as used herein refers to bromine (Br), chlorine (Cl), fluorine (F), or iodine (I).

The term “polypeptide” or “peptide” as used herein refers to two or more amino acids linked by a peptide (i.e., amide) bond between the carboxyl terminus of one amino acid and the amino terminus of another. The term “peptide” may be combined with a prefix indicating the number of amino acids in the peptide, e.g., a “pentapeptide” is a peptide of five amino acids.

The term “amino acid” is recognized in the art and generally refers to a natural or unnatural alpha or beta amino acid. The term “amino acid” includes, but is not limited to, any one of the standard L-amino acids commonly found in naturally occurring peptides or to unnatural amino acids, the D-isomers of amino acids or racemic amino acids.

The term “amino acid residue with hydrophobic side chain” as used herein refers to the following amino acids: alanine (Ala), valine (Val), isoleucine (Ile), leucine (Leu), methionine (Met), phenylalanine (Phe), tyrosine (Tyr), tryptophan (Trp); or to unnatural amino acids including but not limited to, norleucine, norvaline, cyclohexylalanine, cyclohexylglycine, cyclopentylglycine and the like. In some embodiments, the amino acid residue with hydrophobic side chain is valine (Val). In other embodiments, the amino acid residue may be racemic or chiral (an L-amino acid (S-configuration) or a D-amino acid (R-configuration)), such as L-valine ((S)-valine) or D-valine ((R)-valine)).

The term “amino acid residue derivative” as used herein refers to an amino acid residue that is covalently linked to a functional group or a protecting group. In some embodiments, the amino acid residue is covalently attached to a protecting group. In some embodiments, the amino acid residue is covalently attached to a functional group or a protecting group through the amino or carboxyl group attached to its alpha-carbon or through a functional group present on its side chain (such as the side chain carboxyl of aspartic acid or the side chain amino group of lysine).

The term “acetyl” as used herein refers to a methyl group bonded to a carbonyl group (CH₃CO—).

The term “tert-butyloxycarbonyl protecting group” or “tert-butoxycarbonyl protecting group” (Boc group) as used herein refers to a protecting group used in organic synthesis.

The term “piperazine” or “piperazinyl” as used herein refers to an organic isoindole derivative that consists of a six-membered ring containing two nitrogen atoms at opposite positions in the ring.

The term “pyridine” group as used herein refers to a group with the heterocyclic organic moiety with the chemical formula C₅H₅N.

Pharmaceutically acceptable salts of compounds described herein are within the scope of the present technology and include acid or base addition salts which retain the desired pharmacological activity and are not biologically undesirable (e.g., the salt is not unduly toxic, allergenic, or irritating, and is bioavailable). When the compound of the present technology has a basic group, such as for example, an amino group, pharmaceutically acceptable salts can be formed with inorganic acids (such as hydrochloric acid, hydroboric acid, nitric acid, sulfuric acid, and phosphoric acid), organic acids (e.g., alginic acid, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, or Zn²⁺), ammonia or organic amines (e.g., dicyclohexylamine, trimethylamine, triethylamine, pyridine, picoline, ethanolamine, diethanolamine, or triethanolamine) or basic amino acids (e.g. arginine, lysine, or ornithine). Such salts can be prepared in situ during isolation and purification of the isoindole derivatives or by separately reacting the purified isoindole derivative in its free base or free acid form with a suitable acid or base, respectively, and isolating the salt thus formed.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized to be substantially free of their enantiomeric or diastereomeric partners, and these stereoisomers are all within the scope of the present technology.

The term “pharmaceutically acceptable excipient” refers to those substances that are well accepted by the pharmaceutical industry and regulatory agencies. These substances may be listed in monographs published in compendia such as USP—NF, Food Chemicals Codex, Code of Federal Regulations (CFR), FDA Inactive Ingredients Database and in 21 CFR parts 182 and 184 that lists substances that are generally regarded as safe (GRAS) food ingredients.

In various embodiments, the compound represented by Formula I is one or more of the following isoindole derivatives, with the understanding that where chiral centers are present for each representation, these embodiments include any R, S, or racemic structures as well, represented by derivatives I-1 through I-23:

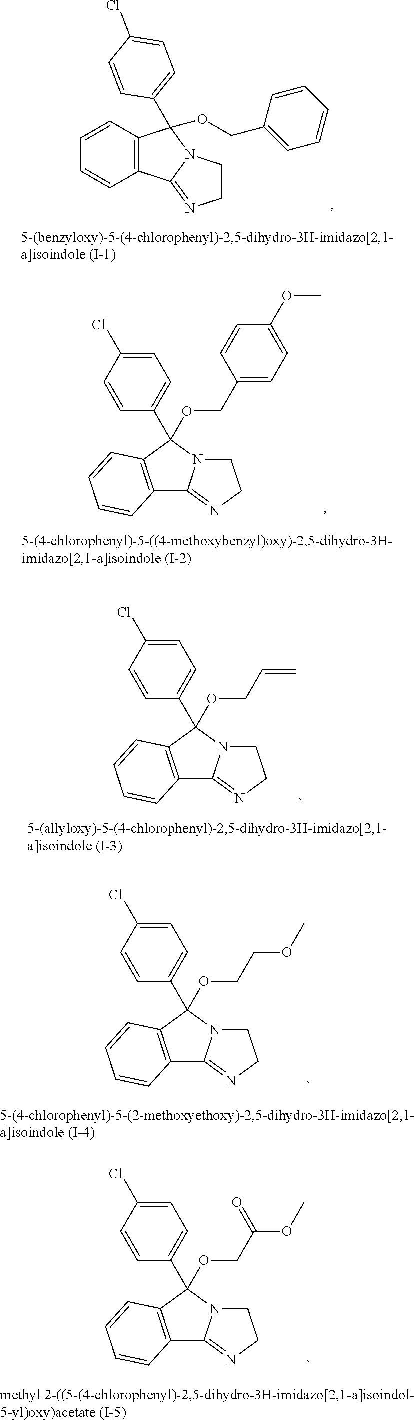 [SMILES: Clc1ccc(C2(OCc3ccccc3)c3ccccc3C3=NCCN32)cc1 ; COc1ccc(COC2(c3ccc(Cl)cc3)c3ccccc3C3=NCCN32)cc1 ; C=CCOC1(c2ccc(Cl)cc2)c2ccccc2C2=NCCN21 ; COCCOC1(c2ccc(Cl)cc2)c2ccccc2C2=NCCN21 ; COC(=O)COC1(c2ccc(Cl)cc2)c2ccccc2C2=NCCN21]

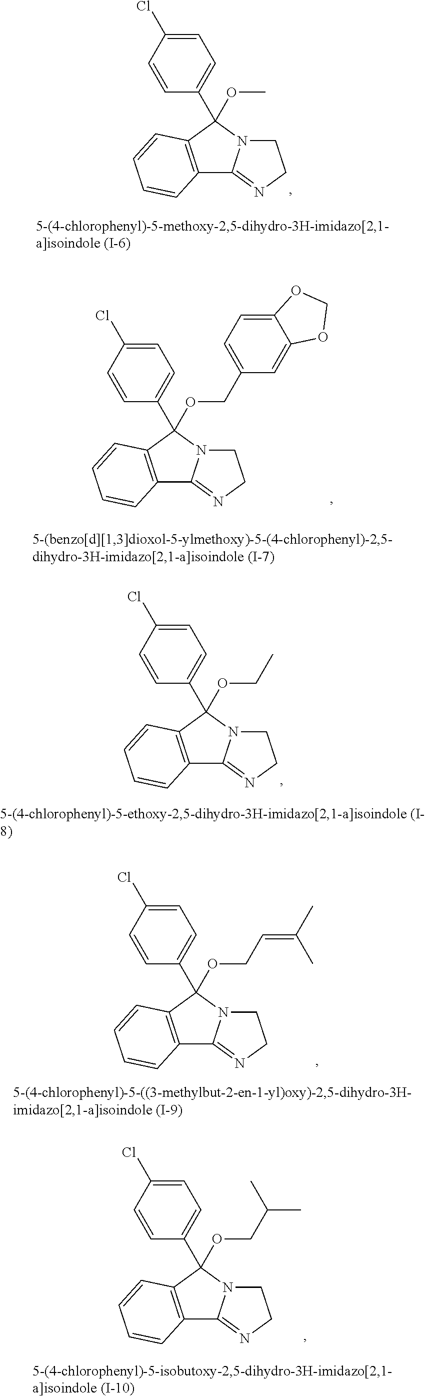 [SMILES: COC1(c2ccc(Cl)cc2)c2ccccc2C2=NCCN21 ; Clc1ccc(C2(OCc3ccc4c(c3)OCO4)c3ccccc3C3=NCCN32)cc1 ; CCOC1(c2ccc(Cl)cc2)c2ccccc2C2=NCCN21 ; CC(C)=CCOC1(c2ccc(Cl)cc2)c2ccccc2C2=NCCN21 ; CC(C)COC1(c2ccc(Cl)cc2)c2ccccc2C2=NCCN21]

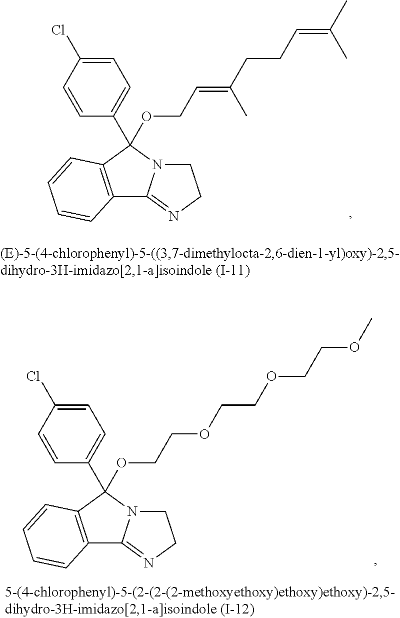 [SMILES: CC(C)=CCC/C(C)=C/COC1(c2ccc(Cl)cc2)c2ccccc2C2=NCCN21 ; COCCOCCOCCOC1(c2ccc(Cl)cc2)c2ccccc2C2=NCCN21]

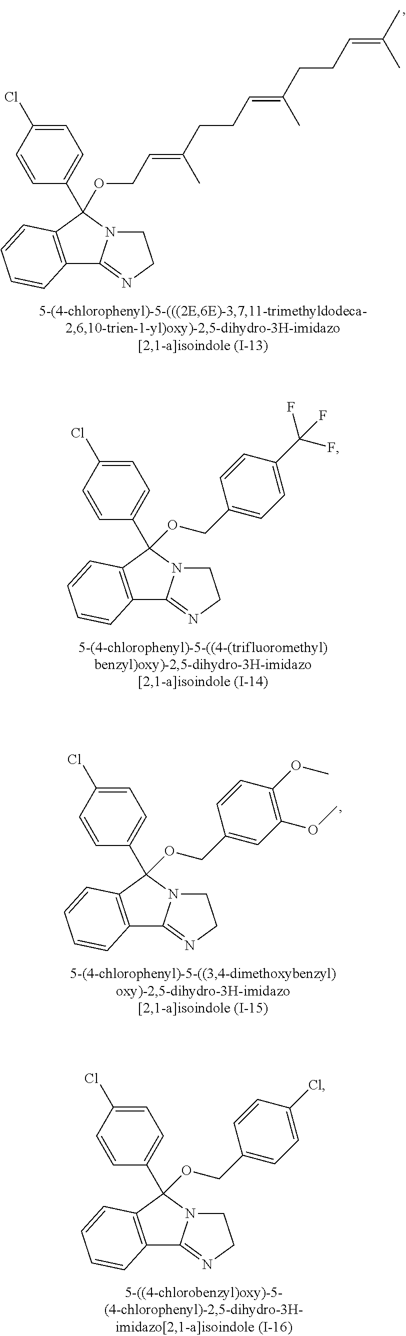 [SMILES: CC(C)=CCC/C(C)=C/CC/C(C)=C/COC1(c2ccc(Cl)cc2)c2ccccc2C2=NCCN21 ; FC(F)(F)c1ccc(COC2(c3ccc(Cl)cc3)c3ccccc3C3=NCCN32)cc1 ; COc1ccc(COC2(c3ccc(Cl)cc3)c3ccccc3C3=NCCN32)cc1OC ; Clc1ccc(COC2(c3ccc(Cl)cc3)c3ccccc3C3=NCCN32)cc1]

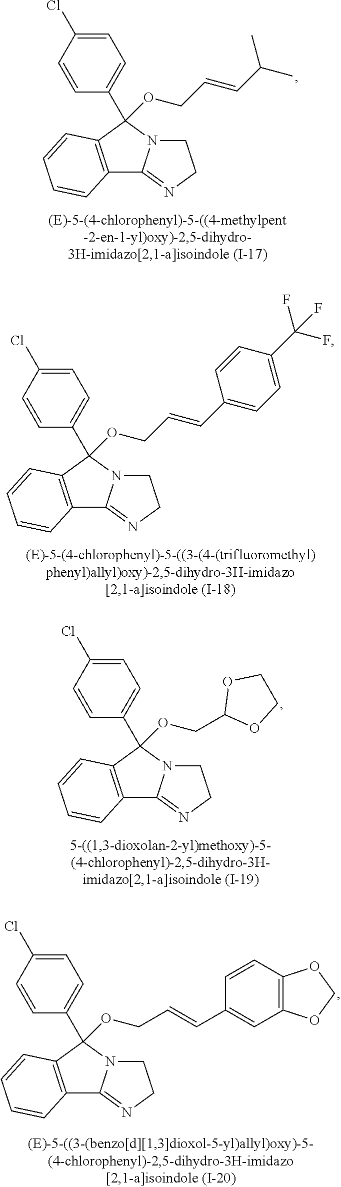 [SMILES: CC(C)/C=C/COC1(c2ccc(Cl)cc2)c2ccccc2C2=NCCN21 ; FC(F)(F)c1ccc(/C=C/COC2(c3ccc(Cl)cc3)c3ccccc3C3=NCCN32)cc1 ; Clc1ccc(C2(OCC3OCCO3)c3ccccc3C3=NCCN32)cc1 ; Clc1ccc(C2(OC/C=C/c3ccc4c(c3)OCO4)c3ccccc3C3=NCCN32)cc1]

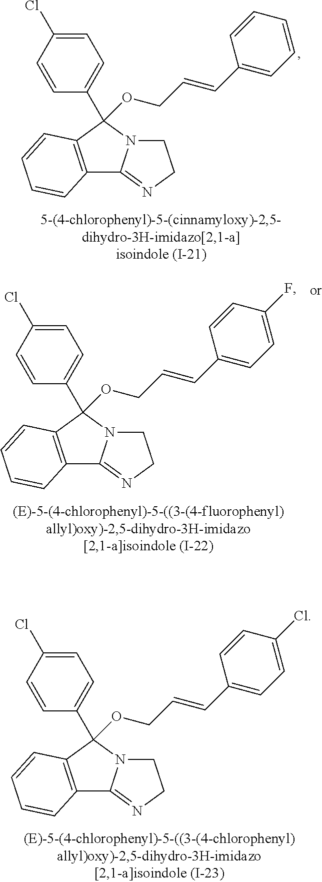 [SMILES: Clc1ccc(C2(OC/C=C/c3ccccc3)c3ccccc3C3=NCCN32)cc1 ; Fc1ccc(/C=C/COC2(c3ccc(Cl)cc3)c3ccccc3C3=NCCN32)cc1 ; Clc1ccc(/C=C/COC2(c3ccc(Cl)cc3)c3ccccc3C3=NCCN32)cc1]

In various embodiments, the compound represented by Formula II is one or more of the following isoindole derivatives, with the understanding that where chiral centers are present for each representation, these embodiments include any R, S, or racemic structures as well, represented by derivatives II-1 through II-9:

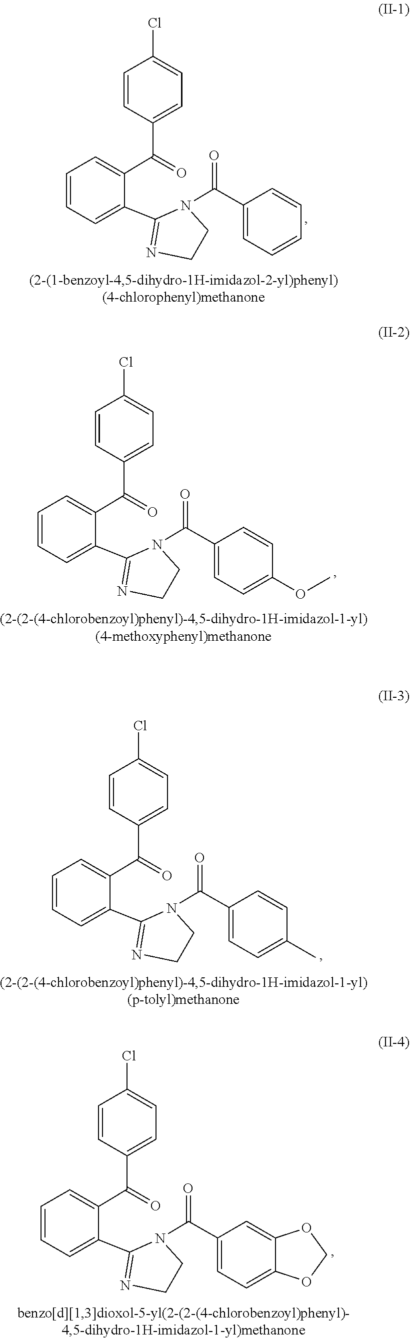 [SMILES: O=C(c1ccc(Cl)cc1)c1ccccc1C1=NCCN1C(=O)c1ccccc1 ; COc1ccc(C(=O)N2CCN=C2c2ccccc2C(=O)c2ccc(Cl)cc2)cc1 ; Cc1ccc(C(=O)N2CCN=C2c2ccccc2C(=O)c2ccc(Cl)cc2)cc1 ; O=C(c1ccc(Cl)cc1)c1ccccc1C1=NCCN1C(=O)c1ccc2c(c1)OCO2]

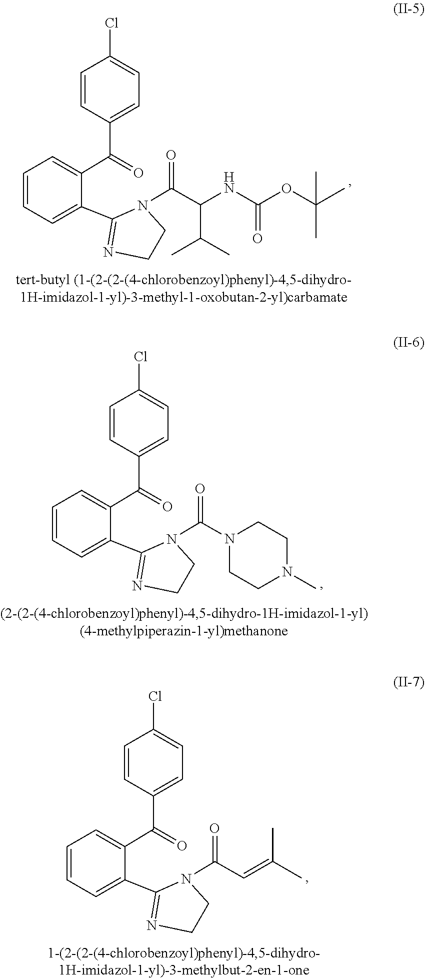 [SMILES: CC(C)C(NC(=O)OC(C)(C)C)C(=O)N1CCN=C1c1ccccc1C(=O)c1ccc(Cl)cc1 ; CN1CCN(C(=O)N2CCN=C2c2ccccc2C(=O)c2ccc(Cl)cc2)CC1 ; CC(C)=CC(=O)N1CCN=C1c1ccccc1C(=O)c1ccc(Cl)cc1]

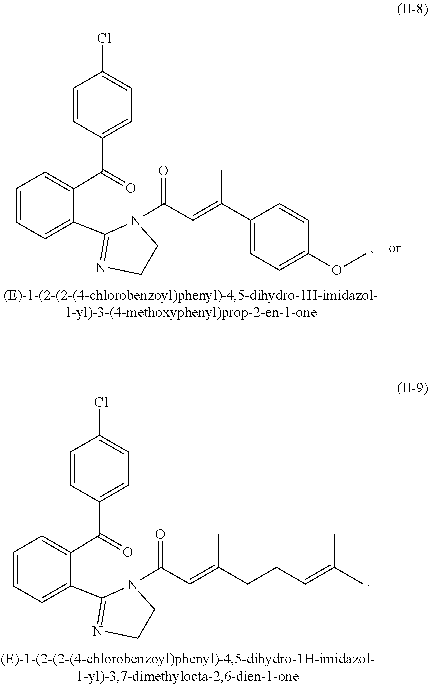 [SMILES: COc1ccc(/C(C)=C/C(=O)N2CCN=C2c2ccccc2C(=O)c2ccc(Cl)cc2)cc1 ; CC(C)=CCC/C(C)=C/C(=O)N1CCN=C1c1ccccc1C(=O)c1ccc(Cl)cc1]

In one embodiment, the compound represented by Formula III is the following isoindoline derivative, with the understanding that where chiral centers are present for each representation, these embodiments include any R, S, or racemic structures as well:

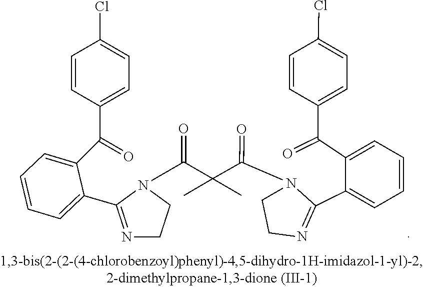 [SMILES: CC(C)(C(=O)N1CCN=C1c1ccccc1C(=O)c1ccc(Cl)cc1)C(=O)N1CCN=C1c1ccccc1C(=O)c1ccc(Cl)cc1]

In various embodiments, a Compound A ring-opened product (Compound B) is provided:

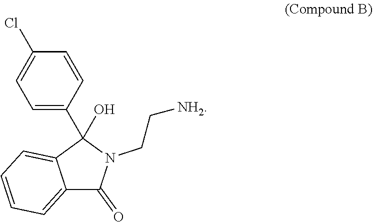 [SMILES: NCCN1C(=O)c2ccccc2C1(O)c1ccc(Cl)cc1]

In some embodiments, a composition includes a compound derivative of Formula I, II, or III, a stereoisomer thereof, or a pharmaceutically acceptable salt thereof, and at least one pharmaceutically acceptable excipient or carrier.

In some embodiments, a pharmaceutical composition comprises a compound of Formula I, II, or III, a stereoisomer thereof, or a pharmaceutically acceptable salt thereof, with a pharmaceutically acceptable carrier or excipient. The pharmaceutical composition may be in an appropriate dosage form. Illustrative dosage forms include, but are not limited to, injections, tablets, capsules, sprinkles, solutions, suspensions, suppositories, caches, pouches, oral, nasal, rectal, transdermal, implants, and the like.

In another aspect, the treatment of diseases or disorders including the treatment of CNS disorders is provided by administering a composition including a compound of Formula I, II, or III, a stereoisomer thereof, or a pharmaceutically acceptable salt thereof, as described herein to a subject in need thereof.

In some embodiments, the CNS disorder may be selected from the group consisting of an autism spectrum disorder, attention deficit hyperactivity disorder (ADHD), an anxiety disorder, a movement disorder, an impulse control disorder, a personality disorder, a reward deficiency disorder, a somatoform disorder, a dementia disorder and an obsessive compulsive spectrum disorder, or a symptom of such a disorder. In one embodiment, the CNS disorder may be ADHD.

In some embodiments, the CNS disorder may be or include one of the following: Attention-Deficit/Hyperactivity Disorder (ADHD), Binge Eating Disorder, Chemotherapy-induced Pain, Chronic Fatigue Syndrome, Dopamine Beta-Hydrolase Deficiency, Fibromyalgia, Idiopathic Hypersomnia, Kleine-Levin Syndrome, Major Depressive Disorder, Narcolepsy Type I, Neuroendocrine Carcinoma, Neuropathic Pain, Obesity, Obsessive Compulsive Disorder, Orthostatic Hypotension and/or Orthostatic Intolerance, non-motor symptoms of Parkinson's Disease, Prader Willi Syndrome, Progressive Supranuclear Palsy, Smith Magenis Syndrome, and Spinocerebellar Ataxia.

In some embodiments, the CNS disorder may be a CNS disorder for which Compound A and/or a prodrug of Compound A is known to be effective.

In some embodiments, the disclosed above compounds alone or with Compound A may be used for reducing an impulsivity-like and/or compulsivity-like behavior. For example, in some embodiments, the disclosed above compounds alone or with Compound A may be used for reducing premature response(s), perseverative response(s), and food magazine entrie(s). In some embodiments, the disclosed above compounds may be used for reducing an impulsivity-like and/or compulsivity-like behavior caused by and/or associated with a disease or disorder, e.g. a CNS disease or disorder, such as Prader Willi Syndrome, Smith Magenis Syndrome, Obsessive Compulsive Disorder, Binge Eating Disorder, Obesity, Attention-Deficit/Hyperactivity Disorder (ADHD), Kleine-Levin Syndrome, Substance Use Disorder, Parkinson's Disease, Spinocerebellar Ataxia, and Progressive Supranuclear Palsy.

In some embodiments, the disclosed above compounds alone or with Compound A may be used for reducing at least one of hyperactivity, inattention, impulsivity, and challenges with concentration. For example, in some embodiments, the disclosed above compounds alone or with Compound A may be used for reducing at least one of hyperactivity, inattention, impulsivity, and challenges with concentration, in a subject, in whom such symptoms are caused by and/or associated with a CNS disease or disorder, such as ADHD, Kleine-Levin Syndrome Smith Magenis Syndrome. Kleine-Levin and Smith Magenis syndromes have some overlapping features with ADHD, such as hyperactivity, inattention, impulsivity and challenges with concentration, see e.g. Korteling et al., 2022; Shah and Gupta, 2023 (for these and other citations in this disclosure, see REFERENCES, infra). See also, Sagvolden et al., 1993; Sagvolden, 2000.

In some embodiments, the disclosed above compounds alone or with Compound A may be used for decreasing an excessive food consumption, altering mitochondria activity in an adipose tissue, such as brown adipose tissue, decreasing white adipose tissue, increasing resting metabolic rate, decreasing blood glucose level and/or decreasing serum insulin levels. For example, in some embodiments, the disclosed above compounds alone or with Compound A may be used for decreasing an excessive food consumption, altering mitochondria activity in an adipose tissue, such as brown adipose tissue, decreasing white adipose tissue, increasing resting metabolic rate, decreasing blood glucose level and/or decreasing serum insulin level caused by and/or associated with a disease or disorder, e.g. a CNS disease or disorder, such as Prader Willi Syndrome, Smith Magenis Syndrome, Kleine-Levin Syndrome, Binge Eating Disorder, and Obesity. a subset of the diseases mentioned above, such as Prader Willi Syndrome, Smith Magenis Syndrome, Kleine-Levin Syndrome, Binge Eating Disorder, and Obesity, is characterized by excessive food consumption. Compound A is an anorectic and has been shown to alter mitochondria activity in brown adipose tissue, decrease white adipose tissue, increase resting metabolic rate, and decrease blood glucose and serum insulin levels in rats (Rothwell et al., 1981; Yoshida et al., 1996).

In some embodiments, the disclosed above compounds alone or with Compound A may be used for improving a food consumption level, weight management, and/or metabolic function. For example, in some embodiments, the disclosed above compounds may be used for improving a food consumption level, weight management, and/or metabolic function caused by and/or associated with a disease or disorder, e.g. a CNS disease or disorder, such as Prader Willi Syndrome, Smith Magenis Syndrome, Kleine-Levin Syndrome, Binge Eating Disorder, and Obesity. See e.g. Rothwell et al., 1981; Yoshida et al., 1996; Suplicy et al., 2013. For beneficial effects of Compound A in individuals with Prader Willi syndrome, see Inoue, 1995; Itoh et al., 1995.

In some embodiments, the disclosed above compounds may be used reducing depression symptoms and/or anxiety symptoms. For example, in some embodiments, the disclosed above compounds may be used reducing depression symptoms and/or anxiety symptoms caused by and/or associated with a disease or disorder, e.g. a CNS disease or disorder, such as Major Depressive Disorder and/or depressive and apathetic symptoms observed in Spinocerebellar Ataxia, Progressive Supranuclear Palsy, and Parkinson's disease. Anorectic effects for Compound A correlated with reduced depression and anxiety symptoms, see e.g. Suplicy et al., 2013.

In some embodiments, the disclosed above compounds may be used for modulating sleeping parameter(s), treating and/or reducing sleep disturbances and/or excessive daytime sleepiness. For example, in some embodiments, the disclosed above compounds may be used for modulating sleeping parameter(s), treating and/or reducing sleep disturbances and/or excessive daytime sleepiness caused by and/or associated with a disease or disorder, e.g. a CNS disease or disorder, such as ADHD, Prader Willi Syndrome, Smith Magenis Syndrome, Parkinson's disease and Kleine-Levin Syndrome.

In some embodiments, the disclosed above compounds alone or with Compound A may be used for treating and/or reducing excessive daytime sleepiness, cataplexy, hypocretin deficiency and/or altered nocturnal sleep polysomnography characterized by altered rapid eye movement sleep phase transitions and onset. For example, in some embodiments, the disclosed above compounds may be used for treating and/or reducing excessive daytime sleepiness, cataplexy, hypocretin deficiency and/or altered nocturnal sleep polysomnography characterized by altered rapid eye movement sleep phase transitions and onset caused by and/or associated with a disease or disorder, e.g. a CNS disease or disorder, such as Narcolepsy, e.g. Narcolepsy Type I.

In some embodiments, the disclosed above compounds alone or with Compound A may be used for treating and/or reducing excessive daytime sleepiness, which in some embodiments, may be caused by and/or associated with a disease or disorder, e.g. a CNS disease or disorder, such as hypersomnia, e.g. idiopathic hypersomnia or symptomatic hypersomnia.

In some embodiments, the disclosed above compounds alone or with Compound A may be used for mitigating cataplexy and/or modifying sleep parameters. For example, in some embodiments, the disclosed above compounds alone or with Compound A may be used for mitigating cataplexy and/or modifying sleep parameters caused by and/or associated with a disease or disorder, e.g. a CNS disease or disorder, such as narcolepsy, e.g. narcolepsy type I.

In some embodiments, the disclosed above compounds alone or with Compound A may be used for improving wakefulness and/or cataplexy measures. For example, in some embodiments, the disclosed above compounds alone or with Compound A may be used for improving wakefulness and/or cataplexy measures caused by and/or associated with a disease or disorder, e.g. a CNS disease or disorder, such as narcolepsy, e.g. refractory narcolepsy, and hypersomnia, e.g. idiopathic hypersomnia or symptomatic hypersomnia. Compound A been shown to improve wakefulness and cataplexy measures in patients with refractory narcolepsy, idiopathic, and symptomatic hypersomnia, Nittur et al., 2013.

In some embodiments, the disclosed above compounds alone or with Compound A may be used for alleviating pain and/or pain symptoms, and/or for mood regulation. For example, in some embodiments, the disclosed above compounds alone or with compound A may be used for alleviating pain and/or pain symptoms, and/or for mood regulation cause by and/or associated with a disease or condition, such as a chronic pain disorder, e.g. fibromyalgia, arthritis, e.g. knee arthritis, and cancer. The norepinephrine transporter (NET) plays a critical role in the regulation of norepinephrine, a neurotransmitter involved in pain perception and mood regulation. In the context of fibromyalgia, a chronic pain disorder often associated with mood disturbances, the role of the NET gene, SLC6A2, may involve in the disease pathology through dysregulation of norepinephrine signaling (Kanodia et al., 2021). Compound A has also been implicated in alleviating pain symptoms in preclinical models of pain (formalin-induced pain and a model of knee arthritis; Bittencourt & Takahashi, 1997; Robledo-Gonzalez et al., 2017) and in a small sample of patients with cancer (Bruera et al., 1986).

In some embodiments, the disclosed above compounds alone or with Compound A may be used to provide anti-nociceptive effect(s).

In some embodiments, the disclosed above compounds alone or with Compound A may inhibit norepinephrine and dopamine transporter. In some embodiments, the disclosed above compounds alone or with Compound A may be used to treat a disease or disorder, for which noradrenergic modulation could be beneficial. In some embodiments, such disease or disorder may be a disease or disorder caused by autonomic failure, where the noradrenergic system plays a central role, such as Dopamine Beta-Hydrolase Deficiency, orthostatic hypotension and/or orthostatic intolerance. The above disclosed compounds and/or Compound A may inhibit the norepinephrine and dopamine transporter. Thus, the above disclosed compounds alone or with Compound A may treat a disease where noradrenergic modulation could be beneficial. Dopamine Beta-Hydrolase Deficiency, orthostatic hypotension and/or orthostatic intolerance are conditions caused by autonomic failure where the noradrenergic system plays a central role. The autonomic nervous system controls involuntary body functions, such as heart rate, blood pressure, sweating, and bowel and bladder control. In the context of primary orthostatic hypotension, a condition characterized by a reduction in blood pressure upon standing, the highest expression of NET might be expected in tissues involved in blood pressure regulation. This could potentially include the adrenal gland, which is involved in the production of hormones that regulate blood pressure, and the cerebral cortex and cerebellum, which are part of the central nervous system that controls cardiovascular function. Noradrenergic drugs have been evaluated in these conditions, see e.g. Kanodia et al., 2021).

In some embodiments, the disclosed above compounds alone or in combination with Compound A may be used to alleviate fatigue symptoms. In some embodiments, the disclosed above compounds alone or in combination with Compound A may be used to treat a chronic fatigue syndrome. In chronic fatigue syndrome, gene expression data from the software-omics database Pandaomics (from Insilico Medicine) revealed a significant downregulation of SLC6A2, which results in imbalanced NE homeostasis in patients with this disorder. The use of NET inhibitors could alleviate the fatigue symptoms.

In some embodiments, the disclosed above compounds alone or in combination with Compound A may be used for treating and/or diagnosing neuroendocrine carcinoma or related disease. Neuroendocrine cells express NET and compounds that target NET can be used as therapeutics or in diagnosing neuroendocrine carcinoma or related disease (Lopez Quinones et al 2022).

In some embodiments, administration is oral, anal, nasal, ocular, parenteral, intraperitoneal, intramuscular, or intravenous.

In one aspect, the subject is a mammal. In further embodiments, the mammalian subject is a human. In particular embodiments, the human subject is an adult or a child or an adolescent or an infant.

In some embodiments, the methods described herein include administering the compound of Formula I, II, or III, a stereoisomer thereof, or a pharmaceutically acceptable salt thereof, along with at least one additional active pharmaceutical agent. In some embodiments, the additional active pharmaceutical agent is another agent for the treatment CNS disorders. In further embodiments, the additional pharmaceutical agent is an isoindoline derivative.

### EXAMPLES

Provided herein are novel compositions and methods of manufacture of isoindole derivatives, analogs, prodrugs, and pharmaceutically acceptable salts thereof suitable for pharmaceutical applications. The isoindole derivatives described herein may themselves be pharmacologically active for the treatment of CNS diseases and may also act as prodrugs producing active metabolites in the body when administered to a human child, adolescent, or adult.

Shown below are examples of the transformation of the prodrugs described herein into the parent drug:

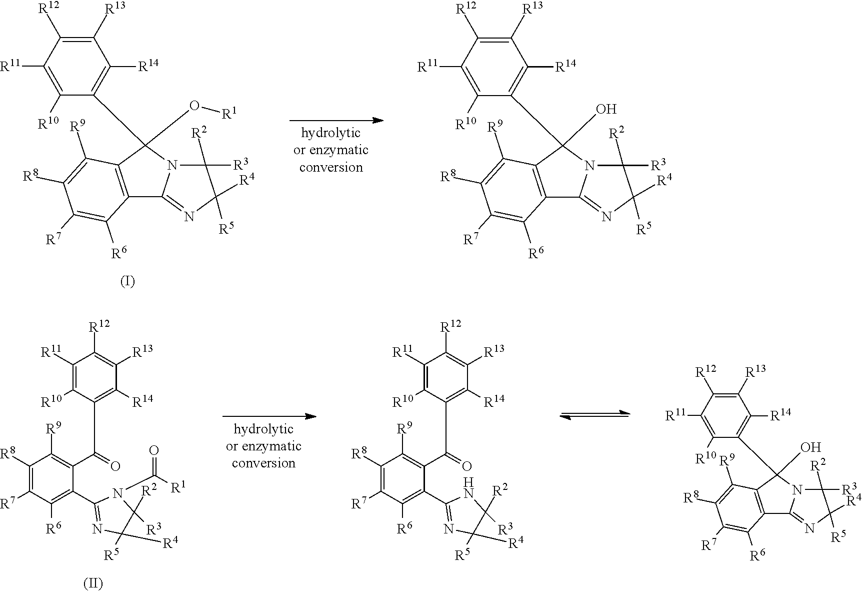

### Example 1: In Vitro Stability Studies in Intestinal, Whole Blood, and Blood Components Using Compound I-19 and Compound I-4

Many drugs are unstable in gut milieu, bacterial enzymes, enterocyte enzymes, circulating enzymes, and erythrocyte enzymes. This is because drugs that are not absorbed entirely by the small intestine will be exposed to the microbiota which are responsible for some metabolism. Metabolism of drugs by the microbiota will in turn affect drug absorption and changes in pharmacological effects. Therefore, it is imperative to make sure drugs are stable in blood, gut bacteria, enterocytes, and at gut pH and gastric pH. In vitro methods offer an easy way to test drug stability because they are less complicated than in vivo systems.

Human 3D EpiIntestinal tissue model (MatTek, SMI-100, Lot #33921 Kit B) was used to model the intestine and was used for permeability studies. This model offers man advantages over other permeability testing models such as Caco-2 cells. This model is able to mimic many aspects of normal intestinal function including barrier, metabolism, inflammatory, and toxicity responses.

### Materials and Methods

Phosphate buffered saline (PBS) at 7.4 was purchased from Corning, Cat #21-040-CV, Lot #28920001. PBS at pH 6.0 was made by titrating PBS at pH 7.4 with 1 M HCl (Sigma-Aldrich, 225184-100ML, Lot SHB64097V).

Simulated Gastric Fluid (SGF) was prepared with 34.2 mM NaCl (Acros, Cat #327300025, Lot #A0412465), 80 μM sodium taurocholate (Sigma-Aldrich, Cat #86339-5G, Lot #BCCC5327), 20 μM of L-alpha Phosphatidylcholine/lecithin (Sigma-Aldrich, Cat #P3556-1G, Lot #SLCDS5861), and 0.1 mg/mL of Pepsin (Sigma-Aldrich, Cat #P6887-250MG, Lot #SLCG8343). 1 M HCl (Sigma-Aldrich, Cat #225184-100ML, Lot #SHBG4097V) was added dropwise to adjust the pH to 1.96.

Simulated Intestinal Fluid (SIF) was prepared with sodium phosphate monobasic (JT Baker, Cat #3818-01, Lot #245188), 105.9 mM NaCl (Acros, Cat #327300025, Lot #A0412465), 3 μM sodium taurocholate (Sigma-Aldrich, Cat #86339-5G, Lot #BCCC5327), and 750 μM of L-alpha Phosphatidylcholine/lecithin (Sigma-Aldrich, Cat #P3556-1G, Lot #SLCDS 861). 1 N NaOH (BDH, Cat #BDH7222-1, Lot #174045003) was added dropwise to adjust the pH to 5.98.

Isotonic saline was made by dissolving 0.8766 g NaCl (Acros, Cat #327300025, Lot #A0412465) in dH₂O to make a 0.15 M solution.

3D human EpiIntestinal tissue model was purchased from MatTek Corp (Cat #SMI-100, Lot #33921 Kit B). Media used was also from MatTek (Cat #SMI-100 M/M, Lot #021521HSA).

Fresh human (Cat #HUMANWBK2UZN, Lot #HMN568778) and rat blood (Cat #RATOOPITGLFZN, Lot #RAT453696-RAT453700) collected into K2EDTA tubes were purchased from Biol VT. Once blood arrived at IONTOX, it was centrifuged to prepare plasma.

Rat feces purchased from Biol VT (Cat #RATOOFECESUNN, Lot #RAT459804) and were used for microbe stability experiments.

Raw Probiotics capsules purchased from Garden of Life (Cat #658010123327, Lot #50220853) and were used for microbe stability experiments.

Dimethyl sulfoxide (DMSO) was purchased from Sigma-Aldrich (Cat #D6550-100ML, Lot #RNBF5782). Amphotericin B was purchased from Sigma-Aldrich (Cat #A2411-250MG, Lot #108M4042V) and was used as the positive control for hemolysis studies. Triton X-100 was purchased from Sigma-Aldrich (Cat #T8787, Lot #SLCD323 7). Acetonitrile was purchased from Sigma-Aldrich (Cat #271004-1L, Lot #SHBLO479). Formic acid was purchased from Fisher Chemical (Cat #A117-50, Lot #195725).

### Stability of Test Articles at Different pH Values

10 mM stock solutions of test articles were prepared in DMSO. From this stock solution, a single concentration in a 10 μL aliquot was added to three fluid matrices: SGF at pH 2.0 (gastric), SIF at pH 6.0 (upper intestine), and PBS at pH 7.4 (systemic fluid). The final volume was 1 mL (0.1 mM). The preparations were then incubated at 37° C. and samples were collected at 0, 15, 30, 60, and 120 min. for analysis of parent material by LCMSMS. The collected sample was quenched in acetonitrile/0.1% formic acid mixture.

### Intestinal Permeability and Metabolic Study

The test articles (100 wL of 100 μM solution, or 4.25 ug) was applied to the apical surface of the EpiIntestinal model. Samples were collected from the basolateral compartment of the EpiIntestinal model at 0, 15, 30, 60, and 120 min. Metabolic stability was determined by loss of parent compound using LCMSMS. The collected sample was quenched in acetonitrile/0.1% formic acid mixture.

### Stability in Human and Rat Red Blood

### Blood Matrix Separation

Human and rat blood were centrifuged to separate out red blood cells at 1174×g for 15 min. The upper plasma level was aspirated with a micropipette and placed in a bleach solution for disposal. The RBC pellet was resuspended in sterile isotonic saline. The pellet was gently resuspended by rocking the tube. The sample was then centrifuged again to collect the RBC pellet and then resuspended and washed one more time following the same procedure. A total of three was done. Following the last wash and centrifugation, the RBCs were resuspended with PBS; and mixed by inversion. Aliquots of the resuspended and washed RBCs were then placed into 4 reaction tubes. These tubes were centrifuged to collect the RBCs. The fluid level was marked on each tube, then the supernatant was aspirated off and the cells resuspended with room temperature PBS pH 7.4 to the original mark. An aliquot of the RBC suspension was added to four sterile tubes containing PBS pH 7.4 at room temperature.

### Stability Experiments

The stability of test articles in whole blood, red blood cells only, and plasma was done to determine stability in each blood compartment. 50 μL of test article was placed in each blood matrix. The final volume was 1 mL (0.5 mM). Samples were taken at 0, 15, 30, 60, and 120 min. LCMSMS of parent compound was used to determine stability. The collected sample was quenched in acetonitrile/0.1% formic acid mixture.

### Hemolysis Studies

To a clear 96-well V-bottom polystyrene plate, the test articles (10 μL) and the diluted RBCs (190 μL) from human or rat blood were mixed for final concentrations of 25, 50, and 100 μM. The reaction mixture was incubated at 37° C. for 60 min. Following the exposure period, the plates were centrifuged. After centrifugation, 70 μL of supernatant (without disturbing the pellet) was transferred to a clean and clear polystyrene flat bottomed 96-well plate and the absorbance of the sample at a single wavelength was measured using a BioTech Synergy H1 Plate Reader at a wavelength of 410 nm. The positive control for this assay was amphotericin B test at concentrations of 1, 10, 30, and 100 μM.

### Stability in Rat and Human Gut Microbiota

Rat fecal material was obtained and weighed. One capsule of the raw probiotics was crushed and weighed prior to use. The weighed samples were then solubilized in PBS at pH 6.0 and 7.4 to extract microorganisms. The samples were centrifuged at 18,000×g for 10 min. to remove particulates and the supernatant was used to evaluate test article stability. An aliquot (50 μL) of the test article stock solutions were added to the fecal extract (final concentration 0.5 mM) and allowed to incubate at 37° C. Aliquots were collected at 0, 15, 30, 60, and 120 min. and analyzed by LCMSMS to determine stability of the parent molecule. The collected sample was quenched in acetonitrile/0.1% formic acid mixture.

### LCMSMS Methods

LC-MS/MS analysis of test articles was performed on a Waters Acquity UPLC system in-line with a Waters TQ-S triple quadrupole mass spectrometer via an electrospray interface. Test articles were optimized for SRM (Single Reaction Monitoring) detection by infusing 200 ng/mL Compound I-19 and 200 ng/mL Compound I-4 compound in 75% methanol/0.15% formic acid into the mass spectrometer and optimizing for sensitivity using positive or negative ion detection, cone voltage, and collision energy.

Analysis was also performed on a Waters/Micromass Quattro Micro mass spectrometer via an electrospray interface. Test articles were optimized for SRM (Single Reaction Monitoring) detection by infusing 1 μg/mL Compound I-19 and 1 μg/ml Compound I-4 compound in 75% methanol/0.15% formic acid into the Quattro Micro mass spectrometer and optimizing for sensitivity using positive or negative ion detection, cone voltage, and collision energy.

UPLC separation of samples was performed on a Waters BEH Phenyl 2.1×50 column at 40 C. Mobile phase was 0.1% formic acid in water for mobile phase A and 0.1% formic acid in acetonitrile as mobile phase B. Gradient elution was 1% B initial, 65% B to 95% B at 2 minutes, 95% B at 4 minutes, and 1% B at 4.1 minutes. Flow rate was 0.4 mL/min and 5 μL of samples was injected.

### Data Analysis

All data was compiled and organized in Excel. Mean, standard deviation, and standard deviation of the mean were calculated using Excel. All data was organized into tables and graphed using GraphPad Prism 9. The permeability coefficient (Papp) was calculated using the following equation:

$${P}_{app} = \frac{dQ}{dt} \times \frac{1}{{m}_{0}} \times \frac{1}{A} \times {V}_{D}$$

where dQ/dt is the steady-state transport rate (μg/s) determined by linear regression of amount of drug in basolateral fluid (11 g, will be taken from LC/MS/MS data) vs. exposure time (s) graphs in GraphPad Prism 9; m₀ is the mass of drug added in donor compartment (μg); A is surface area (cm²) of intestinal tissue; and Vₚ is donor volume (cm²).

### Results and Discussion

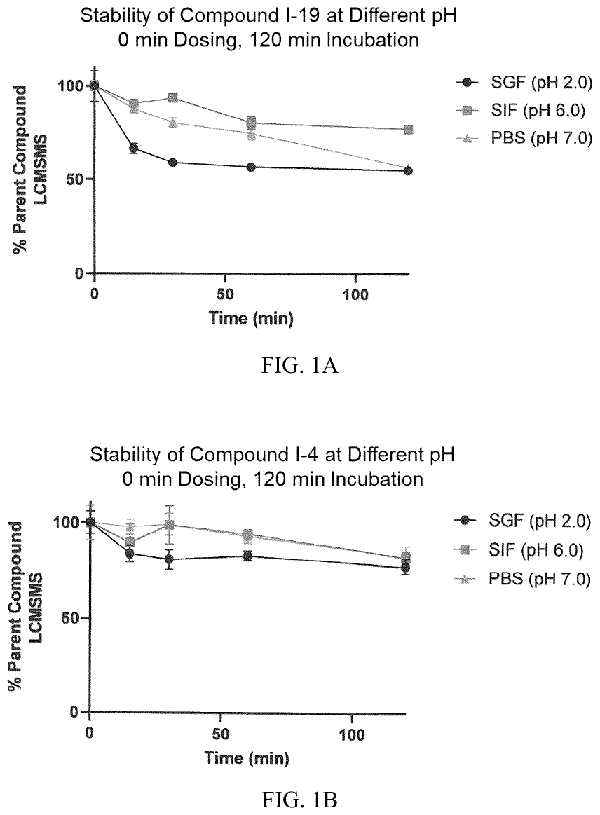

FIGS. 1A-1B show the stability of Compound I-19 (FIG. 1A) and Compound I-4 (FIG. 1B) in different matrices representing different body fluids. After Compound I-19 was incubated in SGF (pH 2.0), there was a time-dependent decrease in the LCMSMS peak area until 20 min. After 20 min, the amount of Compound I-19 reached a steady-state in SGF. After Compound I-19 was incubated in SIF (pH 6.0) and PBS (pH 7.4), there was a time-dependent decrease in the amount. The apparent loss of material in PBS may be due to non-specific binding to plastic. These data suggest that there is no difference in stability between PBS and SIF. Compound I-19 was less stable in SGF. After Compound I-4 was incubated in all three matrices, there was no significant change in the amount of parent compound.

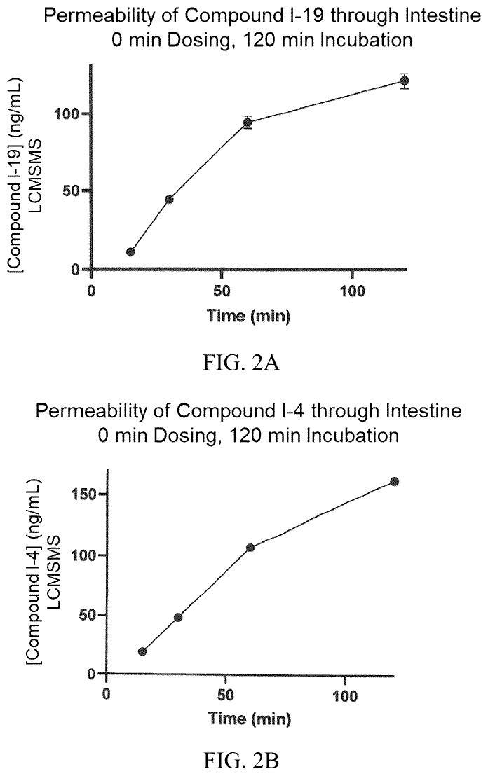

FIGS. 2A-2B show the intestinal permeability Compound I-19 and Compound I-4. After Compound I-19 was dosed apically to the intestinal tissue, there was a time-dependent increase in the amount of Compound I-19 present (FIG. 2A). After Compound I-4 was dosed apically to the intestinal tissue, there was a time-dependent increase in the amount of Compound I-4 present (FIG. 2B). The apparent permeability coefficient (Papp) for Compound I-19 and Compound I-4 is shown in Table 1.

TABLE 1
Relative Permeability of Test Articles

| | Stock Solution | Dose Volume | Papp |
|---|---|---|---|
| Test Article | (mM) | (μL) | (cm/s) |
| Compound I-19 | 10 | 100 | 1.06 × 10⁻⁷ |
| Compound I-4 | 10 | 100 | 1.55 × 10⁻⁷ |

Papp was calculated according to the equation shown above and using FIGS. 2A-2B.

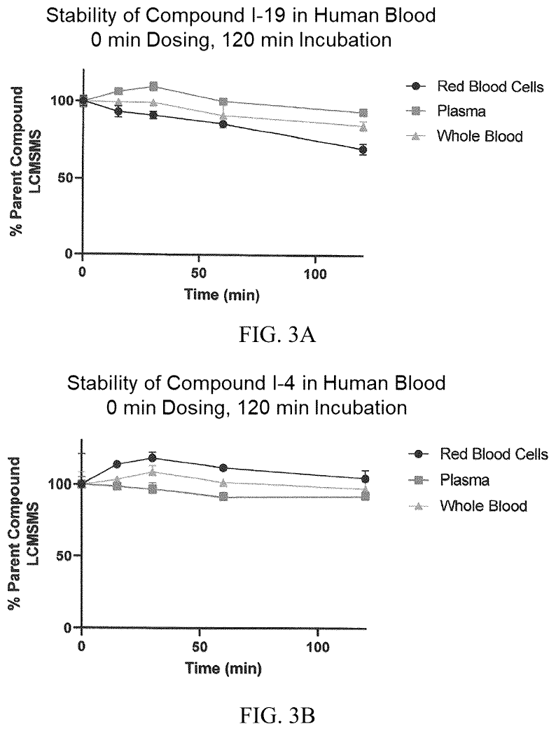

FIGS. 3A-3B show the stability of Compound I-19 and Compound I-4 in human blood. After Compound I-19 was incubated in red blood cells and whole blood, there was a time-dependent decrease in the amount of parent compound present (FIG. 3A). After incubation in plasma, the amount of parent compound remained at or above 100% for all time points. After Compound I-4 was incubated in all three matrices, the amount of parent compound remained at or above 100% for all time points (FIG. 3B).

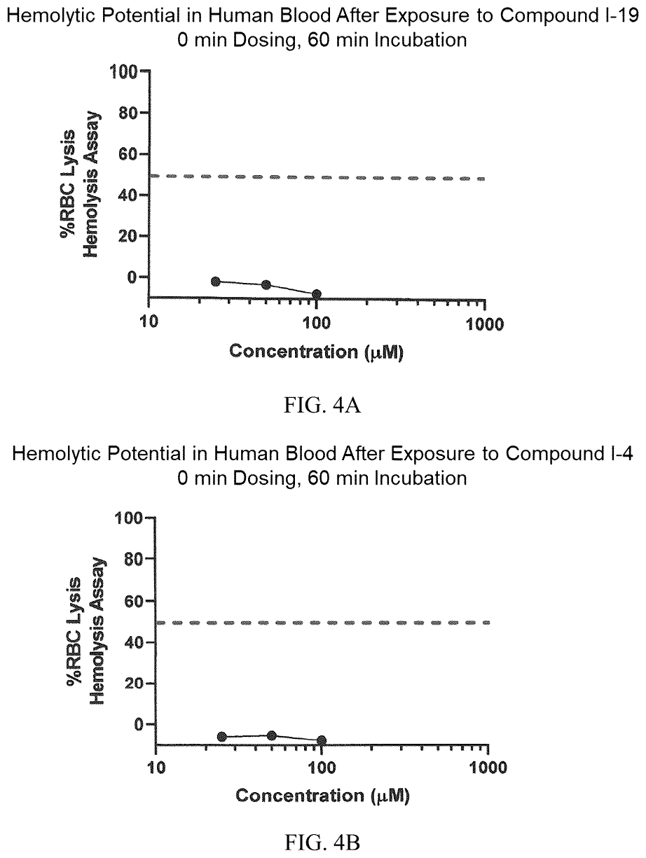

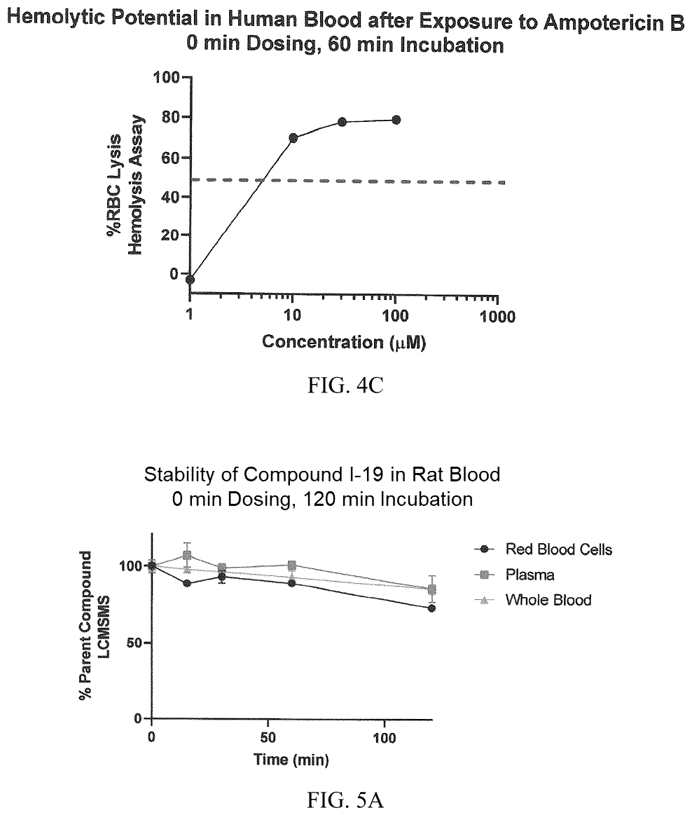

FIGS. 4A-4C show the hemolytic potential of Compound I-19 and Compound I-4 in human blood. After exposure to Compound I-19, for 1 hr, there was no dose-dependent response in % RBC lysis in human blood (FIG. 4A). There was no detectable hemolysis under the conditions used in this test. The EC50 for this sample is outside the concentration range tested. After exposure to Compound I-4, for 1 hr, there was no dose-dependent response in % RBC lysis in human blood (FIG. 4B). There was no detectable hemolysis under the conditions used in this test. The EC50 for this sample is outside the concentration range tested. After exposure to amphotericin B for 60 min, there was a dose-dependent increase in % RBC lysis (FIG. 4C). After 10 μg/mL of amphotericin B was dosed, there was a response above 50% and EC50 was calculated to be 7.06 μM.

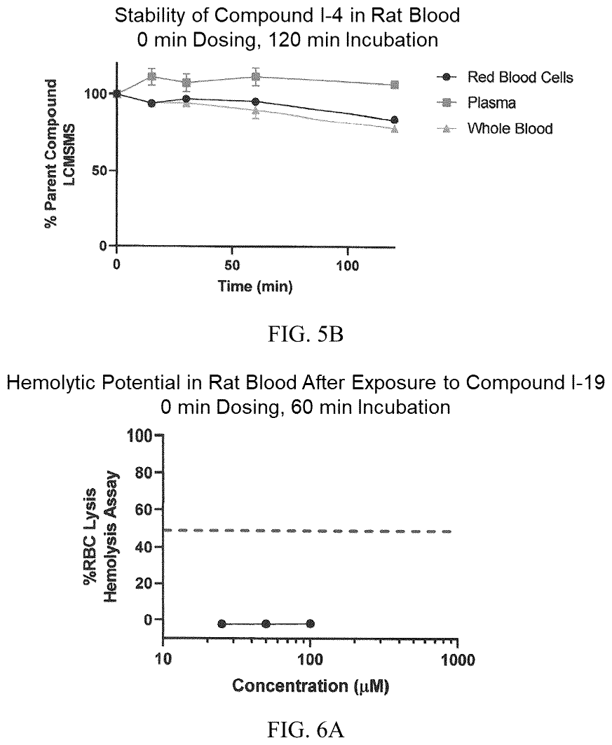

FIGS. 5A-5B show the stability of Compound I-19 and Compound I-4 in rat blood. After Compound I-19 was incubated in all three matrices, there was a small time-dependent decrease in the amount of parent compound present (FIG. 5A). After Compound I-4 was incubated in rat red blood cells and whole blood, there was a small time-dependent decrease in the amount of parent compound remaining (FIG. 5B). After Compound I-4 was incubated in rat plasma, there was no significant change in the amount of parent compound.

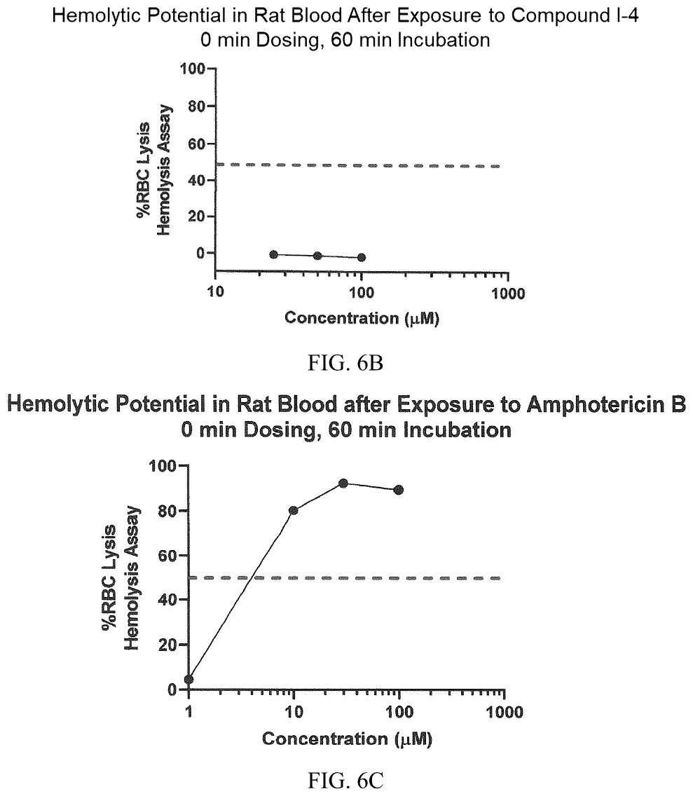

FIGS. 6A-6C show the hemolytic potential of Compound I-19 and Compound I-4 in rat blood. After exposure to Compound I-19 for 1 hr. in rat blood, there was no dose-dependent response in % RBC lysis (FIG. 6A). There was no detectable hemolysis under the conditions used in this test. The EC50 for this sample is outside the concentration range tested. After exposure to Compound I-4 for 1 hr. in rat blood, there was no dose-dependent response in % RBC lysis (FIG. 6B). There was no detectable hemolysis under the conditions used in this test. The EC50 for this sample is outside the concentration range tested. After exposure to amphotericin B for 60 min, there was a dose-dependent increase in % RBC lysis (FIG. 6C). After 10 pg/mL of amphotericin B was dosed, there was a response above 50% and EC50 was calculated to be 4.73 μM.

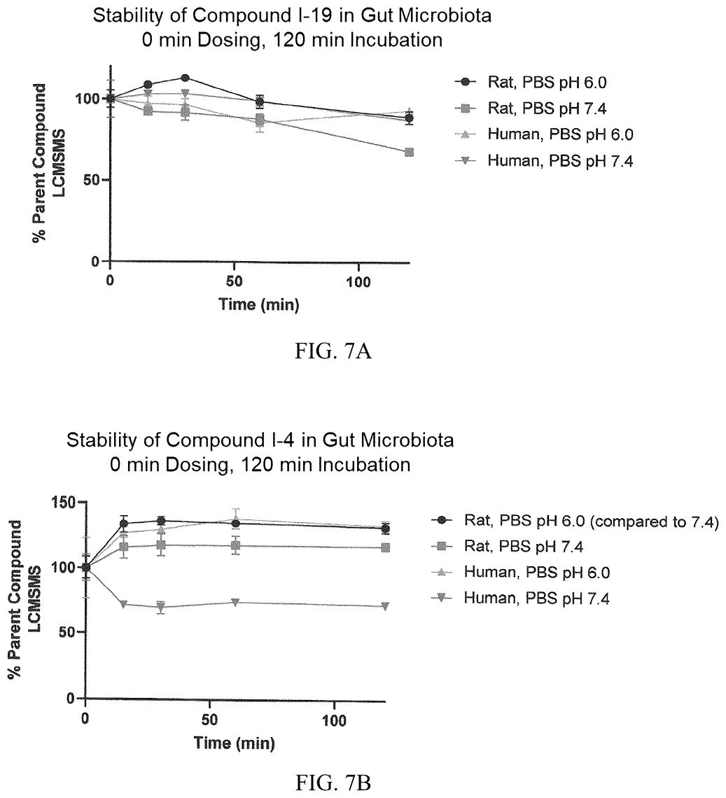

FIGS. 7A-7B show the stability of Compound I-19 and Compound I-4 in human and rat gut microbiota. After Compound I-19 was incubated with rat fecal matter at pH 6 and human fecal matter at pH 7.4 (FIG. 7A), the amount of parent compound remained at or above 100% for all time points except 120 min. After Compound I-19 was incubated with rat fecal matter at pH 7.4, there was a time-dependent decrease in the amount of parent compound present. After Compound I-19 was incubated with human fecal matter at pH 6, there was no significant change in the amount of parent compound. Differences in degradation rates between species is due to different composition of their microbiome and to pH in the intestinal tract. Protease activity can vary based on pH and pH changes throughout the intestine. The data would suggest that in human microbiome degradation increases with higher pH. After Compound I-4 was incubated with rat fecal matter at pH 6 and pH 7.4 and human fecal matter at pH 6 (FIG. 7B), the amount of parent compound remained at or above 100% for all time points. This change however could be due to experimental error with the zero-time point. After Compound I-4 was incubated with human fecal matter at pH 7.4, the amount of parent compound decreases after 10 min. and reached a steady-state until 120 min.

### Conclusion

Compound A derivatives Compound I-19 and Compound I-4 were tested for stability in a variety of matrices. Compound I-19 showed an indication of some lack of stability over 15 min. in gastric fluid and over 2 hr. in PBS at pH 7.4 but was stable in upper intestinal fluid. Compound I-4 on the other hand was stable in all three fluid matrices. Both compounds permeated the intestine with Compound I-4 showing slightly more permeability. Both compounds were also stable in both human and rat blood matrices. In addition, there was no hemolytic activity detected in human or rat blood under the conditions tested. In comparison, the positive control (amphotericin B) produced a clear dose related increase in hemolysis in human and rat blood. Compound I-19 was unstable in rat microbiota at pH 7.4 but was stable in the other microbiota matrices. And finally, Compound I-4 may be slightly unstable in human microbiota at pH 7.4, but stable in the microbiota matrices.

### Example 2: In Vitro Metabolic Stability of Compound I-4 and Compound I-19 in Human Intestinal Homogenate

The metabolic stability of Compound I-4 and Compound I-19 was assessed in human intestinal homogenate by monitoring both the disappearance of prodrug and formation of metabolite.

### Materials and Methods

TABLE 2
Test Articles and Metabolites

| | | | | Storage |
|---|---|---|---|---|
| Compound ID | Batch No. | MW/FW | Purity % | Condition |
| Compound I-4 | 113-069 | 342.82 | 96.7 | Room |
| Compound I-19 | 113-158 | 370.83 | 98 | temperature, |
| Compound A | 10C0021 | 284.74 | 100 | protect from |
| (metabolite) | | | | light |

TABLE 3
Positive Controls

| Compound ID | Application | MW | Catalog No. | Vendor |
|---|---|---|---|---|
| Testosterone | Positive control | 288.42 | T1500 | Sigma- |
| | | | | Aldrich |
| 7-hydroxycoumarin | Positive control | 162.14 | CAS93-35-6 | Sigma- |
| | | | | Aldrich |

Water was purified in-house through a Barnstead GenPure water purification system (Thermo Scientific, Dubuque, IA) with 18.2 MΩ-cm resistance was used. Solvents were obtained from Fisher Scientific or Sigma-Aldrich.

Pooled human intestinal homogenate (pooled male) was purchased from BioIVT, NY, USA (Cat. No. S00721, Lot No. FQW) or other validated vendors. Upon receipt, all samples are stored at ca −70° C.

### Preparation of Solutions

### Incubation Buffer (PBK)

50 mM potassium phosphate buffer was prepared from 1 M potassium phosphate buffer, pH 7.2.

### Preparation of Test Article Solutions

Compound I-4 and Compound I-19 stock solutions at 10 mM was prepared in DMSO. The dilution of 1 mM intermediate was prepared by 70% methanol/water, then 10 M working solutions were prepared by diluting the 1 mM intermediates with 50 mM PBK. All working solutions were freshly prepared on the day of experiment and disposed after use.

### Preparation of Positive Control Stock and Working Solutions

The positive control stock solutions, testosterone and 7-hydroxycoumarin were prepared at 10 mM in DMSO and stored at ca. −20° C. Prior to use, the stock solutions were brought to room temperature and thoroughly mixed. The working solutions were freshly prepared similarly as described for the test article working solution.

### Preparation of Internal Standard Solutions and Stop Solutions

The stock solution of internal standards, tolbutamide and labetalol, were prepared in DMSO and stored at ca. −20° C. The stop solution was prepared by spiking the stock solution (1 mg/mL) into acetonitrile to achieve a 200 ng/mL final concentration.

### Preparation of Human Intestinal Homogenate Solution

The human intestinal homogenate was diluted with 50 mM PBK buffer to make mixture solution of 0.625 mg/mL protein.

### Preparation of Cofactors Solutions

The cofactor solution was made at 10 mM of NADPH in 50 mM PBK buffer.

### Assay Procedures

The intestinal homogenate mixture solution was added to 96-well plates (80 L/well) in duplicate. The plates were pre-incubated for 10-min. at 37° C. in a water bath and then spiked 10 μL each of 10 μM Compound I-4 and Compound I-19 or positive control working solution into corresponding wells, separately. Reaction was initiated by adding cofactor solution at 10 μL/well. The plates were incubated at 37° C. in a water bath with shaking. The final incubation mixture contained Compound I-4 or Compound I-19, or positive control at 1 μM, and 0.5 mg/mL of human intestinal homogenate, and 1 mM NADPH. The final organic solvent content in the incubation was ≤1%.

At specified time points, i.e., 5, 10, 20, 30 and 60 minutes, NCF60 (without adding NADPH replaced with 50 mM PBK buffer) reaction was stopped by adding three volumes of stop solution. The time zero (T₀) samples were prepared by adding three volumes of stop solution to intestinal homogenate samples, followed by addition of test article or control, and cofactor solution 10 μL/well.

All sample plates were mixed well by shaking for approximately 10 minutes and centrifuged at 3220×g for 20 minutes. Subsequently, 100 μL of supernatant was removed from each well, diluted with 100 μL pure water and analyzed by LC/MS/MS.

### Bioanalytical Analysis

Concentrations of test articles and control compounds in the samples were analyzed by LC/MS/MS using semi-quantitation. Plotting of the chromatograms and peak area integrations were carried out by Analyst (AB Sciex, Framingham, Massachusetts, USA). The peak area ratios of analyte/internal standard were used to semi-quantitatively determine the concentrations.

### Data Analysis

The software provided with the LC/MS/MS system (Analyst® software, v.1.6.2, AB Sciex) was used to integrate the area under the mass ion peaks of the analytes and internal standards. The calculated concentrations were then exported to Microsoft Excel (v.2016) and the data were organized and tabulated. Calculated values may be slightly different due to rounding of reported raw data to a designated number of decimals.

The peak area ratios (PAR) of analyte/internal standard were used to semi-quantitatively determine the concentration of test article and control compounds, and calculate the percent remaining with the following equation:

$$\% Remaining = \frac{PAR of analyte to internal standard at each time point}{PAR of analyte to internal standard at {T}_{0}} \times 100 \%$$

The elimination rate (ke) was calculated from a log linear plot of % remaining versus time. The half-life was calculated using the following equation:

$${T}_{1 / 2} ( min ) = 0.693 / ke$$

The intrinsic clearances were calculated using the following equations:

$${CL}_{int ( HIH )} ( μ L / min / mg ) = 0.693 / {T}_{1 / 2} / mg protein per mL$$

### Results and Discussion

### Metabolism Stability of Compound I-4 and Compound I-19 in Human Intestinal Homogenate

The results of metabolic stability of Compound I-4 and Compound I-19 in human intestinal homogenate, including percent remaining, T_{1/2} (min) and intrinsic clearance values can be found in Table 3. Compound A formation is summarized in Table 4. Results of the positive controls, testosterone and 7-hydroxycoumarin, are summarized in Table 5.

TABLE 3
Metabolic Stability of Compound I-4 and Compound I-19
in Human Intestinal Homogenate at 60-Minute Incubation

| | | Time | Aa/Aiª | % | | CL_{int(HIH)} |
|---|---|---|---|---|---|---|
| Compound ID | Species | (min) | Mean (n = 2) | Remaining^{b} | T_{1/2} (min) | (μL/min/mg) |
| Compound | Human | 0 | 1.34 | 100 | 165 | 8.40 |
| I-4 | | 5 | 1.31 | 97.6 | | |
| | | 10 | 1.17 | 87.3 | | |
| | | 20 | 1.18 | 88.0 | | |
| | | 30 | 1.17 | 87.4 | | |
| | | 60 | 1.01 | 75.8 | | |
| | | NCF60 | 1.08 | 80.4 | NA | |
| Compound | | 0 | 0.90 | 100 | 69.3 | 20.0 |
| I-19 | | 5 | 0.77 | 85.6 | | |
| | | 10 | 0.75 | 82.8 | | |
| | | 20 | 0.62 | 68.4 | | |
| | | 30 | 0.59 | 65.2 | | |
| | | 60 | 0.47 | 52.6 | | |
| | | NCF60 | 0.50 | 55.5 | | |

^{a}Peak area of analyte (Aa) and peak area of internal standard (Ai)
^{b}%Remaining is calculated from the Aa/Ai ratio at each time point to the Aa/Ai ratio at time zero
NCF60: No cofactors were added at 60 min. incubation samples.

TABLE 4
Formation of Compound A in Human Intestinal
Homogenate at 60-Minute Incubation

| | Time | Compound A Formed (Aa/Ai^{a}) | |
|---|---|---|---|
| Species | (min) | From Compound I-4 | From Compound I-19 |
| Human | 0 | 0.46 | 0.76 |
| | 5 | 0.54 | 0.67 |
| | 10 | 0.50 | 0.67 |
| | 20 | 0.51 | 0.65 |
| | 30 | 0.58 | 0.61 |
| | 60 | 0.50 | 0.53 |
| | NCF60 | 0.69 | 0.58 |

^{a}Peak area of analyte (Aa) and peak area of internal standard (Ai)
Mean = 2
NCF60: No cofactors were added at 60 min. incubation samples.

TABLE 5
Metabolic Stability of Positive Controls
in Human Intestinal Homogenate

| | | Time | T_{1/2} | CL_{int(HIH)} |
|---|---|---|---|---|
| Compound | Species | (min) | (min) | (μL/min/mg) |
| Testosterone | Human | 28.7 | 33.2 | 41.8 |
| 7-hydroxycoumarin | | 91.1 | >187 | <7.4 |

### Conclusion

Compound I-4 and Compound I-19 were metabolized in human intestinal homogenate with 75.8% and 52.6% remaining at end of the 60-min incubation. The peak area ratio of metabolite Compound A did not change significantly during the incubation, suggesting there may be other metabolites formed.

### Example 3: In Vitro Stability of Compound I-19 and Compound I-4 in Human and Sprague Dawley Rat

The metabolic stability of Compound I-4 and Compound I-19 was assessed in SD rat, beagle dog, and human plasma by monitoring disappearance of prodrug and formation of metabolite.

### Materials and Methods

TABLE 6
Test Articles and Metabolites

| | | | | Storage |
|---|---|---|---|---|
| Compound ID | Batch No. | MW/FW | Purity % | Condition |
| Compound I-4 | 113-069 | 342.82 | 96.7 | Room |
| Compound I-19 | 113-158 | 370.83 | 98 | temperature, |
| Compound A | 10C0021 | 284.74 | 100 | protect from |
| (metabolite) | | | | light |

TABLE 7
Positive Controls

| Compound ID | Application | MW | Catalog No. | Vendor |
|---|---|---|---|---|
| Enapril | Positive control | 492.52 | E6888 | Sigma-Aldrich |
| Bisacodyl | Positive control | 361.39 | B1390 | Sigma-Aldrich |
| Propantheline | Positive control | 448.39 | P8891 | Sigma-Aldrich |

### Test System

Pooled (N≥3) male SD rat, beagle dog and mixed gender human plasma with EDTA-K2 as anticoagulant were purchased from BioIVT (NY, USA) and stored in a −70° C. freezer before use.

### Preparation of Test Article Solutions

Compound I-4 and Compound I-19 stock solutions at 10 mM were prepared in dimethyl sulfoxide (DMSO). Further dilution with DMSO was made to prepare 2.0 mM. The working solutions of 50 μM was prepared with 20% MeOH/water.

### Preparation of Positive Control Stock and Working Solutions

All positive control stock solutions were prepared in DMSO and stored at ca. −20° C. Prior to use, the stock solutions were brought to room temperature and thoroughly mixed and prepared same as described above.

### Preparation of Internal Standard Solutions and Stop Solutions

The stock solutions of internal standards, tolbutamide (1 mg/mL) and labetalol (1 mg/mL), were prepared in DMSO and stored at ca. −20° C. The stop solution was prepared by spiking the stock solution to acetonitrile to achieve a 200 ng/mL final concentration.

### Assay Procedures

### Preparation of Plasma

Frozen plasma of all species were thawed in a water bath at ca. 37° C., and centrifuged at 3220×g for 5 minutes to remove any debris.

### Incubation

Incubations were conducted in 96-well plate format. The time points defined for this study are 0, 5, 15, 30 and 60 minutes.

An appropriate volume of plasma from test species were added to a 96-deep well plate and Compound I-4 and Compound I-19 working solution was spiked into the plasma in duplicate to achieve a final concentration of 2.0 μM. For positive controls, the rat plasma was spiked with enalapril, the dog plasma spiked with bisacodyl and the human plasma was spiked with propantheline. All spiked plasma sample plates were incubated in a water bath at 37° C. with shaking.

At end of each time point, samples were immediately quenched with three volume of cold quenching solution. All sample plates were mixed thoroughly by shaking for approximately 10 minutes and centrifuged at 3220×g for 15 minutes. Subsequently, 100 μL of supernatant was removed from each well, mixed with 100 μL water in a new 96-well plate and subjected to LC-MS/MS analysis.

### Sample Analysis

Concentrations of test article, metabolite, and control compounds in the samples were analyzed by LC-MS/MS semi-quantitatively. Plotting of the chromatograms and peak area integrations were carried out by Analyst (AB Sciex, Framingham, Massachusetts, USA). The peak area ratios of analyte/internal standard were used to semi-quantitatively determine the concentrations.

### Data Analysis

The software provided with the LC-MS/MS system (Analyst® software, v.1.6.3, AB Sciex) was used to integrate the area under the mass ion peaks of the analytes and internal standards. The calculated concentrations were then exported to Microsoft Excel (v.2016) and the data were organized and tabulated.

The peak area ratios (PAR) of analyte/internal standard were used to semi-quantitatively determine the concentration of test article and control compounds, and calculate the percent remaining with the following equation:

$$\% Remaining = \frac{PAR of analyte to internal standard at each time point}{PAR of analyte to internal standard at {T}_{0}} \times 100 \%$$

The elimination rate (ke) was calculated from a log linear plot of % remaining versus time. The half-life was calculated using the following equation:

$${t}_{1 / 2} ( min ) = 0.693 / ke$$

### Results and Discussion

### Stability of Compound I-4 in SD Rat, Dog and Human Plasma and Compound I-19 in SD Rat and Human Plasma

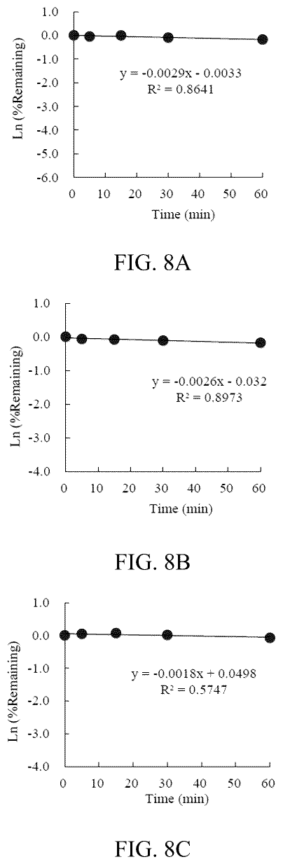

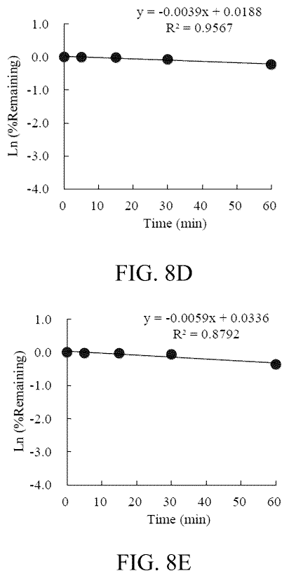

Results of the in vitro stability of Compound I-4 and Compound I-19 and formation of Compound A are summarized in Table 8 and illustrated in FIGS. 8A-8E.

TABLE 8
Stability of Compound I-4 in SD Rat, Beagle Dog and Human Plasma and
Compound I-19 in SD Rat and Human Plasma after 60 Minutes Incubation

| | | | | | | Compound |
|---|---|---|---|---|---|---|
| | | Time | Aa/Aiª | | | A formed |
| Compound ID | Species | (min) | Mean (N = 2) | % Remaining^{c} | T_{1/2}^{b} (min) | (Aa/Aiª) |
| Compound | SD Rat | 0 | 3.25 | 100 | >187 | 0.57 |
| I-4 | | 5 | 3.08 | 95.0 | | 0.66 |
| | | 15 | 3.23 | 99.6 | | 0.58 |
| | | 30 | 2.95 | 90.8 | | 0.52 |
| | | 60 | 2.72 | 83.6 | | 0.42 |
| | Dog | 0 | 2.24 | 100 | >187 | 0.48 |
| | | 5 | 2.10 | 93.5 | | 0.46 |
| | | 15 | 2.07 | 92.0 | | 0.46 |
| | | 30 | 2.00 | 89.2 | | 0.42 |
| | | 60 | 1.88 | 83.7 | | 0.39 |
| | Human | 0 | 3.23 | 100 | >187 | 0.55 |
| | | 5 | 3.39 | 105 | | 0.61 |
| | | 15 | 3.46 | 107 | | 0.60 |
| | | 30 | 3.27 | 101 | | 0.53 |
| | | 60 | 2.99 | 92.6 | | 0.42 |
| | SD Rat | 0 | 1.62 | 100 | 178 | 0.60 |
| | | 5 | 1.61 | 99.1 | | 0.59 |
| | | 15 | 1.59 | 98.2 | | 0.57 |
| | | 30 | 1.50 | 92.8 | | 0.52 |
| | | 60 | 1.29 | 79.6 | | 0.47 |
| | Human | 0 | 1.80 | 100 | 117 | 0.63 |
| | | 5 | 1.76 | 97.6 | | 0.65 |
| | | 15 | 1.75 | 97.2 | | 0.63 |
| | | 30 | 1.69 | 93.9 | | 0.53 |
| | | 60 | 1.25 | 69.4 | | 0.38 |

^{a}Peak area of analyte (Aa) and peak area of internal standard (Ai).
^{b}Note:
If the remaining is > 80% at 60 min. incubation, the half-life will be reported as >187 min.
^{c}% remaining at 60 min. is calculated from the Aa/Ai ratio at each time point to the Aa/Ai ratio at time zero.

### Stability of Positive Controls in Plasma

Results of the positive controls, enalapril, bisacodyl and propantheline are summarized in Table 9.

TABLE 9
Stability of Positive Controls in SD Rat, Beagle
Dog and Human Plasma after 60 Minutes Incubation

| | | | Aa/Aj ^{a} | | |
|---|---|---|---|---|---|
| Compound | | Time | Mean | % | T_{1/2} |
| ID | Species | (min) | (N = 2) | Remaining | (min) |
| Enalapril | SD Rat | 0 | 0.58 | 100 | 7.5 |
| | | 5 | 0.39 | 67.9 | |
| | | 15 | 0.17 | 29.8 | |
| | | 30 | 0.05 | 8.14 | |
| | | 60 | 0.00 | 0.39 | |
| Bisacodyl | Dog | 0 | 1.49 | 100 | 3.6 |
| | | 5 | 0.68 | 45.9 | |
| | | 15 | 0.13 | 8.79 | |
| | | 30 | 0.01 | 0.34 | |
| | | 60 | 0.00 | 0.00 | |
| Propantheline | Human | 0 | 0.88 | 100 | 18.4 |
| | | 5 | 0.77 | 87.0 | |
| | | 15 | 0.55 | 62.2 | |
| | | 30 | 0.34 | 38.4 | |
| | | 60 | 0.09 | 10.5 | |

^{a} Peak area of analyte (Aa) and peak area of internal standard (Ai)

### Conclusion

Compound I-4 was very slightly metabolized in rat, dog and human plasma during a 1-hour incubation with >80% remaining, and Compound I-19 was metabolized in rat and human plasma with about 70% remaining in human plasma after an hour. Formation of metabolite Compound A was monitored, however there was no significant changes in the peak area ratios among all the samples compared to time zero.

### Example 4: In Vitro Metabolic Stability of Compound I-4 and Compound I-19 in Sprague Dawley Rat, Beagle Dog and Human Liver S9

The metabolic stability of Compound I-4 and Compound I-19 was assessed in rat, dog and human liver S9 by monitoring both the disappearance of prodrug and formation of metabolite.

### Materials and Methods

TABLE 10
Test Articles and Metabolites

| | | | | Storage |
|---|---|---|---|---|
| Compound ID | Batch No. | MW/FW | Purity % | Condition |
| Compound I-4 | 113-069 | 342.82 | 96.7 | Room |
| Compound I-19 | 113-158 | 370.83 | 98 | temperature, |
| Compound A | 10C0021 | 284.74 | 100 | protect |
| (metabolite) | | | | from light |

TABLE 11
Positive Controls

| Compound ID | Application | MW | Catalog No. | Vendor |
|---|---|---|---|---|
| 7-ethoxycoumarin | Positive control | 190.2 | E1379 | Sigma- |
| | | | | Aldrich |
| 7-hydroxycoumarin | Positive control | 162.14 | CAS93-35-6 | Sigma- |
| | | | | Aldrich |

### Solutions and Chemicals

Water purified in-house through a Barnstead GenPure water purification system (Thermo Scientific, Dubuque, IA) with 18.2 MΩ-cm resistance was used. Solvents were obtained from Fisher Scientific or Sigma-Aldrich.

### Test System

SD rat, beagle dog and human liver S9 are purchased from BioIVT, NY, USA or other validated vendors.

### Incubation Buffer (PBK)

50 mM potassium phosphate buffer was prepared from 1 M potassium phosphate buffer, pH 7.2.

### Preparation of Test Article Solutions

Compound I-4 and Compound I-19 stock solutions at 10 mM was prepared in DMSO. The dilution of 1 mM intermediate was prepared by 70% methanol/water, then 10 M working solutions were prepared by diluting the 1 mM intermediates with 50 mM PPK. All working solutions were freshly prepared on the day of experiment and disposed after use.

### Preparation of Positive Control Stock and Working Solutions

The positive control stock solutions, 7-ethoxycumarin and 7-hydroxycoumarin were prepared at 10 mM in DMSO and stored at ca. −20° C. Prior to use, the stock solutions were brought to room temperature and thoroughly mixed. The working solutions were freshly prepared similarly as described for the test article working solution.

### Preparation of Internal Standard Solutions and Stop Solutions

The stock solution of internal standards, tolbutamide and labetalol, were prepared in DMSO and stored at ca. −20° C. The stop solution was prepared by spiking the stock solution (1 mg/mL) into acetonitrile to achieve a 200 ng/mL final concentration.

### Preparation of S9 Working Solutions

The liver S9 from tested species were diluted with PBK buffer to make working solutions of 0.625 mg/mL.

### Preparation of Cofactors Solutions

The mixture of cofactors solution was made at 10 mM of NADPH and 10 mM of UDPGA in PBK buffer.

### Assay Procedures

The liver S9 working solutions were added to 96-well plates (80 μL/well) in duplicate. The plates were pre-incubated for 10-min. at 37° C. in a water bath and then spiked 10 μL each of 10 μM Compound I-4 and Compound I-19 or positive control working solution into correspondence wells, separately. Reaction was initiated by adding cofactor mixture 10 μL/well. The plates were incubated at 37° C. in a water bath with shaking. The final incubation mixture contained Compound I-4 or Compound I-19, or positive control at 1 μM, and 0.5 mg/mL of liver S9, and 1 mM NADPH, 1 mM of UDPGA. The final organic solvent content in the incubation was ≤1%.

At specified time points, i.e., 5, 10, 20, 30 and 60 minutes, NCF60 (without adding NADPH and UDPGA replaced with PBK buffer) reaction was stopped by adding three volumes of stop solution. The time zero (TO) samples were prepared by adding three volumes of stop solution to liver S9 samples, followed by addition of test article or control, and cofactor mixture solution 10 μL/well.

All sample plates were mixed well by shaking for approximately 10 minutes and centrifuged at 3220×g for 20 minutes. Subsequently, 100 L of supernatant was removed from each well, diluted with 100 μL pure water and analyzed by LC/MS/MS.

### Bioanalytical Analysis

Concentrations of test articles and control compounds in the samples were analyzed by LC/MS/MS using semi-quantitation. Plotting of the chromatograms and peak area integrations were carried out by Analyst (AB Sciex, Framingham, Massachusetts, USA). The peak area ratios of analyte/internal standard were used to semi-quantitatively determine the concentrations.

### Data Analysis

The software provided with the LC/MS/MS system (Analyst® software, v.1.6.2, AB Sciex) was used to integrate the area under the mass ion peaks of the analytes and internal standards. The calculated concentrations were then exported to Microsoft Excel (v.2016) and the data were organized and tabulated.

The peak area ratios (PAR) of analyte/internal standard were used to semi-quantitatively determine the concentration of test article and control compounds, and calculate the percent remaining with the following equation:

$$\% Remaining = \frac{PAR of analyte to internal standard at each time point}{PAR of analyte to internal standard at {T}_{0}} \times 100 \%$$

The elimination rate (ke) was calculated from a log linear plot of % remaining versus time. The half-life was calculated using the following equation:

$${t}_{1 / 2} ( min ) = 0.693 / ke$$

The intrinsic clearances were calculated using the following equations:

$${CL}_{int ( HIH )} ( μ L / min / mg ) = 0.693 / {t}_{1 / 2} / mg protein per mL$$

### Results and Discussion

### Metabolism Stability of Compound I-4 and Compound I-19 in Liver S9

The results of metabolic stability of Compound I-4 and Compound I-19 in liver S9 of the tested species, including percent remaining, t_{1/2} (min) and intrinsic clearance values can be found in Table 12 to Table 14, and Table 15 for the formation of metabolite. Results of the positive control are summarized in Table 16.

TABLE 12
Summary of Metabolic Stability in SD Rat, Dog and Human Liver S9

| | Stability at 1 μM | | | | Compound A formed | |
|---|---|---|---|---|---|---|
| | | | CL_{int (LS9.)} | | (Mean of Ratio) | |
| | | | (μL/min/mg) | % Remaining | At | At |
| Compound ID | Species | t_{1/2} (min) | Mean (n = 2) | at 60 min | 0 min | 60 min |
| Compound | SD Rat | 20.1 | 69.0 | 12.2 | 0.39 | 0.68 |
| I-4 | Dog | 46.2 | 30.0 | 39.7 | 0.08 | 0.13 |
| | Human | 80.6 | 17.2 | 57.7 | 0.35 | 0.51 |
| Compound | SD Rat | 8.86 | 156 | 0.87 | 0.43 | 0.28 |
| I-19 | Dog | 57.4 | 24.1 | 46.1 | 0.42 | 0.35 |
| | Human | 36.4 | 38.1 | 30.8 | 042 | 0.34 |

Ratio: analyte peak area/internal standard peak area
Mean = 2

TABLE 13
Metabolic Stability of Compound I-4 in Liver S9 at 60-Minute Incubation

| Compound | | | Aa/Aiª | % | | CL_{int(LS9)} |
|---|---|---|---|---|---|---|
| ID | Species | Time (min) | Mean (n = 2) | Remaining | t_{1/2} (min) | (μL/min/mg) |
| Compound | Rat | 0 | 1.28 | 100 | 20.1 | 69.0 |
| I-4 | | 5 | 0.99 | 78 | | |
| | | 10 | 0.92 | 72 | | |
| | | 20 | 0.65 | 51 | | |
| | | 30 | 0.47 | 36 | | |
| | | 60 | 0.16 | 12 | | |
| | | NCF60 | 0.99 | 77 | NA | |
| | Dog | 0 | 1.11 | 100 | 46.2 | 30.0 |
| | | 5 | 1.10 | 99 | | |
| | | 10 | 0.86 | 77 | | |
| | | 20 | 0.93 | 84 | | |
| | | 30 | 0.79 | 71 | | |
| | | 60 | 0.44 | 40 | | |
| | | NCF60 | 0.99 | 89 | NA | |
| | Human | 0 | 1.24 | 100 | 80.6 | 17.2 |
| | | 5 | 1.14 | 92 | | |
| | | 10 | 1.08 | 87 | | |
| | | 20 | 0.98 | 79 | | |
| | | 30 | 0.97 | 78 | | |
| | | 60 | 0.72 | 58 | | |
| | | NCF60 | 1.03 | 83 | NA | |

^{a}Peak area of analyte (Aa) and peak area of internal standard (Ai)
^{b}%remaining is calculated from the Aa/Ai ratio at each time point to the Aa/Ai ratio at time zero NCF60: No cofactors were added in the 60 min. incubation samples.

TABLE 14
Metabolic Stability of Compound I-19 in Liver S9 at 60-Minute Incubation

| | | | Aa/Aiª | % | | CL_{int(LS9)} |
|---|---|---|---|---|---|---|
| Compound ID | Species | Time (min) | Mean (n = 2) | Remaining^{b} | t_{1/2} (min) | (μL/min/mg) |
| Compound | Rat | 0 | 0.82 | 100 | 8.86 | 156 |
| I-19 | | 5 | 0.46 | 57 | | |
| | | 10 | 0.33 | 40 | | |
| | | 20 | 0.14 | 18 | | |
| | | 30 | 0.06 | 7.3 | | |
| | | 60 | 0.01 | 0.9 | | |
| | | NCF60 | 0.53 | 65 | NA | |
| | Dog | 0 | 0.83 | 100 | 57.4 | 24.1 |
| | | 5 | 0.69 | 84 | | |
| | | 10 | 0.61 | 74 | | |
| | | 20 | 0.63 | 76 | | |
| | | 30 | 0.47 | 57 | | |
| | | 60 | 0.38 | 46 | | |
| | | NCF60 | 0.55 | 66 | | |
| | Human | 0 | 0.83 | 100 | 36.4 | 38.1 |
| | | 5 | 0.65 | 78 | | |
| | | 10 | 0.63 | 76 | | |
| | | 20 | 0.53 | 63 | | |
| | | 30 | 0.38 | 46 | | |
| | | 60 | 0.26 | 31 | | |

| | | | |
|---|---|---|---|
| NCF60 | 0.53 | 64 | NA |

^{a}Peak area of analyte (Aa) and peak area of internal standard (Ai)
^{b}%remaining is calculated from the Aa/Ai ratio at each time point to the Aa/Ai ratio at time zero NCF60: No cofactors were added in the 60 min. incubation samples.

TABLE 15
Formation of Compound A in Liver S9 at 60-Minute Incubation

| | Time | Compound A formed (Aa/Ai ^{a}) | |
|---|---|---|---|
| Species | (min) | from Compound I-4 | from Compound I-19 |
| Rat | 0 | 0.39 | 0.43 |
| | 5 | 0.53 | 0.40 |
| | 10 | 0.52 | 0.38 |
| | 20 | 0.62 | 0.39 |
| | 30 | 0.67 | 0.35 |
| | 60 | 0.68 | 0.28 |
| | NCF60 | 0.58 | 0.37 |
| Human | 0 | 0.35 | 0.42 |
| | 5 | 0.41 | 0.37 |
| | 10 | 0.46 | 0.39 |
| | 20 | 0.46 | 0.38 |
| | 30 | 0.48 | 0.35 |
| | 60 | 0.51 | 0.34 |
| | NCF60 | 0.55 | 0.34 |
| Dog | 0 | 0.08 | 0.42 |
| | 5 | 0.10 | 0.39 |
| | 10 | 0.09 | 0.37 |
| | 20 | 0.11 | 0.38 |
| | 30 | 0.11 | 0.35 |
| | 60 | 0.13 | 0.35 |
| | NCF60 | 0.13 | 0.36 |

^{a} Peak area of analyte (Aa) and peak area of internal standard (Ai)
NCF60: No cofactors were added in the 60 min. incubation samples.
ND: not detectable
Values were presented as Mean = 2

TABLE 16
Metabolic Stability of Positive Control in Liver S9

| | | % Remaining | t_{1/2} | CL_{int(LS9.)} |
|---|---|---|---|---|
| Compound | Species | at 60 min | (min) | (μL/min/mg) |
| 7-ethoxycoumarin | Rat | 29.9 | 34.8 | 39.8 |
| 7-hydroxycoumarin | | 33.4 | 38.0 | 36.4 |
| 7-ethoxycoumarin | Dog | 24.9 | 30.2 | 46.0 |
| 7-hydroxycoumarin | | 35.0 | 39.2 | 35.3 |
| 7-ethoxycoumarin | Human | 45.9 | 53.4 | 26.0 |
| 7-hydroxycoumarin | | 30.2 | 34.5 | 40.2 |

Values were presented as Mean = 2

### Conclusion

The percent remaining of Compound I-4 was 12.2, 39.7 and 59.7%; Compound I-19 was 0.87, 46.1 and 30.8% in rat, dog and human liver S9 at 60 minutes. However, the primary metabolite Compound A formation did not change significantly from the level at time zero. Compound I-19 had 64-66% remaining in the absence of NADPH, suggesting there might be some non-CYP mediated metabolism.

### Example 5: Evaluation of Pharmacokinetics of Compound a Analogs and Compound a Following Single Oral Administration of the Analogs to Male and Female Beagle Dogs

The pharmacokinetic (PK) properties of Compound I-17, Compound I-19, and Compound A was evaluated following single oral (PO) administration of the analogs to male and female beagle dogs.

### Materials and Methods

### Test Article

| | | Molecular | Formula | | Storage |
|---|---|---|---|---|---|
| Test Article | Batch | Weight | Weight | Purity | Condition |
| Compound I-17 | 115-113 | 366.89 | 366.89 | 98.85 | 4° C. |
| Compound I-19 | 115-108 | 370.83 | 370.83 | 96.20 | 4° C. |

### Study Design

The two Compound A analogs, Compound I-17 and Compound I-19, each was given to two male and two female beagle dogs at 10 mg/kg by oral gavage. Blood samples were collected from each animal at 0.5, 1, 2, 4, 6, 8 and 24 hours post-dose, and then centrifuged to extract plasma for the determination of the concentrations of the analogs and Compound A as metabolite. The concentrations of the analytes in the plasma were quantified by liquid chromatography tandem mass spectrometry (LC-MS/MS).

### Compound Administration Details

| | | | Nominal | Dose | Dose | | |
|---|---|---|---|---|---|---|---|
| Test | n | | Dose | Volume | Conc. | | Dosing |
| Article | Male | Female | (mg/kg) | (mL/kg) | (mg/mL) | Vehicle | Route |
| Compound | 2 | 2 | 10 | 5 | 2 | 4% DMSO, | PO |
| I-17 | | | | | | 30% PEG400, | |
| | | | | | | 66% HP-β-CD | |
| | | | | | | (30% in H₂O) | |
| Compound | 2 | 2 | 10 | 5 | 2 | 4% DMSO, | PO |
| I-19 | | | | | | 30% PEG400, | |
| | | | | | | 66% HP-β-CD | |
| | | | | | | (30% in H₂O) | |

### Sample Collection Details

| | Dosing | | Sampling Schedule |
|---|---|---|---|
| Test Article | Route | Matrix | (h post dosing) |
| Compound I-17 | PO | Plasma | 0.5, 1, 2, 4, 6, 8, 24 |
| Compound I-19 | PO | Plasma | 0.5, 1, 2, 4, 6, 8, 24 |

Anticoagulant: K₂EDTA

The bioanalytical assay provided a lower limit of quantification (LLOQ) of 1.0 ng/mL and an upper limit of quantification (ULOQ) of 3000 ng/mL for all the analytes. The data of plasma concentration vs. time were analyzed using Phoenix WinNonlin 8.3 to determine the PK properties of the analytes. The non-compartmental analysis model and the linear log trapezoidal method were applied to the PK calculation. The details of compound administrations and sample collection are summarized in tables 17-24 below.

### Test Article Formulation

### Formulation Preparation

Dose solutions were prepared freshly on the day of study prior to dosing. The formulations of the two analogs were prepared the same way using 4% DMSO, 30% PEG 400, and 66% HP-β-CD (30% in H₂O, w/v) as vehicle. The target concentration of Compound I-17 and Compound I-19 was 2 mg/mL; when administered at the nominal dose volume of 5 mL/kg, it yielded a target dosage of 10 mg/kg of each analog.

### Formulation Verification

Two 50 μL aliquots of each dose solution were set aside at the completion of formulation preparation. The concentrations of Compound I-17 and Compound I-19 in the dosing solutions were determined by LC-MS/MS. All formulation samples were analyzed in duplicate and quantified against a calibration curve consisting of six concentrations of the analyte.

### In Life Procedures

### Animal Husbandry

Six male and six female dogs were purchased from Marshall Bioresources, Beijing, China. The animals were pair-housed; the room(s) were controlled and monitored for relative humidity (targeted mean range 40% to 70%) and temperature (targeted mean range 18° to 26° C.) with 10 to 20 air changes/hour. The room was on a 12-hour light/dark cycle except when interruptions were necessitated by study activities. The animals were fed approximately 220 grams of Certified Dog Diet (Beijing Keao Xieli Feed Co., Ltd. Beijing, P. R. China) twice daily. All animals were confirmed healthy prior to being assigned to the study.

### Feeding Condition

The animals were fasted overnight prior to dosing. Food was returned 4 hours post-dose. Animals had access to water freely the entire time throughout the study.

### Test Article Administration

The animals were weighed prior to dosing and the dose volume was calculated individually based the obtained body weight and the nominal dose volume. The test articles were administered orally by gavage. The nominal dose volume was 5 mL/kg for both analogs.

### Clinical Observation

Cage side observation was performed before and after dosing as well as at each scheduled sample collection. The observation included appearance, behavior, morbidity and mortality. Abnormal findings, if any, were recorded in the study notebook and communicated with study director, veterinarian, and Supernus if necessary.

### Blood Collection and Plasma Preparation

Approximately 0.5 mL blood was collected at each scheduled time point from the peripheral veins. The actual sample collection time was within 1 min. of the nominal time for collections prior to or at 1 hour, and within 5% of the nominal time for collections post 1 hour.

The blood samples were placed in pre-labeled micro-centrifuge tubes containing K2EDTA as anticoagulant and kept on ice, and then processed to extract plasma within 60 minutes of collection by centrifugation at 2-8° C., 3000 g for 10 minutes. The plasma samples were placed in microcentrifuge tubes and stored at −70+10° C. until analysis.

### Bioanalytical Assay

The concentrations of Compound I-17, Compound I-19 and Compound A in the plasma were determined using qualified LC-MS/MS method.

### Pharmacokinetic Analysis

The data of plasma concentration versus time of each animal was analyzed using Phoenix WinNonlin 8.3 to determine the PK properties of the two analogs and Compound A. The non-compartmental analysis model and the linear/log trapezoidal method were applied to the PK calculation.

Plasma concentrations below the LLOQ prior to Tₘₐₓ were set to zero, and those after Tₘₐₓ were excluded from PK calculation. The nominal dose and the nominal sample collection time were used for the calculation. The values of plasma concentrations and PK parameters are reported to three significant figures.

### Results and Discussion

### Clinical Observation

The two female dogs receiving Compound I-19 appeared to have diarrhea at 24 hours post-dose. Otherwise, the animals looked normal.

### Dose Accuracy Verification

The concentrations of Compound I-17 and Compound I-19 in the dosing solutions were determined by LC-MS/MS. The measured concentrations of Compound I-17 and Compound I-19 were 1.97 and 2.00 mg/mL, respectively. Compared to their nominal value of 2.00 mg/mL, the accuracy of the Compound I-17 formulation was 98.5% and that of Compound I-19 was 99.9%, both were well within the acceptable range of 20% of its nominal concentration value.

### Pharmacokinetics of Compound I-17, Compound I-19, and S(−)- and R(+)-Compound A

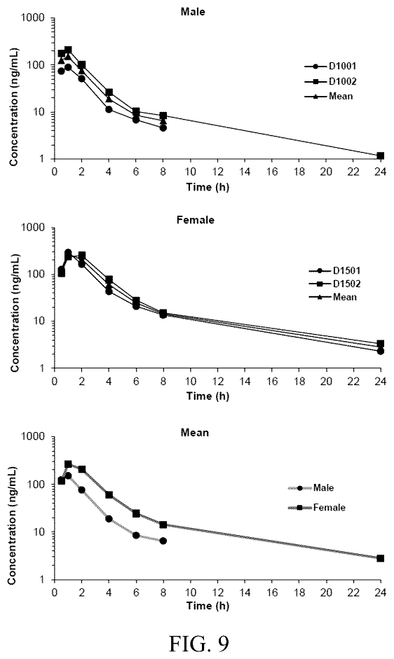

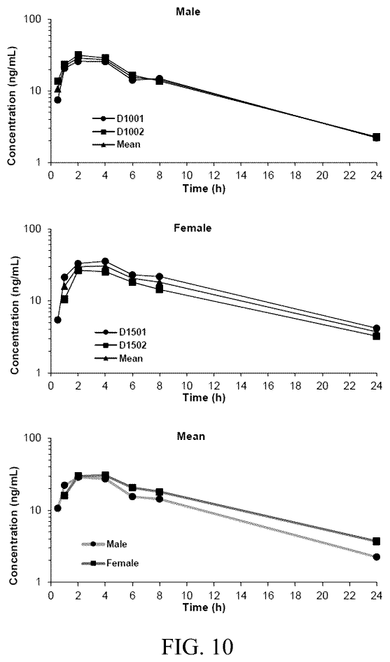

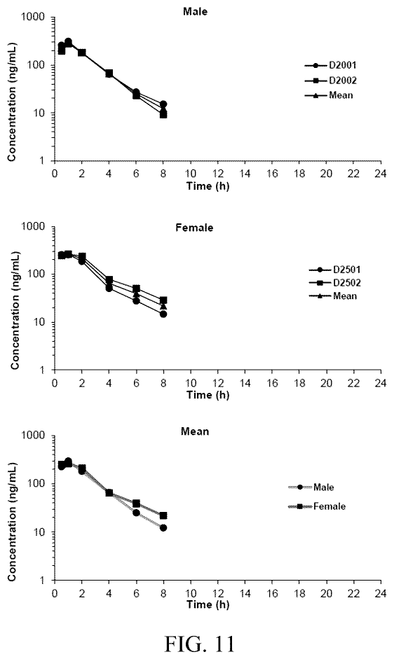

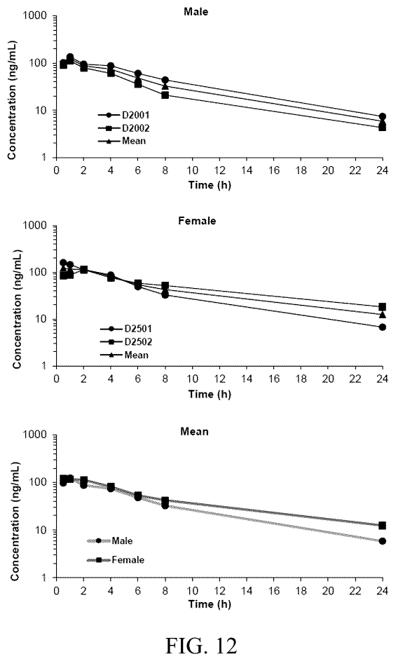

The individual and mean plasma concentrations of Compound I-17, Compound I-19 and Compound A following PO administrations of the analogs are tabulated in Tables 17, 18, 19, and 20. The corresponding PK parameters are summarized in Tables 21, 22, 23 and 24. The plasma concentration-time profiles of Compound I-17, Compound I-19 and Compound A are shown in FIGS. 9, 10, 11, and 12.

TABLE 17
Individual and mean plasma concentrations (ng/mL) of Compound I-17
following single PO administration to male and female beagle dogs at 10 mg/kg

| | Male | | | Female | | |
|---|---|---|---|---|---|---|
| Time (h) | D1001 | D1002 | Mean | D1501 | D1502 | Mean |
| 0.500 | 73.5 | 176 | 125 | 128 | 106 | 117 |
| 1.00 | 89.0 | 212 | 151 | 293 | 236 | 265 |
| 2.00 | 51.0 | 101 | 75.8 | 164 | 252 | 208 |
| 4.00 | 11.2 | 26.5 | 18.9 | 43.0 | 77.4 | 60.2 |
| 6.00 | 6.77 | 10.2 | 8.47 | 20.8 | 28.2 | 24.5 |
| 8.00 | 4.57 | 8.39 | 6.48 | 13.6 | 15.0 | 14.3 |
| 24.0 | BQL | 1.16 | ND | 2.29 | 3.32 | 2.81 |

BQL: below the quantifiable limit;
ND: not determine due to insufficient number of values

TABLE 18
Individual and mean plasma concentrations (ng/mL) of Compound I-17 following
single PO administration to male and female beagle dogs at 80 mg/kg

| | Male | | | Female | | |
|---|---|---|---|---|---|---|
| Time (h) | D1001 | D1002 | Mean | D1501 | D1502 | Mean |
| 0.500 | 7.45 | 13.8 | 10.6 | 5.44 | BQL | ND |
| 1.00 | 20.8 | 23.7 | 22.2 | 21.3 | 10.5 | 15.9 |
| 2.00 | 25.9 | 31.6 | 28.8 | 33.0 | 26.4 | 29.7 |
| 4.00 | 25.6 | 29.0 | 27.3 | 35.5 | 25.4 | 30.5 |
| 6.00 | 14.2 | 16.7 | 15.4 | 22.9 | 18.1 | 20.5 |
| 8.00 | 14.9 | 13.7 | 14.3 | 21.8 | 14.4 | 18.1 |
| 24.0 | 2.20 | 2.28 | 2.24 | 4.17 | 3.23 | 3.70 |

TABLE 19
Individual and mean plasma concentrations (ng/mL) of Compound I-19 following
single PO administration to male and female beagle dogs at 10 mg/kg

| | Male | | | Female | | |
|---|---|---|---|---|---|---|
| Time (h) | D2001 | D2002 | Mean | D2501 | D2502 | Mean |
| 0.500 | 259 | 195 | 227 | 257 | 246 | 252 |
| 1.00 | 312 | 281 | 297 | 258 | 262 | 260 |
| 2.00 | 181 | 181 | 181 | 186 | 240 | 213 |
| 4.00 | 64.2 | 67.7 | 66.0 | 50.7 | 78.2 | 64.4 |
| 6.00 | 27.3 | 22.7 | 25.0 | 27.8 | 50.5 | 39.1 |
| 8.00 | 15.2 | 9.16 | 12.2 | 14.6 | 28.7 | 21.7 |
| 24.0 | BQL | BQL | ND | BQL | BQL | ND |

BQL: below the quantifiable limit;
ND: not determine due to insufficient number of values

TABLE 20
Individual and mean plasma concentrations (ng/mL) of Compound I-19 following
single PO administration to male and female beagle dogs at 80 mg/kg

| | Male | | | Female | | |
|---|---|---|---|---|---|---|
| Time (h) | D2001 | D2002 | Mean | D2501 | D2502 | Mean |
| 0.500 | 102 | 91.6 | 96.8 | 160 | 83.0 | 121 |
| 1.00 | 136 | 111 | 123 | 145 | 86.7 | 116 |
| 2.00 | 94.9 | 78.9 | 86.9 | 113 | 113 | 113 |
| 4.00 | 87.4 | 60.3 | 73.8 | 87.3 | 76.1 | 81.7 |
| 6.00 | 60.4 | 35.4 | 47.9 | 49.1 | 57.9 | 53.5 |
| 8.00 | 43.8 | 20.9 | 32.4 | 32.5 | 52.0 | 42.3 |
| 24.0 | 7.39 | 4.28 | 5.83 | 6.70 | 18.2 | 12.4 |

TABLE 21
Individual and mean PK parameters of Compound A following single PO
administration of Compound I-17 to male and female beagle dogs at 10 mg/kg

| | Male | | | Female | | |
|---|---|---|---|---|---|---|
| Time (h) | D1001 | D1002 | Mean | D1501 | D1502 | Mean |
| Rsq_adj | 0.989 | 0.999 | 0.994 | 0.986 | 0.929 | 0.958 |
| Cₘₐₓ (ng/mL) | 89.0 | 212 | 151 | 293 | 252 | 272 |
| Tₘₐₓ (h) | 1.00 | 1.00 | 1.00 | 1.00 | 2.00 | 1.50 |
| T_{1/2} (h) | 3.08 | 5.70 | 4.39 | 5.87 | 6.33 | 6.10 |
| Tₗₐₛₜ (h) | 8.00 | 24.0 | 16.0 | 24.0 | 24.0 | 24.0 |
| AUC₀₋ₗₐₛₜ (ng · h/mL) | 209 | 513 | 361 | 736 | 915 | 826 |
| AUC_{0-inf} (ng · h/mL) | 229 | 522 | 376 | 756 | 946 | 851 |
| MRT₀₋ₗₐₛₜ (h) | 2.15 | 3.33 | 2.74 | 3.88 | 4.14 | 4.01 |
| MRT_{0-inf} (h) | 3.07 | 3.86 | 3.46 | 4.62 | 5.07 | 4.84 |

TABLE 22
Individual and mean PK parameters of Compound A following single PO
administration of Compound I-17 to male and female beagle dogs at 80 mg/kg

| | Male | | | Female | | |
|---|---|---|---|---|---|---|
| Time (h) | D1001 | D1002 | Mean | 0.989 | 0.999 | 0.994 |
| Rsq_adj | 0.969 | 1.000 | 0.984 | 35.5 | 26.4 | 31.0 |
| Cₘₐₓ (ng/mL) | 25.9 | 31.6 | 28.8 | 4.00 | 2.00 | 3.00 |
| Tₘₐₓ (h) | 2.00 | 2.00 | 2.00 | 7.07 | 7.31 | 7.19 |
| T_{1/2} (h) | 6.33 | 6.23 | 6.28 | 24.0 | 24.0 | 24.0 |
| Tₗₐₛₜ (h) | 24.0 | 24.0 | 24.0 | 376 | 271 | 323 |
| AUC₀₋ₗₐₛₜ (ng · h/mL) | 258 | 278 | 268 | 419 | 305 | 362 |
| AUC_{0-inf} (ng · h/mL) | 278 | 298 | 288 | 8.56 | 8.55 | 8.56 |
| MRT₀₋ₗₐₛₜ (h) | 7.90 | 7.43 | 7.66 | 11.2 | 11.5 | 11.3 |
| MRT_{0-inf} (h) | 9.72 | 9.19 | 9.45 | 0.989 | 0.999 | 0.994 |

TABLE 23
Individual and mean PK parameters of Compound A following single PO
administration of Compound I-19 to male and female beagle dogs at 10 mg/kg

| | Male | | | Female | | |
|---|---|---|---|---|---|---|
| Time (h) | D2001 | D2002 | Mean | D2501 | D2502 | Mean |
| Rsq_adj | 0.977 | 0.994 | 0.986 | 0.999 | 0.990 | 0.994 |
| Cₘₐₓ (ng/mL) | 312 | 281 | 297 | 258 | 262 | 260 |
| Tₘₐₓ (h) | 1.00 | 1.00 | 1.00 | 1.00 | 1.00 | 1.00 |
| T_{1/2} (h) | 1.93 | 1.39 | 1.66 | 2.23 | 2.77 | 2.50 |
| Tₗₐₛₜ (h) | 8.00 | 8.00 | 8.00 | 8.00 | 8.00 | 8.00 |
| AUC₀₋ₗₐₛₜ (ng · h/mL) | 801 | 737 | 769 | 739 | 932 | 836 |
| AUC_{0-inf} (ng · h/mL) | 843 | 756 | 799 | 786 | 1047 | 916 |
| MRT₀₋ₗₐₛₜ (h) | 2.29 | 2.31 | 2.30 | 2.29 | 2.65 | 2.47 |
| MRT_{0-inf} (h) | 2.72 | 2.50 | 2.61 | 2.82 | 3.68 | 3.25 |

TABLE 24
Individual and mean PK parameters of Compound A following single PO
administration of Compound I-19 to male and female beagle dogs at 80 mg/kg

| | Male | | | Female | | |
|---|---|---|---|---|---|---|
| Time (h) | D2001 | D2002 | Mean | D2501 | D2502 | Mean |
| Rsq_adj | 0.997 | 0.961 | 0.979 | 0.982 | 0.999 | 0.991 |
| Cₘₐₓ (ng/mL) | 136 | 111 | 123 | 160 | 113 | 137 |
| Tₘₐₓ (h) | 1.00 | 1.00 | 1.00 | 0.500 | 2.00 | 1.25 |
| T_{1/2} (h) | 6.05 | 6.28 | 6.16 | 6.54 | 10.7 | 8.61 |
| Tₗₐₛₜ (h) | 24.0 | 24.0 | 24.0 | 24.0 | 24.0 | 24.0 |
| AUC₀₋ₗₐₛₜ (ng · h/mL) | 958 | 622 | 790 | 919 | 1108 | 1013 |
| AUC_{0-inf} (ng · h/mL) | 1022 | 661 | 841 | 982 | 1387 | 1185 |
| MRT₀₋ₗₐₛₜ (h) | 7.01 | 6.08 | 6.54 | 6.23 | 8.76 | 7.49 |
| MRT_{0-inf} (h) | 8.63 | 7.66 | 8.14 | 7.98 | 14.9 | 11.5 |

Following oral administration of Compound I-17 at 10 mg/kg, the analog achieved a Cₘₐₓ of 151 ng/mL in the male dogs and 272 ng/mL in the female dogs. The Tₘₐₓ of Compound I-17 achieved was 1 hour in the male dogs and 1.5 hours in the female dogs post-dose. Compound I-17 achieved AUC₀₋ₗₐₛₜ of 361 ng·h/mL in the male dogs and 826 ng·h/mL in the female dogs. The T_{1/2} of Compound I-17 following PO administration was 4.39 hours in the male dogs and 6.10 hours in the female dogs. The MRT₀₋ₗₐₛₜ of Compound I-17 was 2.74 hours in the male dogs and 4.01 hours in the female dogs.

The Cₘₐₓ of Compound A following PO administration of Compound I-17 at 80 mg/kg measured 28.8 ng/mL in the male dogs and 31.0 ng/mL in the female dogs. The Tₘₐₓ for Compound A following PO administration of Compound I-17 was 2.00 hours in the male dogs and 3.00 hour in the female dogs. The AUC₀₋ₗₐₛₜ of Compound A was 268 ng·h/mL in the male dogs and 323 ng·h/mL in the female dog. The T_{1/2} of Compound A following PO administration of Compound I-17 was 6.28 hours in the male dogs and 7.19 hours in the female dogs. The MRT₀₋ₗₐₛₜ of Compound A was 7.66 hours in the male dogs and 8.56 hours in the female dogs.

Following oral administration of Compound I-19 at 10 mg/kg, the analog achieved a Cₘₐₓ of 297 ng/mL in the male dogs and 260 ng/mL in the female dogs. The Tₘₐₓ of Compound I-19 was 1 hour in both the male and female dogs. Compound I-17 achieved an AUC₀₋ₗₐₛₜ of 769 ng·h/mL in the male dogs and 836 ng·h/mL in the female dogs. The T_{1/2} of Compound I-19 following PO administration was 1.66 hours in the male dogs and 2.50 hours in the female dogs. The MRT₀₋ₗₐₛₜ of Compound I-17 was 2.30 hours in the male dogs and 2.47 hours in the female dogs.

The Cₘₐₓ of Compound A following PO administration of Compound I-19 at 80 mg/kg measured 123 ng/mL in the male dogs and 137 ng/mL in the female dogs. The Tₘₐₓ for Compound A following PO administration of Compound I-19 at 80 mg/kg was 1.00 hour in the male dogs and 1.25 hour in the female dogs. The AUC₀₋ₗₐₛₜ of Compound A was 790 ng·h/mL in the male dogs and 1013 ng·h/mL in the female dog. The T_{1/2} of Compound A following PO administration of Compound I-19 was 6.16 hours in the male dogs and 8.61 hours in the female dogs. The MRT₀₋ₗₐₛₜ of Compound A was 6.54 hours in the male dogs and 7.49 hours in the female dogs.

The data showed that the Cₘₐₓ and AUC₀₋ₗₐₛₜ of Compound I-17 were about doubled in the female dogs compared the male dogs. The T_{1/2} and MRT₀₋ₗₐₛₜ were also longer in the female dogs compared to the male dogs. However, the PK properties of Compound A following PO administration of Compound I-17 did not seem different between the male and female dogs. In contrast, the Cₘₐₓ, AUC₀₋ₗₐₛₜ, T_{1/2} and MRT₀₋ₗₐₛₜ of Compound I-19 were comparable between the male and female dogs, while the AUC₀₋ₗₐₛₜ of Compound A as the result of PO administration of Compound I-19 was greater in the female dogs than that in the male dogs. The T_{1/2} and MRT₀₋ₗₐₛₜ of Compound A were not much different between the male and female dogs.

The Cₘₐₓ and AUC₀₋ₗₐₛₜ of Compound I-17 and Compound I-19 in the female dogs were comparable, but the Cₘₐₓ and AUC₀₋ₗₐₛₜ of Compound I-19 in the male dogs were approximately doubled compared to those of Compound I-17. The T_{1/2} of Compound I-17 was longer than that of Compound I-19 in both male and female dogs. Compared to Compound I-17, Compound I-19 consistently yielded a greater (about 2.5×) Cₘₐₓ and AUC₀₋ₗₐₛₜ of Compound A in both the male and female dogs. The T_{1/2} of Compound A did not seem different between animal genders or between analogs.

### Example 6: Pharmacokinetics of Compound I-19, Compound a, and Compound B Following Single Oral Administration of Compound a or Compound I-19, or Intraperitoneal Administration of Compound I-19 to Male CD-1 Mice

The studies were carried out to evaluate the pharmacokinetic (PK) properties of Compound I-19, Compound A, and Compound B following single oral (PO) administration of Compound A or Compound I-19, or intraperitoneal (IP) administration of Compound I-19 to male CD-1 mice.

### Materials and Methods

### Study Design

Three groups of male CD-1 mice, n=3 per group, were given a PO dose of Compound A and a PO and an IP dose of Compound I-19, respectively. The PO dose of Compound A was 5.102 mg/kg, equal to 5 mg/kg of Compound A after correction for impurity. The PO and IP doses of Compound I-19 were 6.645 and 4.061 mg/kg, equivalent to 5 mg/kg and 3 mg/kg of free base Compound A after corrections for salt and impurity, respectively. Blood samples were collected from each animal at 0.25, 0.5, 1, 2, 4, 8 and 24 hours post-dose, and then processed to extract plasma for the determination of the concentrations of Compound I-19, Compound A, and Compound B (only for the IP group by liquid chromatography tandem mass spectrometry (LC-MS/MS). The non-compartmental analysis model and the linear log trapezoidal method were applied to the PK calculation using Phoenix WinNonlin 6.3. Details of the compound administration and blood sample collection are summarized in the tables below.

### Compound Administration Details

| | | | Nominal | Verified | Dose | | | |
|---|---|---|---|---|---|---|---|---|
| | | Test | Dose | Dose | Volume | | Dosing | |
| Study ID | n | Article | (mg/kg) | (mg/kg) | (mL/kg) | Vehicle | Route | Analytes |
| SUPER- | 3 | Compound | 5.102 | 4.80 | 10 | 4% | PO | Compound |
| 20190517- | | A | | | | DMSO, | | A |
| MPK | | | | | | 30% | | |
| SUPER- | 3 | Compound | 6.645 | 6.741 | 10 | PEG400, | PO | Compound |
| 20190517- | | I-19 | | | | 66% HP- | | I-19 and |
| MPK | | | | | | β-CD | | Compound |
| | | | | | | | | A |
| SUPER- | 3 | Compound | 4.061 | 5.406 | 10 | (30% in | IP | Compound |
| 20201021- | | I-19 | | | | H₂O) | | I-19, |
| MPK | | | | | | | | Compound |
| | | | | | | | | A, and |
| | | | | | | | | Compound |
| | | | | | | | | B |

### Sample Collection Details

| | | Sampling schedule |
|---|---|---|
| Study ID | Matrix | (h post dosing) |
| SUPER-20190517-MPK | Plasma | 0.25, 0.5, 1, 2, 4, 8, 24 |
| SUPER-20201021-MPK | Plasma | 0.25, 0.5, 1, 2, 4, 8, 24 |

### Test Article Formulation

### Formulation Preparation

Dose solutions were prepared freshly on each day of studies prior to dosing. All formulations were prepared in 4% DMSO, 30% PEG 400, and 66% HP-β-CD (30% in H₂O, w/v). The concentration of Compound A in the formulation was 0.510 mg/mL, equivalent to 0.5 mg/mL of free base Compound A after correction for impurity. The concentration of Compound I-19 in the PO formulation was 0.664 mg/mL and that in the IP formulation was 0.406 mg/mL, equivalent to 0.5 mg/mL and 0.3 mg/mL of free base Compound A, respectively. Both the PO and IP formulations were administered at 10 mL/kg to achieve 5 mg/kg and 3 mg/kg of Compound A, respectively.

### Formulation Verification

Two 50 μL aliquots of each dose solution were set aside at the completion of formulation preparation. The concentrations of Compound A and Compound I-19 in the dosing solutions were determined by LC-MS/MS. All formulation samples were analyzed in duplicate and quantified against a calibration curve consisting of six concentrations of the analyte.

### In-Life Procedures

### Animal Husbandry

The animals used in studies were sourced from Hilltop Lab Animals, Inc. (Scottdale, PA 15683). The animals were fed certified pellet diet (Certified Rodent Diet #5002, LabDiet) and allowed access to water (reverse osmosis) ad libitum. The animals were acclimated to the facility for 3 days prior to the commencement of the study.

All animals were confirmed healthy prior to being assigned to studies. Each animal was given a unique identification number, which was marked on the tail and written on the cage card as well. The 3 mice receiving Compound A in study “SUPER-20190517-MPK” were identified as M1, M2 and M3 and those receiving Compound I-19 as M19, M20 and M21. The mice receiving Compound I-19 IP in study “SUPER-20201021-MPK” as M7, M8 and M9.

All animal procedures mentioned in this report were approved by the Institutional Animal Care and Use Committee of WuXi AppTec, Inc. New Jersey site and were in accordance with all applicable federal and local regulations.

### Feeding Condition

The animals were fasted overnight prior to dosing. Food was returned 2 hours post-dose. Animals had access to water freely the entire time throughout the study.

### Test Article Administration

The animals were weighed immediately prior to dosing and the dose volume was calculated individually based the obtained body weight and the nominal dose volume. The animals had an average body weight of 27.4 g, ranging from 24.8 to 29.7 g. The test articles in study “SUPER-20190517-MPK” were administered orally by gavage, and by IP injection in study “SUPER-20201021-MPK”. The nominal dose volume was 10 mL/kg in both studies.

### Clinical Observation

Cage side observation was performed before and after dosing as well as at each scheduled sample collection time. The observation included appearance, posture, behavior, morbidity and mortality. Abnormal findings, if any, are required to be recorded in the study record and communicated with study director, veterinarian, and Supernus if necessary.

### Blood Collection and Plasma Preparation

Approximately 40 μL blood was collected at each scheduled time point from saphenous veins. The actual sample collection time was within 1 min. of the nominal time for collections prior to or at 1 hour, and within 5% of the nominal values for collections post 1 hour.

The blood samples were placed in pre-labeled micro-centrifuge tubes containing K2EDTA as anticoagulant and kept on ice before they were processed to plasma within 30 minutes of collection by centrifugation at 4° C., 3000 g for 5 minutes. The plasma samples were stored at −70±10° C. until LC-MS/MS analysis.

### Bioanalytical Assay

The concentrations of Compound I-19, Compound A, and Compound B in the plasma were determined using qualified LC-MS/MS methods.

### Pharmacokinetic Analysis

The data of plasma concentration versus time of each animal was analyzed using Phoenix WinNonlin 6.3 to determine the PK properties of Compound I-19, Compound A, and Compound B. The non-compartmental model and the linear/log trapezoidal method were applied to the PK calculation.

Plasma concentrations below the LLOQ prior to Tₘₐₓ were set to zero, and those after Tₘₐₓ were excluded from PK calculation. The nominal dose and the nominal sample collection time were used for the calculation. The values of plasma concentrations and PK parameters are reported to three significant figures. The average values of each treatment group are presented as mean±SD.

### Results and Discussion

### Clinical Observation

Compound A and Compound I-19 at the administered dosages were well tolerated by all animals. No adverse effects were observed throughout the study.

### Dose Accuracy Verification

The concentrations of Compound A and Compound I-19 in the dosing solutions were determined by LC-MS/MS (Table 25). The measured concentrations of Compound A and Compound I-19 in the PO and IP formulations were 0.470, 0.507 and 0.541 mg/mL, respectively. The data showed that the accuracy of the Compound A formulation was 94% and that of the PO formulation of Compound I-19 was 101%, well within the acceptable range of ±20% of its nominal concentration value. However, the accuracy of the IP formulation of Compound I-19 was 133%, greater than the acceptable range as stated in the study protocol.

TABLE 25
Concentrations of Compound A and Compound I-19
in the dose solutions

| | | Sample Conc. | | Mean | Nominal | |
|---|---|---|---|---|---|---|
| | Dose | (mg/mL) | | Conc. | Conc. | Accuracy ^{a} |
| TA | Route | Sample 1 | Sample 2 | (mg/mL) | (mg/mL) | (%) |
| Compound | PO | 0.469 | 0.471 | 0.470 | 0.500 | 94 |
| A | | | | | | |
| Compound | PO | 0.495 | 0.520 | 0.507 | 0.500 | 101 |
| I-19 | | | | | | |
| Compound | IP | 0.549 | 0.532 | 0.541 | 0.406 | 133 |
| I-19 | | | | | | |

^{a} Accuracy (%) = Mean Concentration (mg/mL)/Nominal Concentration (mg/mL) × 100

### Pharmacokinetics of Compound I-19, Compound A and Compound B

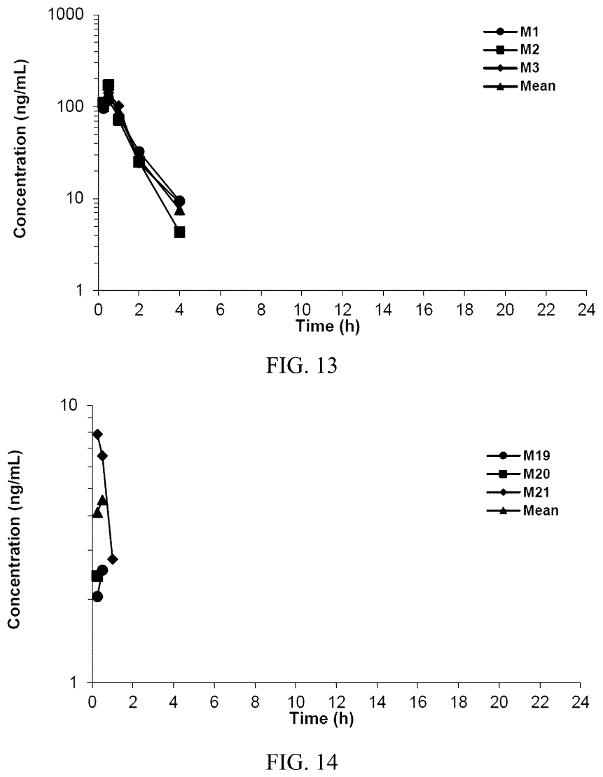

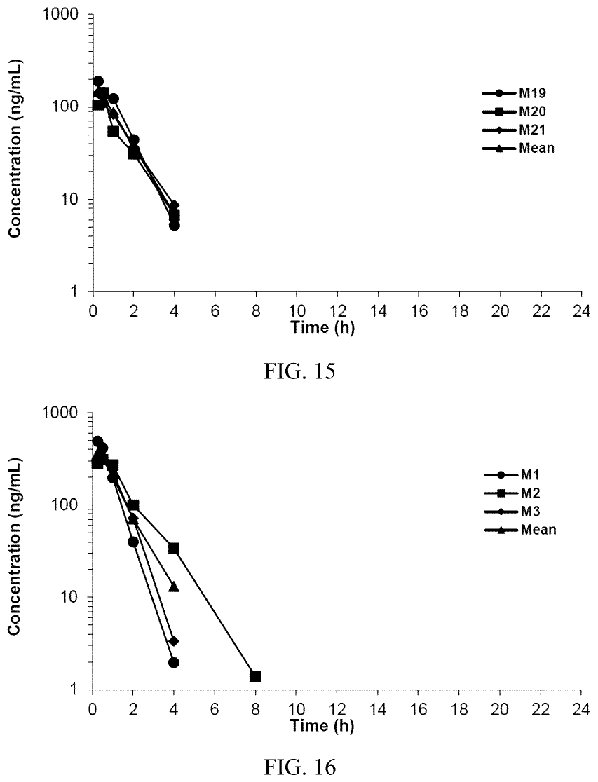

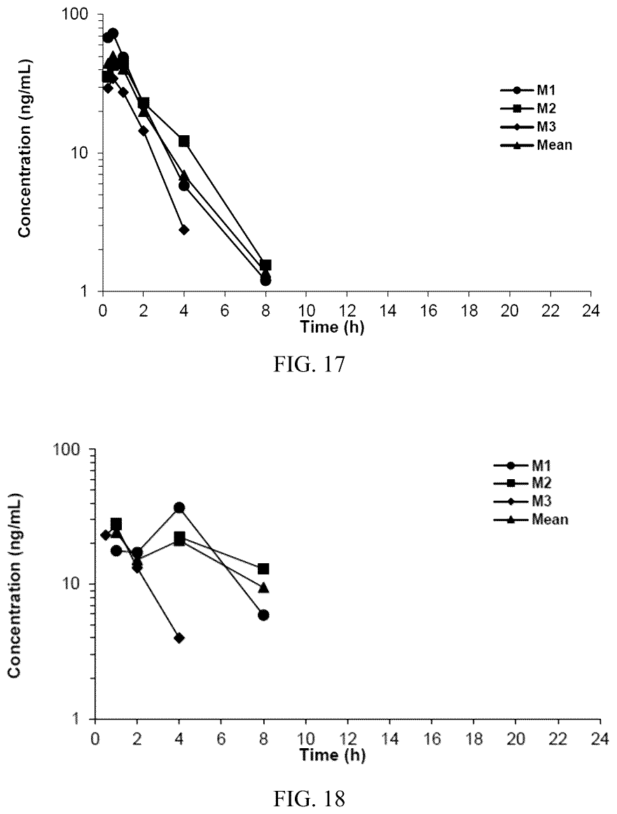

The individual and mean plasma concentrations of Compound A, Compound I-19 and Compound B following PO and IP administrations are tabulated in Tables 26 to 31. The corresponding PK parameters are summarized in Tables 32-36. The plasma concentration-time profiles are shown in FIGS. 13-18.

TABLE 26
Individual and mean plasma concentrations (ng/mL) of Compound A following
single PO administration to male CD-1 mice at 5.102 mg/kg

| Time (h) | M1 | M2 | M3 | Mean | SD | CV (%) |
|---|---|---|---|---|---|---|
| 0.250 | 95.5 | 110 | 95.6 | 100 | 8.34 | 8.31 |
| 0.500 | 117 | 172 | 141 | 143 | 27.6 | 19.2 |
| 1.00 | 77.8 | 71.7 | 102 | 83.8 | 16.0 | 19.1 |
| 2.00 | 32.3 | 25.2 | 24.4 | 27.3 | 4.35 | 15.9 |
| 4.00 | 9.42 | 4.31 | 8.99 | 7.57 | 2.83 | 37.4 |
| 8.00 | BQL | BQL | BQL | ND | ND | ND |
| 24.0 | BQL | BQL | BQL | ND | ND | ND |

BQL: below the quantifiable limit;
ND: not determine due to insufficient number of values

TABLE 27
Individual and mean plasma concentrations (ng/mL) of Compound I-19
following single PO administration to male CD-1 mice at 6.654 mg/kg

| Time (h) | M19 | M20 | M21 | Mean | SD | CV (%) |
|---|---|---|---|---|---|---|
| 0.250 | 2.04 | 2.42 | 7.84 | 4.10 | 3.24 | 79.1 |
| 0.500 | 2.54 | BQL | 6.56 | 4.55 | ND | ND |
| 1.00 | BQL | BQL | 2.78 | ND | ND | ND |
| 2.00 | BQL | BQL | BQL | ND | ND | ND |
| 4.00 | BQL | BQL | BQL | ND | ND | ND |
| 8.00 | BQL | BQL | BQL | ND | ND | ND |
| 24.0 | BQL | BQL | BQL | ND | ND | ND |

BQL: below the quantifiable limit;
ND: not determine due to insufficient number of values

TABLE 28
Individual and mean plasma concentrations (ng/mL) of Compound A following
single PO administration of Compound I-19 to male CD-1 mice at 6.645 mg/kg

| Time (h) | M19 | M20 | M21 | Mean | SD | CV (%) |
|---|---|---|---|---|---|---|
| 0.250 | 189 | 105 | 141 | 145 | 42.1 | 29.1 |
| 0.500 | BQL | 143 | 108 | 126 | ND | ND |
| 1.00 | 123 | 54.4 | 83.2 | 86.9 | 34.4 | 39.7 |
| 2.00 | 44.1 | 31.3 | 36.7 | 37.4 | 6.43 | 17.2 |
| 4.00 | 5.24 | 6.75 | 8.65 | 6.88 | 1.71 | 24.8 |
| 8.00 | BQL | BQL | BQL | ND | ND | ND |
| 24.0 | BQL | BQL | BQL | ND | ND | ND |

BQL below the quantifiable limit;
ND: not determine due to insufficient number of values

TABLE 29
Individual and mean plasma concentrations (ng/mL) of Compound I-19 following
single IP administration to male CD-1 mice at 4.061 mg/kg

| Time (h) | M1 | M2 | M3 | Mean | SD | CV (%) |
|---|---|---|---|---|---|---|
| 0.250 | 489 | 280 | 308 | 359 | 113 | 31.6 |
| 0.500 | 415 | 311 | 318 | 348 | 58.1 | 16.7 |
| 1.00 | 196 | 269 | 201 | 222 | 40.8 | 18.4 |
| 2.00 | 39.7 | 100 | 71.6 | 70.4 | 30.2 | 42.8 |
| 4.00 | 1.96 | 34.0 | 3.35 | 13.1 | 18.1 | 138 |
| 8.00 | BQL | 1.39 | BQL | ND | ND | ND |
| 24.0 | BQL | BQL | BQL | ND | ND | ND |

BQL: below the quantifiable limit;
ND: not determine due to insufficient number of values

TABLE 30
Individual and mean plasma concentrations (ng/mL) of Compound A following
single IP administration of Compound I-19 to male CD-1 mice at 4.061 mg/kg

| Time (h) | M1 | M2 | M3 | Mean | SD | CV (%) |
|---|---|---|---|---|---|---|
| 0.250 | 68.1 | 35.6 | 29.3 | 44.3 | 20.8 | 47.0 |
| 0.500 | 73.0 | 42.9 | 34.5 | 50.1 | 20.2 | 40.4 |
| 1.00 | 49.2 | 44.1 | 27.4 | 40.2 | 11.4 | 28.3 |
| 2.00 | 22.3 | 23.1 | 14.4 | 19.9 | 4.81 | 24.1 |
| 4.00 | 5.80 | 12.2 | 2.78 | 6.93 | 4.81 | 69.4 |
| 8.00 | 1.20 | 1.55 | BQL | 1.38 | ND | ND |
| 24.0 | BQL | BQL | BQL | ND | ND | ND |

BQL: below the quantifiable limit;
ND: not determine due to insufficient number of values

TABLE 31
Individual and mean plasma concentrations (ng/mL) of Compound B following
single IP administration of Compound I-19 to male CD-1 mice at 4.061 mg/kg

| Time (h) | M1 | M2 | M3 | Mean | SD | CV (%) |
|---|---|---|---|---|---|---|
| 0.250 | BQL | BQL | BQL | ND | ND | ND |
| 0.500 | BQL | BQL | 23.1 | ND | ND | ND |
| 1.00 | 17.7 | 28.0 | 27.1 | 24.3 | 5.70 | 23.5 |
| 2.00 | 17.2 | BQL | 13.2 | 15.2 | ND | ND |
| 4.00 | 36.9 | 22.4 | 3.98 | 21.1 | 16.5 | 78.2 |
| 8.00 | 5.89 | 13.0 | BQL | 9.45 | ND | ND |
| 24.0 | BQL | BQL | BQL | ND | ND | ND |

BQL: below the quantifiable limit;
ND: not determine due to insufficient number of values

TABLE 32
Individual and mean PK parameters of Compound I-19 following
single PO administration to male CD-1 mice at 6.645 mg/kg

| Time (h) | M19 | M20 | M21 | Mean | SD | CV (%) |
|---|---|---|---|---|---|---|
| Rsq_adj | ND | ND | 0.941 | ND | ND | ND |
| Cₘₐₓ (ng/mL) | 2.54 | 2.42 | 7.84 | 4.27 | 3.10 | 72.5 |
| Tₘₐₓ (h) | 0.500 | 0.250 | 0.250 | 0.333 | 0.144 | 43.3 |
| T_{1/2} (h) | ND | ND | 0.485 | ND | ND | ND |
| Tₗₐₛₜ (h) | 0.500 | 0.250 | 1.00 | 0.583 | 0.382 | 65.5 |
| AUC₀₋ₗₐₛₜ (ng · h/mL) | 0.828 | 0.303 | 4.98 | 2.04 | 2.56 | 126 |
| AUC_{0-inf} (ng · h/mL) | ND | ND | 6.92 | ND | ND | ND |
| MRT₀₋ₗₐₛₜ (h) | 0.346 | 0.250 | 0.499 | 0.365 | 0.126 | 34.4 |
| MRT_{0-inf} (h) | ND | ND | 0.836 | ND | ND | ND |

ND: not determine due to insufficient number of values

TABLE 33
Individual and mean PK parameters of Compound A following single PO
administration of Compound I-19 to male CD-1 mice at 6.645 mg/kg

| Time (h) | M19 | M20 | M21 | Mean | SD | CV (%) |
|---|---|---|---|---|---|---|
| Rsq_adj | 1.000 | 0.989 | 0.998 | 0.996 | 0.00592 | 0.595 |
| Cₘₐₓ (ng/mL) | 189 | 143 | 141 | 158 | 27.2 | 17.2 |
| Tₘₐₓ (h) | 0.250 | 0.500 | 0.250 | 0.333 | 0.144 | 43.3 |
| T_{1/2} (h) | 0.658 | 0.982 | 0.924 | 0.855 | 0.173 | 20.2 |
| Tₗₐₛₜ (h) | 4.00 | 4.00 | 4.00 | 4.00 | 0 | 0 |
| AUC₀₋ₗₐₛₜ (ng · h/mL) | 252 | 164 | 192 | 203 | 45.2 | 22.3 |
| AUC_{0-inf} (ng · h/mL) | 257 | 173 | 203 | 211 | 42.5 | 20.1 |
| MRT₀₋ₗₐₛₜ (h) | 1.11 | 1.20 | 1.25 | 1.19 | 0.0690 | 5.80 |
| MRT_{0-inf} (h) | 1.19 | 1.44 | 1.48 | 1.37 | 0.158 | 11.5 |

TABLE 34
Individual and mean PK parameters of Compound I-19 following
single IP administration to male CD-1 mice at 4.061 mg/kg

| Time (h) | M1 | M2 | M3 | Mean | SD | CV (%) |
|---|---|---|---|---|---|---|
| Rsq_adj | 1.000 | 0.992 | 0.984 | 0.992 | 0.00773 | 0.779 |
| Cₘₐₓ (ng/mL) | 489 | 311 | 318 | 373 | 101 | 27.1 |
| Tₘₐₓ (h) | 0.250 | 0.500 | 0.500 | 0.417 | 0.144 | 34.6 |
| T_{1/2} (h) | 0.452 | 0.942 | 0.499 | 0.631 | 0.270 | 42.8 |
| Tₗₐₛₜ (h) | 4.00 | 8.00 | 4.00 | 5.33 | 2.31 | 43.3 |
| AUC₀₋ₗₐₛₜ (ng · h/mL) | 443 | 588 | 414 | 482 | 92.9 | 19.3 |
| AUC_{0-inf} (ng · h/mL) | 444 | 589 | 417 | 483 | 92.9 | 19.2 |
| MRT₀₋ₗₐₛₜ (h) | 0.815 | 1.60 | 1.02 | 1.15 | 0.407 | 35.5 |
| MRT_{0-inf} (h) | 0.826 | 1.62 | 1.04 | 1.16 | 0.413 | 35.4 |

TABLE 35
Individual and mean PK parameters of Compound A following single IP
administration of Compound I-19 to male CD-1 mice at 4.061 mg/kg

| Time (h) | M1 | M2 | M3 | Mean | SD | CV (%) |
|---|---|---|---|---|---|---|
| Rsq_adj | 0.962 | 0.978 | 0.993 | 0.978 | 0.0156 | 1.59 |
| Cₘₐₓ (ng/mL) | 73.0 | 44.1 | 34.5 | 50.5 | 20.0 | 39.7 |
| Tₘₐₓ (h) | 0.500 | 1.00 | 0.500 | 0.667 | 0.289 | 43.3 |
| T_{1/2} (h) | 1.33 | 1.51 | 0.899 | 1.25 | 0.314 | 25.2 |
| Tₗₐₛₜ (h) | 8.00 | 8.00 | 4.00 | 6.67 | 2.31 | 34.6 |
| AUC₀₋ₗₐₛₜ (ng · h/mL) | 126 | 123 | 61.4 | 104 | 36.7 | 35.4 |
| AUC_{0-inf} (ng · h/mL) | 129 | 127 | 65.0 | 107 | 36.2 | 33.9 |
| MRT₀₋ₗₐₛₜ (h) | 1.68 | 2.25 | 1.36 | 1.76 | 0.454 | 25.8 |
| MRT_{0-inf} (h) | 1.82 | 2.46 | 1.58 | 1.95 | 0.458 | 23.4 |

TABLE 36
Individual and mean PK parameters of Compound B following single IP
administration of Compound I-19 to male CD-1 mice at 4.061 mg/kg

| Time (h) | M1 | M2 | M3 | Mean | SD | CV (%) |
|---|---|---|---|---|---|---|
| Rsq_adj | ND | 0.953 | 0.996 | 0.974 | ND | ND |
| Cₘₐₓ (ng/mL) | 36.9 | 28.0 | 27.1 | 30.7 | 5.42 | 17.7 |
| Tₘₐₓ (h) | 4.00 | 1.00 | 1.00 | 2.00 | 1.73 | 86.6 |
| T_{1/2} (h) | ND | 6.24 | 1.09 | 3.67 | ND | ND |
| Tₗₐₛₜ (h) | 8.00 | 8.00 | 4.00 | 6.67 | 2.31 | 34.6 |
| AUC₀₋ₗₐₛₜ (ng · h/mL) | 148 | 158 | 53.0 | 120 | 58.1 | 48.5 |
| AUC_{0-inf} (ng · h/mL) | ND | 275 | 59.3 | 167 | ND | ND |
| MRT₀₋ₗₐₛₜ (h) | 3.94 | 3.79 | 1.58 | 3.10 | 1.32 | 42.7 |
| MRT_{0-inf} (h) | ND | 9.41 | 2.00 | 5.70 | ND | ND |

ND: not determine due to insufficient number of values

Oral administration of Compound A at 5.102 mg/kg yielded a Cₘₐₓ of 143±27.6 ng/mL at 0.5±0 hour post-dose and an AUC0-last of 179±6.91 ng·h/mL. Compound A had a T_{1/2} of 0.885±0.13 hour and a MRT₀₋ₗₐₛₜ of 1.18±0.113 hour.

Oral administration of Compound I-19 at 6.645 mg/kg yielded a Cₘₐₓ of 4.27±3.1 ng/mL at 0.333±0.144 hour post-dose and an AUC0-last of 2.04±2.56 ng·h/mL. Compound I-19 had MRT₀₋ₗₐₛₜ of 0.365±0.126 hour. The T_{1/2} of Compound I-19 following PO administration was not determined due to insufficient number of concentration values. On the other hand, the Cₘₐₓ of Compound A following PO administration of Compound I-19 measured 158±27.2 ng/mL. The Tₘₐₓ of Compound A was 0.333±0.144 hour post-dose, the same as that of Compound I-19, and the AUC₀₋ₗₐₛₜ was 203±45.2 ng·h/mL. Compound A had a T_{1/2} of 0.855±0.173 hour and a MRT₀₋ₗₐₛₜ of 1.19±0.069 hour following PO administration of Compound I-19.

Intraperitoneal administration of Compound I-19 at 4.061 mg/kg yielded a Cₘₐₓ of 373±101 ng/mL with the Tₘₐₓ at 0.417±0.144 hour post-dose and an AUC₀₋ₗₐₛₜ of 482±92.9 ng·h/mL. The T_{1/2} and MRT₀₋ₗₐₛₜ of Compound I-19 following IP administration was 0.631±0.27 hour and 1.15±0.407 hour, respectively. The Cₘₐₓ and AUC0-last of Compound A following IP administration of Compound I-19 were 50.5±20 ng/mL and 104±36.7 ng·h/mL, respectively. The Tₘₐₓ, T_{1/2} and MRT₀₋ₗₐₛₜ were 0.667±0.289, 1.25±0.314 and 1.76±0.454 hours, respectively.

The Cₘₐₓ and AUC₀₋ₗₐₛₜ of Compound B following IP administration of Compound I-19 were 30.7±5.42 ng/mL and 120±58.1 ng·h/mL, respectively. The Tₘₐₓ, T_{1/2} and MRT₀₋ₗₐₛₜ of Compound B following IP administration were averaged at 2.00, 3.67 and 3.10 hours, respectively.

The data indicated that the plasma PK profiles of Compound A were comparable following oral administrations of the derivative Compound I-19 or its metabolite Compound A at equivalent dose of the free base. By the comparison of Cₘₐₓ and AUC₀₋ₗₐₛₜ of Compound I-19 between IP and PO administrations, the data suggested that IP administration of Compound I-19 had a significantly greater relative bioavailability compared to PO administration. By the comparison of Compound A to Compound I-19 ratios between PO and IP administrations, the data indicated that the conversion rate of Compound I-19 to Compound A following IP administration was not as efficient as that of PO administration. The Tₘₐₓ and T_{1/2} data of both Compound I-19 and Compound A suggested that both compounds exhibited rapid absorbance and fast clearance/elimination.

Compound I-19 at the administered dosage were well tolerated. No adverse effects were evident throughout the study.

### Conclusion

The data indicated that the plasma PK profiles of Compound A were comparable following equivalent oral doses of Compound I-19 or Compound A directly. By the comparison of Cₘₐₓ and AUC₀₋ₗₐₛₜ between IP and PO administration, the data suggested that IP administration of Compound I-19 had a significantly greater relative bioavailability compared to PO administration. However, the ratios of Cₘₐₓ and AUC₀₋ₗₐₛₜ between Compound A and Compound I-19 indicated that the conversion of Compound I-19 to Compound A following IP administration was not as efficient as that of PO administration. The Tₘₐₓ and T_{1/2} data of both Compound I-19 and Compound A suggested that both compounds exhibited rapid absorption and fast clearance/elimination.

Compound A and Compound I-19 at the administered dosages were well tolerated by the animals. No adverse effects were evident throughout the study.

### Example 7: PHARMACOKINETICS OF Compound I-4, Compound I-19 AND Compound a Following Single Oral and Intraperitoneal Administration Of Compound I-4 and Compound I-19 to Male Wister Han Rats

The study was carried out to evaluate the PK properties of Compound I-4 and Compound I-19, and their associated metabolites Compound A and Compound B following single PO and IP administration of Compound I-4 or Compound I-19 to male Wistar Han rats.

### Materials and Methods

### Test Article

| | | Molecular | Formula | | Storage |
|---|---|---|---|---|---|
| Test Article | Batch | Weight | Weight | Purity | Condition |
| Compound I-4 | 115-121 | 342.82 | 342.82 | 97.4 | 4° C. |
| Compound I-19 | 115-108 | 370.83 | 370.83 | 96.2 | 4° C. |

### Study Design

Twelve male Wistar Han rats were divided into 4 groups, n=3 per group. Groups 1 and 2 were treated with Compound I-4 at 3.708 mg/kg via PO and IP administrations, respectively. Groups 3 and 4 were given Compound I-19 at 4.061 mg/kg via PO and IP administrations, respectively. The doses of both Compound I-4 and Compound I-19 were equivalent to 3 mg/kg of free base Compound A. Blood samples were collected from each animal at 0.25, 0.5, 1, 2, 4, 8 and 24 hours post-dose, and then centrifuged to extract plasma for the determination of the concentrations of Compound I-4, Compound I-19, Compound A, and the common metabolite Compound B by (LC-MS/MS and the data of plasma concentration versus time from each animal was analyzed based on the non-compartmental analysis model using Phoenix WinNonlin 6.3 to calculate the PK parameters. Details of the compound administration and blood sample collection are summarized in the tables below.

### Compound Administration Details

| | | Nominal | Verified | Volume | | |
|---|---|---|---|---|---|---|
| Test | | Dose | Dose | Dose | | Dosing |
| Article | n | (mg/kg) | (mg/kg) | (mL/kg) | Vehicle | Route |
| Compound | 3 | 3.708 | 4.41 | 10 | 4% DMSO, | PO |
| I-4 | | | | | 30% | |
| Compound | 3 | 3.708 | 4.41 | 10 | PEG400, | IP |
| I-4 | | | | | 66% | |
| Compound | 3 | 4.061 | 5.38 | 10 | HP-β-CD | PO |
| I-19 | | | | | (30% | |
| Compound | 3 | 4.061 | 5.38 | 10 | in H₂O) | IP |
| I-19 | | | | | | |

### Sample Collection Details

| | Dosing | | Sampling schedule |
|---|---|---|---|
| Test Article | Route | Matrix | (h post dosing) |
| Compound I-4 | PO | Plasma | 0.25, 0.5, 1, 2, 4, 8, 24 |
| Compound I-4 | IP | Plasma | 0.25, 0.5, 1, 2, 4, 8, 24 |
| Compound I-19 | PO | Plasma | 0.25, 0.5, 1, 2, 4, 8, 24 |
| Compound I-19 | IP | Plasma | 0.25, 0.5, 1, 2, 4, 8, 24 |

Anticoagulant: K₂EDTA

### Test Article Formulation

### Formulation Preparation

Dose solutions were prepared freshly on the day of study prior to dosing. Both the PO and IP formulations were prepared using 4% DMSO, 30% PEG 400, and 66% HP-β-CD (30% in H2O, w/v) as vehicle. The target concentration of Compound I-4 for both the PO and IP administrations was 0.371 mg/mL and the target concentration of Compound I-19 for both routes was 0.406 mg/mL, both were equivalent to 0.3 mg/mL of free base Compound A. The nominal dose volume was 10 mL/kg for both compounds and both dose routes to achieve a target dose of 3 mg/kg of free base Compound A. Both formulations appeared as clear solutions when dosed.

### Formulation Verification

Two 50 μL aliquots of each dose solution were set aside at the completion of formulation preparation. The concentrations of Compound I-4 and Compound I-19 in the dosing solution were determined by LC-MS/MS. All formulation samples were analyzed in duplicate and quantified against a calibration curve consisting of six concentrations of the analyte.

### In-Life Procedures

### Animal Husbandry

The animals used in studies were sourced from Hilltop Lab Animals, Inc. (Scottdale, PA 15683). The animals were fed certified pellet diet (Certified Rodent Diet #5002, LabDiet) and allowed access to water (reverse osmosis) ad libitum. The animals were acclimated to the facility for 5 days prior to the commencement of the study.

All animals were confirmed healthy prior to being assigned to studies. Each animal was given a unique identification number, which was marked on the tail and written on the cage card as well. Animals assigned to groups 1-4 were identified as R1 through R12 in an ascending manner.

All the animal procedures performed in this report were approved by the Institutional Animal Care and Use Committee of WuXi AppTec, Inc. New Jersey site and were in accordance with all applicable federal and local regulations.

### Feeding Condition

The animals were fasted overnight prior to dosing. Food was returned 2 hours post-dose. Animals had access to water freely the entire time throughout the study.

### Test Article Administration

The animals were weighed immediately prior to dosing and the dose volume was calculated individually based the obtained body weight and the nominal dose volume. The animals had an average body weight of 253 g, ranging 232-278 g. The test articles were administered either orally by gavage, or intraperitoneally by injection. The nominal dose volume was 10 mL/kg for both compounds and both routes.

### Clinical Observation

Cage side observation was performed before and after dosing as well as at each scheduled sample collection. The observation included appearance, posture, behavior, morbidity and mortality. Abnormal findings, if any, were recorded in the study notebook and communicated with study director, veterinarian, and Supernus if necessary.

### Blood Collection and Plasma Preparation

Approximately 100 μL blood was collected at each scheduled time point from jugular veins. The actual sample collection time was within 1 min. of the nominal time for collections prior to or at 1 hour, and within 5% of the nominal time for collections post 1 hour.

The blood samples were placed in pre-labeled micro-centrifuge tubes containing K2EDTA as anticoagulant and kept on ice, and then were processed to plasma within 30 minutes of collection by centrifugation at 4° C., 3000 g for 5 minutes, and then stored at −70±10° C. until analysis by LC-MS/MS.

### Bioanalytical Assay

The concentrations of Compound I-4, Compound I-19, Compound A and Compound B in the plasma were determined using qualified LC-MS/MS method.

### Pharmacokinetic Analysis

The data of plasma concentration versus time of each animal was analyzed using Phoenix WinNonlin 6.3 to determine the PK properties of Compound I-4, Compound I-19, Compound A and Compound B. The non-compartmental model and the linear/log trapezoidal method were applied to PK calculation.

Plasma concentrations below the LLOQ prior to Tₘₐₓ were set to zero, and those after Tₘₐₓ were excluded from PK calculation. The nominal dose and the nominal sample collection time were used for the calculation. The values of plasma concentrations and PK parameters are reported to three significant figures. The average values of each treatment group are presented as mean±SD.

### Results and Discussion

### Clinical Observation

Compound I-4 and Compound I-19 at the administered dosages was well tolerated by all animals. No adverse effects were observed throughout the study.

### Dose Accuracy Verification

The concentrations of Compound I-4 and Compound I-19 in the dosing solutions were determined by LC-MS/MS. The measured concentrations (Table 37) of both compounds in the formulations were 0.441 and 0.538 mg/kg, respectively. The data showed that the accuracy of the Compound I-4 formulation was 119%, within the acceptable range of ±20% of its nominal concentration value. However, the accuracy of the Compound I-19 formulation was at 132%, greater than the acceptable range as stated in the study protocol.

TABLE 37
Concentrations of Compound I-4 and Compound I-19 in the dose solutions

| | | | | Mean | Nominal | |
|---|---|---|---|---|---|---|
| Test | Dose | Sample Conc. (mg/mL) | | Conc. | Conc. | Accuracy ^{a} |
| Article | Route | Sample 1 | Sample 2 | (mg/mL) | (mg/mL) | (%) |
| Compound | PO/IP | 0.425 | 0.457 | 0.441 | 0.371 | 119 |
| I-4 | | | | | | |
| Compound | PO/IP | 0.539 | 0.536 | 0.538 | 0.406 | 132 |
| I-19 | | | | | | |

^{a} Accuracy (%) = Mean Concentration (mg/mL)/Nominal Concentration (mg/mL) × 100

### Pharmacokinetics of Compound I-4, Compound I-19 and Compound A

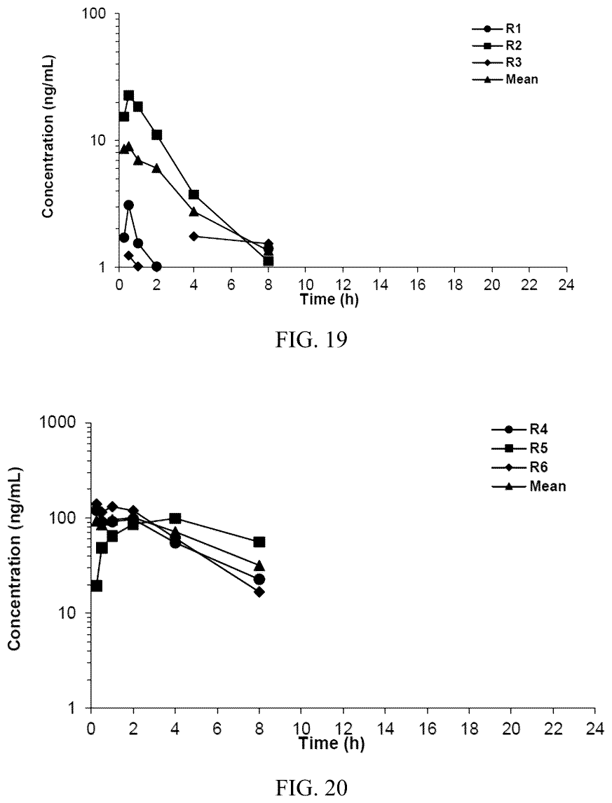

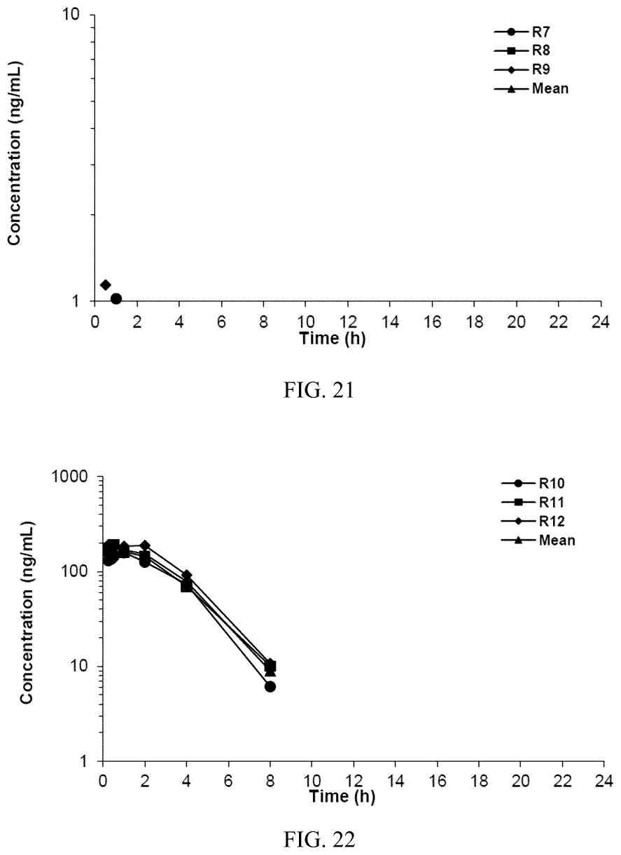

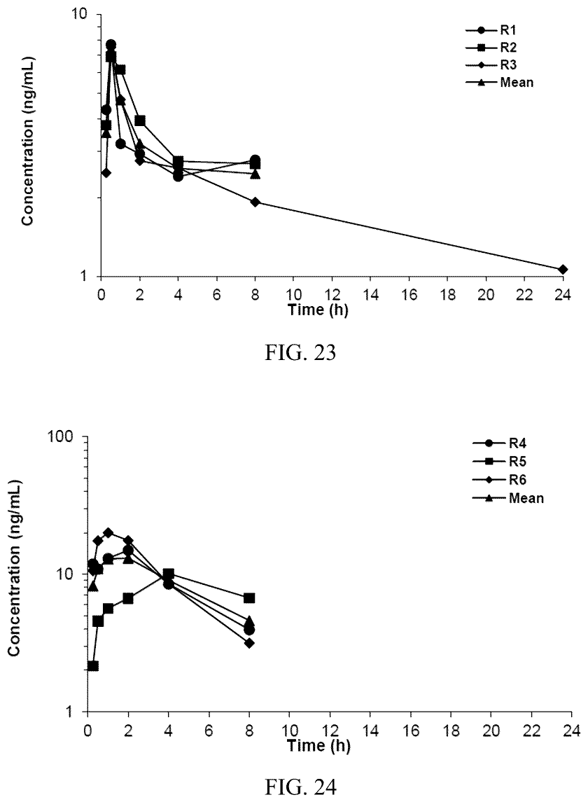

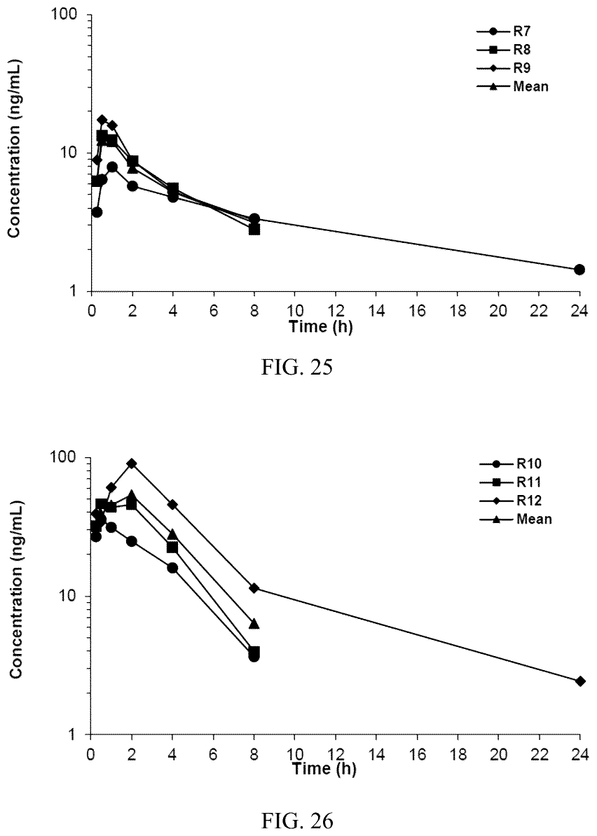

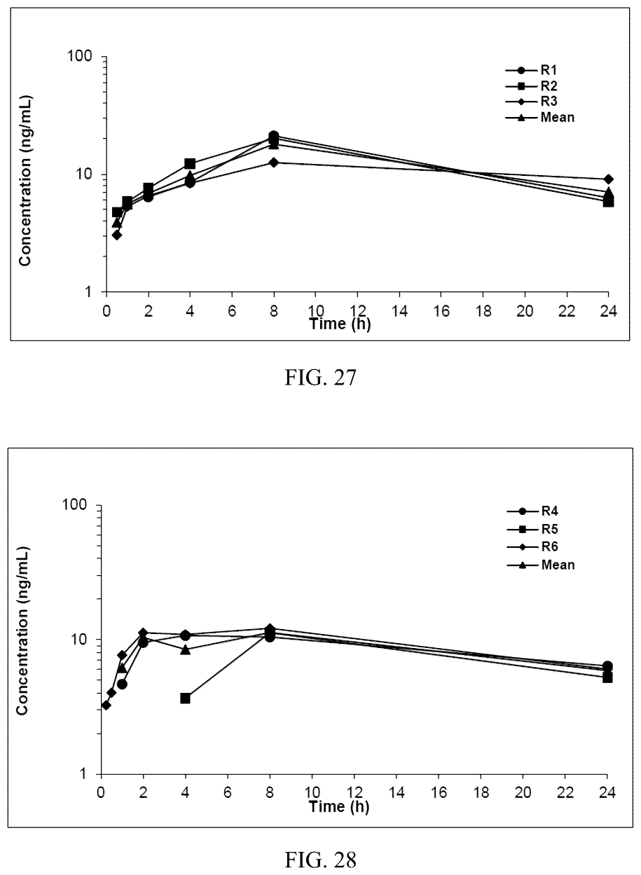

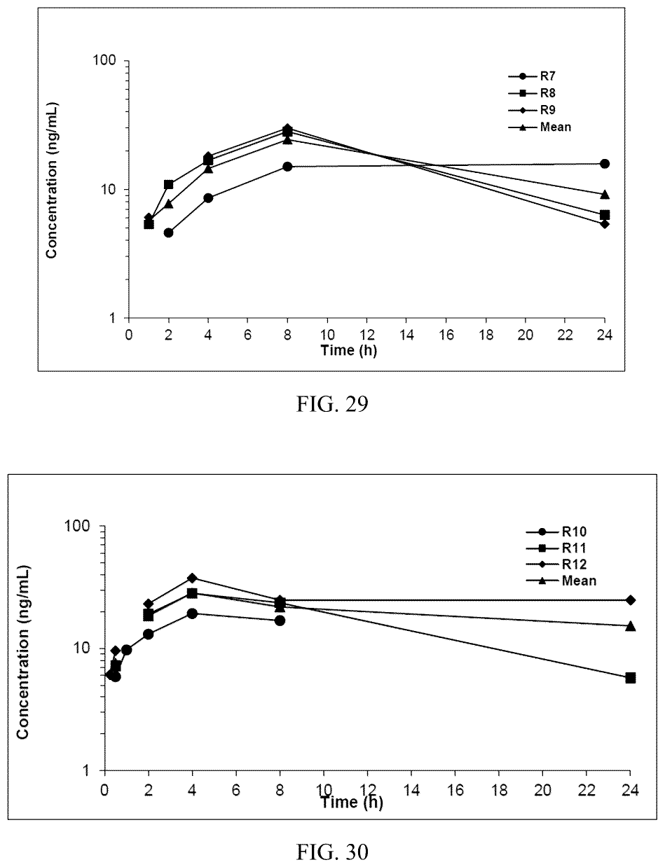

The individual and mean plasma concentrations of Compound I-4, Compound I-19, Compound A and Compound B following PO and IP administrations are tabulated in Tables 38 to 49. The corresponding PK parameters are summarized in Tables 50 to 61, respectively. The plasma concentration-time profiles of Compound I-4, Compound A and Compound B following PO and IP administrations are shown in FIGS. 19 to 30, respectively.

TABLE 38
Individual and mean plasma concentrations (ng/mL) of Compound I-4
following single PO administration to male Wistar Han rats at 3.708 mg/kg

| Time (h) | R1 | R2 | R3 | Mean | SD | CV (%) |
|---|---|---|---|---|---|---|
| 0.250 | 1.71 | 15.4 | BQL | 8.56 | ND | ND |
| 0.500 | 3.09 | 22.7 | 1.23 | 9.01 | 11.9 | 132 |
| 1.00 | 1.54 | 18.4 | 1.01 | 6.98 | 9.89 | 142 |
| 2.00 | 1.01 | 11.1 | BQL | 6.06 | ND | ND |
| 4.00 | BQL | 3.75 | 1.75 | 2.75 | ND | ND |
| 8.00 | 1.40 | 1.11 | 1.53 | 1.35 | 0.215 | 16.0 |
| 24.0 | BQL | BQL | BQL | ND | ND | ND |

BQL: below the quantifiable limit;
ND: not determine due to insufficient number of values

TABLE 39
Individual and mean plasma concentrations (ng/ml) of Compound I-4 following
single IP administration to male Wistar Han rats at 3.708 mg/kg

| Time (h) | R4 | R5 | R6 | Mean | SD | CV (%) |
|---|---|---|---|---|---|---|
| 0.250 | 120 | 19.3 | 140 | 93.1 | 64.7 | 69.5 |
| 0.500 | 90.3 | 48.4 | 115 | 84.6 | 33.7 | 39.8 |
| 1.00 | 91.2 | 64.2 | 131 | 95.5 | 33.6 | 35.2 |
| 2.00 | 96.7 | 85.7 | 119 | 100 | 17.0 | 16.9 |
| 4.00 | 54.6 | 98.6 | 61.1 | 71.4 | 23.8 | 33.2 |
| 8.00 | 22.6 | 55.9 | 16.7 | 31.7 | 21.1 | 66.6 |
| 24.0 | BQL | BQL | BQL | ND | ND | ND |

BQL: below the quantifiable limit;
ND: not determine due to insufficient number of values

TABLE 40
Individual and mean plasma concentrations (ng/ml) of Compound I-19
following single PO administration to male Wistar Han rats at 4.061 mg/kg

| Time (h) | R7 | R8 | R9 | Mean | SD | CV (%) |
|---|---|---|---|---|---|---|
| 0.250 | BQL | BQL | BQL | ND | ND | ND |
| 0.500 | BQL | BQL | 1.14 | ND | ND | ND |
| 1.00 | 1.02 | BQL | BQL | ND | ND | ND |
| 2.00 | BQL | BQL | BQL | ND | ND | ND |
| 4.00 | BQL | BQL | BQL | ND | ND | ND |
| 8.00 | BQL | BQL | BQL | ND | ND | ND |
| 24.0 | BQL | BQL | BQL | ND | ND | ND |

BQL: below the quantifiable limit;
ND: not determine due to insufficient number of values

TABLE 41
Individual and mean plasma concentrations (ng/ml) of Compound I-19
following single IP administration to male Wistar Han rats at 4.061 mg/kg

| Time (h) | R10 | R11 | R12 | Mean | SD | CV (%) |
|---|---|---|---|---|---|---|
| 0.250 | 130 | 175 | 192 | 166 | 32.0 | 19.3 |
| 0.500 | 150 | 193 | 136 | 160 | 29.7 | 18.6 |
| 1.00 | 158 | 161 | 184 | 168 | 14.2 | 8.48 |
| 2.00 | 126 | 144 | 188 | 153 | 31.9 | 20.9 |
| 4.00 | 73.3 | 69.9 | 91.6 | 78.3 | 11.7 | 14.9 |
| 8.00 | 6.11 | 10.1 | 10.7 | 8.97 | 2.49 | 27.8 |
| 24.0 | BQL | BQL | BQL | ND | ND | ND |

BQL: below the quantifiable limit;
ND: not determine due to insufficient number of values

TABLE 42
Individual and mean plasma concentrations (ng/ml) of Compound A following
single PO administration of Compound I-4 to male Wistar Han rats at 3.708 mg/kg

| Time (h) | R1 | R2 | R3 | Mean | SD | CV (%) |
|---|---|---|---|---|---|---|
| 0.250 | 4.32 | 3.78 | 2.48 | 3.53 | 0.946 | 26.8 |
| 0.500 | 7.67 | 6.90 | 7.47 | 7.35 | 0.400 | 5.44 |
| 1.00 | 3.20 | 6.15 | 4.73 | 4.69 | 1.48 | 31.4 |
| 2.00 | 2.93 | 3.93 | 2.76 | 3.21 | 0.632 | 19.7 |
| 4.00 | 2.40 | 2.75 | 2.59 | 2.58 | 0.175 | 6.79 |
| 8.00 | 2.78 | 2.69 | 1.92 | 2.46 | 0.473 | 19.2 |
| 24.0 | BQL | BQL | 1.06 | ND | ND | ND |

BQL: below the quantifiable limit;
ND: not determine due to insufficient number of values

TABLE 43
Individual and mean plasma concentrations (ng/ml) of Compound A following
single IP administration of Compound I-4 to male Wistar Han rats at 3.708 mg/kg

| Time (h) | R4 | R5 | R6 | Mean | SD | CV (%) |
|---|---|---|---|---|---|---|
| 0.250 | 11.9 | 2.15 | 10.6 | 8.22 | 5.29 | 64.4 |
| 0.500 | 10.9 | 4.55 | 17.5 | 11.0 | 6.48 | 59.0 |
| 1.00 | 13.0 | 5.64 | 20.0 | 12.9 | 7.18 | 55.8 |
| 2.00 | 14.9 | 6.66 | 17.6 | 13.1 | 5.70 | 43.7 |
| 4.00 | 8.49 | 10.1 | 8.47 | 9.02 | 0.935 | 10.4 |
| 8.00 | 3.95 | 6.71 | 3.15 | 4.60 | 1.87 | 40.6 |
| 24.0 | BQL | BQL | BQL | ND | ND | ND |

BQL: below the quantifiable limit;
ND: not determine due to insufficient number of values

TABLE 44
Individual and mean plasma concentrations (ng/ml) of Compound A following
single PO administration of Compound I-19 to male Wistar Han rats at 4.061 mg/kg

| Time (h) | R7 | R8 | R9 | Mean | SD | CV (%) |
|---|---|---|---|---|---|---|
| 0.250 | 3.72 | 6.23 | 8.88 | 6.28 | 2.58 | 41.1 |
| 0.500 | 6.41 | 13.3 | 17.3 | 12.3 | 5.51 | 44.7 |
| 1.00 | 7.90 | 12.5 | 15.8 | 12.1 | 3.97 | 32.9 |
| 2.00 | 5.75 | 8.74 | 8.78 | 7.76 | 1.74 | 22.4 |
| 4.00 | 4.78 | 5.56 | 5.25 | 5.20 | 0.393 | 7.56 |
| 8.00 | 3.34 | 2.79 | 3.26 | 3.13 | 0.297 | 9.49 |
| 24.0 | 1.43 | BQL | BQL | ND | ND | ND |

BQL: below the quantifiable limit;
ND: not determine due to insufficient number of values

TABLE 45
Individual and mean plasma concentrations (ng/ml) of Compound A following
single IP administration of Compound I-19 to male Wistar Han rats at 4.061 mg/kg

| Time (h) | R10 | R11 | R12 | Mean | SD | CV (%) |
|---|---|---|---|---|---|---|
| 0.250 | 26.8 | 31.9 | 39.3 | 32.7 | 6.29 | 19.2 |
| 0.500 | 35.8 | 46.4 | 34.1 | 38.8 | 6.67 | 17.2 |
| 1.00 | 31.3 | 43.5 | 60.6 | 45.1 | 14.7 | 32.6 |
| 2.00 | 24.8 | 45.7 | 90.3 | 53.6 | 33.5 | 62.4 |
| 4.00 | 16.0 | 22.5 | 45.6 | 28.0 | 15.6 | 55.5 |
| 8.00 | 3.66 | 4.00 | 11.4 | 6.35 | 4.37 | 68.8 |
| 24.0 | BQL | BQL | 2.43 | ND | ND | ND |

BQL: below the quantifiable limit;
ND: not determine due to insufficient number of values

TABLE 46
Individual and mean plasma concentrations (ng/mL) of
Compound B following single PO administration of
Compound I-4 to male Wistar Han rats at 3.708 mg/kg

| Time (h) | R1 | R2 | R3 | Mean | SD | CV (%) |
|---|---|---|---|---|---|---|
| 0.250 | BQL | BQL | BQL | ND | ND | ND |
| 0.500 | BQL | 4.75 | 3.03 | 3.89 | ND | ND |
| 1.00 | BQL | 5.81 | 5.30 | 5.56 | ND | ND |
| 2.00 | 6.40 | 7.57 | 6.54 | 6.84 | 0.639 | 9.35 |
| 4.00 | 8.50 | 12.3 | 8.35 | 9.72 | 2.24 | 23.0 |
| 8.00 | 21.1 | 20.1 | 12.5 | 17.9 | 4.70 | 26.3 |
| 24.0 | 6.28 | 5.85 | 9.06 | 7.06 | 1.74 | 24.7 |

BQL: below the quantifiable limit;
ND: not determine due to insufficient number of values

TABLE 47
Individual and mean plasma concentrations (ng/ml) of
Compound B following single IP administration of
Compound I-4 to male Wistar Han rats at 3.708 mg/kg

| Time (h) | R4 | R5 | R6 | Mean | SD | CV (%) |
|---|---|---|---|---|---|---|
| 0.250 | BQL | BQL | 3.25 | ND | ND | ND |
| 0.500 | BQL | BQL | 4.02 | ND | ND | ND |
| 1.00 | 4.65 | BQL | 7.64 | 6.15 | ND | ND |
| 2.00 | 9.46 | BQL | 11.2 | 10.3 | ND | ND |
| 4.00 | 10.7 | 3.67 | 10.9 | 8.42 | 4.12 | 48.9 |
| 8.00 | 10.4 | 11.2 | 12.1 | 11.2 | 0.850 | 7.57 |
| 24.0 | 6.35 | 5.22 | 6.02 | 5.86 | 0.581 | 9.91 |

BQL: below the quantifiable limit;
ND: not determine due to insufficient number of values

TABLE 48
Individual and mean plasma concentrations (ng/ml) of
Compound B following single PO administration of
Compound I-19 to male Wistar Han rats at 4.061 mg/kg

| Time (h) | R7 | R8 | R9 | Mean | SD | CV (%) |
|---|---|---|---|---|---|---|
| 0.250 | BQL | BQL | BQL | ND | ND | ND |
| 0.500 | BQL | BQL | BQL | ND | ND | ND |
| 1.00 | BQL | 5.35 | 6.06 | 5.71 | ND | ND |
| 2.00 | 4.60 | 10.9 | BQL | 7.75 | ND | ND |
| 4.00 | 8.56 | 16.8 | 18.1 | 14.5 | 5.17 | 35.7 |
| 8.00 | 15.0 | 28.1 | 29.9 | 24.3 | 8.13 | 33.4 |
| 24.0 | 15.8 | 6.32 | 5.36 | 9.16 | 5.77 | 63.0 |

BQL: below the quantifiable limit;
ND: not determine due to insufficient number of values

TABLE 49
Individual and mean plasma concentrations (ng/ml) of
Compound B following single IP administration of
Compound I-19 to male Wistar Han rats at 4.061 mg/kg

| Time (h) | R10 | R11 | R12 | Mean | SD | CV (%) |
|---|---|---|---|---|---|---|
| 0.250 | BQL | BQL | 6.11 | ND | ND | ND |
| 0.500 | 5.89 | 7.24 | 9.58 | 7.57 | 1.87 | 24.7 |
| 1.00 | 9.72 | BQL | BQL | ND | ND | ND |
| 2.00 | 13.1 | 19.1 | 23.1 | 18.4 | 5.03 | 27.3 |
| 4.00 | 19.3 | 28.2 | 37.6 | 28.4 | 9.15 | 32.3 |
| 8.00 | 16.9 | 23.6 | 24.8 | 21.8 | 4.26 | 19.6 |
| 24.0 | BQL | 5.75 | 24.8 | 15.3 | ND | ND |

BQL: below the quantifiable limit;
ND: not determine due to insufficient number of values

TABLE 50
Individual and mean PK parameters of Compound I-4
following single PO administration to male
Wistar Han rats at 3.708 mg/kg

| Time (h) | R1 | R2 | R3 | Mean | SD | CV (%) |
|---|---|---|---|---|---|---|
| Rsq_adj | ND | 0.968 | ND | ND | ND | ND |
| Cₘₐₓ (ng/mL) | 3.09 | 22.7 | 1.75 | 9.18 | 11.7 | 128 |
| Tₘₐₓ (h) | 0.500 | 0.500 | 4.00 | 1.67 | 2.02 | 121 |
| T_{1/2} (h) | ND | 1.74 | ND | ND | ND | ND |
| Tₗₐₛₜ (h) | 8.00 | 8.00 | 8.00 | 8.00 | 0 | 0 |
| AUC₀₋ₗₐₛₜ | 10.3 | 53.6 | 11.6 | 25.2 | 24.6 | 97.8 |
| (ng · h/mL) | | | | | | |
| AUC_{0-inf} | ND | 56.4 | ND | ND | ND | ND |
| (ng · h/mL) | | | | | | |
| MRT₀₋ₗₐₛₜ (h) | 3.86 | 2.20 | 4.46 | 3.51 | 1.17 | 33.4 |
| MRT_{0-inf} (h) | ND | 2.61 | ND | ND | ND | ND |

ND: not determine due to insufficient number of values

TABLE 51
Individual and mean PK parameters of Compound
I-4 following single IP administration to male
Wistar Han rats at 3.708 mg/kg

| Time (h) | R4 | R5 | R6 | Mean | SD | CV (%) |
|---|---|---|---|---|---|---|
| Rsq_adj | 0.991 | ND | 1.000 | 0.995 | ND | ND |
| Cₘₐₓ (ng/mL) | 120 | 98.6 | 140 | 120 | 20.7 | 17.3 |
| Tₘₐₓ (h) | 0.250 | 4.00 | 0.250 | 1.50 | 2.17 | 144 |
| T_{1/2} (h) | 2.90 | ND | 2.12 | 2.51 | ND | ND |
| Tₗₐₛₜ (h) | 8.00 | 8.00 | 8.00 | 8.00 | 0 | 0 |
| AUC₀₋ₗₐₛₜ | 473 | 599 | 546 | 539 | 63.5 | 11.8 |
| (ng · h/mL) | | | | | | |
| AUC_{0-inf} | 567 | ND | 597 | 582 | ND | ND |
| (ng · h/mL) | | | | | | |
| MRT₀₋ₗₐₛₜ (h) | 3.06 | 4.10 | 2.77 | 3.31 | 0.701 | 21.2 |
| MRT_{0-inf} (h) | 4.58 | ND | 3.48 | 4.03 | ND | ND |

TABLE 52
Individual and mean PK parameters of Compound
I-19 following single PO administration to male
Wistar Han rats at 4.061 mg/kg

| Time (h) | R7 | R8 | R9 | Mean | SD | CV (%) |
|---|---|---|---|---|---|---|
| Rsq_adj | ND | ND | ND | ND | ND | ND |
| Cₘₐₓ (ng/ml) | 1.02 | ND | 1.14 | 1.08 | ND | ND |
| Tₘₐₓ (h) | 1.00 | ND | 0.500 | 0.750 | ND | ND |
| T_{1/2} (h) | ND | ND | ND | ND | ND | ND |
| Tₗₐₛₜ (h) | 1.00 | ND | 0.500 | 0.750 | ND | ND |
| AUC₀₋ₗₐₛₜ | 0.510 | ND | 0.285 | 0.398 | ND | ND |
| (ng · h/mL) | | | | | | |
| AUC_{0-inf} | ND | ND | ND | ND | ND | ND |
| (ng · h/mL) | | | | | | |
| MRT0-laₛₜ (h) | 1.00 | ND | 0.500 | 0.750 | ND | ND |
| MRT_{0-inf} (h) | ND | ND | ND | ND | ND | ND |

TABLE 53
Individual and mean PK parameters of Compound
I-19 following single IP administration to male
Wistar Han rats at 4.061 mg/kg

| Time (h) | R10 | R11 | R12 | Mean | SD | CV (%) |
|---|---|---|---|---|---|---|
| Rsq_adj | 0.946 | 0.991 | 0.984 | 0.974 | 0.0241 | 2.47 |
| Cₘₐₓ (ng/ml) | 158 | 193 | 192 | 181 | 19.9 | 11.0 |
| Tₘₐₓ (h) | 1.00 | 0.500 | 0.250 | 0.583 | 0.382 | 65.5 |
| T_{1/2} (h) | 1.33 | 1.54 | 1.43 | 1.43 | 0.107 | 7.50 |
| Tₗₐₛₜ (h) | 8.00 | 8.00 | 8.00 | 8.00 | 0.00 | 0.0 |
| AUC₀₋ₗₐₛₜ | 572 | 637 | 749 | 653 | 89.3 | 13.7 |
| (ng · h/mL) | | | | | | |
| AUC_{0-inf} | 584 | 660 | 771 | 672 | 93.9 | 14.0 |
| (ng · h/mL) | | | | | | |
| MRT₀₋ₗₐₛₜ (h) | 2.48 | 2.47 | 2.59 | 2.51 | 0.0655 | 2.61 |
| MRT_{0-inf} (h) | 2.63 | 2.73 | 2.80 | 2.72 | 0.0867 | 3.19 |

TABLE 54
Individual and mean PK parameters of Compound
A following single PO administration of Compound I-4 to
male Wistar Han rats at 3.708 mg/kg

| Time (h) | R1 | R2 | R3 | Mean | SD | CV (%) |
|---|---|---|---|---|---|---|
| Rsq_adj | −0.182 | 0.639 | 0.975 | 0.477 | 0.595 | 125 |
| Cₘₐₓ (ng/mL) | 7.67 | 6.90 | 7.47 | 7.35 | 0.400 | 5.44 |
| Tₘₐₓ (h) | 0.500 | 0.500 | 0.500 | 0.500 | 0 | 0 |
| T_{1/2} (h) | 38.6 | 7.00 | 16.0 | 20.5 | 16.3 | 79.4 |
| Tₗₐₛₜ (h) | 8.00 | 8.00 | 24.0 | 13.3 | 9.24 | 69.3 |
| AUC₀₋ₗₐₛₜ | 23.3 | 27.5 | 45.7 | 32.2 | 11.9 | 36.9 |
| (ng · h/mL) | | | | | | |
| AUC_{0-inf} | 178 | 54.7 | 70.1 | 101 | 67.4 | 66.7 |
| (ng · h/mL) | | | | | | |
| MRT₀₋ₗₐₛₜ (h) | 3.67 | 3.45 | 9.40 | 5.51 | 3.37 | 61.3 |
| MRT_{0-inf} (h) | 55.9 | 10.7 | 22.5 | 29.7 | 23.4 | 78.8 |

TABLE 55
Individual and mean PK parameters of Compound
A following single IP administration of Compound I-4 to
male Wistar Han rats at 3.708 mg/kg

| Time (h) | R4 | R5 | R6 | Mean | SD | CV (%) |
|---|---|---|---|---|---|---|
| Rsq_adj | 0.979 | ND | 0.979 | 0.979 | ND | ND |
| Cₘₐₓ (ng/mL) | 14.9 | 10.1 | 20.0 | 15.0 | 4.95 | 33.0 |
| Tₘₐₓ (h) | 2.00 | 4.00 | 1.00 | 2.33 | 1.53 | 65.5 |
| T_{1/2} (h) | 3.19 | ND | 2.47 | 2.83 | ND | ND |
| Tₗₐₛₜ (h) | 8.00 | 8.00 | 8.00 | 8.00 | 0 | 0 |
| AUC₀₋ₗₐₛₜ | 70.8 | 59.7 | 79.5 | 70.0 | 9.90 | 14.1 |
| (ng · h/mL) | | | | | | |
| AUC_{0-inf} | 89.0 | ND | 90.7 | 89.8 | ND | ND |
| (ng · h/mL) | | | | | | |
| MRT₀₋ₗₐₛₜ (h) | 3.25 | 4.35 | 2.91 | 3.50 | 0.757 | 21.6 |
| MRT_{0-inf} (h) | 5.16 | ND | 3.97 | 4.57 | ND | ND |

TABLE 56
Individual and mean PK parameters of Compound A
following single PO administration of Compound I-19 to male
Wistar Han rats at 4.061 mg/kg

| Time (h) | R7 | R8 | R9 | Mean | SD | CV (%) |
|---|---|---|---|---|---|---|
| Rsq_adj | 0.979 | 0.990 | 0.911 | 0.960 | 0.0425 | 4.43 |
| Cₘₐₓ (ng/ml) | 7.90 | 13.3 | 17.3 | 12.8 | 4.72 | 36.8 |
| Tₘₐₓ (h) | 1.00 | 0.500 | 0.500 | 0.667 | 0.289 | 43.3 |
| T_{1/2} (h) | 11.9 | 3.69 | 4.37 | 6.65 | 4.56 | 68.5 |
| Tₗₐₛₜ (h) | 24.0 | 8.00 | 8.00 | 13.3 | 9.24 | 69.3 |
| AUC₀₋ₗₐₛₜ | 74.7 | 50.3 | 55.0 | 60.0 | 12.9 | 21.5 |
| (ng · h/mL) | | | | | | |
| AUC_{0-inf} | 99.2 | 65.2 | 75.6 | 80.0 | 17.4 | 21.8 |
| (ng · h/mL) | | | | | | |
| MRT₀₋ₗₐₛₜ (h) | 9.04 | 3.09 | 2.96 | 5.03 | 3.48 | 69.1 |
| MRT_{0-inf} (h) | 17.0 | 5.42 | 6.04 | 9.49 | 6.51 | 68.6 |

TABLE 57
Individual and mean PK parameters of Compound A
following single IP administration of Compound I-19 to male
Wistar Han rats at 4.061 mg/kg

| Time (h) | R10 | R11 | R12 | Mean | SD | CV (%) |
|---|---|---|---|---|---|---|
| Rsq_adj | 0.978 | 0.996 | 0.981 | 0.985 | 0.00941 | 0.955 |
| Cₘₐₓ (ng/ml) | 35.8 | 46.4 | 90.3 | 57.5 | 28.9 | 50.3 |
| Tₘₐₓ (h) | 0.500 | 0.500 | 2.00 | 1.00 | 0.866 | 86.6 |
| T_{1/2} (h) | 2.24 | 1.69 | 4.41 | 2.78 | 1.44 | 51.7 |
| Tₗₐₛₜ (h) | 8.00 | 8.00 | 24.0 | 13.3 | 9.24 | 69.3 |
| AUC₀₋ₗₐₛₜ | 129 | 189 | 436 | 251 | 162 | 64.5 |
| (ng · h/mL) | | | | | | |
| AUC_{0-inf} | 141 | 199 | 451 | 264 | 165 | 62.5 |
| (ng · h/mL) | | | | | | |
| MRT₀₋ₗₐₛₜ (h) | 2.78 | 2.70 | 5.44 | 3.64 | 1.56 | 42.9 |
| MRT_{0-inf} (h) | 3.49 | 3.08 | 6.30 | 4.29 | 1.75 | 40.8 |

TABLE 58
Individual and mean PK parameters of Compound
B following single PO administration to male
Wistar Han rats at 3.708 mg/kg

| Time (h) | R1 | R2 | R3 | Mean | SD | CV (%) |
|---|---|---|---|---|---|---|
| Rsq_adj | ND | ND | ND | ND | ND | ND |
| Cₘₐₓ (ng/mL) | 21.1 | 20.1 | 12.5 | 17.9 | 4.70 | 26.3 |
| Tₘₐₓ (h) | 8.00 | 8.00 | 8.00 | 8.00 | 0.000 | 0.0 |
| T_{1/2} (h) | ND | ND | ND | ND | ND | ND |
| Tₗₐₛₜ (h) | 24.0 | 24.0 | 24.0 | 24.0 | 0 | 0 |
| AUC₀₋ₗₐₛₜ | 276 | 280 | 236 | 264 | 24.1 | 9.14 |
| (ng · h/mL) | | | | | | |
| AUC_{0-inf} | ND | ND | ND | ND | ND | ND |
| (ng · h/mL) | | | | | | |
| MRT₀₋ₗₐₛₜ (h) | 11.9 | 11.3 | 12.6 | 11.9 | 0.682 | 5.71 |
| MRT_{0-inf} (h) | ND | ND | ND | ND | ND | ND |

ND: not determine due to insufficient number of values

TABLE 59
Individual and mean PK parameters of Compound B
following single IP administration to male
Wistar Han rats at 3.708 mg/kg

| Time (h) | R4 | R5 | R6 | Mean | SD | CV (%) |
|---|---|---|---|---|---|---|
| Rsq_adj | 0.960 | ND | ND | ND | ND | ND |
| Cₘₐₓ (ng/ml) | 10.7 | 11.2 | 12.1 | 11.3 | 0.709 | 6.26 |
| Tₘₐₓ (h) | 4.00 | 8.00 | 8.00 | 6.67 | 2.31 | 34.6 |
| T_{1/2} (h) | 25.3 | ND | ND | ND | ND | ND |
| Tₗₐₛₜ (h) | 24.0 | 24.0 | 24.0 | 24.0 | 0.00 | 0.0 |
| AUC₀₋ₗₐₛₜ | 203 | 162 | 221 | 196 | 30.1 | 15.4 |
| (ng · h/mL) | | | | | | |
| AUC_{0-inf} | 434 | ND | ND | ND | ND | ND |
| (ng · h/mL) | | | | | | |
| MRT₀₋ₗₐₛₜ (h) | 11.5 | 13.0 | 11.2 | 11.9 | 0.993 | 8.34 |
| MRT_{0-inf} (h) | 37.6 | ND | ND | ND | ND | ND |

TABLE 60
Individual and mean PK parameters of Compound
B following single PO administration to male
Wistar Han rats at 4.061 mg/kg

| Time (h) | R7 | R8 | R9 | Mean | SD | CV (%) |
|---|---|---|---|---|---|---|
| Rsq_adj | ND | ND | ND | ND | ND | ND |
| Cₘₐₓ (ng/mL) | 15.8 | 28.1 | 29.9 | 24.6 | 7.67 | 31.2 |
| Tₘₐₓ (h) | 24.0 | 8.00 | 8.00 | 13.3 | 9.24 | 69.3 |
| T_{1/2} (h) | ND | ND | ND | ND | ND | ND |
| Tₗₐₛₜ (h) | 24.0 | 24.0 | 24.0 | 24.0 | 0 | 0 |
| AUC₀₋ₗₐₛₜ | 311 | 362 | 364 | 346 | 29.7 | 8.61 |
| (ng · h/mL) | | | | | | |
| AUC_{0-inf} | ND | ND | ND | ND | ND | ND |
| (ng · h/mL) | | | | | | |
| MRT₀₋ₗₐₛₜ (h) | 14.0 | 11.0 | 10.7 | 11.9 | 1.81 | 15.2 |
| MRT_{0-inf} (h) | ND | ND | ND | ND | ND | ND |

TABLE 61
Individual and mean PK parameters of Compound B following single IP
administration to male Wistar Han rats at 4.061 mg/kg

| Time (h) | R10 | R11 | R12 | Mean | SD | CV (%) |
|---|---|---|---|---|---|---|
| Rsq_adj | ND | 0.985 | ND | ND | ND | ND |
| Cₘₐₓ (ng/mL) | 19.3 | 28.2 | 37.6 | 28.4 | 9.15 | 32.3 |
| Tₘₐₓ (h) | 4.00 | 4.00 | 4.00 | 4.00 | 0.00 | 0 |
| T_{1/2} (h) | ND | 8.45 | ND | ND | ND | ND |
| Tₗₐₛₜ (h) | 8.00 | 24.0 | 24.0 | 18.7 | 9.24 | 49.5 |
| AUC₀₋ₗₐₛₜ (ng · h/ | 121 | 374 | 608 | 368 | 243 | 66.1 |
| mL) | | | | | | |
| AUC_{0-inf} (ng · h/ | ND | 445 | ND | ND | ND | ND |
| mL) | | | | | | |
| MRT₀₋ₗₐₛₜ (h) | 4.58 | 9.79 | 12.0 | 8.79 | 3.82 | 43.4 |
| MRT_{0-inf} (h) | ND | 14.0 | ND | ND | ND | ND |

Oral administration of Compound I-4 at 3.708 mg/kg yielded a peak plasma concentration (Cₘₐₓ) of 9.18±11.7 ng/mL at 1.67±2.02 hour (Tₘₐₓ) post-dose and an area under the plasma concentration-time curve from time 0 to the last quantifiable time (AUC₀₋ₗₐₛₜ) of 25.2±24.6 ng·h/mL. Compound I-4 had a mean residence time from time 0 to the last quantifiable time (MRT₀₋ₗₐₛₜ) of 3.51±1.17 hour. The terminal elimination half-life (T_{1/2}) of Compound I-4 was not determined due to insufficient number of concentration values during the terminal elimination phase. The Cₘₐₓ of Compound A was measured at 7.35±0.4 ng/mL. The Tₘₐₓ of Compound A was 0.500±0 hour post-dose, and the AUC₀₋ₗₐₛₜ was 32.2±11.9 ng·h/mL. Compound A had a T_{1/2} of 20.5±16.3 hour and a MRT₀₋ₗₐₛₜ of 5.51±3.37 hour following PO administration of Compound I-4. The metabolite Compound B had an average Cₘₐₓ of 17.9±4.7 ng/mL at 8.00±0 hour post dose. The AUC₀₋ₗₐₛₜ was 264±24.1 ng·h/mL. There were not sufficient numbers of samples to determine the T_{1/2} for Compound B, while the MRT₀₋ₗₐₛₜ was 11.9±0.682 hour following PO administration of Compound I-4.

Intraperitoneal administration of Compound I-4 at 3.708 mg/kg yielded a Cₘₐₓ of 120±20.7 ng/mL at the Tₘₐₓ of 1.50±2.17 hours post-dose and an AUC₀₋ₗₐₛₜ of 539±63.5 ng·h/mL. The T_{1/2} and MRT₀₋ₗₐₛₜ of Compound I-4 following IP administration was 2.51 hours and 3.31±0.701 hours, respectively. The Cₘₐₓ and AUC₀₋ₗₐₛₜ of Compound A following IP administration of Compound I-4 were 15.0±4.95 ng/mL and 70.0±9.9 ng·h/mL, respectively. The Tₘₐₓ, T_{1/2} and MRT₀₋ₗₐₛₜ of Compound A following IP administration were 2.33±1.53, 2.83 and 3.50±0.757 hours, respectively. The metabolite Compound B had an average Cₘₐₓ of 11.3±0.709 ng/mL at 6.67±2.31 hour post dose. The AUC₀₋ₗₐₛₜ was 196 30.1 ng·h/mL. There were not sufficient numbers of samples to determine the T_{1/2} for Compound B, while the MRT₀₋ₗₐₛₜ was 11.9±0.993 hour following IP administration of Compound I-4.

Oral administration of Compound I-19 at 4.061 mg/kg yielded a Cₘₐₓ of 1.08 ng/mL at 0.750 hour post-dose and an AUC₀₋ₗₐₛₜ of 0.398 ng·h/mL. Compound I-19 had a MRT₀₋ₗₐₛₜ of 0.750 hour. The T_{1/2} of Compound I-19 was not determined due to insufficient number of concentration values during the terminal elimination phase. The Cₘₐₓ of Compound A following PO administration of Compound I-19 measured 12.8±4.72 ng/mL. The Tₘₐₓ of Compound A was 0.667±0.289 hour post-dose, and the AUC₀₋ₗₐₛₜ was 60.0±12.9 ng·h/mL. Compound A had a T_{1/2} of 6.65±4.56 hours and a MRT₀₋ₗₐₛₜ of 5.03±3.48 hours following PO administration of Compound I-19. The metabolite Compound B had an average Cₘₐₓ of 24.6±7.67 ng/mL at 13.3±9.24 hour post dose. The AUC₀₋ₗₐₛₜ was 346±29.7 ng·h/mL. There were not sufficient numbers of samples to determine the T_{1/2} for Compound B, while the MRT₀₋ₗₐₛₜ was 11.9±1.81 hour following PO administration of Compound I-19.

Intraperitoneal administration of Compound I-19 at 4.061 mg/kg yielded a Cₘₐₓ of 181±19.9 ng/mL at the Tₘₐₓ of 0.583±0.382 hour post-dose and an AUC₀₋ₗₐₛₜ of 653±89.3 ng·h/mL. The T_{1/2} and MRT₀₋ₗₐₛₜ of Compound I-19 following IP administration was 1.43±0.107 hours and 2.51±0.0655 hours, respectively. The Cₘₐₓ and AUC₀₋ₗₐₛₜ of Compound A following IP administration of Compound I-19 were 57.5±28.9 ng/mL and 251±162 ng·h/mL, respectively. The Tₘₐₓ, T_{1/2} and MRT₀₋ₗₐₛₜ of Compound A following IP administration of Compound I-19 were 1.00±0.866, 2.78±1.44 and 3.64±1.56 hours, respectively. The metabolite Compound B had an average Cₘₐₓ of 28.4±9.15 ng/mL at 4.00±0 hour post dose. The AUC₀₋ₗₐₛₜ was 368±243 ng·h/mL. There were not sufficient numbers of samples to determine the T_{1/2} for Compound B, while the MRT₀₋ₗₐₛₜ was 8.79±3.82 hour following IP administration of Compound I-19.

The Cₘₐₓ and AUC₀₋ₗₐₛₜ data showed that IP administration of Compound I-4 and Compound I-19 had a greater relative bioavailability compared to PO administration. PO route results in significant biotransformation of the derivative, while IP route exhibits greater amount of the Compound A.

Both Compound I-4 and Compound I-19 at the administered dosage were well tolerated by the animals. No adverse effects were evident throughout the study.

### Conclusion

The Cₘₐₓ and AUC₀₋ₗₐₛₜ data showed that IP administration of Compound I-4 and Compound I-19 had a greater relative bioavailability compared to PO administration. At the equivalent dose of 3 mg/kg free base Compound A, Compound I-19 had a greater conversion efficiency to Compound A metabolite compared to Compound I-4 independent of dose routes, and IP administration had a greater conversion efficiency to Compound A metabolite independent of the parent.

Both Compound I-4 and Compound I-19 at the administered dosage were well tolerated by the animals. No adverse effects were evident throughout the study.

### Example 8: Determination of Plasma and Brain Concentrations of the Novel Compound, Compound I-19, in Mice Following Intraperitoneal Administration and Evaluation of the Compound's Binding to Plasma and Brain Proteins/Lipids for Subsequent Evaluation of Brain to Plasma Unbound Partition Coefficient (Kp,uu)

The first objective of the study was to determine the concentration of the test compound, Compound I-19, Compound A (Compound I-19 metabolite) and Compound B (Compound A ring-opened product) in blood plasma and brain at four time-points (0.0833, 0.25, 0.75 and 2 h) following intraperitoneal (i.p.) administration of 3 mg/kg Compound I-19 to male CD-1 mice. Following dosing, three animals were sacrificed per time-point for the collection of terminal plasma and brain. A liquid chromatography-tandem mass spectrometry (LC-MS/MS) method(s) was developed and qualified for the analysis of Compound I-19, Compound A and Compound B in mouse plasma and brain homogenates. Pharmacokinetics (PK) parameters for plasma and whole brain were estimated from the mean plasma and brain concentration versus time profiles.

The second objective of the study was to determine the fraction of Compound I-19, bound and unbound to mouse plasma proteins and to mouse brain tissue by the equilibrium dialysis method. Mouse plasma and homogenized brain were spiked with Compound I-19 at a concentration of 2 μM (0.742 μg/mL). Samples of spiked plasma and blank buffer (Dulbecco's phosphate-buffered saline, DPBS) and spiked brain homogenate and blank DPBS added to opposing sides of a dialysis membrane were collected from triplicate equilibrium dialysis chambers prior to (t=0) and following 1, 2, 4 and 6 hours of incubation at 37° C. Additionally, DPBS spiked with 2 μM Compound I-19 was dialyzed against blank DPBS for 1, 2, 4 and 6 hours to determine whether equilibrium was attained. Although not included in the protocol, an aliquot of each of the spiked (2 μM Compound I-19) DPBS, plasma and brain homogenate was also incubated at 37° C. (n=1) for 6 hours to assess the stability of Compound I-19 in the three matrices. The LC-MS/MS method(s) for analytes (Compound I-19, Compound A and Compound B) was cross-qualified for the analysis of the DPBS samples. The fraction of Compound I-19 unbound and bound to plasma proteins and brain proteins/lipids was estimated, as well as the recovery of the test compound at the end of the dialysis period. The fraction unbound to plasma and brain proteins was used to estimate the unbound concentrations of Compound I-19 in plasma and brain, respectively, and the brain to plasma unbound partition coefficient (Kp,uu) was calculated.

The calibration dynamic ranges used for the LC-MS/MS method were 1 to 5000 nM for Compound I-19 and Compound B and 1 to 2000 nM for Compound A in plasma, DPBS and brain (for analysis of in vitro samples), and 3 to 15000 nmol/kg for Compound I-19 and Compound B and 3 to 6000 nmol/kg for Compound A in brain (for analysis of in vivo samples). Both the method qualification and sample analysis batches for the 3 analytes in each matrix passed the acceptance criteria.

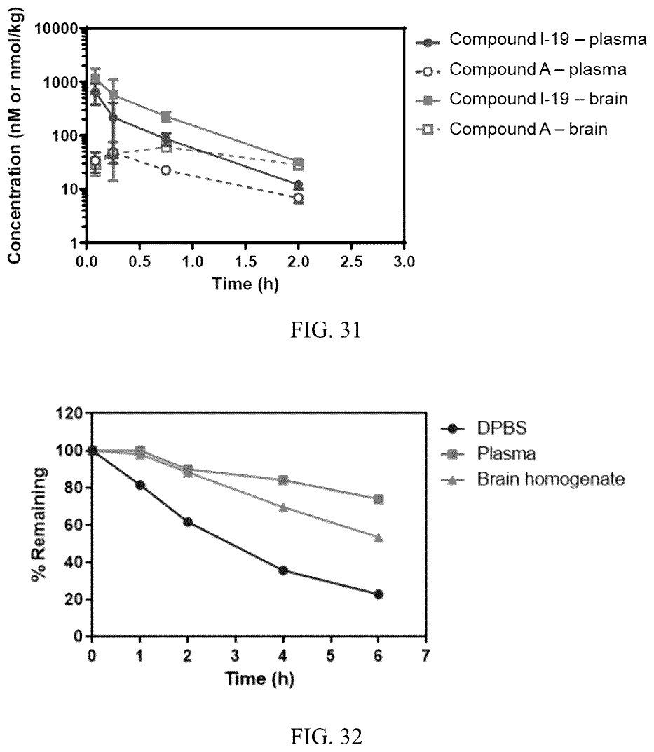

The in vivo PK parameters for Compound I-19 and Compound A following 3 mg/kg i.p. administration of Compound I-19 are summarized in Table 62, and the corresponding plasma and brain concentration versus time profiles are depicted in FIG. 31. The Compound A ring-opened product (Compound B) was below the lower levels of quantitation (BLQ) in both plasma (<1 nM) and brain (<3 nmol/kg) samples. Maximum plasma (648 nmol/L) and brain (1182 nmol/kg) concentrations (Cₘₐₓ) of Compound I-19 peaked at the first sample collection time-point (Tₘₐₓ=0.0833 h) following dosing, with the Cₘₐₓ in brain being 1.8-fold higher than that in plasma.

Brain concentrations of Compound I-19 paralleled plasma concentrations and the apparent half-lives were similar for plasma (0.423 h) and brain (0.427 h). Overall brain exposure (AUC_{0-inf}) was greater than that in plasma; the brain to plasma partitioning coefficient (Kp) of Compound I-19 was 2.38 (Table 62A). Compound A plasma and brain concentrations peaked at 0.25 and 0.75 hours, respectively, and both were much lower in magnitude (48.2 nmol/L and 60.3 nmol/kg, respectively) than those of Compound I-19 (Table 62B, FIG. 31). As there were an insufficient number of time-points following Tₘₐₓ, plasma and brain T_{1/2}'s could not be estimated for Compound A.

TABLE 62
Summary of mean (±SD, n = 3) plasma and brain PK parameters
for Compound I-19 (A) and Compound A (B) following 3 mg/kg i.p.
administration of Compound I-19 to groups of 3 male CD-1 mice.
(A) Compound I-19

Parameter estimate

| Parameter^{a} | Plasma | Brain |
|---|---|---|
| Tₘₐₓ (h) | 0.0833 | 0.0833 |
| Cₘₐₓ (nmol/L) | 648 | 1182 |
| fᵤ* Cₘₐₓ (nmol/L) ^{b} | 24.1 | 39.1 |
| Apparent T_{1/2} (h) | 0.423 | 0.427 |
| AUC_{0-inf} (h*nmol/L) | 218 | 521 |
| fᵤ* AUC_{0-inf} (h*nmol/L) | 8.13 | 17.2 |

| | |
|---|---|
| Brain/plasma AUC_{0-inf} ratio (Kp) | 2.38 |
| (fᵤ* AUC_{0-inf})_{brain}/(fᵤ*AUC_{0-inf})ₚₗₐₛₘₐ ratio (Kp, uu) | 2.12 |

(B) Compound A

Parameter estimate

| Parameter | Plasma | Brain |
|---|---|---|
| Tₘₐₓ (h) | 0.250 | 0.750 |
| Cₘₐₓ (nmol/L) | 48.2 | 60.3 |
| AUC_{0-2 h} (h*nmol/L) | 41.5 | 86.6 |

ₐBrain concentration is expressed as nmol/kg. Since brain density is ~1 g/mL, nmol/kg = nmol/L.
^{b} Fraction unbound (fu) in plasma and brain were determined by equilibrium dialysis after 6 h incubation.

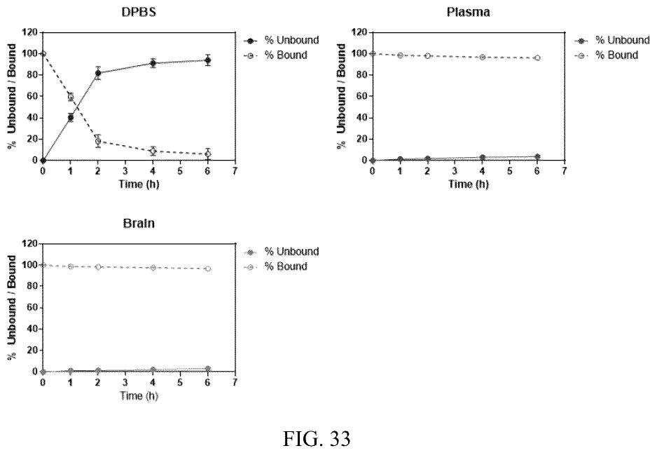

The % unbound and % recovery for Compound I-19 determined in DPBS and mouse plasma and brain as a function of dialysis time are summarized in Table 63. Although Compound I-19 was found to be unstable in all three matrices (FIG. 32, Table 63), it appears that equilibrium was attained by 6 hours as evidenced by essentially equal concentrations of Compound I-19 in the DPBS donor (spiked) and receiver (blank) wells of the equilibrium dialysis cell. Following 6 hours of dialysis, the estimated % unbound in DPBS was 94.0±5.12% (Table 63). The changes in % unbound and bound of Compound I-19 over 6 hours for DPBS, plasma and brain are depicted in FIG. 33. The change in % unbound for Compound I-19 in plasma and brain between 4 and 6 hours of dialysis was very small, again suggesting that equilibrium had been, or was very close to being attained. Both Compound A and Compound B were measurable in the dialyzed DPBS, plasma and brain homogenate samples.

TABLE 63
Summary of mean (±SD, n = 3) % unbound determined in DPBS, mouse plasma
and brain and assay % recovery of Compound I-19 over a 6-hour equilibrium dialysis period.

| Dialysis | DPBS | | Plasma | | Brain | |
|---|---|---|---|---|---|---|
| time (h) | % Unbound | % Recovery | % Unbound | % Recovery | % Unbound | % Recovery |
| 1 | 40.3 ± 3.69 | 64.7 ± 3.04 | 1.43 ± | 87.9 ± 3.05 | 1.33 ± | 87.8 ± 2.40 |
| | | | 0.0376 | | 0.0948 | |
| 2 | 81.9 ± 5.87 | 55.5 ± 2.08 | 1.91 ± 0.158 | 80.1 ± 2.93 | 1.64 ± 0.181 | 83.1 ± 1.58 |
| 4 | 91.1 ± 4.19 | 32.6 ± 1.05 | 3.20 ± 0.700 | 67.6 ± 3.41 | 2.46 ± 0.183 | 65.1 ± 4.05 |
| 6 | 94.0 ± 5.12 | 20.2 ± 0.461 | 3.72 ± 0.564 | 57.1 ± 0.567 | 3.31 ± 0.385 | 51.3 ± 2.58 |

The estimated % unbound for Compound I-19 in plasma and brain following 6 hours of incubation was 3.72±0.564% and 3.31±0.385%, respectively. The % recovery of the compound following 6 hours of dialysis (57.1±0.567% and 51.3±2.58% for plasma and brain homogenate, respectively) was higher than that after dialysis of DPBS alone (20.2±0.461%), consistent with the poorer stability of Compound I-19 in DPBS relative to plasma and brain homogenate when 2 μM Compound I-19 was spiked into these matrices and incubated at 37° C. over 6 hours in the absence of dialysis (FIG. 32). Compound A and Compound B were also measurable in the DPBS, plasma and brain homogenate stability samples. These data indicate that Compound I-19 is relatively more stable in the plasma/homogenate milieu. This may be due to the high % binding since a molecule that is highly bound in plasma or brain homogenate is protected from degradation reactions and, therefore, artificially more stable.

Following correction for the unbound fractions (fu) in plasma and brain, unbound concentrations (fu*Cₘₐₓ) and overall exposure (fu*AUC_{0-inf}) of Compound I-19 in brain are higher than those in plasma, and the estimated Kp,uu was 2.12 (Table 62A). A Kp,uu value greater than unity is suggestive of an active transport process for uptake of the compound into the brain.

### Materials and Methods

### Test Articles

The test articles, Compound I-19 (C₂₀H₁₉ClN₂O₃; MW=370.83 g/mol; Exact mass: 370.1084 g/mol; Purity: 98.77%; Batch No: 115-164.1) and Compound B (C₁₆H₁₅ClN₂O₂; MW: 302.76 g/mol; Exact mass: 302.0822 g/mol; Purity: 96.5%; Batch No: 113-180) were provided as solid material. Compound A hydrolysis product-d4 (2-(2-Aminoethyl)-3-(4-chlorophenyl)-3-hydroxyphthalimidine-d4 Hydrochloride; MFsalt: C₁₆H₁₂D₄Cl₂N₂O₂; MWsalt: 343.2 g/mol; MWfree base: 306.78 g/mol; Exact massfree base: 306.1073 g/mol; Purity: >98%; (Item No: A608912) will be purchased from Toronto Research chemicals. Compound A will be purchased from Lipomed as a certified reference material (1 mg/mL in DMF; C₁₆H₁₃ClN₂O; MW: 284.74 g/mol; Exact mass: 284.0716 g/mol; Purity>99%; Item No: MZD-1274-FB-1LD; Lot No: 1274.1B0.1L5). The test material will be stored with desiccant (solid test material only) and protected from light at 2-8° C. until used. All concentrations of the test article will be based on the free form (free base) of the compound and will be corrected for the salt form and purity.

### In Vivo Study Procedures

### Animals

Twelve male CD-1 mice (25-40 g) from Charles River Laboratories will be used. The animals will be acclimatized to their new environment for a minimum of 5 days prior to study start.

### Health Observations

Only animals in good health will be used for dosing. Since the effects of the test articles on the animals may not be known, animals will be closely observed for 1 h following dosing, at each sample collection time point for the remainder of the study. Any adverse reactions to the administration of the test material will be noted on the data sheets and will be tabulated and included in the report. Should the mouse become moribund or be observed to experience a severe level of pain, Supernus and the facility veterinarian will be notified. A terminal blood sample will be collected, if possible, and the mouse will be humanely euthanized using isoflurane anesthesia and cardiac exsanguination. A necropsy will also be performed, if possible.

### Body Weights

Animals will be weighed on the day of test article administration for dose volume calculations.

### Test Article Formulation

The test article will be freshly prepared at 0.6 mg/mL in 0.8% DMSO, 6% PEG400, 93.2% of 6% 2-Hydroxypropyl-B-cyclodextrin in H₂O. Any unused dosing solutions will be stored at approximately −80° C. until transferred on dry ice to the bioanalytical facility.

### Test Article Administration

The animals will be dosed intra-peritoneally (i.p.) into the lower right quadrant of the abdomen using a 0.5 mL Tuberculin syringe with attached needle 27 G×4″.

### Terminal Blood Collection

Whole blood will be collected via cardiac puncture under isoflurane anesthesia from three mice at each time-point at the collection times outlined in Table 2. Up to 0.8 mL of blood will be collected into 0.8 mL mini-collect K2EDTA coated tubes. Blood tubes will be centrifuged at 3200×g for 5 min. at 4° C. and plasma transferred into 1.5 mL flip top tubes. One vial of plasma for each collection time point will stored at approximately 80° C. until transferred on dry ice to the bioanalytical facility.

### Terminal Blood Collection

Following cardiac puncture, mice will be perfused with 12 mL of ice-cold PBS via the left ventricle. Brains will then be excised, blotted and placed into pre-weighed 5 mL self standing green top sample tubes. Tubes containing brains will be re-weighed and then snap frozen in liquid nitrogen. Brain samples will be stored frozen at approximately −80° C. until transferred on dry ice to the bioanalytical facility.

### In Vitro Study Procedures

### Preparation of Test Article Solution for the In Vitro Studies

A 2 mM (0.742 mg/mL) stock solution of Compound I-19 will be prepared in DMSO. A secondary stock solution of 200 μM (74.2 ug/mL) will be prepared in DMSO. This secondary stock solution will then be diluted 100-fold to 2 μM (0.742 pg/mL) with male CD-1 mouse plasma and brain homogenate (1:2 v/v brain:Dulbecco's phosphate buffered saline, pH 7-7.2) or buffer (DPBS).

### Plasma Protein and Brain Homogenates Binding Assay

Binding will be estimated by the equilibrium dialysis method as follows:

To 2.97 mL aliquots of thawed mouse plasma or brain homogenate warmed to 37° C., 30 ul aliquots of the secondary stock solution of the test compound will be added for final concentrations of 2 μM (0.742 ug/mL). An appropriate volume of warmed buffer (DPBS) will also be spiked with the test compound at a concentration of 2 μM (0.742 g/mL). Triplicate aliquots (50 μL) of each spiked plasma, brain homogenate and buffer solution will be collected prior to dialysis into labeled 1.5 mL polypropylene tubes and then stored at approximately −80° C. until bioanalysis.

Equilibrium dialysis will be performed in a 96-well Teflon dialysis unit (HTDialysis, Gales Ferry, CT). Each well of the unit consists of two chambers separated by a vertically aligned dialysis membrane (regenerated cellulose) with a molecular weight cut-off of 12-14 KDa. The equilibrium dialysis membranes will first be presoaked in deionized water for 30 min, then soaked in 20% ethanol for 20 min, followed by rinsing twice with deionized water.

The equilibrium dialysis apparatus will be assembled according to the manufacturer's directions. One chamber of each well will be immediately filled with 150 μL of blank buffer (to prevent membrane dehydration). Aliquots (150 μL) of spiked plasma, spiked brain homogenate and spiked buffer will then be added to the opposing chamber in each well. Three wells per time point per matrix will be used. The top of the plate will be sealed with an adhesive sealing film to prevent evaporation and maintain constant pH during incubation.

All dialysis experiments with the test compound will be assessed using triplicate samples.

The dialysis apparatus will be incubated at 37° C. in an orbital shaker/incubator (Lab-Line Enviro Shaker) set at 75 rpm.

Following 1, 2, 4 and 6 hours of incubation, samples from each plasma, brain homogenate and buffer compartment will be collected and the sample volume will be documented.

### Conclusion

Following i.p. administration of 3 mg/kg Compound I-19 to male CD-1 mice, maximum plasma and brain concentrations of Compound I-19 peaked at the first sample collection time-point (0.0833 h) following dosing, with the Cₘₐₓ in brain being 1.8-fold higher than that in plasma. Brain concentrations of Compound I-19 paralleled plasma concentrations with similar apparent half-lives (0.42 h). The brain to plasma partitioning coefficient (Kp) of Compound I-19 was 2.38. The estimated fractions of Compound I-19 unbound to plasma and brain (0.0372 and 0.0331, respectively), resulted in an estimated Kp,uu of 2.12, suggesting that the compound is actively transported into the brain. Compound A plasma and brain concentrations peaked at 0.25 and 0.75 hours, respectively, and both were much lower in magnitude than those of Compound I-19. The Compound A ring-opened product (Compound B) was below the lower levels of quantitation in both plasma (<1 nM) and brain (<3 nmol/kg) samples. Compound B has been detected in previous studies with various species. The reason for observing very low concentrations of the metabolite in the present study may be due to differences in species, dose and/or route of administration.

### Example 9: In Vitro Assessment of Compound I-19, Compound a and Compound B as Inhibitor of Human BCRP, BSEP and MDR1 Efflux (ABC) Transporters and Human MATE1, MATE2-K, OAT1, OAT3, OATP1B1, OATP1B3, OCT1 and OCT2 Uptake Transporters

Compound I-19, Compound A and Compound B were evaluated as inhibitors of the human ABC (efflux) transporters: BCRP, BSEP and MDR1 and the human SLC (uptake) transporters: MATE1, MATE2-K, OAT1, OAT3, OATP1B1, OATP1B3, OCT1 and OCT2 (Table 64).

TABLE 64
Test articles (Compound I-19, Compound A
and Compound B) and transporter assays

| | | | Applied nominal |
|---|---|---|---|
| Test article | Transporter | Assay | concentrations |
| Compound I-19 | BCRP* | VT | 10 and 100 μM |
| | BSEP* | inhibition | and |
| | MDR1* | | *0.14, 0.41, 1.24, 3.70, |
| | | | 11.1, 33.3 and 100 μM |
| | MATE1* | Uptake | 10 and 100 μM |
| | MATE2-K* | inhibition | and |
| | OAT1 | | *0.14, 0.41, 1.24, 3.70, |
| | OAT3 | | 11.1, 33.3 and 100 μM |
| | OATP1B1* | | |
| | OATP1B3* | | |
| | OCT1* | | |
| | OCT2 | | |
| Compound A | BCRP* | VT | 5 and 50 μM |
| | MDR1* | inhibition | |
| | BSEP | | 3.5 and 35 μM |
| | MATE1* | Uptake | 3 and 30 μM |
| | MATE2-K* | inhibition | and |
| | OAT1 | | 0.041, 0.123, 0.37, 1.11, |
| | OAT3 | | 3.33, 10 and 30 μM |
| | OATP1B1 | | |
| | OATP1B3 | | |
| | OCT1* | | |
| | OCT2* | | |
| Compound B | BCRP | VT | 10 and 100 μM |
| | BSEP | inhibition | and |
| | MDR1* | | *0.14, 0.41, 1.24, 3.70, |
| | | | 11.1, 33.3 and 100 μM |
| | MATE1* | Uptake | 5 and 50 μM |
| | | inhibition | and |
| | | | *0.069, 0.206, 0.617, 1.85, |
| | | | 5.56, 16.67 and 50 μM |
| | MATE2-K* | | 8 and 80 μM |
| | | | and |
| | | | *0.11, 0.329, 0.988, 2.96, |
| | | | 8.89, 26.67 and 80 μM |
| | OAT1 | | 10 and 100 μM |
| | OAT3 | | and |
| | OATP1B1 | | *0.14, 0.41, 1.24, 3.70, |
| | OATP1B3 | | 11.1, 33.3 and 100 μM |
| | OCT1* | | |
| | OCT2 | | |

VT: vesicular transport assay
*Follow-up assays were carried out to determine the IC₅₀ values.

### Results and Discussion

The tabulated summary of results is provided in Table 65 and Table 66.

TABLE 65
Summary of the obtained results - ABC transporter assays

| | | | |
|---|---|---|---|
| Pharmacokinetics: | Inhibition of BCRP, BSEP | Test Article: | Compound I-19, |
| | and MDR1 | | Compound A and |
| | | | Compound B |
| | | Location in CTD: | |
| | | Report No.: | 811M-ADME2022-017 |
| | | Study No.: | Supernus-01 |

| | |
|---|---|
| Type of Study: | A non-GLP in vitro study to determine whether Compound I-19, Compound A |
| | and Compound B are inhibitors of BCRP, BSEP and MDR1 transporters using |
| | inside-out membrane vesicles prepared from cells overexpressing human ABC |
| | (efflux) transporters. The study design also included solubility and non-specific |
| | binding assessment. |
| Method: | For inhibition assessment, vesicles were incubated in the respective assay |
| | buffer containing the probe substrate in the presence of Compound I-19 and |
| | Compound B (10 and 100 μM or 0.14, 0.41, 1.24, 3.70, 11.1, 33.3 and 100 μM |
| | in the follow-up experiment) and in the presence of Compound A (5 and 50 μM |
| | for BCRP and MDR1 or 3.5 and 35 μM for BSEP). The reaction was initiated |
| | with the addition of ATP or AMP to the solution and following the incubation |
| | time, the reaction was stopped by the addition of 200 μL of ice-cold washing |
| | buffer and immediate filtration via glass fiber filters mounted to a 96-well plate |
| | (filter plate). The filters were washed, dried and the amount of substrate inside |
| | the filtered vesicles was determined by liquid scintillation counting. |
| | E3S (1 μM) for BCRP, TC (0.2 μM) for BSEP and NMQ (1 μM) for MDR1 |
| | were used as positive control substrates. |
| Analysis: | Radiodetection |

Inhibition assessment of ABC transport

| Transporter | Test article | Maximum % inhibition | IC₅₀ (μM) |
|---|---|---|---|
| BCRP | Compound I-19 | 43 | NA |
| BSEP | | 73 | 45.3* |
| MDR1 | | 89 | 21.0* |
| BCRP | Compound A | NIO | NA |
| BSEP | | NIO | NA |
| MDR1 | | NIO | NA |
| BCRP | Compound B | NIO | NA |
| BSEP | | NIO | NA |
| MDR1 | | 53 | 87.8 |

*Bottom constrained to 0.
NA: Not applicable.
NIO: No inhibition observed.

TABLE 66
Summary of the obtained results - SLC transporter assays

| | | | |
|---|---|---|---|
| Pharmacokinetics: | Inhibition of MATE1, | Test Article: | Compound I-19, |
| | MATE2-K, OAT1, OAT3, | | Compound A and |
| | OATP1B1, OATP1B3, | | Compound B |
| | OCT1 and OCT2 | | |
| | | Location in CTD: | |
| | | Report No.: | 811M-ADME2022-017 |
| | | Study No.: | Supernus-01 |

| | |
|---|---|
| Type of Study: | A non-GLP in vitro study to determine whether Compound I-19, Compound A |
| | and Compound B are inhibitors of MATE1, MATE2-K, OAT1, OAT3, |
| | OATP1B1, OATP1B3, OCT1 and OCT2 transporters using human embryonic |
| | kidney mammalian (HEK293) cells expressing MATE1, MATE2-K, OAT1, |
| | OAT3, OATP1B1, OATP1B3, OCT1 and OCT2. The study design also |
| | included solubility, non-specific binding and cytotoxicity assessment. |
| Method: | For inhibition assessment, cells were pre-incubated for 30 minutes in the |
| | respective assay buffer in the presence of Compound I-19 and Compound B (10 |
| | and |
| | 100 μM or 0.14, 0.41, 1.24, 3.70, 11.1, 33.3 and 100 μM in the follow-up |
| | experiment) and in the presence of Compound A (3 and 30 μM and 0.041, |
| | 0.123, 0.37, 1.11, 3.33, 10 and 30 μM in the follow-up assay). Then the probe |
| | substrate was added in a fresh dosing solution of Compound I-19, Compound |
| | A and Compound B and following the incubation time, the reaction was stopped |
| | by washing the cells with ice-cold buffer. The cells were lysed with scintillation |
| | cocktail and the accumulation of the probe substrate in the cells was determined |
| | by liquid scintillation counting. |
| | Metformin (10 μM) for MATE1, MATE2-K, OCT2, Sumatriptan (10 μM) for |
| | OCT1, Tenofovir (5 μM) for OAT1, E3S (1 μM) for OAT3, E₂17βG (1 μM) |
| | for OATP1B1 and CCK-8 (1 μM) for OATP1B3 were used as positive control |
| | substrates. |
| Analysis: | Radiodetection |

Inhibition assessment of SLC transport

| Transporter | Test article | Maximum % inhibition | IC₅₀ (μM) |
|---|---|---|---|
| MATE1 | Compound I-19 | 104 | 8.86* |
| MATE2-K | | 86 | 36.6* |
| OAT1 | | 33 | NA |
| OAT3 | | 42 | NA |
| OATP1B1 | | 83 | 31.4* |
| OATP1B3 | | 61 | 82.8* |
| OCT1 | | 92 | 3.32 |
| OCT2 | | 97 | 8.95 |
| MATE1 | Compound A | 79 | 3.87* ** |
| MATE2-K | | 79 | 12.8 |
| OAT1 | | NIO | NA |
| OAT3 | | NIO | NA |
| OATP1B1 | | NIO | NA |
| OATP1B3 | | NIO | NA |
| OCT1 | | 86 | 2.31* ** |
| OCT2 | | 91 | 3.49** |
| MATE1 | Compound B | 74 | 35.7* |
| MATE2-K | | 64 | 56.8* ** |
| OAT1 | | NIO | NA |
| OAT3 | | NIO | NA |
| OATP1B1 | | 34 | NA |
| OATP1B3 | | NIO | NA |
| OCT1 | | 93 | 4.36 |
| OCT2 | | 36 | NA |

### Kinetic Solubility Assessment

The kinetic solubility of Compound I-19, Compound A and Compound B was assessed in the respective assay buffers used for the selected transporter assays: in transport buffer for BCRP and MDR1 (Transport buffer with sucrose, pH 7.4, 32° C.), for BSEP (BSEP Buffer, pH 7.4) VT inhibition assays, in Krebs-Henseleit (KH, pH 8.0) and Hanks' Balanced Salt Solution (HBSS, pH 7.4) uptake assay buffer (Table 67).

TABLE 67
Experimental conditions of solubility assays for inhibition
assays: Compound I-19, Compound A and Compound B

| | | Incubation | Final | Final nominal | No. of |
|---|---|---|---|---|---|
| Assay type | Assay Buffer | conditions | dilution factor | concentrations | wells |
| Vesicular | Transport buffer for | 32° C. | 100 | 6.25, 12.5, 25, 50 | |
| transport | BCRP, MDR1 | 15 min | | and 100 μM | |
| inhibition | (Transport buffer with | | | and | |
| assay | sucrose, pH 7.4) | | | *50, 60, 70, 80, | |
| | | | | 90 and 100 μM | |
| | | | | in case of | |
| | | | | Compound A in | |
| | | | | the refinement | |
| | | | | assay | |
| | Transport buffer for | 37° C., | 100 | 6.25, 12.5, 25, 50 | n = 3 |
| | BSEP | 100 | | and 100 μM | |
| | (BSEP Buffer, pH 7.4) | | | and | |
| | | | | *25, 30, 35, 40, | |
| | | | | 45 and 50 μM in | |
| | | | | case of | |
| | | | | Compound A in | |
| | | | | the refinement | |
| | | | | assay | |
| Uptake | KH | 37° C., | 100 | 6.25, 12.5, 25, 50 | |
| transporter | (pH 8.0) | 15 min | | and 100 μM | |
| inhibition | HBSS | | | and | |
| assay | (pH 7.4) | | | *25, 30, 35, 40, | |
| | | | | 45 and 50 μM in | |
| | | | | case of | |
| | | | | Compound A in | |
| | | | | the refinement | |
| | | | | assay | |

*A refinement solubility assessment was carried out in case of Compound A for more precise estimation of the solubility limit.

Compound I-19 was soluble up to 100 μM in all tested assay buffers (Table 68), transport buffer with sucrose for BCRP and MDR1 and in transport buffer for BSEP in the VT inhibition assay setup, and in KH (pH 8.0) and HBSS (pH 7.4) assay buffers in the uptake inhibition assay setup.

TABLE 68
Summary of the solubility assays of Compound I-19

| | | | Final | |
|---|---|---|---|---|
| | | | concen- | |
| | | Dilu- | tration | Micros- |
| | | tion | (nominal, | copy |
| Compound | Buffer | factor | μM) | results |
| Compound | Transport buffer with sucrose | 100 | 100.00 | Soluble |
| I-19 | (pH 7.4) | | 50.00 | Soluble |
| | for BCRP and MDR1 VT | | 25.00 | Soluble |
| | inhibition assay | | 12.50 | Soluble |
| | 32° C. | | 6.25 | Soluble |
| | Transport buffer | 100 | 100.00 | Soluble |
| | (pH 7.4) | | 50.00 | Soluble |
| | for BSEP | | 25.00 | Soluble |
| | VT inhibition assay | | 12.50 | Soluble |
| | 37° C. | | 6.25 | Soluble |
| | KH | 100 | 100.00 | Soluble |
| | (pH 8.0) | | 50.00 | Soluble |
| | for MATE1, MATE2-K | | 25.00 | Soluble |
| | uptake inhibition assay | | 12.50 | Soluble |
| | 37° C. | | 6.25 | Soluble |
| | HBSS | 100 | 100.00 | Soluble |
| | (pH 7.4) | | 50.00 | Soluble |
| | for OAT1, OAT3, OATP1B1, | | 25.00 | Soluble |
| | OATP1B3, OCT1, OCT2 | | 12.50 | Soluble |
| | uptake inhibition assay | | 6.25 | Soluble |
| | 37° C. | | | |

Compound A was soluble up to 50 μM in transport buffer with sucrose for BCRP and MDR1 VT inhibition assays and up to 35 μM in transport buffer for BSEP in the VT inhibition assay setup (Table 69). Compound A was soluble up to 30 μM in KH (pH 8.0) and HBSS (pH 7.4) assay buffers in the uptake inhibition assay setup (Table 69).

TABLE 69
Summary of the solubility assays of Compound A

| | | | Final | |
|---|---|---|---|---|
| | | | concen- | |
| | | Dilu- | tration | Micros- |
| | | tion | (nominal, | copy |
| Compound | Buffer | factor | μM) | results |
| Compound A | Transport buffer | 100 | 100.00 | Insoluble |
| | with sucrose | | 90.00 | Insoluble |
| | (pH 7.4) | | 80.00 | Insoluble |
| | for BCRP and MDR1 | | 70.00 | Insoluble |
| | VT inhibition assay | | 60.00 | Insoluble |
| | 32° C. | | 50.00 | Soluble |
| | | | 25.00 | Soluble |
| | | | 12.50 | Soluble |
| | | | 6.25 | Soluble |
| | Transport buffer | 100 | 100.00 | Insoluble |
| | (pH 7.4) | | 50.00 | Insoluble |
| | for BSEP | | 45.00 | Insoluble |
| | VT inhibition assay | | 40.00 | Insoluble |
| | 37° C. | | 35.00 | Soluble |
| | | | 30.00 | Soluble |
| | | | 25.00 | Soluble |
| | | | 12.50 | Soluble |
| | | | 6.25 | Soluble |
| | KH | 100 | 100.00 | Insoluble |
| | (pH 8.0) | | 50.00 | Insoluble |
| | for MATE1, MATE2-K | | 45.00 | Insoluble |
| | uptake inhibition assay | | 40.00 | Insoluble |
| | 37° C. | | 35.00 | Insoluble |
| | | | 30.00 | Soluble |
| | | | 25.00 | Soluble |
| | | | 12.50 | Soluble |
| | | | 6.25 | Soluble |
| | HBSS | 100 | 100.00 | Insoluble |
| | (pH 7.4) | | 50.00 | Insoluble |
| | for OAT1, OAT3, | | 45.00 | Insoluble |
| | OATP1B1, OATP1B3, | | 40.00 | Insoluble |
| | OCT1, OCT2 | | 35.00 | Insoluble |
| | uptake inhibition assay | | 30.00 | Soluble |
| | 37° C. | | 25.00 | Soluble |
| | | | 12.50 | Soluble |
| | | | 6.25 | Soluble |

Compound B was soluble up to 100 μM in all tested assay buffers (Table 70), transport buffer with sucrose for BCRP and MDR1 and in transport buffer for BSEP in the VT inhibition assay setup, and in KH (pH 8.0) and HBSS (pH 7.4) assay buffers in the uptake inhibition assay setup.

TABLE 70
Summary of the solubility assays of Compound B

| | | | Final | |
|---|---|---|---|---|
| | | | concen- | |
| | | Dilu- | tration | Micros- |
| | | tion | (nominal, | copy |
| Compound | Buffer | factor | μM) | results |
| Compound B | Transport buffer with sucrose | 100 | 100.00 | Soluble |
| | (pH 7.4) | | 50.00 | Soluble |
| | for BCRP and MDR1 | | 25.00 | Soluble |
| | VT inhibition assay | | 12.50 | Soluble |
| | 32° C. | | 6.25 | Soluble |
| | Transport buffer | 100 | 100.00 | Soluble |
| | (pH 7.4) | | 50.00 | Soluble |
| | for BSEP | | 25.00 | Soluble |
| | VT inhibition assay | | 12.50 | Soluble |
| | 37° C. | | 6.25 | Soluble |
| | KH | 100 | 100.00 | Soluble |
| | (pH 8.0) | | 50.00 | Soluble |
| | for MATE1, MATE2-K | | 25.00 | Soluble |
| | uptake inhibition assay | | 12.50 | Soluble |
| | 37° C. | | 6.25 | Soluble |
| | HBSS (pH 7.4) | 100 | 100.00 | Soluble |
| | for OAT1, OAT3, OATP1B1, | | 50.00 | Soluble |
| | OATP1B3, OCT1, OCT2 | | 25.00 | Soluble |
| | uptake inhibition assay | | 12.50 | Soluble |
| | 37° C. | | 6.25 | Soluble |

### Resazurin-Based Cytotoxicity Assay

The ability of Compound I-19, Compound A and Compound B to induce cytotoxic effects was investigated in 96-well plates in the presence of the corresponding cell lines, at four concentrations of the compound.

Compound I-19 did not reduce the cell viability of HEK293-MATE1-LV, HEK293-MATE2-K-LV, HEK293-OAT1-LV, HEK293-OAT3-LV, HEK293-OATP1B1-LV, HEK293-OATP1B3-LV, HEK293-OCT1-LV, HEK293-OCT2-LV and respective control cells under the tested assay conditions (Table 71). No cytotoxic effect was observed, as decrease of the relative cell viability was less than 20% compared to the test article's solvent (DMSO).

TABLE 71
Cytotoxic effect of Compound I-19 on the uptake
transporter overexpressing and control cells measured
in the resazurin-based cytotoxicity assay

| | | Nominal | Relative cell viability | |
|---|---|---|---|---|
| | Incubation | concen- | (% of control) | |
| | time | tration | transfected | control |
| Transporter | (min) | (μM) | cells | cells |
| MATE1 | 30 + 3 | 100.00 | 97.35 ± 2.64 | 85.16 ± 2.74 |
| | | 50.00 | 95.88 ± 2.04 | 93.64 ± 2.83 |
| | | 25.00 | 99.42 ± 1.56 | 98.17 ± 2.64 |
| | | 12.50 | 97.90 ± 1.86 | 97.67 ± 3.73 |
| | | 0.00 | 100.00 ± 2.14 | 100.00 ± 3.56 |
| MATE2-K | 30 + 1 | 100.00 | 101.71 ± 1.17 | 84.34 ± 2.47 |
| | | 50.00 | 106.73 ± 1.88 | 104.14 ± 1.46 |
| | | 25.00 | 104.66 ± 1.48 | 102.04 ± 1.69 |
| | | 12.50 | 101.07 ± 1.57 | 104.66 ± 2.14 |
| | | 0.00 | 100.00 ± 1.57 | 100.00 ± 0.61 |
| OAT1 | 30 + 2 | 100.00 | 88.69 ± 3.44 | 90.45 ± 6.49 |
| | | 50.00 | 95.01 ± 3.42 | 97.24 ± 5.90 |
| | | 25.00 | 95.83 ± 3.50 | 97.74 ± 6.23 |
| | | 12.50 | 97.97 ± 3.47 | 98.64 ± 5.98 |
| | | 0.00 | 100.00 ± 4.87 | 100.00 ± 8.57 |
| OAT3 | 30 + 1 | 100.00 | 97.06 ± 1.13 | 83.05 ± 3.00 |
| | | 50.00 | 97.87 ± 1.31 | 92.66 ± 3.32 |
| | | 25.00 | 98.86 ± 1.01 | 96.77 ± 3.72 |
| | | 12.50 | 99.42 ± 0.98 | 102.06 ± 3.84 |
| | | 0.00 | 100.00 ± 1.34 | 100.00 ± 5.05 |
| OATP1B1 | 30 + 3 | 100.00 | 94.19 ± 1.27 | 89.12 ± 4.63 |
| | | 50.00 | 98.71 ± 0.94 | 93.42 ± 4.34 |
| | | 25.00 | 98.85 ± 0.84 | 95.44 ± 4.81 |
| | | 12.50 | 99.91 ± 0.74 | 96.78 ± 4.68 |
| | | 0.00 | 100.00 ± 0.84 | 100.00 ± 6.55 |
| OATP1B3 | 30 + 2 | 100.00 | 97.75 ± 0.86 | 95.06 ± 1.40 |
| | | 50.00 | 101.42 ± 1.04 | 101.22 ± 1.01 |
| | | 25.00 | 103.07 ± 1.39 | 102.92 ± 0.53 |
| | | 12.50 | 102.23 ± 1.09 | 103.94 ± 0.47 |
| | | 0.00 | 100.00 ± 0.92 | 100.00 ± 0.60 |
| OCT1 | 30 + 3 | 100.00 | 86.46 ± 3.25 | 92.32 ± 6.47 |
| | | 50.00 | 92.82 ± 3.41 | 98.64 ± 5.26 |
| | | 25.00 | 94.46 ± 3.93 | 100.36 ± 5.65 |
| | | 12.50 | 92.84 ± 3.54 | 101.25 ± 5.53 |
| | | 0.00 | 100.00 ± 5.08 | 100.00 ± 7.52 |
| OCT2 | 30 + 1 | 100.00 | 96.29 ± 1.73 | 87.66 ± 0.85 |
| | | 50.00 | 99.58 ± 1.02 | 97.55 ± 0.85 |
| | | 25.00 | 99.94 ± 1.03 | 102.60 ± 0.80 |
| | | 12.50 | 99.66 ± 1.41 | 99.87 ± 1.09 |
| | | 0.00 | 100.00 ± 1.12 | 100.00 ± 0.35 |

Data are expressed as mean (n = 3) ± EP.

Compound A did not reduce the cell viability of HEK293-MATE1-LV, HEK293-MATE2-K-LV, HEK293-OAT1-LV, HEK293-OAT3-LV, HEK293-OATP1B1-LV, HEK293-OATP1B3-LV, HEK293-OCT1-LV, HEK293-OCT2-LV and respective control cells under the tested assay conditions (Table 72). No cytotoxic effect was observed, as decrease of the relative cell viability was less than 20% compared to the test article's solvent (DMSO).

TABLE 72
Cytotoxic effect of Compound A on the uptake transporter
overexpressing and control cells measured in
the resazurin-based cytotoxicity assay.

| | | Nominal | Relative cell viability | |
|---|---|---|---|---|
| | Incubation | concen- | (% of control) | |
| | time | tration | transfected | control |
| Transporter | (min) | (μM) | cells | cells |
| MATE1 | 30 + 3 | 30.00 | 100.38 ± 1.18 | 99.32 ± 1.20 |
| | | 20.00 | 101.82 ± 1.13 | 99.30 ± 1.01 |
| | | 10.00 | 100.94 ± 1.13 | 99.15 ± 1.14 |
| | | 5.00 | 100.44 ± 1.23 | 99.21 ± 1.16 |
| | | 0.00 | 100.00 ± 1.11 | 100.00 ± 1.14 |
| MATE2-K | 30 + 1 | 30.00 | 95.00 ± 1.36 | 99.57 ± 0.77 |
| | | 20.00 | 98.93 ± 1.00 | 99.74 ± 0.77 |
| | | 10.00 | 99.35 ± 0.57 | 101.55 ± 0.74 |
| | | 5.00 | 100.17 ± 1.24 | 98.30 ± 0.72 |
| | | 0.00 | 100.00 ± 0.70 | 100.00 ± 0.89 |
| OAT1 | 30 + 2 | 30.00 | 93.28 ± 2.26 | 102.66 ± 2.43 |
| | | 20.00 | 98.34 ± 0.33 | 103.10 ± 2.01 |
| | | 10.00 | 100.28 ± 0.26 | 102.26 ± 1.66 |
| | | 5.00 | 101.06 ± 0.56 | 99.60 ± 2.60 |
| | | 0.00 | 100.00 ± 0.22 | 100.00 ± 0.41 |
| OAT3 | 30 + 1 | 30.00 | 99.16 ± 0.66 | 98.11 ± 1.29 |
| | | 20.00 | 97.97 ± 0.89 | 95.85 ± 2.05 |
| | | 10.00 | 98.73 ± 0.68 | 96.14 ± 2.42 |
| | | 5.00 | 97.17 ± 0.40 | 98.41 ± 1.36 |
| | | 0.00 | 100.00 ± 0.24 | 100.00 ± 1.74 |
| OATP1B1 | 30 + 3 | 30.00 | 98.92 ± 1.07 | 96.60 ± 2.23 |
| | | 20.00 | 99.79 ± 0.88 | 96.00 ± 0.81 |
| | | 10.00 | 102.28 ± 0.69 | 93.21 ± 2.18 |
| | | 5.00 | 101.07 ± 0.61 | 95.83 ± 0.85 |
| | | 0.00 | 100.00 ± 0.76 | 100.00 ± 1.16 |
| OATP1B3 | 30 + 2 | 30.00 | 99.58 ± 0.58 | 100.55 ± 0.31 |
| | | 20.00 | 99.23 ± 0.43 | 98.24 ± 0.33 |
| | | 10.00 | 99.16 ± 1.10 | 93.50 ± 0.99 |
| | | 5.00 | 99.30 ± 0.80 | 96.08 ± 0.72 |
| | | 0.00 | 100.00 ± 0.32 | 100.00 ± 0.39 |
| OCT1 | 30 + 3 | 30.00 | 109.22 ± 8.33 | 102.27 ± 5.38 |
| | | 20.00 | 105.82 ± 8.04 | 98.58 ± 5.65 |
| | | 10.00 | 106.01 ± 8.03 | 99.88 ± 5.10 |
| | | 5.00 | 105.13 ± 8.07 | 95.85 ± 5.15 |
| | | 0.00 | 100.00 ± 10.60 | 100.00 ± 6.19 |
| OCT2 | 30 + 1 | 30.00 | 98.11 ± 0.97 | 96.68 ± 2.77 |
| | | 20.00 | 98.80 ± 0.86 | 96.81 ± 3.07 |
| | | 10.00 | 97.59 ± 1.08 | 93.96 ± 4.78 |
| | | 5.00 | 96.41 ± 1.41 | 95.32 ± 3.75 |
| | | 0.00 | 100.00 ± 1.00 | 100.00 ± 2.45 |

Data are expressed as mean (n = 3) ± EP.

Compound B reduced the cell viability of both HEK293-MATE1-LV and respective control cells at 100 μM under the tested assay conditions. A follow-up refinement assay was carried out to determine the highest non-cytotoxic concentration for further experiment. Compound B reduced the cell viability of HEK293-MATE1-LV cells at all tested concentrations and the cell viability of HEK293-Mock-LV cells at 90 and 100 μM (Table 73), therefore 50 μM Compound B was chosen for further MATE1 assays. Compound B reduced the cell viability of HEK293-Mock-LV cells, but not the viability of the HEK293-MATE2-K-LV cells at 100 μM under the tested MATE2-K assay conditions (Table 73). A follow-up refinement assay was carried out to determine the highest non-cytotoxic concentration for further experiment. Compound B reduced the cell viability of HEK293-Mock-LV cells at 90 and 100 μM (Table 73), therefore 80 μM Compound B was chosen for further MATE2-K assays. Compound B did not reduce the cell viability of HEK293-OAT1-LV, HEK293-OAT3-LV, HEK293-OATP1B1-LV, HEK293-OATP1B3-LV, HEK293-OCT1-LV, HEK293-OCT2-LV and respective control cells under the tested assay conditions (Table 73). No cytotoxic effect was observed, as decrease of the relative cell viability was less than 20% compared to the test article's solvent (DMSO).

TABLE 73
Cytotoxic effect of Compound B on the uptake transporter
overexpressing and control cells measured in
the resazurin-based cytotoxicity assay.

| | | Nominal | Relative cell viability | |
|---|---|---|---|---|
| | Incubation | concen- | (% of control) | |
| | time | tration | transfected | control |
| Transporter | (min) | (μM) | cells | cells |
| MATE1 | 30 + 3 | 100.00 | 73.95 ± 2.11 | 39.68 ± 1.27 |
| | | 50.00 | 96.96 ± 2.73 | 83.69 ± 1.28 |
| | | 25.00 | 93.79 ± 2.47 | 88.21 ± 0.68 |
| | | 12.50 | 100.39 ± 2.59 | 92.45 ± 2.45 |
| | | 0.00 | 100.00 ± 1.90 | 100.00 ± 1.01 |
| | | 100.00* | 29.37 ± 0.54 | 50.79 ± 16.49 |
| | | 90.00* | 52.52 ± 1.96 | 77.52 ± 3.40 |
| | | 80.00* | 61.93 ± 2.99 | 87.76 ± 2.19 |
| | | 70.00* | 75.61 ± 2.34 | 89.77 ± 2.30 |
| | | 0.00* | 100.00 ± 2.38 | 100.00 ± 2.21 |
| MATE2-K | 30 + 1 | 100.00 | 95.74 ± 1.87 | 29.88 ± 2.51 |
| | | 50.00 | 101.73 ± 1.82 | 90.83 ± 2.28 |
| | | 25.00 | 102.15 ± 1.54 | 96.01 ± 2.30 |
| | | 12.50 | 104.93 ± 1.06 | 101.41 ± 2.18 |
| | | 0.00 | 100.00 ± 0.98 | 100.00 ± 3.00 |
| | | 100.00* | 90.31 ± 2.30 | 57.97 ± 4.48 |
| | | 90.00* | 98.73 ± 1.14 | 62.97 ± 11.50 |
| | | 80.00* | 100.44 ± 1.22 | 97.01 ± 7.14 |
| | | 70.00* | 100.09 ± 1.26 | 93.56 ± 9.80 |
| | | 0.00* | 100.00 ± 1.58 | 100.00 ± 8.29 |
| OAT1 | 30 + 2 | 100.00 | 95.47 ± 3.78 | 91.21 ± 1.53 |
| | | 50.00 | 99.89 ± 3.09 | 95.96 ± 3.00 |
| | | 25.00 | 99.76 ± 3.81 | 97.04 ± 2.33 |
| | | 12.50 | 103.32 ± 3.91 | 98.88 ± 2.97 |
| | | 0.00 | 100.00 ± 4.14 | 100.00 ± 0.92 |
| OAT3 | 30 + 1 | 100.00 | 97.32 ± 1.48 | 74.69 ± 1.27 |
| | | 50.00 | 100.32 ± 0.96 | 94.52 ± 1.42 |
| | | 25.00 | 101.10 ± 1.03 | 98.37 ± 1.53 |
| | | 12.50 | 100.55 ± 0.41 | 101.61 ± 2.05 |
| | | 0.00 | 100.00 ± 0.10 | 100.00 ± 2.02 |
| OATP1B1 | 30 + 3 | 100.00 | 97.19 ± 2.22 | 92.55 ± 1.35 |
| | | 50.00 | 104.87 ± 2.47 | 104.12 ± 1.80 |
| | | 25.00 | 103.38 ± 3.37 | 107.53 ± 1.67 |
| | | 12.50 | 104.62 ± 2.75 | 105.81 ± 2.56 |
| | | 0.00 | 100.00 ± 3.16 | 100.00 ± 1.72 |
| OATP1B3 | 30 + 2 | 100.00 | 96.63 ± 1.31 | 89.50 ± 2.17 |
| | | 50.00 | 99.86 ± 1.74 | 101.42 ± 1.05 |
| | | 25.00 | 98.53 ± 1.62 | 98.54 ± 2.09 |
| | | 12.50 | 102.27 ± 3.56 | 101.26 ± 2.30 |
| | | 0.00 | 100.00 ± 1.82 | 100.00 ± 0.63 |
| OCT1 | 30 + 3 | 100.00 | 89.12 ± 2.09 | 91.17± 10.18 |
| | | 50.00 | 91.44 ± 2.71 | 100.29 ± 7.21 |
| | | 25.00 | 91.62 ± 3.80 | 98.57 ± 5.53 |
| | | 12.50 | 98.60 ± 2.46 | 106.24 ± 5.51 |
| | | 0.00 | 100.00 ± 2.93 | 100.00 ± 3.31 |
| OCT2 | 30 + 1 | 100.00 | 98.23 ± 1.77 | 84.08 ± 2.55 |
| | | 50.00 | 99.16 ± 1.54 | 96.71 ± 2.58 |
| | | 25.00 | 98.71 ± 1.58 | 97.28 ± 2.21 |
| | | 12.50 | 102.57 ± 1.67 | 99.98 ± 2.54 |
| | | 0.00 | 100.00 ± 2.18 | 100.0 3.07 |

Data are expressed as mean (n = 3) ± EP.
*Refinement cytotoxicity assay.

### Non-Specific Binding Assays

The non-specific binding (NSB) of Compound I-19, Compound A and Compound B to the surfaces of the plastic ware during the experiments was investigated in the respective assay setups in the absence of cells or membranes. All conditions and stock preparation procedures were identical to the conditions applied in the transporter assays (Table 74, Table 75 and Table 76). Compound I-19 did not show any loss due to plastic binding in the BCRP, BSEP and MDR1 VT inhibition assay setup. While more than 20% NSB was observed in HBSS (pH 7.4) and KH (pH 8.0) uptake assay buffers (Table 77). A follow-up assay was carried out at 7 concentrations in order to correct the nominal concentrations for the IC₅₀ calculations (Table 78). Where NSB was observed (>20% loss) the assay concentrations were corrected according to the result of the respective NSB assay.

TABLE 74
Experimental conditions for the non-specific binding experiments: Compound I-19

| | | Total | Final nominal | Samples taken |
|---|---|---|---|---|
| | | incubation time | concentrations | (n = 3/assay |
| Assay type | Assay buffer | (min) | (μM) | step) |
| Uptake transporter | KH (pH 8.0) | 15 + 30 + 3 | 10 and100 μM | C₀ or Total |
| inhibition assay | | at 37° C. | and | t_{inc} |
| (96-well plate) | HBSS (pH 7.4) | 15 + 30 + 3 | *0.14, 0.41, | |
| | | at 37° C. | 1.24, 3.70, 11.1, | |
| | | | 33.3 and 100 μM | |
| Vesicular transport | Transport buffer for | 15 + 1 | 10 and100 μM | 1.5 × C₀ or |
| inhibition assay | BCRP, MDR1 | at 32° C. | | Total t_{inc} |
| (96-well plate) | (Transport buffer with | | | |
| | sucrose, pH 7.4) | | | |
| | Transport buffer for | 15 + 5 | 10 and100 μM | |
| | BSEP | at 37° C. | | |
| | (BSEP Buffer, pH 7.4) | | | |

*Follow-up experiment for IC₅₀ determination.

TABLE 75
Experimental conditions for the non-specific binding experiments: Compound A

| | | Total | Final nominal | Samples taken |
|---|---|---|---|---|
| | | incubation time | concentrations | (n = 3/assay |
| Assay type | Assay buffer | (min) | (μM) | step) |
| Uptake transporter | KH (pH 8.0) | 15 + 30 + 3 | 3 and | C₀ or Total |
| inhibition assay | | at 37° C. | 30 μM | t_{inc} |
| (96-well plate) | HBSS (pH 7.4) | 15 + 30 + 3 | | |
| | | at 37° C. | | |
| Vesicular transport | Transport buffer for | 15 + 1 | 5 and | 1.5 × C₀ or |
| inhibition assay | BCRP, MDR1 | at 32° C. | 50 μM | Total t_{inc} |
| (96-well plate) | (Transport buffer with | | | |
| | sucrose, pH 7.4) | | | |
| | Transport buffer for | 15 + 5 | 3.5 and | |
| | BSEP | at 37° C. | 35 μM | |
| | (BSEP Buffer, pH 7.4) | | | |

TABLE 76
Experimental conditions for the non-specific binding experiments: Compound B.

| | | Total | Final nominal | |
|---|---|---|---|---|
| | | incubation time | concentrations | Samples taken |
| Assay type | Assay buffer | (min) | (μM) | (n = 3/assay step) |
| Uptake | KH (pH 8.0) | 15 + 30 + 3 | 5, 8, 50 and | C₀ or Total |
| transporter | | at 37° C. | 80 μM | t_{inc} |
| inhibition assay | HBSS (pH 7.4) | 15 + 30 + 3 | 10 and100 μM | |
| (96-well plate) | | at 37° C. | | |
| Vesicular | Transport buffer for | 15 + 1 | 10 and100 μM | 1.5 × C₀ or Total |
| transport | BCRP, MDR1 | at 32° C. | | t_{inc} |
| inhibition assay | (Transport buffer with | | | |
| (96-well plate) | sucrose, pH 7.4) | | | |
| | Transport buffer for | 15 + 5 | 10 and100 μM | |
| | BSEP | at 37° C. | | |
| | (BSEP Buffer, pH 7.4) | | | |

TABLE 77
Summary of the non-specific binding (NSB) experiments
in VT inhibition assay setup for Compound I-19.

| | | *Measured | | Loss of |
|---|---|---|---|---|
| | Nominal | dosing | Concentration | Compound I-19 |
| | concentrations | concentrations | after incubation | during the NSB |
| Transport buffer | (C₀, μM) | (1.5 × C₀, μM) | (t_{inc}, μM) | assay (%) |
| Transport buffer with | 100.00 | 167.14 ± 11.84 | 124.55 ± 5.96 | −12 |
| sucrose (pH 7.4) for | 10.00 | 17.68 ± 0.42 | 13.10 ± 1.26 | −11 |
| BCRP and MDR1 VT | | | | |
| inhibition assay | | | | |
| 32° C. | | | | |
| (LLOQ: 1.52 nM) | | | | |
| Transport buffer | 100.00 | 162.83 ± 11.51 | 122.52 ± 9.60 | −13 |
| (pH 7.4) for BSEP VT | 10.00 | 18.55 ± 0.06 | 12.92 ± 0.21 | −4 |
| inhibition assay | | | | |
| 37° C. | | | | |
| (LLOQ: 1.52 nM) | | | | |

Data are expressed as mean (n = 3) ± SD. LLOQ: Lowest limit of quantification.
*The experimental setup in the VT assay makes it necessary to perform the NSB assay with 1.5 × higher C₀ concentrations than the actual final concentration in the VT assay.

TABLE 78
Summary of the non-specific binding (NSB) experiments
in uptake inhibition assay buffers for Compound I-19

| | | Measured | | Loss of |
|---|---|---|---|---|
| | Nominal | dosing | Concentration | Compound I-19 |
| | concentrations | concentrations | after incubation | during the NSB |
| Transport buffer | (μM) | (μM) | (μM) | assay (%) |
| KH (pH 8.0) for | 100.000 | 30.52 ± 1.33 | 20.35 ± 1.94 | 33 |
| MATE1, MATE2-K | 10.000 | 8.68 ± 0.34 | 5.54 ± 0.13 | 36 |
| uptake inhibition assay | | | | |
| 37° C. | | | | |
| (LLOQ: 1.52 nM) | | | | |
| HBSS (pH 7.4) for | 100.00 | 76.99 ± 6.62 | 59.43 ± 5.59 | 23 |
| OAT1, OAT3, | 10.00 | 8.99 ± 0.60 | 5.98 ± 0.13 | 34 |
| OATPIB1, OATP1B3, | | | | |
| OCT1, OCT2 uptake | | | | |
| inhibition assay; 37° C. | | | | |
| (LLOQ: 4.57 nM) | | | | |

Data are expressed as mean (n = 3) ± SD. LLOQ: Lowest limit of quantification.

TABLE 79
Summary of the non-specific binding (NSB) experiments in uptake
inhibition assay buffers for Compound I-19 - IC₅₀ determination.

| | | Measured | | Loss of |
|---|---|---|---|---|
| | Nominal | dosing | Concentration | Compound I-19 |
| | concentrations | concentrations | after incubation | during the NSB |
| Transport buffer | (μM) | (μM) | (μM) | assay (%) |
| KH (pH 8.0) for | 100.00 | 45.32 ± 9.94 | 34.06 ± 2.30 | 25 |
| MATEI, MATE2-K | 33.33 | 20.72 ± 2.88 | 12.58 ± 1.30 | 39 |
| uptake inhibition | 11.11 | 7.32 ± 3.86 | 4.05 ± 0.49 | 45 |
| assay 37° C. | 3.704 | 2.63 ± 1.34 | 1.68 ± 0.26 | 36 |
| (LLOQ: 0.056 nM) | 1.235 | 1.10 ± 0.10 | 0.73 ± 0.04 | 34 |
| | 0.412 | 0.44 ± 0.10 | 0.27 ± 0.02 | 39 |
| | 0.137 | 0.17 ± 0.02 | 0.10 ± 0.01 | 41 |
| HBSS (pH 7.4) for | 100.00 | 38.99 ± 3.97 | 34.49 ± 3.00 | 12 |
| OAT1, OAT3, | | | | |
| OATP1B1, | 33.33 | 23.77 ± 2.59 | 17.44 ± 3.56 | 27 |
| OATP1B3, OCT1, | 11.11 | 9.59 ± 1.50 | 6.86 ± 0.20 | 29 |
| OCT2 uptake | 3.704 | 4.84 ± 0.73 | 2.49 ± 0.18 | 49 |
| inhibition assay | 1.235 | 1.18 ± 0.09 | 0.83 ± 0.07 | 30 |
| 37° C. | 0.412 | 0.57 ± 0.14 | 0.35 ± 0.03 | 39 |
| (LLOQ: 0.056 nM) | 0.137 | 0.23 ± 0.02 | 0.15 ± 0.01 | 33 |

Data are expressed as mean (n = 3) ± SD. LLOQ: Lowest limit of quantification.

Compound A did not show any NSB in the BCRP, BSEP and MDR1 VT inhibition assay setup (Table 80) and in HBSS (pH 7.4) and KH (pH 8.0) uptake assay buffers (Table 81).

TABLE 80
Summary of the non-specific binding (NSB) experiments
in VT inhibition assay setup for Compound A

| | | *Measured | | Loss of |
|---|---|---|---|---|
| | Nominal | dosing | Concentration | Compound A |
| | concentrations | concentrations | after incubation | during the NSB |
| Transport buffer | (C₀, μM) | (1.5 × C₀, μM) | (t_{inc}, μM) | assay (%) |
| Transport buffer with | 50.00 | 82.20 ± 3.76 | 55.92 ± 2.44 | −2 |
| sucrose (pH 7.4) for | | | | |
| BCRP and MDR1 VT | 5.00 | 9.65 ± 1.52 | 6.27 ± 0.69 | 3 |
| inhibition assay; 32° C. | | | | |
| (LLOQ: 4.572 nM) | | | | |
| Transport buffer (pH | 35.00 | 63.99 ± 2.33 | 38.39 ± 0.28 | 10 |
| 7.4) for BSEP VT | | | | |
| inhibition assay; 37° C. | 3.50 | 7.31 ± 0.42 | 4.86 ± 0.45 | 0 |
| (LLOQ: 1.524 nM) | | | | |

Data are expressed as mean (n = 3) ± SD. LLOQ: Lowest limit of quantification.
*The experimental setup in the VT assay makes it necessary to perform the NSB assay with 1.5 × higher C₀ concentrations than the actual final concentration in the VT assay.

TABLE 81
Summary of the non-specific binding (NSB) experiments
in uptake inhibition assay buffers for Compound A

| | | | | Loss of |
|---|---|---|---|---|
| | Nominal | Measured dosing | Concentration | Compound A |
| | concentrations | concentrations | after incubation | during the NSB |
| Transport buffer | (μM) | (μM) | (μM) | assay (%) |
| KH (pH 8.0) for | 30.00 | 22.13 ± 0.61 | 18.15 ± 0.42 | 18 |
| MATEI, MATE2-K | 3.00 | 2.31 ± 0.11 | 1.86 ± 0.06 | 20 |
| uptake inhibition assay | | | | |
| 37° C. | | | | |
| (LLOQ: 0.508 nM) | | | | |
| HBSS (pH 7.4) for | 30.00 | 20.71 ± 0.67 | 17.66 ± 0.35 | 15 |
| OATI, | 3.00 | 2.16 ± 0.05 | 1.85 ± 0.13 | 14 |
| OAT3,OATP1B1, | | | | |
| OATP1B3, OCT1, | | | | |
| OCT2 uptake | | | | |
| inhibition assay 37° C. | | | | |
| (LLOQ: 0.508 nM) | | | | |

Data are expressed as mean (n = 3) ± SD. LLOQ: Lowest limit of quantification.

Compound B did not show any NSB in the BCRP, BSEP and MDR1 VT inhibition assay setup (Table 82) and in HBSS (pH 7.4) and KH (pH 8.0) uptake assay buffers (Table 83).

TABLE 82
Summary of the non-specific binding (NSB) experiments
in VT inhibition assay setup for Compound B

| | | *Measured | | Loss of |
|---|---|---|---|---|
| | Nominal | dosing | Concentration | Compound B |
| | concentrations | concentrations | after incubation | during the NSB |
| Transport buffer | (C₀, μM) | (1.5 × C₀, μM) | (t_{inc}, μM) | assay (%) |
| Transport buffer with | 100.00 | 107.84 ± 7.18 | 73.75 ± 0.85 | −3 |
| sucrose (pH 7.4) for | 10.00 | 11.44 ± 0.50 | 8.66 ± 0.98 | −13 |
| BCRP and MDR1 VT | | | | |
| inhibition assay 32° C. | | | | |
| (LLOQ: 1.52 nM) | | | | |
| Transport buffer (pH | 100.00 | 134.32 ± 10.11 | 91.89 ± 1.31 | −3 |
| 7.4) for BSEP VT | 10.00 | 16.21 ± 1.01 | 11.71 ± 0.98 | −8 |
| inhibition assay 37° C. | | | | |
| (LLOQ: 1.52 nM) | | | | |

Data are expressed as mean (n = 3) ± SD. LLOQ: Lowest limit of quantification.
*The experimental setup in the VT assay makes it necessary to perform the NSB assay with 1.5 × higher C₀ concentrations than the actual final concentration in the VT assay.

TABLE 83
Summary of the non-specific binding (NSB) experiments
in uptake inhibition assay buffers for Compound B.

| | | | | Loss of |
|---|---|---|---|---|
| | Nominal | Measured dosing | Concentration | Compound B |
| | concentrations | concentrations | after incubation | during the NSB |
| Transport buffer | (μM) | (μM) | (μM) | assay (%) |
| KH (pH 8.0) for | 80.00 | 56.68 ± 4.96 | 54.09 ± 3.80 | 5 |
| MATE1, MATE2-K | 50.00 | 38.65 ± 0.42 | 37.44 ± 3.56 | 3 |
| uptake inhibition assay | 8.00 | 7.21 ± 0.28 | 6.37 ± 0.22 | 12 |
| 37° C. | 5.00 | 4.31 ± 0.24 | 3.70 ± 0.19 | 14 |
| (LLOQ: 1.52 nM) | | | | |
| HBSS (pH 7.4) for | 100.00 | 100.89 ± 7.90 | 76.84 ± 0.90 | 24 |
| OATI, OAT3, | 10.00 | 8.83 ± 0.64 | 8.25 ± 0.12 | 7 |
| OATP1B1, | | | | |
| OATP1B3, OCT1, | | | | |
| OCT2 uptake | | | | |
| inhibition assay 37° C. | | | | |
| (LLOQ: 1.52 nM) | | | | |

Data are expressed as mean (n = 3) ± SD. LLOQ: Lowest limit of quantification.

### Vesicular Transport Inhibition Assays

The in vitro interaction potential of Compound I-19, Compound A and Compound B with the human BCRP, BSEP and MDR1 efflux (ABC) transporters was tested at 2 concentrations of the test compounds (Table 84). Follow-up assays were carried out at seven concentrations to determine the IC₅₀ values in those cases where more than 50% of inhibition was observed.

TABLE 84
Vesicular transport inhibition assay conditions, controls
Treatment groups in 96-well plate format
Transporters: BCRP, BSEP and MDR1

| Assay Conditions | Controls |
|---|---|
| Compound I-19 and Compound B: Solvent | 4 mM Mg-ATP or 4 mM AMP |
| control, at 10 and 100 μM and at 0.14, 0.41, | Functionality control: |
| 1.24, 3.70, 11.1, 33.3 and 100 μM in the follow- | Reference inhibitor |
| up assays | (positive control for |
| Compound A: 3.5 and 35 μM for BSEP and 5 | inhibition) |
| and 50 μM for BCRP and MDR1 | No. of replicates: 3 |
| 4 mM Mg-ATP or 4 mM AMP | |
| No. of replicates: 3 | |

Compound I-19 inhibited the BCRP-mediated estrone-3-sulfate (E3S) accumulation with a maximum inhibition of 54% at 100 μM (Table 85). In the follow-up assay the maximum inhibition was 43% (Table 86), therefore no IC₅₀ was calculated.

TABLE 85
Effect of Compound I-19 on the BCRP-mediated transport of estrone-3-
sulfate (E3S) measured in the vesicular transport inhibition assay

| | Concentration | | Relative ATP- | Relative |
|---|---|---|---|---|
| | (μM) | ATP-dependent | dependent transport | inhibition |
| Compound | nominal | transport (cpm) | (% of control) | (%) |
| Compound I-19 | 100.00 | 7,418.33 ± 222.69 | 45.74 ± 3.45 | 54 |
| | 10.00 | 15,363.00 ± 602.94 | 94.72 ± 7.53 | 5 |
| DMSO | 0.00 | 16,219.33 ± 1,121.72 | 100.00 ± 9.78 | 0 |
| Ko143 | 0.20 | 19.00 ± 23.70 | 0.12 ± 0.15 | 100 |

Data are expressed as mean (n = 3) ± EP.

TABLE 86
Effect of Compound I-19 on the BCRP-mediated transport of estrone-3-sulfate (E3S)
measured in the vesicular transport inhibition assay - IC₅₀ determination

| | Concentration | | Relative ATP- | Relative |
|---|---|---|---|---|
| | (μM) | ATP-dependent | dependent transport | inhibition |
| Compound | nominal | transport (cpm) | (% of control) | (%) |
| Compound I-19 | 100.00 | 7,718.00 ± 183.19 | 56.54 ± 4.97 | 43 |
| | 33.33 | 10,739.00 ± 289.94 | 78.67 ± 6.99 | 21 |
| | 11.11 | 12,797.67 ± 194.88 | 93.75 ± 8.06 | 6 |
| | 3.70 | 12,367.67 ± 999.00 | 90.60 ± 10.60 | 9 |
| | 1.24 | 12,617.00 ± 500.27 | 92.42 ± 8.64 | 8 |
| | 0.41 | 11,874.33 ± 219.87 | 86.98 ± 7.54 | 13 |
| | 0.14 | 13,445.33 ± 1,138.41 | 98.49 ± 11.79 | 2 |
| DMSO | 0.00 | 13,651.33 ± 1,155.79 | 100.00 ± 11.97 | 0 |
| Ko143 | 0.20 | 205.33 ± 40.97 | 1.50 ± 0.33 | 98 |

Data are expressed as mean (n = 3) ± EP.

Compound I-19 inhibited the BSEP-mediated taurocholate (TC) accumulation with a maximum inhibition of 77% at 100 μM (Table 87). In the follow-up assay the calculated IC₅₀ value was 45.3 μM with a bottom constrained to 0 and with a maximum inhibition of 73% (Table 88).

TABLE 87
Effect of Compound I-19 on the BSEP-mediated transport of taurocholate
measured in the vesicular transport inhibition assay

| | Concentration | | Relative ATP- | Relative |
|---|---|---|---|---|
| | (μM) | ATP-dependent | dependent transport | Inhibition |
| Compound | nominal | transport (cpm) | (% of control) | (%) |
| Compound I-19 | 100.00 | 1,103.00 ± 205.45 | 22.53 ± 4.22 | 77 |
| | 10.00 | 4,410.33 ± 297.54 | 90.09 ± 6.36 | 10 |
| DMSO | 0.00 | 4,895.67 ± 101.74 | 100.00 ± 2.94 | 0 |
| Cyclosporin A | 10.00 | 84.00 ± 16.07 | 1.72 ± 0.33 | 98 |

Data are expressed as mean (n = 3) ± EP.

TABLE 88
Effect of Compound I-19 on the BSEP-mediated transport of taurocholate measured
in the vesicular transport inhibition assay - IC₅₀ determination

| | Concentration | | Relative ATP- | Relative |
|---|---|---|---|---|
| | (μM) | ATP-dependent | dependent transport | Inhibition |
| Compound | nominal | transport (cpm) | (% of control) | (%) |
| Compound I-19 | 100.00 | 1,333.33 ± 146.13 | 26.92 ± 3.20 | 73 |
| | 33.33 | 3,105.00 ± 99.98 | 62.69 ± 3.53 | 37 |
| | 11.11 | 4,442.00 ± 98.02 | 89.68 ± 4.59 | 10 |
| | 3.70 | 4,958.00 ± 406.46 | 100.09 ± 9.42 | 0 |
| | 1.24 | 4,919.33 ± 50.10 | 99.31 ± 4.70 | 1 |
| | 0.41 | 4,781.00 ± 131.36 | 96.52 ± 5.19 | 3 |
| | 0.14 | 5,664.33 ± 228.93 | 114.35 ± 6.07 | −14 |
| DMSO | 0.00 | 4,953.33 ± 228.93 | 100.00 ± 6.54 | 0 |
| Cyclosporin A | 10.00 | 142.67 ± 14.24 | 2.88 ± 0.32 | 97 |

Data are expressed as mean (n = 3) ± EP.

Compound I-19 inhibited the MDR1-mediated N-methyl quinidine (NMQ) accumulation with a maximum inhibition of 92% at 100 μM (Table 89). In the follow-up assay the calculated IC₅₀ value was 21.0 μM with a maximum inhibition of 89% (Table 90) and with a bottom constrained to 0.

TABLE 89
Effect of Compound I-19 on the MDR1-mediated transport of N-methyl quinidine
(NMQ) measured in the vesicular transport inhibition assay

| | Concentration | | Relative ATP- | Relative |
|---|---|---|---|---|
| | (μM) | ATP-dependent | dependent transport | inhibition |
| Compound | nominal | transport (cpm) | (% of control) | (%) |
| Compound I-19 | 100.00 | 2.84 ± 21.41 | 7.89 ± 0.61 | 92 |
| | 10.00 | 1,845.67 ± 148.09 | 51.19 ± 4.18 | 49 |
| DMSO | 0.00 | 3,605.67 ± 55.93 | 100.00 ± 2.19 | 0 |
| valspodar | 1.00 | 6.67 ± 24.65 | 0.18 ± 0.68 | 100 |

Data are expressed as mean (n = 3) ± EP.

TABLE 90
Effect of Compound I-19 on the MDR1-mediated transport of N-methyl quinidine
(NMQ) measured in the vesicular transport inhibition assay

| | Concentration | | Relative ATP- | Relative |
|---|---|---|---|---|
| | (μM) | ATP-dependent | dependent transport | Inhibition |
| Compound | nominal | transport (cpm) | (% of control) | (%) |
| Compound I-19 | 100.00 | 407.00 ± 29.39 | 10.79 ± 0.84 | 89 |
| | 33.33 | 1,363.00 ± 46.72 | 36.12 ± 1.64 | 64 |
| | 11.11 | 2,730.67 ± 83.62 | 72.36 ± 3.10 | 28 |
| | 3.70 | 3,268.33 ± 26.42 | 86.61 ± 2.69 | 13 |
| | 1.24 | 3,744.33 ± 56.10 | 99.23 ± 3.33 | 1 |
| | 0.41 | 3,847.67 ± 71.06 | 101.97 ± 3.59 | −2 |
| | 0.14 | 3,812.00 ± 42.21 | 101.02 ± 3.23 | −1 |
| DMSO | 0.00 | 3,773.50 ± 113.16 | 100.00 ± 4.24 | 0 |
| Valspodar | 1.00 | 70.67 ± 25.49 | 1.87 ± 0.68 | 98 |

Data are expressed as mean (n = 3) ± EP.

Compound A did not inhibit the BCRP-mediated E3S accumulation at the tested concentrations, up to 50 μM (Table 91).

TABLE 91
Effect of Compound A on the BCRP-mediated transport of estrone-3-
sulfate (E3S) measured in the vesicular transport inhibition assay

| | Concentration | | Relative ATP- | Relative |
|---|---|---|---|---|
| | (μM) | ATP-dependent | dependent transport | inhibition |
| Compound | nominal | transport (cpm) | (% of control) | (%) |
| Compound A | 50.00 | 19,613.00 ± 758.83 | 97.83 ± 3.82 | 2 |
| | 5.00 | 20,104.00 ± 1,164.63 | 100.28 ± 5.84 | 0 |
| DMSO | 0.00 | 20,048.17 ± 112.37 | 100.00 ± 0.79 | 0 |
| Ko143 | 0.20 | 324.67 ± 21.61 | 1.62 ± 0.11 | 98 |

Data are expressed as mean (n = 3) ± EP.

Compound A did not inhibit the BSEP-mediated TC accumulation at the tested concentrations, up to 35 μM (Table 92).

TABLE 92
Effect of Compound A on the BSEP-mediated transport of taurocholate
measured in the vesicular transport inhibition assay

| | Concentration | | Relative ATP- | Relative |
|---|---|---|---|---|
| | (μM) | ATP-dependent | dependent transport | inhibition |
| Compound | nominal | transport (cpm) | (% of control) | (%) |
| Compound A | 35.00 | 8,577.33 ± 37.91 | 98.22 ± 2.92 | 2 |
| | 3.50 | 8,732.67 ± 295.43 | 100.00 ± 4.48 | 0 |
| DMSO | 0.00 | 8,732.33 ± 256.41 | 100.00 ± 4.15 | 0 |
| Cyclosporin A | 10.00 | 287.67 ± 20.28 | 3.29 ± 0.25 | 97 |

Data are expressed as mean (n = 3) ± EP.

Compound A did not inhibit the MDR1-mediated NMQ accumulation at the tested concentrations, up to 50 μM (Table 93).

TABLE 93
Effect of Compound A on the MDR1-mediated transport of N-methyl quinidine
(NMQ) measured in the vesicular transport inhibition assay

| | Concentration | | Relative ATP- | Relative |
|---|---|---|---|---|
| | (μM) | ATP-dependent | dependent transport | inhibition |
| Compound | nominal | transport (cpm) | (% of control) | (%) |
| Compound A | 50.00 | 2,735.00 ± 118.87 | 97.83 ± 5.19 | 2 |
| | 5.00 | 2,770.67 ± 248.31 | 99.11 ± 9.38 | 1 |
| DMSO | 0.00 | 2,795.67 ± 85.28 | 100.00 ± 4.31 | 0 |
| Valspodar | 1.00 | 41.00 ± 38.54 | 1.47 ± 1.38 | 99 |

Data are expressed as mean (n = 3) ± EP.

Compound B did not inhibit the BCRP-mediated E3S accumulation at the tested concentrations, up to 100 μM (Table 94).

TABLE 94
Effect of Compound B on the BCRP-mediated transport of estrone-3-
sulfate (E3S) measured in the vesicular transport inhibition assay

| | Concentration | | Relative ATP- | Relative |
|---|---|---|---|---|
| | (μM) | ATP-dependent | dependent transport | inhibition |
| Compound | nominal | transport (cpm) | (% of control) | (%) |
| Compound B | 100.00 | 13,933.00 ± 1,283.63 | 92.95 ± 8.76 | 7 |
| | 10.00 | 16,421.00 ± 786.73 | 109.55 ± 5.68 | −10 |
| DMSO | 0.00 | 14,989.00 ± 296.43 | 100.00 ± 2.80 | 0 |
| Ko143 | 0.20 | −32.33 ± 41.85 | −0.22 ± 0.28 | 100 |

Data are expressed as mean (n = 3) ± EP.

Compound B did not inhibit the BSEP-mediated TC accumulation at the tested concentrations, up to 100 μM (Table 95).

TABLE 95
Effect of Compound B on the BSEP-mediated transport of taurocholate
measured in the vesicular transport inhibition assay

| | Concentration | | Relative ATP- | Relative |
|---|---|---|---|---|
| | (μM) | ATP-dependent | dependent transport | inhibition |
| Compound | nominal | transport (cpm) | (% of control) | (%) |
| Compound B | 100.00 | 4,662.00 ± 138.31 | 94.31 ± 6.09 | 6 |
| | 10.00 | 4,765.67 ± 226.61 | 96.41 ± 7.18 | 4 |
| DMSO | 0.00 | 4,943.33 ± 283.30 | 100.00 ± 8.10 | 0 |
| Cyclosporin A | 10.00 | 87.33 ± 6.54 | 1.77 ± 0.17 | 98 |

Data are expressed as mean (n = 3) ± EP.

Compound B inhibited the MDR1-mediated NMQ accumulation with a maximum inhibition of 58% at 100 μM (Table 96). In the follow-up assay the calculated IC₅₀ value was 87.8 μM with a maximum inhibition of 53% (Table 97) and with a bottom constrained to 0.

TABLE 96
Effect of Compound B on the MDR1-mediated transport of N-methyl quinidine
(NMQ) measured in the vesicular transport inhibition assay

| | Concentration | | Relative ATP- | Relative |
|---|---|---|---|---|
| | (μM) | ATP-dependent | dependent transport | inhibition |
| Compound | nominal | transport (cpm) | (% of control) | (%) |
| Compound B | 100.00 | 1,331.67 ± 71.86 | 41.60 ± 2.41 | 58 |
| | 10.00 | 3,045.00 ± 79.38 | 95.13 ± 3.20 | 5 |
| DMSO | 0.00 | 3,201.00 ± 67.96 | 100.00 ± 3.00 | 0 |
| Valspodar | 1.00 | 22.33 ± 27.08 | 0.70 ± 0.85 | 99 |

Data are expressed as mean (n = 3) ± EP.

TABLE 97
Effect of Compound B on the MDR1-mediated transport of N-methyl quinidine (NMQ)
measured in the vesicular transport inhibition assay - IC₅₀ determination

| | Concentration | | Relative ATP- | Relative |
|---|---|---|---|---|
| | (μM) | ATP-dependent | dependent transport | Inhibition |
| Compound | nominal | transport (cpm) | (% of control) | (%) |
| Compound B | 100.00 | 1,744.67 ± 46.06 | 47.50 ± 2.65 | 53 |
| | 33.33 | 2,920.33 ± 26.95 | 79.51 ± 3.97 | 20 |
| | 11.11 | 3,634.67 ± 140.22 | 98.96 ± 6.18 | 1 |
| | 3.70 | 3,679.33 ± 40.87 | 100.17 ± 5.04 | 0 |
| | 1.24 | 3,734.33 ± 153.62 | 101.67 ± 6.51 | −2 |
| | 0.41 | 3,852.00 ± 72.84 | 104.87 ± 5.52 | −5 |
| | 0.14 | 3,765.67 ± 208.14 | 102.52 ± 7.58 | −3 |
| DMSO | 0.00 | 3,673.00 ± 180.43 | 100.00 ± 6.95 | 0 |
| Valspodar | 1.00 | 141.00 ± 77.77 | 3.84 ± 2.13 | 96 |

Data are expressed as mean (n = 3) ± EP.

### Uptake Transporter Inhibition Assays

The in vitro interaction potential of Compound I-19, Compound A and Compound B with the human MATE1, MATE2-K, OAT1, OAT3, OATP1B1, OATP1B3, OCT1 and OCT2 SLC transporters was tested at 2 concentrations of the test compounds (Table 98). Follow-up assays were carried out at seven concentrations to determine the IC₅₀ values in those cases where more than 50% of inhibition was observed.

TABLE 98
Uptake transporter inhibition assay conditions, controls
Transporters: MATE1, MATE2-K, OAT1,
OAT3, OATP1B1, OATP1B3, OCT1 and OCT2

| Assay Conditions | Controls |
|---|---|
| In transporter expressing | In transporter |
| and control cells | expressing and |
| Compound I-19 and Compound B: Solvent | control cells |
| control, at 10 and 100 μM and | Functionality control: |
| 0.14, 0.41, 1.24, 3.70, 11.1, | Reference inhibitor |
| 33.3 and 100 μM in the | (positive control for |
| follow-up assays | inhibition) |
| Compound A: Solvent control, | No. of replicates: 3 |
| at 3 and 30 μM and at | |
| 0.041, 0.123, 0.37, 1.11, | |
| 3.33, 10 and 30 μM in the | |
| follow-up assays | |
| No. of replicates: 3 | |

Compound I-19 completely inhibited the MATE1-mediated metformin accumulation at 100 μM (Table 99). In the follow-up assay the calculated IC₅₀ value was 8.86 μM using the NSB-corrected concentrations (Table 100) with a bottom constraint to 0.

TABLE 99
Effect of Compound I-19 on the MATE1-mediated transport of metformin
measured in the uptake transporter inhibition assay

| | | | | Relative | |
|---|---|---|---|---|---|
| | | | Transporter | transporter | |
| | Concentration (μM) | | specific | specific | |
| | | NSB | accumulation | accumulation | Relative |
| Compound | nominal | Corrected | (cpm) | (% of control) | inhibition (%) |
| Compound I-19 | 100.00 | 67.00 | −5.67 ± 2.36 | −1.49 ± 0.63 | 101 |
| | 10.00 | 6.50 | 173.67 ± 6.31 | 45.62 ± 4.36 | 54 |

| | | | | |
|---|---|---|---|---|
| DMSO | 0.00 | 380.67 ± 33.67 | 100.00 ± 12.51 | 0 |
| Pyrimethamine | 5.00 | −6.00 ± 4.71 | −1.58 ± 1.25 | 102 |

Data are expressed as mean (n = 3) ± EP. Concentrations were corrected according to the result of the NSB assays at those concentration points where the loss of Compound I-19 during the NSB experiment was more than 20%.

TABLE 100
Effect of Compound I-19 on the MATE1-mediated transport of metformin
measured in the uptake transporter inhibition assay

| | | | | Relative | |
|---|---|---|---|---|---|
| | | | Transporter | transporter | |
| | Concentration (μM) | | specific | specific | |
| | | NSB | accumulation | accumulation (%) | Relative |
| Compound | nominal | Corrected | (cpm) | of control) | inhibition (%) |
| Compound I-19 | 100.00 | 75.00 | −22.00 ± 1.89 | −4.47 ± 0.42 | 104 |
| | 33.33 | 20.33 | 57.67 ± 8.46 | 11.73 ± 1.78 | 88 |
| | 11.11 | 6.11 | 182.00 ± 22.40 | 37.02 ± 4.79 | 63 |
| | 3.70 | 2.37 | 344.33 ± 16.76 | 70.03 ± 4.41 | 30 |
| | 1.24 | 0.79 | 400.00 ± 11.06 | 81.36 ± 3.96 | 19 |
| | 0.41 | 0.25 | 401.00 ± 20.84 | 81.56 ± 5.35 | 18 |
| | 0.14 | 0.08 | 468.00 ± 24.27 | 95.19 ± 6.24 | 5 |

| | | | | |
|---|---|---|---|---|
| DMSO | 0.00 | 491.67 ± 19.69 | 100.00 ± 5.66 | 0 |
| Pyrimethamine | 5.00 | 0.00 ± 3.25 | 0.00 ± 0.66 | 100 |

Data are expressed as mean (n = 3) ± EP. Concentrations were corrected according to the result of the NSB assays at those concentration points where the loss of Compound I-19 during the NSB experiment was more than 20%.

Compound I-19 inhibited the MATE2-K-mediated metformin accumulation with a maximum inhibition of 93% at 100 μM (Table 101). In the follow-up assay the calculated IC₅₀ value was 36.6 μM using the NSB-corrected concentrations with a bottom constraint to 0 and with a maximum inhibition of 86% (Table 102).

TABLE 101
Effect of Compound I-19 on the MATE2-K-mediated transport of metformin
measured in the uptake transporter inhibition assay

| | | | | Relative | |
|---|---|---|---|---|---|
| | | | Transporter | transporter | |
| | Concentration (μM) | | specific | specific | |
| | | NSB | accumulation | accumulation (% | Relative |
| Compound | nominal | Corrected | (cpm) | of control) | inhibition (%) |
| Compound I-19 | 100.00 | 67.00 | 12.67 ± 8.97 | 7.06 ± 5.01 | 93 |
| | 10.00 | 6.50 | 154.00 ± 7.39 | 85.87 ± 5.57 | 14 |

| | | | | |
|---|---|---|---|---|
| DMSO | 0.00 | 179.33 ± 7.82 | 100.00 ± 6.17 | 0 |
| Pyrimethamine | 5.00 | 8.67 ± 6.51 | 4.83 ± 3.64 | 95 |

Data are expressed as mean (n = 3) ± EP. Concentrations were corrected according to the result of the NSB assays at those concentration points where the loss of Compound I-19 during the NSB experiment was more than 20%.

TABLE 102
Effect of Compound I-19 on the MATE2-K-mediated transport of metformin
measured in the uptake transporter inhibition assay

| | | | | Relative | |
|---|---|---|---|---|---|
| | | | Transporter | transporter | |
| | Concentration (μM) | | specific | specific | |
| | | NSB | accumulation | accumulation (% | Relative |
| Compound | nominal | Corrected | (cpm) | of control) | inhibition (%) |
| Compound I-19 | 100.00 | 75.00 | 14.67 ± 0.75 | 14.01 ± 1.15 | 86 |
| | 33.33 | 20.33 | 58.67 ± 0.67 | 56.05 ± 3.67 | 44 |
| | 11.11 | 6.11 | 88.67 ± 1.33 | 84.71 ± 5.61 | 15 |
| | 3.70 | 2.37 | 104.00 ± 1.89 | 99.36 ± 6.66 | 1 |
| | 1.24 | 0.79 | 105.00 ± 5.96 | 100.32 ± 8.62 | 0 |
| | 0.41 | 0.25 | 109.33 ± 3.71 | 104.46 ± 7.61 | −4 |
| | 0.14 | 0.08 | 99.67 ± 6.15 | 95.22 ± 8.50 | 5 |

| | | | | |
|---|---|---|---|---|
| DMSO | 0.00 | 104.67 ± 6.75 | 100.00 ± 9.12 | 0 |
| Pyrimethamine | 5.00 | −0.67 ± 2.11 | −0.64 ± 2.01 | 101 |

Data are expressed as mean (n = 3) ± EP. Concentrations were corrected according to the result of the NSB assays at those concentration points where the loss of Compound I-19 during the NSB experiment was more than 20%.

Compound I-19 inhibited the OAT1-mediated tenofovir accumulation with a maximum inhibition of 33% at 100 μM (Table 103).

TABLE 103
Effect of Compound I-19 on the OAT1-mediated transport of tenofovir measured
in the uptake transporter inhibition assay

| | | | | Relative | |
|---|---|---|---|---|---|
| | | | Transporter | transporter | |
| | Concentration (μM) | | specific | specific | |
| | | NSB | accumulation | accumulation (% | Relative |
| Compound | nominal | Corrected | (cpm) | of control) | inhibition (%) |
| Compound I-19 | 100.00 | 77.00 | 4,070.33 ± 51.74 | 66.61 ± 1.15 | 33 |
| | 10.00 | 6.60 | 5,462.33 ± 30.98 | 89.39 ± 1.16 | 11 |

| | | | | |
|---|---|---|---|---|
| DMSO | 0.00 | 6,111.00 ± 71.03 | 100.00 ± 1.64 | 0 |
| Probenecid | 300.00 | 97.00 ± 12.83 | 1.59 ± 0.21 | 98 |

Data are expressed as mean (n = 3) ± EP. Concentrations were corrected according to the result of the NSB assays at those concentration points where the loss of Compound I-19 during the NSB experiment was more than 20%.

Compound I-19 inhibited the OAT3-mediated E3S accumulation with a maximum inhibition of 42% at 100 μM (Table 104).

TABLE 104
Effect of Compound I-19 on the OAT3-mediated transport of estrone-3-sulfate
(E3S) measured in the uptake transporter inhibition assay

| | | | | Relative | |
|---|---|---|---|---|---|
| | | | Transporter | transporter | |
| | Concentration (μM) | | specific | specific | |
| | | NSB | accumulation | accumulation | Relative |
| Compound | nominal | Corrected | (cpm) | (% of control) | inhibition (%) |
| Compound I-19 | 100.00 | 77.00 | 6,272.33 ± 68.97 | 57.83 ± 0.99 | 42 |
| | 10.00 | 6.60 | 10,323.00 ± 126.84 | 95.18 ± 1.71 | 5 |

| | | | | |
|---|---|---|---|---|
| DMSO | 0.00 | 10,845.33 ± 142.67 | 100.00 ± 1.86 | 0 |
| Probenecid | 500.00 | 286.67 ± 9.12 | 2.64 ± 0.09 | 97 |

Data are expressed as mean (n = 3) ± EP. Concentrations were corrected according to the result of the NSB assays at those concentration points where the loss of Compound I-19 during the NSB experiment was more than 20%.

Compound I-19 inhibited the OATP1B1-mediated estradiol-17-β-glucuronide (E217βG) accumulation with a maximum of 87% at 100 μM (Table 105). In the follow-up assay the calculated IC₅₀ value was 31.4 μM using the NSB-corrected concentrations with a bottom constraint to 0 and with a maximum inhibition of 83% (Table 106).

TABLE 105
Effect of Compound I-19 on the OATP1B1-mediated transport of estradiol-17-
β-glucuronide (E₂17βG) measured in the uptake transporter inhibition assay

| | | | | Relative | |
|---|---|---|---|---|---|
| | | | Transporter | transporter | |
| | Concentration (μM) | | specific | specific | |
| | | NSB | accumulation | accumulation | Relative |
| Compound | nominal | Corrected | (cpm) | (% of control) | inhibition (%) |
| Compound I-19 | 100.00 | 77.00 | 395.67 ± 8.05 | 12.92 ± 0.41 | 87 |
| | 10.00 | 6.60 | 2,303.00 ± 45.84 | 75.21 ± 2.39 | 25 |

| | | | | |
|---|---|---|---|---|
| DMSO | 0.00 | 3,062.00 ± 76.04 | 100.00 ± 3.51 | 0 |
| Rifampicin | 50.00 | 15.67 ± 9.33 | 0.51 ± 0.31 | 99 |

Data are expressed as mean (n = 3) ± EP. Concentrations were corrected according to the result of the NSB assays at those concentration points where the loss of Compound I-19 during the NSB experiment was more than 20%.

TABLE 106
Effect of Compound I-19 on the OATP1B1-mediated transport of estradiol-17-
β-glucuronide (E₂17βG) measured in the uptake transporter inhibition assay

| | | | | Relative | |
|---|---|---|---|---|---|
| | | | Transporter | transporter | |
| | Concentration (μM) | | specific | specific | |
| | | NSB | accumulation | accumulation | Relative |
| Compound | nominal | corrected | (cpm) | (% of control) | inhibition (%) |
| Compound I-19 | 100.00 | 100.00 | 381.67 ± 5.50 | 16.93 ± 0.58 | 83 |
| | 33.33 | 24.33 | 1,123.33 ± 43.53 | 49.83 ± 2.48 | 50 |
| | 11.11 | 7.89 | 1,743.00 ± 16.09 | 77.32 ± 2.52 | 23 |
| | 3.70 | 1.89 | 2,152.67 ± 36.09 | 95.49 ± 3.39 | 5 |
| | 1.24 | 0.86 | 2,178.33 ± 50.48 | 96.63 ± 3.76 | 3 |
| | 0.41 | 0.25 | 2,271.00 ± 74.47 | 100.74 ± 4.56 | −1 |
| | 0.14 | 0.09 | 2,252.33 ± 16.07 | 99.91 ± 3.20 | 0 |

| | | | | |
|---|---|---|---|---|
| DMSO | 0.00 | 2,254.33 ± 70.46 | 100.00 ± 4.42 | 0 |
| Rifampicin | 50.00 | 19.33 ± 8.75 | 0.86 ± 0.39 | 99 |

Data are expressed as mean (n = 3) ± EP. Concentrations were corrected according to the result of the NSB assays at those concentration points where the loss of Compound I-19 during the NSB experiment was more than 20%.

Compound I-19 inhibited the OATP1B3-mediated cholecystokinin octapeptide (CCK-8) accumulation with a maximum of 50% at 100 μM (Table 107). In the follow-up assay the calculated IC₅₀ value was 82.8 μM using the NSB-corrected concentrations with a bottom constraint to 0 and with a maximum inhibition of 61% (Table 108).

TABLE 107
Effect of Compound I-19 on the OATP1B3-mediated transport of cholecystokinin
octapeptide (CCK-8) measured in the uptake transporter inhibition assay

| | | | | Relative | |
|---|---|---|---|---|---|
| | | | Transporter | transporter | |
| | Concentration (μM) | | specific | specific | |
| | | NSB | accumulation | accumulation | Relative |
| Compound | nominal | corrected | (cpm) | (% of control) | inhibition (%) |
| Compound I-19 | 100.00 | 77.00 | 1,057.00 ± 45.14 | 50.40 ± 2.21 | 50 |
| | 10.00 | 6.60 | 1,953.33 ± 8.60 | 93.13 ± 0.99 | 7 |

| | | | | |
|---|---|---|---|---|
| DMSO | 0.00 | 2,097.33 ± 20.38 | 100.00 ± 1.37 | 0 |
| Rifampicin | 50.00 | 29.67 ± 5.30 | 1.41 ± 0.25 | 99 |

Data are expressed as mean (n = 3) ± EP. Concentrations were corrected according to the result of the NSB assays at those concentration points where the loss of Compound I-19 during the NSB experiment was more than 20%.

TABLE 108
Effect of Compound I-19 on the OATP1B3-mediated transport of cholecystokinin
octapeptide (CCK-8) measured in the uptake transporter inhibition assay

| | | | | Relative | |
|---|---|---|---|---|---|
| | | | Transporter | transporter | |
| | Concentration (μM) | | specific | specific | |
| | | NSB | accumulation | accumulation | Relative |
| Compound | nominal | corrected | (cpm) | (% of control) | inhibition (%) |
| Compound I-19 | 100.00 | 100.00 | 459.67 ± 23.17 | 39.13 ± 2.03 | 61 |
| | 33.33 | 24.33 | 907.00 ± 27.52 | 77.21 ± 2.52 | 23 |
| | 11.11 | 7.89 | 941.00 ± 47.56 | 80.11 ± 4.16 | 20 |
| | 3.70 | 1.89 | 1,129.67 ± 9.53 | 96.17 ± 1.42 | 4 |
| | 1.24 | 0.86 | 1,076.33 ± 39.37 | 91.63 ± 3.53 | 8 |
| | 0.41 | 0.25 | 1,051.33 ± 32.64 | 89.50 ± 2.98 | 10 |
| | 0.14 | 0.09 | 1,167.00 ± 32.35 | 99.35 ± 3.01 | 1 |

| | | | | |
|---|---|---|---|---|
| DMSO | 0.00 | 1,174.67 ± 14.23 | 100.00 ± 1.71 | 0 |
| Rifampicin | 50.00 | 6.33 ± 3.64 | 0.54 ± 0.31 | 99 |

Data are expressed as mean (n = 3) ± EP. Concentrations were corrected according to the result of the NSB assays at those concentration points where the loss of Compound I-19 during the NSB experiment was more than 20%.

Compound I-19 inhibited the OCT1-mediated sumatriptan accumulation with a maximum inhibition of 92% at 100 μM (Table 109). In the follow-up assay the calculated IC₅₀ value was 3.32 μM using the NSB-corrected concentrations with a maximum inhibition of 92% (Table 110).

TABLE 109
Effect of Compound I-19 on the OCT1-mediated transport of sumatriptan
measured in the uptake transporter inhibition assay

| | | | | Relative | |
|---|---|---|---|---|---|
| | | | Transporter | transporter | |
| | Concentration (μM) | | specific | specific | |
| | | NSB | accumulation | accumulation | Relative |
| Compound | nominal | corrected | (cpm) | (% of control) | inhibition (%) |
| Compound I-19 | 100.00 | 77.00 | 259.00 ± 14.94 | 8.17 ± 0.48 | 92 |
| | 10.00 | 6.60 | 930.00 ± 10.03 | 29.35 ± 0.48 | 71 |

| | | | | |
|---|---|---|---|---|
| DMSO | 0.00 | 3,168.67 ± 38.42 | 100.00 ± 1.71 | 0 |
| Verapamil | 300.00 | 6.00 ± 5.44 | 0.19 ± 0.17 | 100 |

Data are expressed as mean (n = 3) ± EP. Concentrations were corrected according to the result of the NSB assays at those concentration points where the loss of Compound I-19 during the NSB experiment was more than 20%.

TABLE 110
Effect of Compound I-19 on the OCT1-mediated transport of sumatriptan
measured in the uptake transporter inhibition assay

| | | | | Relative | |
|---|---|---|---|---|---|
| | | | Transporter | transporter | |
| | Concentration (μM) | | specific | specific | |
| | | NSB | accumulation | accumulation | Relative |
| Compound | nominal | corrected | (cpm) | (% of control) | inhibition (%) |
| Compound I-19 | 100.00 | 100.00 | 83.67 ± 11.38 | 7.84 ± 1.11 | 92 |
| | 33.33 | 24.33 | 154.33 ± 8.03 | 14.47 ± 0.94 | 86 |
| | 11.11 | 7.89 | 289.33 ± 15.11 | 27.13 ± 1.77 | 73 |
| | 3.70 | 1.89 | 485.00 ± 17.73 | 45.47 ± 2.44 | 55 |
| | 1.24 | 0.86 | 652.33 ± 10.37 | 61.16 ± 2.59 | 39 |
| | 0.41 | 0.25 | 777.00 ± 10.82 | 72.84 ± 3.03 | 27 |
| | 0.14 | 0.09 | 896.33 ± 19.14 | 84.03 ± 3.76 | 16 |

| | | | | |
|---|---|---|---|---|
| DMSO | 0.00 | 1,066.67 ± 41.87 | 100.00 ± 5.55 | 0 |
| Verapamil | 300.00 | 21.00 ± 3.50 | 1.97 ± 0.34 | 98 |

Data are expressed as mean (n = 3) ± EP. Concentrations were corrected according to the result of the NSB assays at those concentration points where the loss of Compound I-19 during the NSB experiment was more than 20%.

Compound I-19 inhibited the OCT2-mediated metformin accumulation with a maximum inhibition of 98% at 100 μM (Table 111). In the follow-up assay the calculated IC₅₀ value was 8.95 μM using the NSB-corrected concentrations with a maximum inhibition of 97% (Table 112).

TABLE 111
Effect of Compound I-19 on the OCT2-mediated transport of metformin
measured in the uptake transporter inhibition assay

| | | | | Relative | |
|---|---|---|---|---|---|
| | | | Transporter | transporter | |
| | Concentration (μM) | | specific | specific | |
| | | NSB | accumulation | accumulation | Relative |
| Compound | nominal | corrected | (cpm) | (% of control) | inhibition (%) |
| Compound I-19 | 100.00 | 77.00 | 7.33 ± 2.67 | 1.78 ± 0.65 | 98 |
| | 10.00 | 6.60 | 126.00 ± 14.46 | 30.61 ± 3.54 | 69 |

| | | | | |
|---|---|---|---|---|
| DMSO | 0.00 | 411.67 ± 6.01 | 100.00 ± 2.06 | 0 |
| Verapamil | 300.00 | 4.00 ± 2.13 | 0.97 ± 0.52 | 99 |

Data are expressed as mean (n = 3) ± EP. Concentrations were corrected according to the result of the NSB assays at those concentration points where the loss of Compound I-19 during the NSB experiment was more than 20%.

TABLE 112
Effect of Compound I-19 on the OCT2-mediated transport of metformin
measured in the uptake transporter inhibition assay

| | Concentration | | | Relative | |
|---|---|---|---|---|---|
| | (μM) | | Transporter | transporter | Relative |
| | | NSB | specific | specific | inhibi- |
| Com- | | cor- | accumulation | accumulation | tion |
| pound | nominal | rected | (cpm) | (% of control) | (%) |
| Com- | 100.00 | 100.00 | 10.83 ± 4.69 | 3.16 ± 1.38 | 97 |
| pound | 33.33 | 24.33 | 43.33 ± 11.20 | 12.63 ± 3.35 | 87 |
| I-19 | 11.11 | 7.89 | 136.00 ± 9.32 | 39.65 ± 3.63 | 60 |
| | 3.70 | 1.89 | 243.33 ± 20.22 | 70.94 ± 7.30 | 29 |
| | 1.24 | 0.86 | 279.33 ± 8.96 | 81.44 ± 5.58 | 19 |
| | 0.41 | 0.25 | 309.67 ± 3.77 | 90.28 ± 5.58 | 10 |
| | 0.14 | 0.09 | 339.33 ± 6.18 | 98.93 ± 6.26 | 1 |
| DMSO | 0.00 | | 343.00 ± 20.79 | 100.00 ± 8.57 | 0 |
| Verap- | 300.00 | | 4.67 ± 1.76 | 1.36 ± 0.52 | 99 |
| amil | | | | | |

Data are expressed as mean (n = 3) ± EP. Concentrations were corrected according to the result of the NSB assays at those concentration points where the loss of Compound I-19 during the NSB experiment was more than 20%.

Compound A inhibited the MATE1-mediated metformin accumulation with a maximum inhibition of 80% at 30 μM (Table 113). In the follow-up assay the calculated IC₅₀ value was 3.87 μM with a bottom constraint to 0 and a top constraint to 100 and with a maximum inhibition of 79% (Table 114).

TABLE 113
Effect of Compound A on the MATE1-mediated transport of metformin
measured in the uptake transporter inhibition assay

| | | | Relative | |
|---|---|---|---|---|
| | Concen- | Transporter | transporter | |
| | tration | specific | specific | Relative |
| | (μM) | accumulation | accumulation | inhibition |
| Compound | nominal | (cpm) | (% of control) | (%) |
| Compound A | 30.00 | 66.67 ± 4.78 | 20.18 ± 1.49 | 80 |
| | 3.00 | 181.33 ± 14.06 | 54.89 ± 4.37 | 45 |
| DMSO | 0.00 | 330.33 ± 5.99 | 100.00 ± 2.56 | 0 |
| Pyrimethamine | 5.00 | −5.33 ± 1.80 | −1.61 ± 0.54 | 102 |

Data are expressed as mean (n = 3) ± EP.

TABLE 114
Effect of Compound A on the MATE1-mediated transport
of metformin measured in the uptake transporter inhibition
assay - IC₅₀ determination

| | | | Relative | |
|---|---|---|---|---|
| | Concen- | Transporter | transporter | |
| | tration | specific | specific | Relative |
| | (μM) | accumulation | accumulation | Inhibition |
| Compound | nominal | (cpm) | (% of control) | (%) |
| Compound A | 30.00 | 91.33 ± 1.76 | 20.65 ± 0.69 | 79 |
| | 10.00 | 172.33 ± 5.44 | 38.96 ± 1.63 | 61 |
| | 3.33 | 208.67 ± 10.62 | 47.17 ± 2.73 | 53 |
| | 1.11 | 309.00 ± 8.08 | 69.86 ± 2.64 | 30 |
| | 0.37 | 358.33 ± 10.25 | 81.01 ± 3.21 | 19 |
| | 0.12 | 411.00 ± 20.23 | 92.92 ± 5.23 | 7 |
| | 0.04 | 471.00 ± 14.24 | 106.48 ± 4.34 | −6 |
| DMSO | 0.00 | 442.33 ± 12.10 | 100.00 ± 3.87 | 0 |
| Pyrimethamine | 5.00 | −2.67 ± 2.33 | −0.60 ± 0.53 | 101 |

Data are expressed as mean (n = 3) ± EP.

Compound A inhibited the MATE2-K-mediated metformin accumulation with a maximum inhibition of 73% at 30 μM (Table 115). In the follow-up assay the calculated IC₅₀ value was 12.8 μM with a maximum inhibition of 79% (Table 116).

TABLE 115
Effect of Compound A on the MATE2-K-mediated
transport of metformin measured in the
uptake transporter inhibition assay

| | | | Relative | |
|---|---|---|---|---|
| | Concen- | Transporter | transporter | |
| | tration | specific | specific | Relative |
| | (μM) | accumulation | accumulation | inhibition |
| Compound | nominal | (cpm) | (% of control) | (%) |
| Compound A | 30.00 | 24.00 ± 3.54 | 27.07 ± 4.56 | 73 |
| | 3.00 | 86.33 ± 7.06 | 97.37 ± 11.23 | 3 |
| DMSO | 0.00 | 88.67 ± 7.22 | 100.00 ± 11.51 | 0 |
| Pyrimethamine | 5.00 | −2.00 ± 3.79 | −2.26 ± 4.27 | 102 |

Data are expressed as mean (n = 3) ± EP.

TABLE 116
Effect of Compound A on the MATE2-K-mediated transport
of metformin measured in the uptake transporter inhibition
assay - IC₅₀determination

| | | | Relative | |
|---|---|---|---|---|
| | Concen- | Transporter | transporter | |
| | tration | specific | specific | Relative |
| | (μM) | accumulation | accumulation | Inhibition |
| Compound | nominal | (cpm) | (% of control) | (%) |
| Compound A | 30.00 | 24.67 ± 2.26 | 20.61 ± 1.94 | 79 |
| | 10.00 | 62.00 ± 4.35 | 51.81 ± 3.80 | 48 |
| | 3.33 | 92.00 ± 3.37 | 76.88 ± 3.27 | 23 |
| | 1.11 | 107.33 ± 7.45 | 89.69 ± 6.53 | 10 |
| | 0.37 | 109.00 ± 8.48 | 91.09 ± 7.36 | 9 |
| | 0.12 | 116.00 ± 9.43 | 96.94 ± 8.16 | 3 |
| | 0.04 | 108.00 ± 2.98 | 90.25 ± 3.17 | 10 |
| DMSO | 0.00 | 119.67 ± 2.60 | 100.00 ± 3.08 | 0 |
| Pyrimethamine | 5.00 | 0.00 ± 3.83 | 0.00 ± 3.20 | 100 |

Data are expressed as mean (n = 3) ± EP.

Compound A did not inhibit the OAT1-mediated tenofovir accumulation up to 30 μM (Table 117).

TABLE 117
Effect of Compound A on the OAT1-mediated transport of tenofovir
measured in the uptake transporter inhibition assay

| | | | Relative | |
|---|---|---|---|---|
| | Concen- | Transporter | transporter | |
| | tration | specific | specific | Relative |
| | (μM) | accumulation | accumulation | inhibition |
| Compound | nominal | (cpm) | (% of control) | (%) |
| Compound A | 30.00 | 2,121.67 ± 88.79 | 88.39 ± 4.23 | 12 |
| | 3.00 | 2,392.67 ± 13.09 | 99.68 ± 2.39 | 0 |
| DMSO | 0.00 | 2,400.33 ± 55.91 | 100.00 ± 3.29 | 0 |
| Probenecid | 300.00 | 28.00 ± 5.07 | 1.17 ± 0.21 | 99 |

Data are expressed as mean (n = 3) ± EP.

Compound A did not inhibit the OAT3-mediated E3 S accumulation up to 30 μM (Table 118). Compound A did not inhibit the OATP1B1-mediated E217βG accumulation up to 30 μM (Table 119).

TABLE 118
Effect of Compound A on the OAT3-mediated transport of estrone-3-
sulfate (E3S) measured in the uptake transporter inhibition assay

| | | | Relative | |
|---|---|---|---|---|
| | Concen- | Transporter | transporter | |
| | tration | specific | specific | Relative |
| | (μM) | accumulation | accumulation | inhibition |
| Compound | nominal | (cpm) | (% of control) | (%) |
| Compound | 30.00 | 3,566.67 ± 111.53 | 89.65 ± 3.21 | 10 |
| A | 3.00 | 3,883.00 ± 158.50 | 97.60 ± 4.33 | 2 |
| DMSO | 0.00 | 3,978.33 ± 69.08 | 100.00 ± 2.46 | 0 |
| Probenecid | 500.00 | 67.00 ± 12.26 | 1.68 ± 0.31 | 98 |

Data are expressed as mean (n = 3) ± EP.

TABLE 119
Effect of Compound A on the OATP1B1-mediated transport
of estradiol-17-β-glucuronide (E₂17βG) measured
in the uptake transporter inhibition assay

| | | | Relative | |
|---|---|---|---|---|
| | Concen- | Transporter | transporter | |
| | tration | specific | specific | Relative |
| | (μM) | accumulation | accumulation | inhibition |
| Compound | nominal | (cpm) | (% of control) | (%) |
| Compound A | 30.00 | 3,499.33 ± 22.14 | 94.38 ± 2.14 | 6 |
| | 3.00 | 3,588.00 ± 48.11 | 96.77 ± 2.48 | 3 |
| DMSO | 0.00 | 3,707.67 ± 80.88 | 100.00 ± 3.08 | 0 |
| Rifampicin | 50.00 | 31.00 ± 8.48 | 0.84 ± 0.23 | 99 |

Data are expressed as mean (n = 3) ± EP.

Compound A did not inhibited the OATP1B3-mediated CCK-8 accumulation up to 30 μM (Table 120).

TABLE 120
Effect of Compound A on the OATP1B3-mediated transport
of cholecystokinin octapeptide (CCK-8) measured
in the uptake transporter inhibition assay

| | | | Relative | |
|---|---|---|---|---|
| | Concen- | Transporter | transporter | |
| | tration | specific | specific | Relative |
| | (μM) | accumulation | accumulation | inhibition |
| Compound | nominal | (cpm) | (% of control) | (%) |
| Compound A | 30.00 | 806.33 ± 33.04 | 90.63 ± 4.66 | 9 |
| | 3.00 | 825.67 ± 24.09 | 92.81 ± 3.95 | 7 |
| DMSO | 0.00 | 889.67 ± 27.58 | 100.00 ± 4.38 | 0 |
| Rifampicin | 50.00 | 6.33 ± 2.54 | 0.71 ± 0.29 | 99 |

Data are expressed as mean (n = 3) ± EP.

Compound A inhibited the OCT1-mediated sumatriptan accumulation with a maximum inhibition of 85% at 30 μM (Table 121). In the follow-up assay the calculated IC₅₀ value was 2.31 μM with a bottom constraint to 0 and a top constraint to 100 and with a maximum inhibition of 86% (Table 122).

TABLE 121
Effect of Compound A on the OCT1-mediated transport of sumatriptan
measured in the uptake transporter inhibition assay

| | | | Relative | |
|---|---|---|---|---|
| | Concen- | Transporter | transporter | |
| | tration | specific | specific | Relative |
| | (μM) | accumulation | accumulation | inhibition |
| Compound | nominal | (cpm) | (% of control) | (%) |
| Compound A | 30.00 | 141.67 ± 10.22 | 15.00 ± 1.09 | 85 |
| | 3.00 | 484.33 ± 16.61 | 51.27 ± 1.85 | 49 |
| DMSO | 0.00 | 944.67 ± 10.62 | 100.00 ± 1.59 | 0 |
| Verapamil | 300.00 | 13.00 ± 5.47 | 1.38 ± 0.58 | 99 |

Data are expressed as mean (n = 3) ± EP.

TABLE 122
Effect of Compound A on the OCT1-mediated transport
of sumatriptan measured in the uptake transporter inhibition
assay - IC₅₀determination

| | | | Relative | |
|---|---|---|---|---|
| | Concen- | Transporter | transporter | |
| | tration | specific | specific | Relative |
| | (μM) | accumulation | accumulation | Inhibition |
| Compound | nominal | (cpm) | (% of control) | (%) |
| Compound A | 30.00 | 135.33 ± 45.14 | 14.03 ± 4.75 | 86 |
| | 10.00 | 243.33 ± 32.16 | 25.23 ± 3.64 | 75 |
| | 3.33 | 466.67 ± 15.60 | 48.39 ± 3.21 | 52 |
| | 1.11 | 628.00 ± 55.27 | 65.12 ± 6.84 | 35 |
| | 0.37 | 769.33 ± 59.12 | 79.78 ± 7.65 | 20 |
| | 0.12 | 713.17 ± 36.62 | 73.95 ± 5.69 | 26 |
| | 0.04 | 881.00 ± 27.83 | 91.36 ± 5.98 | 9 |
| DMSO | 0.00 | 964.33 ± 55.27 | 100.00 ± 8.11 | 0 |
| verapamil | 300.00 | 19.00 ± 18.96 | 1.97 ± 1.97 | 98 |

Data are expressed as mean (n = 3) ± EP.

Compound A inhibited the OCT2-mediated metformin accumulation with a maximum inhibition of 86% at 30 μM (Table 123). In the follow-up assay the calculated IC₅₀ value was 3.49 μM (Table 72) with a top constraint to 100 and with a maximum inhibition of 91% (Table 124).

TABLE 123
Effect of Compound A on the OCT2-mediated transport of metformin
measured in the uptake transporter inhibition assay

| | | | Relative | |
|---|---|---|---|---|
| | Concen- | Transporter | transporter | |
| | tration | specific | specific | Relative |
| | (μM) | accumulation | accumulation | inhibition |
| Compound | nominal | (cpm) | (% of control) | (%) |
| Compound A | 30.00 | 39.67 ± 3.38 | 13.54 ± 1.38 | 86 |
| | 3.00 | 150.67 ± 6.96 | 51.42 ± 3.71 | 49 |
| DMSO | 0.00 | 293.00 ± 16.21 | 100.00 ± 7.83 | 0 |
| Verapamil | 300.00 | −4.83 ± 1.83 | −1.65 ± 0.63 | 102 |

Data are expressed as mean (n = 3) ± EP.

TABLE 124
Effect of Compound A on the OCT2-mediated transport
of metformin measured in the uptake transporter inhibition
assay - IC₅₀ determination

| | | | Relative | |
|---|---|---|---|---|
| | Concen- | Transporter | transporter | |
| | tration | specific | specific | Relative |
| | (μM) | accumulation | accumulation | Inhibition |
| Compound | nominal | (cpm) | (% of control) | (%) |
| Compound A | 30.00 | 22.33 ± 2.26 | 9.42 ± 0.99 | 91 |
| | 10.00 | 61.33 ± 7.84 | 25.88 ± 3.39 | 74 |
| | 3.33 | 127.67 ± 11.08 | 53.87 ± 4.92 | 46 |
| | 1.11 | 200.00 ± 7.72 | 84.39 ± 4.06 | 16 |
| | 0.37 | 234.33 ± 9.21 | 98.87 ± 4.81 | 1 |
| | 0.12 | 252.00 ± 5.57 | 106.33 ± 3.85 | −6 |
| | 0.04 | 259.00 ± 13.36 | 109.28 ± 6.45 | −9 |
| DMSO | 0.00 | 237.00 ± 6.80 | 100.00 ± 4.06 | 0 |
| verapamil | 300.00 | 5.00 ± 1.60 | 2.11 ± 0.68 | 98 |

Data are expressed as mean (n = 3) ± EP.

Compound B inhibited the MATE1-mediated metformin accumulation with a maximum inhibition of 80% at 50 μM (Table 125). In the follow-up assay the calculated IC₅₀ value was 35.7 μM with a bottom constraint to 0 and with a maximum inhibition of 74% (Table 126).

TABLE 125
Effect of Compound B on the MATE1-mediated transport of metformin
measured in the uptake transporter inhibition assay

| | | | Relative | |
|---|---|---|---|---|
| | Concen- | Transporter | transporter | |
| | tration | specific | specific | Relative |
| | (μM) | accumulation | accumulation | Inhibition |
| Compound | nominal | (cpm) | (% of control) | (%) |
| Compound B | 50.00 | 102.33 ± 11.78 | 20.28 ± 2.47 | 80 |
| DMSO | 5.00 | 536.67 ± 5.14 | 106.34 ± 4.36 | −6 |
| | 0.00 | 504.67 ± 20.11 | 100.00 ± 5.64 | 0 |
| Pyrimethamine | 5.00 | −3.33 ± 8.46 | −0.66 ± 1.68 | 101 |

Data are expressed as mean (n = 3) ± EP.

TABLE 126
Effect of Compound B on the MATE1-mediated transport
of metformin measured in the uptake transporter inhibition
assay - IC₅₀ determination

| | | | Relative | |
|---|---|---|---|---|
| | Concen- | Transporter | transporter | |
| | tration | specific | specific | Relative |
| | (μM) | accumulation | accumulation | Inhibition |
| Compound | nominal | (cpm) | (% of control) | (%) |
| Compound B | 50.00 | 78.00 ± 5.07 | 26.44 ± 2.30 | 76 |
| | 16.67 | 276.00 ± 6.75 | 93.56 ± 5.88 | 6 |
| | 5.56 | 309.50 ± 8.52 | 104.92 ± 6.73 | −5 |
| | 1.85 | 301.67 ± 6.36 | 102.26 ± 6.30 | −2 |
| | 0.62 | 304.67 ± 4.77 | 103.28 ± 6.20 | −3 |
| | 0.21 | 293.33 ± 9.40 | 99.44 ± 6.58 | 1 |
| | 0.07 | 300.00 ± 11.43 | 101.69 ± 7.05 | −2 |
| DMSO | 0.00 | 295.00 ± 17.08 | 100.00 ± 8.19 | 0 |
| Pyrimethamine | 5.00 | 0.33 ± 0.88 | 0.11 ± 0.30 | 100 |

Data are expressed as mean (n = 3) ± EP.

Compound B inhibited the MATE2-K-mediated metformin accumulation with a maximum inhibition of 68% at 80 μM (Table 127). In the follow-up assay the calculated I IC₅₀ C50 value was 56.8 μM with a bottom constraint to 0 and with a top constraint to 100 and with a maximum inhibition of 64% (Table 128).

TABLE 127
Effect of Compound B on the MATE2-K-mediated transport of metformin
measured in the uptake transporter inhibition assay

| | | Transporter | | |
|---|---|---|---|---|
| | Concentration | specific | Relative transporter | Relative |
| | (μM) | accumulation | specific accumulation | Inhibition |
| Compound | nominal | (cpm) | (% of control) | (%) |
| Compound B | 80.00 | 68.67 ± 4.29 | 31.99 ± 2.62 | 68 |
| | 8.00 | 208.67 ± 7.13 | 97.20 ± 6.13 | 3 |
| DMSO | 0.00 | 214.67 ± 11.39 | 100.00 ± 7.50 | 0 |
| Pyrimethamine | 5.00 | 5.00 ± 4.65 | 2.33 ± 2.17 | 98 |

Data are expressed as mean (n = 3) ± EP.

TABLE 128
Effect of Compound B on the MATE2-K-mediated transport of metformin measured
in the uptake transporter inhibition assay - IC₅₀ determination

| | | Transporter | | |
|---|---|---|---|---|
| | Concentration | specific | Relative transporter | Relative |
| | (μM) | accumulation | specific accumulation | Inhibition |
| Compound | nominal | (cpm) | (% of control) | (%) |
| Compound B | 80.00 | 40.33 ± 2.49 | 36.28 ± 2.53 | 64 |
| | 26.67 | 86.33 ± 5.30 | 77.66 ± 5.39 | 22 |
| | 8.89 | 97.33 ± 2.73 | 87.56 ± 3.76 | 12 |
| | 2.96 | 100.00 ± 4.57 | 89.96 ± 5.04 | 10 |
| | 0.99 | 101.67 ± 0.67 | 91.45 ± 3.03 | 9 |
| | 0.33 | 101.33 ± 1.56 | 91.15 ± 3.28 | 9 |
| | 0.11 | 107.33 ± 3.20 | 96.55 ± 4.25 | 3 |
| DMSO | 0.00 | 111.17 ± 3.61 | 100.00 ± 4.59 | 0 |
| Pyrimethamine | 5.00 | −1.33 ± 1.25 | −1.20 ± 1.12 | 101 |

Data are expressed as mean (n = 3) ± EP.

Compound B did not inhibit the OAT1-mediated tenofovir accumulation up to 100 μM (Table 129).

TABLE 129
Effect of Compound B on the OAT1-mediated transport of tenofovir
measured in the update transporter inhibition assay

| | | Transporter | | |
|---|---|---|---|---|
| | Concentration | specific | Relative transporter | Relative |
| | (μM) | accumulation | specific accumulation | Inhibition |
| Compound | nominal | (cpm) | (% of control) | (%) |
| Compound B | 100.00 | 4,432.33 ± 41.51 | 94.18 ± 1.52 | 6 |
| | 10.00 | 4,688.67 ± 36.27 | 99.63 ± 1.52 | 0 |
| DMSO | 0.00 | 4,706.00 ± 62.05 | 100.00 ± 1.86 | 0 |
| Probenecid | 300.00 | 92.67 ± 16.36 | 1.97 ± 0.35 | 98 |

Data are expressed as mean (n = 3) ± EP.

Compound B did not inhibit the OAT3-mediated E3S accumulation up to 100 μM (Table 130).

TABLE 130
Effect of Compound B on the OAT3-mediated transport of estrone-3-
sulfate (E3S) measured in the uptake transporter inhibition assay

| | | Transporter | | |
|---|---|---|---|---|
| | Concentration | specific | Relative transporter | Relative |
| | (μM) | accumulation | specific accumulation | Inhibition |
| Compound | nominal | (cpm) | (% of control) | (%) |
| Compound B | 100.00 | 9,774.33 ± 110.84 | 91.62 ± 1.09 | 8 |
| | 10.00 | 10,763.00 ± 82.76 | 100.88 ± 0.85 | −1 |
| DMSO | 0.00 | 10,668.67 ± 37.00 | 100.00 ± 0.49 | 0 |
| Probenecid | 500.00 | 291.67 ± 17.04 | 2.73 ± 0.16 | 98 |

Data are expressed as mean (n = 3) ± EP.

Compound B inhibited the OATP1B1-mediated E217βG accumulation with a maximum inhibition of 34% at 100 μM (Table 131).

TABLE 131
Effect of Compound B on the OATP1B1-mediated transport
of estradiol-17-β-glucuronide (E₂17βG) measured
in the uptake transporter inhibition assay

| | | Transporter | | |
|---|---|---|---|---|
| | Concentration | specific | Relative transporter | Relative |
| | (μM) | accumulation | specific accumulation | Inhibition |
| Compound | nominal | (cpm) | (% of control) | (%) |
| Compound B | 100.00 | 3,103.00 ± 9.09 | 65.74 ± 0.40 | 34 |
| | 10.00 | 4,498.67 ± 32.61 | 95.31 ± 0.86 | 5 |
| DMSO | 0.00 | 4,720.00 ± 25.13 | 100.00 ± 0.75 | 0 |
| Rifampicin | 50.00 | 52.67 ± 5.36 | 1.12 ± 0.11 | 99 |

Data are expressed as mean (n = 3) ± EP.

Compound B did not inhibited the OATP1B3-mediated CCK-8 accumulation up to 100 μM (Table 132).

TABLE 132
Effect of Compound B on the OATP1B3-mediated transport of cholecystokinin
octapeptide (CCK-8) measured in the uptake transporter inhibition assay

| | | Transporter | | |
|---|---|---|---|---|
| | Concentration | specific | Relative transporter | Relative |
| | (μM) | accumulation | specific accumulation | Inhibition |
| Compound | nominal | (cpm) | (% of control) | (%) |
| Compound B | 100.00 | 1,780.00 ± 44.20 | 83.31 ± 2.26 | 17 |
| | 10.00 | 2,037.67 ± 25.06 | 95.37 ± 1.58 | 5 |
| DMSO | 0.00 | 2,136.67 ± 23.59 | 100.00 ± 1.56 | 0 |
| Rifampicin | 50.00 | 32.00 ± 5.06 | 1.50 ± 0.24 | 99 |

Data are expressed as mean (n = 3) ± EP.

Compound B inhibited the OCT1-mediated sumatriptan accumulation with a maximum inhibition of 65% at 100 μM (Table 133). In the follow-up assay the calculated IC₅₀ value was 4.36 μM with a maximum inhibition of 93% (Table 134).

TABLE 133
Effect on Compound B on the OCT1-mediated transport of sumatriptan
measured in the uptake transporter inhibition assay

| | | Transporter | | |
|---|---|---|---|---|
| | Concentration | specific | Relative transporter | Relative |
| | (μM) | accumulation | specific accumulation | Inhibition |
| Compound | nominal | (cpm) | (% of control) | (%) |
| Compound B | 100.00 | 927.00 ± 9.09 | 35.19 ± 1.25 | 65 |
| | 10.00 | 2,054.33 ± 22.65 | 77.98 ± 1.27 | 22 |
| DMSO | 0.00 | 2,634.33 ± 31.44 | 100.00 ± 1.69 | 0 |
| Verapamil | 300.00 | 26.00 ± 17.82 | 0.99 ± 0.68 | 99 |

Data are expressed as mean (n = 3) ± EP.

TABLE 134
Effect of Compound B on the OCT1-mediated transport of sumatriptan measured
in the uptake transporter inhibition assay - IC₅₀ determination

| | | Transporter | | |
|---|---|---|---|---|
| | Concentration | specific | Relative transporter | Relative |
| | (μM) | accumulation | specific accumulation | Inhibition |
| Compound | nominal | (cpm) | (% of control) | (%) |
| Compound B | 100.00 | 63.67 ± 4.22 | 6.95 ± 0.50 | 93 |
| | 33.33 | 162.33 ± 11.60 | 17.72 ± 1.36 | 82 |
| | 11.11 | 320.67 ± 8.92 | 34.99 ± 1.37 | 65 |
| | 3.70 | 470.67 ± 17.22 | 51.36 ± 2.35 | 49 |
| | 1.24 | 624.00 ± 20.01 | 68.10 ± 2.88 | 32 |
| | 0.41 | 789.67 ± 37.27 | 86.18 ± 4.71 | 14 |
| | 0.14 | 850.33 ± 38.18 | 92.80 ± 4.88 | 7 |
| DMSO | 0.00 | 916.33 ± 25.17 | 100.00 ± 3.89 | 0 |
| Verapamil | 300.00 | 17.00 ± 3.74 | 1.86 ± 0.41 | 98 |

Data are expressed as mean (n = 3) ± EP.

Compound B inhibited the OCT2-mediated metformin accumulation with a maximum inhibition of 36% at 100 μM (Table 135).

TABLE 135
Effect of Compound B on the OCT2-mediated transport of metformin
measured in the uptake transporter inhibition assay

| | | Transporter | | |
|---|---|---|---|---|
| | Concentration | specific | Relative transporter | Relative |
| | (μM) | accumulation | specific accumulation | Inhibition |
| Compound | nominal | (cpm) | (% of control) | (%) |
| Compound B | 100.00 | 285.00 ± 5.19 | 64.33 ± 3.50 | 36 |
| | 10.00 | 481.67 ± 11.64 | 108.73 ± 6.17 | −9 |
| DMSO | 0.00 | 443.00 ± 22.73 | 100.00 ± 7.26 | 0 |
| Verapamil | 300.00 | 3.67 ± 3.33 | 0.83 ± 0.75 | 99 |

Data are expressed as mean (n = 3) ± EP.

For the IC₅₀ calculations the concentration values were corrected according to the result of the NSB assays by correcting the nominal concentrations if the loss of Compound I-19 during the NSB experiment was more than 20%.

### Conclusions

Compound I-19 is an in vitro inhibitor of the human BSEP and MDR1 (ABC) transporters with a calculated IC₅₀ value of 45.3 and 21.0 μM, respectively. Compound I-19 is an in vitro inhibitor of the human BCRP (ABC) transporter by less than 50% up to 100 μM. Compound I-19 is an in vitro inhibitor of the human MATE1, MATE2-K, OATP1B1, OATP1B3, OCT1 and OCT2 (SLC) transporters with a calculated IC₅₀ value of 8.86, 36.6, 31.4, 82.8, 3.32 and 8.95 μM, respectively. Compound I-19 is an in vitro inhibitor of the human OAT1 and OAT3 (SLC) transporters by less than 50% up to 100 μM.

Compound A is not an in vitro inhibitor of the human BCRP, MDR1 and BSEP (ABC) transporters up to 50 and 35 μM, respectively. Compound A is an in vitro inhibitor of the human MATE1, MATE2-K, OCT1 and OCT2 (SLC) transporters with a calculated IC₅₀ value of 3.87, 12.8, 2.31 and 3.49 μM, respectively. Compound A is not an in vitro inhibitor of the human OAT1, OAT3, OATP1B1 and OATP1B3 (SLC) transporters up to 30 μM.

Compound B is an in vitro inhibitor of the human MDR1 (ABC) transporter with a calculated IC₅₀ value of 87.8 μM. Compound B is not an in vitro inhibitor of the human BCRP and BSEP (ABC) transporters up to 100 μM. Compound B is an in vitro inhibitor of the human MATE1, MATE2-K and OCT1 (SLC) transporters with a calculated IC₅₀ value of 35.7, 56.8 and 4.36 μM, respectively. Compound B is an in vitro inhibitor of the human OATP1B1 and OCT2 (SLC) transporters by less than 50% up to 100 μM. Compound B is not an in vitro inhibitor of the human OAT1, OAT3 and OATP1B3 (SLC) transporters up to 100 μM.

### Materials and Methods

### Test Articles and Stock Solutions, Chemicals and Instruments

Compound I-19, Compound A and Compound B were supplied by Supernus Pharmaceuticals, Inc.

The serial dilution used in the assays was prepared in DMSO and used as the test solutions in the different assays at 100-fold dilution, the dilution factor in the preliminary assays matched that of the respective transporter assay. The solvent concentration in the assay buffer did not exceed 1.5% (v/v) in the assays.

### Reagents and Instruments

All reagents and solvents were of analytical grade. The purified water described in this report was prepared with a Millipore Milli-Q Reference system.

Instruments used for detection include a Perkin Elmer MicroBeta2 liquid scintillation counter (Perkin Elmer, Waltham MA) and a BMG Labtech FluoStar Omega multifunctional microplate reader (BMG Labtech, Offenburg, Germany).

### Quantitation of Compound I-19, Compound a and Compound B by LC-MS/MS

The bioanalysis of Compound I-19, Compound A and Compound B was performed with the instrumentation and method described above.

### Cell Viability Assessment

The acute cytotoxic effect of Compound I-19, Compound A and Compound B was evaluated using the resazurin-based cytotoxicity assay. Resazurin itself is not fluorescent; however, it is converted to resorufin, a highly fluorescent compound, via reduction reactions in metabolically active cells.

The cells were plated on 96-well plates. Prior to the experiment the medium was removed, and cells were rinsed with 2×100 μL of the respective buffer for uptake transporter transfected and control cell lines. Compound I-19, Compound A or Compound B were applied in 50 μL of appropriate uptake buffer for uptake inhibition assays. The organic solvent content was equal in each well and did not exceed 1% (v/v). After incubation with Compound I-19, Compound A or Compound B, the cells were washed and 70 μM resazurin was added (100 μL/well) in the respective assay buffer. Fluorescence of resorufin was measured after two hours incubation at λex=544 nm, λem=620 nm excitation and emission. Cell viability values were presented on a relative scale with 100% defined as the maximal cell viability in the presence of the solvent and without Compound I-19, Compound A or Compound B.

### Non-Specific Binding Assays

NSB experiments were aimed to determine the percentage of Compound I-19, Compound A and Compound B bound to the surfaces of the plastic ware during the experiment. The NSB of Compound I-19, Compound A and Compound B (at 2 or 4 concentrations and 7 concentrations in the follow-up assay) was determined by incubating the compounds in 96-well plates in the absence of cells or membranes. All conditions and stock preparation procedures were identical to the conditions applied in the transporter assays. Samples in triplicates were taken after the appropriate incubation time and the amount of Compound I-19, Compound A and Compound B in the wells was determined using LC-MS/MS.

### Assay Steps in 96-Well Plate for VT Inhibition Assays

DMSO stock solutions of Compound I-19, Compound A and Compound B were mixed with the appropriate transport buffer in a 96-well plate (assay mix), to set 1.5-times of the final nominal concentrations. Samples (1.5×C₀ or Total) were immediately taken after mixing to determine the total initial concentrations. The assay mixes were pre-incubated for 15 minutes at 37° C. and at 32° C. in case of BCRP and MDR1. After the pre-incubation step the assay mixes were 1.5-times diluted with (pre-warmed) transport buffer reaching the final nominal concentrations of the compounds in the VT assay and then were further incubated at 37° C. or 32° C. for BCRP and MDR1 and sampled in the end of incubation (timc).

### Assay Steps in 96-Well Plate for Uptake Inhibition Assays

DMSO stock solutions of Compound I-19, Compound A and Compound B were added to the respective assay buffer in 96-well plate (helper plate) at the desired nominal concentrations. The aqueous buffer was mixed, and samples (C₀ or Total) were immediately taken for determining the total initial concentrations. The helper plate was pre-warmed for 15 minutes at 37° C. The pre-warmed aliquots of the aqueous buffers with the test articles (50 μL) were transferred from the helper plate into the wells of another 96-well plate (assay plate) and incubated for 30 minutes (pre-incubation) then removed from the wells. Thereafter the same wells of the assay plate were dosed again with other aliquots of the aqueous buffers with the test articles and were further incubated in the assay plate at 37° C. and finally sampled (t_{inc}).

### Vesicular Transport Inhibition Assays

Vesicular transport assays were performed with inside-out membrane vesicles prepared from cells overexpressing human ABC (efflux) transporters. The transporters were expressed by SOLVO Biotechnology in mammalian (HEK293) cells. The mammalian cells were stably transfected with the ABC transporter of interest. The assays were conducted according to the actual SOLVO VT assay protocols (PR-ASY-VT-General Protocol for Vesicular Transport Inhibition Assays).

The membrane vesicle suspension was mixed with the ice-cold transport buffer, then the probe substrate solution was added. This reaction mixture was distributed at 50 μL per well (total protein: 50 μg/well or 12.5 μg/well for BCRP) and pre-incubated with Compound I-19, Compound A and Compound B or the positive control at 1.5-times the final assay concentrations for 15 minutes at 37° C. or 32° C. (for BCRP and MDR1). The reactions were initiated by the addition of 25 μL per well pre-warmed 12 mM MgATP (or 12 mM AMP as a background control) to the 50 μL per well reaction mixtures, diluting the ATP and AMP to 4 mM and Compound I-19, Compound A and Compound B to the final assay concentrations. Incubations were conducted under assay parameters specified in established protocols listed in Table 12. Reactions were quenched by the addition of 200 μL of ice-cold washing buffer and immediate filtration via glass fiber filters mounted to a 96-well plate (filter plate). The filters were washed (5×200 μL of ice-cold washing buffer), dried and the amount of substrate inside the filtered vesicles was determined by liquid scintillation counting.

Incubation with AMP provided background activity values for all data points. Incubations with a respective probe substrate (100% activity values) in the presence and absence of a reference inhibitor were performed for each efflux transporter to confirm the transporter function.

### Uptake Transporter Inhibition Assays

Uptake experiments were performed using HEK293 cells stably expressing the respective uptake transporters. The assays were conducted according to the actual SOLVO uptake transporter assay protocols (PR-ASY-UPT-General Protocol for Uptake Transporter Inhibition Assays).

Cells were cultured at 37° C. in a humidified atmosphere containing 5% CO2 and were plated onto standard 96-well tissue culture plates.

Before the experiment, the medium was removed, and the cells were washed twice with 100 μL of pre-warmed (37° C.) assay buffer listed in Table 15. The cells were pre-incubated at 37° C. for 30 minutes in assay buffer containing the test article at appropriate concentrations (Table 16), appropriate solvents for solvent controls and the reference inhibitor for reference inhibitor wells. After the pre-incubation step solutions were removed. Uptake experiments were performed at 37° C. in 50 μL of assay buffer containing the probe substrate and the test article or solvent and controls. The organic solvent concentration was equal in all wells and did not exceed 1.5% (v/v).

After the experiment, cells were washed twice with 100 μL of appropriate cold buffer and lysed with 150 μL scintillation cocktail. Radiolabeled probe substrate transport was determined by liquid scintillation counting.

Uptake of the probe substrate in control cells provided background activity values for all data points. Incubations with a respective probe substrate (100% activity values) in the presence and absence of a reference inhibitor were performed for each uptake transporter to confirm the transporter function.

### Example 10: P450 Enzyme Inhibition of Compound I-19, Compound B, and Compound A

### Materials and Methods

### Human Cytochrome P450 Enzyme Inhibition Assays

Compound I-19 and Compound B were dissolved at 50 mM in DMSO followed by further serial dilutions in DMSO down to 10 mM. Compound A was dissolved at 10 mM in DMSO followed by further serial dilutions in DMSO down to 2 mM.

Substrate concentrations approximated their Kₘ for each respective isozyme and were expected to fall within the linear range of P450-mediated metabolism.

The P450 microsomes were submerged in a 37° C. water bath until just thawed and then placed on ice. NADPH was dissolved to 2 mM (2× stock) in 100 mM potassium phosphate buffer, pH 7.4. The substrates in DMSO (40 mM Phenacetin, 25 mM bupropion, 1 mM amodiaquine, 10 mM diclofenac, 40 mM mephenytoin, 10 mM dextromethorphan, 3.06 midazolam and 125 mM testosterone) were added to the NADPH solution to make a (2×) buffer/cofactor/substrate (BCS) solution. The test and positive control article (diluted in DMSO) were diluted 5-fold in acetonitrile followed by a further 100-fold dilution into BCS solution (to make a 2× compound solution); the final concentrations of DMSO and acetonitrile were less than 0.5% (v/v) each. Compound I-19, Compound B, fluvoxamine, ticlopidine, quercetin, sulfaphenazole, omeprazole, and ketoconazole concentrations were 50, 33.3, 22.2, 5.56, 1.39, 0.347, 0.0868, 0.0217, 0.00543, and 0 μM. Compound A concentrations were 10, 6.7, 4.4, 1.11, 0.28, 0.069, 0.0174, 0.0043, 0.00109, and 0 μM. Quinidine concentrations were 5, 3.33, 2.22, 0.556, 0.139, 0.0347, 0.00868, 0.00217, 0.000543, and 0 μM. Pooled human liver microsomes were diluted in 100 mM potassium phosphate, pH 7.4 to 0.2 mg/mL (a 2× solution). Prior to initiating the assay, the buffer/cofactor/substrate solutions containing the test/control articles and diluted microsomes were warmed to 37° C. Replicate 75-μL aliquots were added together to initiate the reactions; the final reaction volume was 150 μL.

For the T₀ control condition, 50-μL aliquots (from the samples containing 0 μM and the highest concentration of the test or control article) were immediately removed and quenched with 200 μL of acetonitrile containing internal standard. The assay plate was sealed and incubated at 37° C. with very gentle agitation. After a 30-minute incubation period, 50-μL aliquots were removed and quenched with 200 μL of acetonitrile containing internal standard.

The quenched samples were then centrifuged at 3100 rpm for 10 minutes at 4° C. Unpooled 2C19 supernatant (50 μL) was removed, transferred to a new 96-well plate and diluted with 100 μL of water. Pooled P450 supernatant (30 μL) was removed, transferred to a new 96-well plate and diluted with 180 μL of water. The sample plate was sealed, mixed and then stored refrigerated until analysis.

### Bioanalysis

For bioanalysis of CYP1A2, CYP2B6, CYP2C8, CYP2C9, CYP2C19, CYP2D6, and CYP3A4 samples, the LC system used a Waters XSELECT HSS T3 2.5 μm, 50×2.1 mm column. The column was set to a temperature of 55° C. The gradients were run at 0.8 mL/min from mobile phase A (0.1% formic acid in water) to mobile phase B (0.1% formic acid in acetonitrile), as follows:
CYP1A2: 3 to 95% mobile phase B
CYP2B6: 3 to 95% mobile phase B
CYP2C8: 3 to 95% mobile phase B
CYP2C9: 20 to 95% mobile phase B
CYP2C19: 25 to 95% mobile phase B
CYP2D6: 20 to 95% mobile phase B
CYP3A4: 20 to 95% mobile phase B

Analytes and internal standards were detected using an Applied Biosystems Sciex API-5500 triple quadrupole mass spectrometer with Waters Acquity UPLC System. The instrument was equipped with an electrospray ionization source (600° C.) operated in the positive-ion mode.

Analytes and internal standards were monitored in the multiple-reaction-monitoring scan mode as follows:
Acetaminophen (1A2): 152.1/110.2
d4-Acetaminophen (1A2 IS): 156.0/114.0
Hydroxybupropion (2B6): 238.1/139.0
d6-Hydroxybupropion (2B6 IS): 244.1/139.0
Desethylamodiaquine (2C8): 328.2/283.0
d5-Desethylamodiaquine (2C8 IS): 333.2/283.0
Hydroxydiclofenac (2C9): 312.1/230.2
¹³C6-Hydroxydiclofenac (2C9 IS): 318.1/236.1
4-Hydroxymephenytoin (2C19): 235.1/150.2
d3-4-Hydroxymephenytoin (2C19 IS): 238.1/150.2
Dextrorphan (2D6): 258.0/157.1
d3-Dextrorphan (2D6 IS): 261.0/157.1
Hydroxymidazolam (3A4): 342.1/203.1
Hydroxymidazolam (3A4 IS): 346.1/203.1
Hydroxytestosterone (3A4): 305.2/269.1
d3-Hydroxytestosterone (3A4 IS): 308.4/272.1

### Data Collection and Analysis

Data were captured and processed using Analyst v.1.7.2. Data were analyzed and results were calculated using Microsoft Excel and GraphPad Prism v.5.02 (for curve fitting and IC₅₀ calculations).

Percent inhibition was calculated by the following equation:

$$\% Inhibition = \frac{100 - ( Area Ratio - No Activity Background )}{Net Signal} \times 100 \%$$

Where:
No Activity Background (i.e., full inhibition) is the T0 quenched condition
Full Activity is T30 without inhibitor (i.e., non-inhibited) condition

Net Signal=Full Activity−No Activity Background

$$Percent Activity = 100 \% - \% Inhibition$$

### Results and Discussion

Compound I-19, Compound B and Compound A were tested for inhibition of human P450 isozymes in a single lot of pooled human liver microsomes, mixed gender, using an IC₅₀ method. To achieve a full dose-response, the data needs to define both the top plateau (representing full inhibition) and the bottom plateau (representing no inhibition). Generally, a dose-response curve is considered adequate for estimating IC₅₀ if the test article exceeds 70% inhibition. Human cytochrome P450 isoenzyme inhibition results are summarized in Table 136.

TABLE 136
Summary of Compound I-19 and Compound B Human
Cytochrome P450 Enzyme Inhibition Results

| | | | Inhibition at Highest Test | IC₅₀ |
|---|---|---|---|---|
| Compound ID | P450 Isozyme | P450 Substrate | Article Concentration (%) | (μM) |
| Compound I-19 | 1A2 | Phenacetin | 18.2 at 50 μM | ND |
| | 2B6 | Bupropion | 77.2 at 50 μM | 7.44 |
| | 2C8 | Amodiaquine | 73.7 at 50 μM | 2.38 |
| | 2C9 | Diclofenac | 53.3 at 50 μM | ND |
| | 2C19 | Mephenytoin | 83.2 at 50 μM | 17.3 |
| | 2D6 | Dextromethorphan | 84.3 at 50 μM | 8.70 |
| | 3A4 | Midazolam | 28.4 at 50 μM | ND |
| | 3A4 | Testosterone | 57.5 at 50 μM | ND |
| Compound B | 1A2 | Phenacetin | −24.3 at 50 μM | ND |
| | 2B6 | Bupropion | 78.3 at 50 μM | 4.73 |
| | 2C8 | Amodiaquine | 62.4 at 50 μM | ND |
| | 2C9 | Diclofenac | 50.5 at 50 μM | ND |
| | 2C19 | Mephenytoin | 44.5 at 50 μM | ND |
| | 2D6 | Dextromethorphan | 82.9 at 50 μM | 10.8 |
| | 3A4 | Midazolam | 39.5 at 50 μM | ND |
| | 3A4 | Testosterone | 57.7 at 50 μM | ND |
| Compound A | 1A2 | Phenacetin | 13.7 at 50 μM | ND |
| | 2B6 | Bupropion | 62.0 at 10 μM | 0.0920 |
| | 2C8 | Amodiaquine | 48.9 at 10 μM | ND |
| | 2C9 | Diclofenac | 10.2 at 10 μM | ND |
| | 2C19 | Mephenytoin | 16.5 at 10 μM | ND |
| | 2D6 | Dextromethorphan | 70.8 at 10 μM | 4.16 |
| | 3A4 | Midazolam | 11.7 at 10 μM | ND |
| | 3A4 | Testosterone | 14.0 at 10 μM | ND |
| Controls: | | | | |
| Fluvoxamine | 1A2 | Phenacetin | 101 at 50 μM | 0.112 |
| Ticlopidine | 2B6 | Bupropion | 99.7 at 50 μM | 0.572 |
| Quercetin | 2C8 | Amodiaquine | 101.8 at 50 μM | 0.668 |
| Sulfaphenazole | 2C9 | Diclofenac | 98.7 at 50 μM | 0.473 |
| Omeprazole | 2C19 | Mephenytoin | 89.9 at 50 μM | 7.36 |
| Quinidine | 2D6 | Dextromethorphan | 96.2 at 5 μM | 0.0850 |
| Ketoconazole | 3A4 | Midazolam | 103.4 at 50 μM | 0.0825 |
| Ketoconazaole | 3A4 | Testosterone | 101.9 at 50 μM | 0.0373 |

ND: Not determined

Compound I-19 displayed concentration dependent inhibition of four P450 isozymes: 2B6 (IC₅₀ 7.44 μM), 2C8 (IC₅₀ 2.38 μM), 2C19 (IC₅₀ 17.3 μM), and 2D6 (IC₅₀8.70 μM). Compound I-19 did not inhibit P450 isozymes 1A2, 2C9 or 3A4 (midazolam or testosterone) appreciably enough to establish IC₅₀ values.

Compound B displayed concentration dependent inhibition of two P450 isozymes: 2B6 (IC₅₀4.73 μM) and 2D6 (IC₅₀10.8 μM). Compound B did not inhibit P450 isozymes 1A2, 2C8, 2C9, 2C19, or 3A4 (midazolam or testosterone) appreciably enough to establish IC₅₀ values.

Compound A displayed concentration dependent inhibition of two P450 isozymes: 2B6 (IC₅₀0.0920 μM) and 2D6 (IC₅₀ 4.16 μM). Compound A did not inhibit P450 isozymes 1A2, 2C8, 2C9, 2C19, or 3A4 (midazolam or testosterone) appreciably enough to establish IC₅₀ values.

### Example 11. Evaluating the Effect of Compound I-19 on Time-Restricted Feeding and the Behavioral Satiety Sequence in the Long Evans Rat

The Behavioural Satiety Sequence (BSS) is a technique used to evaluate physiological satiety. It is based on the expression of a sequence of behaviors including eating, grooming, locomotor activity, followed by resting, which are typically observed in rodents post-ingestion (Halford et al, 1998, Pharmacology Biochemistry and Behaviour. 61(2): 159-168). The BSS is a widely used model for determining the effects of novel pharmaceuticals on food intake and measures of satiety in rodents (Oliveira et al, 2011, Revista de Nutrição. 24(4) 619-628), and to discriminate the cause of reduced food intake following pharmacological manipulation. For example, treatments that may reduce food intake by non-specific mechanisms, such as immobility, malaise, and taste adulteration, will disrupt this sequence (Halford et al, 1998, Pharmacology Biochemistry and Behaviour. 61(2): 159-168).

The primary objective of this study was to evaluate the effect of Compound I-19 on deprivation-induced feeding and the rodent behavioral satiety sequence (BSS) in Long Evans rats. Inclusion of the BSS was used to assist in differentiating any potential sedative, or other non-specific effect (e.g. malaise) of Compound I-19 on feeding behavior.

The primary objective of this study was to evaluate the effect of test article Compound I-19 (0.1, 0.3, 1, 3 mg/kg) on feeding behavior and BSS. Food consumption was limited to 2 h/day and so was motivated by hunger caused by 22 h food deprivation. Water was freely available 24 h/day.

Specifically, 12 male Long-Evans rats were food-deprived for 22 h with a 2 h feeding period each day. Rats were trained to consume daily food during the 2 h period over 25 days. The testing phase was initiated once the rats consistently consumed 16-18 g of food per day with ≤10% variance in amount of feed consumed. Feeding took place in designated individual boxes in a quiet room separate from the housing room. Continuous remote monitoring via video camera during feeding was used to assess the BSS. Food consumption was measured by weighing the total available food before and after the 2 h feeding window, correcting for spillage. Lorcaserin (1 mg/kg) was included as a reference control (Higgins et al, 2011, Neuropsychopharmacology. 37(5): 1177-1191). Subjects were tested using a repeated-measures, Latin square design with 2-3 days wash-out between treatment cycles.

### Materials and Methods

### Test Articles

Test article Compound I-19, Batch #115-164.1 (manufactured Provid Pharmaceuticals Inc.) was suspended in 0.8% DMSO, 6% PEG400, 93.2% HβCD (6% H₂O) and sonicated until fully dissolved. Drug was administered at a volume of 1 mL/kg, intraperitoneal (IP). Three doses were tested: 0.1, 0.3, 1, 3 mg/kg. There was a pretreatment time of 30 minutes.

Lorcaserin hydrochloride (Lot #FV-267) (manufactured Fluorinov Pharma Inc.) had a dosage form of: 0.9% Saline. Drug was administered at a volume of 1 mL/kg, subcutaneous (SC) route. One dose was tested: 1 mg/kg (expressed as base). There was a pretreatment time of 10 minutes.

### Test System

12 male Long Evans rats. All animals from this study were obtained from Charles River Laboratories, Quebec.

### Selection and Allocation of Animals

Any animal deemed unsatisfactory for the purpose of the study, such as poor health, was eliminated from the study.

### Housing and Management of Test System

Following a five day period of familiarization to the test facility, animals were placed on a restricted diet regimen, in which they were allotted approximately 40 g of standard laboratory chow once per day (between 10:00 h and 14:00 h). Rats were trained to consume the daily food (approximately 16-18 g of feed consumed) over a 2 h period. Daily food intakes were stable by study Day 25, with the quantity of feed consumed varying by ≤10% per day. Water was available ad-libitum. Subjects were pair-housed for the duration of the study and were maintained on a 12 h/12 h light/dark cycle with all testing conducted during the animals' light cycle. All animal use procedures were performed in accordance with the principles of the Canadian Council on Animal Care (CCAC).

### Behavioral Satiety Sequence

On scheduled test days, animals were monitored remotely via video camera during the feeding access period. The occurrence of four mutually exclusive behaviors (eating/drinking, grooming, active, rest) were individually scored. Over the 2 h feeding period, behaviors were assessed every 30 seconds. Scores were compiled into 23 5 min timebins (timebin 24 was removed due to early removal of animals) and the frequency of each behavior within each timebin was calculated to determine a temporal frequency of each measure (Rodgers et al, 2010, Pharmacology Biochemistry and Behaviour. 97: 3-14). Percentage time was plotted for each behavioral sequence in each timebin to compare between treatment groups (Higgins et al, 2011, Neuropsychopharmacology. 37(5): 1177-1191).

Feed was measured using a certified scale according to standard operating procedures prior to testing. Residual feed was weighed following completion of the 2 h feeding window to track total intake (g).

### Administration of Test Article

Compound I-19 and Lorcaserin were prepared fresh on each test day. Test article was suspended in 0.8% DMSO, 6% PEG400, and 93.2% HβCD (6% in H₂O) and sonicated until fully dissolved. Compound I-19 was administered at a volume of 1 mL/kg via the intraperitoneal route. Pretreatment time (ptt) was 30 minutes prior to commencement of testing. Lorcaserin was dissolved in 0.9% saline and administered via the subcutaneous route with a dose volume of 1 mL/kg with a ppt of 10 minutes.

### Statistical Analysis

The primary measure from the BSS test was total measured food intake (g), expressed as mean+SEM. Data were analysed by repeated-measures ANOVA (with treatment as a factor) (Statistica Version 11, Statsoft Inc. [2012]). Planned post-hoc comparisons were made in the event of a significant main effect using Dunnett's test. Significance was set at P<0.05.

### Results and Discussion

Food intake: Analysis of variance revealed a main effect of treatment on food intake (F5,55=11.84, P<0.01). Post-hoc analysis of treatment groups against vehicle (control) intake showed a modest dose-related decrease in food intake following Compound I-19 (1-3 mg/kg; Table 137). At the 3 mg/kg dose, food intake was reduced from 17.8±0.7 g (vehicle) to 15.9±0.5 g (3 mg/kg Compound I-19), i.e., a decrease of 10.6%. The magnitude of this change was similar to that produced by the reference control, lorcaserin (1 mg/kg), from 17.8±0.7 g (vehicle) to 14.6±0.7 g, i.e., a decrease of 17.4%.

TABLE 137
Food Consumption-Summary Data Compound I-19 and Lorcaserin. Data is
presented in grams. Compound I-19 is in doses according to mg/kg.

| | | Compound I-19 | | | | |
|---|---|---|---|---|---|---|
| Animal ID | VEH | 0.1 | 0.3 | 1 | 3 | Lorcaserin |
| LT-1 | 15.6 | 14.5 | 14.9 | 13.8 | 15 | 13.5 |
| LT-2 | 17 | 14.1 | 14.5 | 14.9 | 13.1 | 12 |
| LT-3 | 18.3 | 19.7 | 16.5 | 16.3 | 16.8 | 12.7 |
| LT-4 | 20.9 | 22.2 | 22.1 | 18.7 | 19 | 17.9 |
| LT-5 | 13.8 | 13.4 | 15 | 13.2 | 14.1 | 12.7 |
| LT-6 | 18.4 | 17.8 | 18.8 | 17.8 | 18.3 | 12.5 |
| LT-7 | 18.2 | 18.2 | 15.4 | 15.8 | 14 | 18 |
| LT-8 | 20.7 | 19.4 | 19 | 19.3 | 17.7 | 17.7 |
| LT-9 | 19.4 | 20.7 | 18.2 | 17.7 | 16.6 | 16.3 |
| LT-10 | 19.7 | 19.4 | 18.1 | 13.4 | 16.3 | 15.7 |
| LT-11 | 16.9 | 18 | 15.8 | 14 | 14.8 | 12.9 |
| LT-12 | 14.6 | 15.7 | 17.8 | 14.9 | 15.5 | 13.1 |
| AVG | 17.8 | 17.8 | 17.2 | 15.8 | 15.9 | 14.6 |
| SEM | 0.7 | 0.8 | 0.6 | 0.6 | 0.5 | 0.7 |

Behavioral satiety sequence: Characteristically, when food restricted rats are given free access to food, they show a typical sequence of behaviors typified by an initial predominance of eating/drinking with the exclusion of other behaviors. Behavior transitions over time to bouts of activity, followed by resting as they feed to satiety. However, in the present study, eating/drinking remained the primary behavior in vehicle pretreated subjects at the later 1.5-2 h timepoints (Tables 138-141). As the scoring method utilized did not differentiate between eating and drinking, it is unknown whether the association between the two ingestive behaviors varied over time. Generally speaking, the Compound I-19 doses that reduced food intake did not affect the BSS compared to vehicle pretreatment, i.e., eating/drinking was the dominant behavior at the onset of food access following each dose of Compound I-19, with a gradual emergence of active/resting at the later timepoints. This is consistent with the interpretation of Compound I-19 reducing food intake by enhancing the onset of satiety, rather than as a consequence of non-specific motor effects, induction of malaise, or taste adulteration of the food. There were no overt behavioral changes reported in any study subject following Compound 1-19 pretreatment. Similarly, lorcaserin preserved the BSS at the 1 mg/kg dose, consistent with an enhancement of satiety.

TABLE 138
BSS (Behavioral Satiety Sequence) Scoring - Raw Data 0-0.5 hours. E is
eating/drinking; G is grooming; A is active; and R is resting.

| | Ani- | Time Bin 1 (0-5 | | | | Time Bin 2 (5-10 | | | | Time Bin 3 (10-15 | | | | Time Bin 4 (15-20 | | | | Time Bin 5 (20-25 | | | | Time Bin 6 (25-30 | | | |
|---|---|---|---|---|---|---|---|---|---|---|---|---|---|---|---|---|---|---|---|---|---|---|---|---|---|
| Treat- | mal | TOTAL) | | | | TOTAL) | | | | TOTAL) | | | | TOTAL) | | | | TOTAL) | | | | TOTAL) | | | |
| ment | ID | E | G | A | R | E | G | A | R | E | G | A | R | E | G | A | R | E | G | A | R | E | G | A | R |
| Ve- | LT-1 | 10 | 0 | 0 | 0 | 10 | 0 | 0 | 0 | 10 | 0 | 0 | 0 | 9 | 0 | 1 | 0 | 8 | 1 | 1 | 0 | 9 | 1 | 0 | 0 |
| hicle | LT-2 | 7 | 1 | 2 | 0 | 9 | 1 | 0 | 0 | 10 | 0 | 0 | 0 | 10 | 0 | 0 | 0 | 7 | 1 | 2 | 0 | 9 | 0 | 1 | 0 |
| | LT-3 | 7 | 1 | 1 | 1 | 9 | 1 | 0 | 0 | 7 | 0 | 1 | 2 | 10 | 0 | 0 | 0 | 6 | 1 | 1 | 2 | 8 | 0 | 1 | 1 |
| | LT-4 | 9 | 0 | 1 | 0 | 8 | 1 | 1 | 0 | 10 | 0 | 0 | 0 | 9 | 1 | 0 | 0 | 10 | 0 | 0 | 0 | 10 | 0 | 0 | 0 |
| | LT-5 | 10 | 0 | 0 | 0 | 9 | 0 | 0 | 1 | 9 | 0 | 1 | 0 | 9 | 0 | 0 | 1 | 10 | 0 | 0 | 0 | 9 | 0 | 0 | 1 |
| | LT-6 | 9 | 0 | 1 | 0 | 9 | 1 | 0 | 0 | 10 | 0 | 0 | 0 | 9 | 1 | 0 | 0 | 10 | 0 | 0 | 0 | 10 | 0 | 0 | 0 |
| | LT-7 | 7 | 0 | 3 | 0 | 10 | 0 | 0 | 0 | 10 | 0 | 0 | 0 | 10 | 0 | 0 | 0 | 9 | 1 | 0 | 0 | 10 | 0 | 0 | 0 |
| | LT-8 | 9 | 1 | 0 | 0 | 10 | 0 | 0 | 0 | 9 | 0 | 1 | 0 | 7 | 2 | 0 | 1 | 8 | 2 | 0 | 0 | 9 | 0 | 1 | 0 |
| | LT-9 | 9 | 0 | 1 | 0 | 9 | 0 | 1 | 0 | 9 | 0 | 0 | 1 | 9 | 0 | 1 | 0 | 10 | 0 | 0 | 0 | 9 | 0 | 1 | 0 |
| | LT- | 10 | 0 | 0 | 0 | 10 | 0 | 0 | 0 | 10 | 0 | 0 | 0 | 9 | 1 | 0 | 0 | 10 | 0 | 0 | 0 | 7 | 1 | 2 | 0 |
| | 10 | | | | | | | | | | | | | | | | | | | | | | | | |
| | LT- | 9 | 0 | 1 | 0 | 10 | 0 | 0 | 0 | 10 | 0 | 0 | 0 | 9 | 1 | 0 | 0 | 10 | 0 | 0 | 0 | 7 | 1 | 2 | 0 |
| | 11 | | | | | | | | | | | | | | | | | | | | | | | | |
| | LT- | 10 | 0 | 0 | 0 | 10 | 0 | 0 | 0 | 10 | 0 | 0 | 0 | 10 | 0 | 0 | 0 | 9 | 1 | 0 | 0 | 7 | 0 | 3 | 0 |
| | 12 | | | | | | | | | | | | | | | | | | | | | | | | |
| AVG | | 0.88 | 0.03 | 0.08 | 0.01 | 0.94 | 0.03 | 0.02 | 0.01 | 0.95 | 0.00 | 0.03 | 0.03 | 0.92 | 0.05 | 0.02 | 0.02 | 0.89 | 0.06 | 0.03 | 0.02 | 0.87 | 0.03 | 0.09 | 0.02 |
| SEM | | 0.34 | 0.13 | 0.27 | 0.08 | 0.19 | 0.14 | 0.11 | 0.08 | 0.26 | 0.00 | 0.13 | 0.18 | 0.24 | 0.19 | 0.11 | 0.11 | 0.40 | 0.19 | 0.19 | 0.17 | 0.33 | 0.13 | 0.29 | 0.11 |
| Lor- | LT-1 | 7 | 0 | 3 | 0 | 6 | 3 | 1 | 0 | 8 | 1 | 1 | 0 | 7 | 0 | 3 | 0 | 9 | 1 | 0 | 0 | 7 | 0 | 2 | 1 |
| caserin | LT-2 | 6 | 2 | 2 | 0 | 9 | 1 | 0 | 0 | 8 | 2 | 0 | 0 | 8 | 1 | 1 | 0 | 8 | 2 | 0 | 0 | 9 | 1 | 0 | 0 |
| (1 | LT-3 | 7 | 1 | 2 | 0 | 8 | 0 | 2 | 0 | 5 | 2 | 2 | 1 | 6 | 1 | 3 | 0 | 7 | 2 | 1 | 0 | 9 | 0 | 1 | 0 |
| mg/ | LT-4 | 8 | 1 | 1 | 0 | 8 | 2 | 0 | 0 | 8 | 1 | 1 | 0 | 10 | 0 | 0 | 0 | 7 | 0 | 3 | 0 | 10 | 0 | 0 | 0 |
| kg) | LT-5 | 10 | 0 | 0 | 0 | 7 | 1 | 2 | 0 | 7 | 2 | 1 | 0 | 9 | 0 | 0 | 1 | 4 | 2 | 4 | 0 | 8 | 1 | 1 | 0 |
| | LT-6 | 8 | 1 | 1 | 0 | 10 | 0 | 0 | 0 | 6 | 2 | 1 | 0 | 9 | 1 | 0 | 0 | 6 | 0 | 4 | 0 | 1 | 1 | 7 | 1 |
| | LT-7 | 7 | 0 | 2 | 1 | 9 | 0 | 1 | 0 | 8 | 1 | 1 | 0 | 7 | 2 | 0 | 1 | 8 | 2 | 0 | 0 | 8 | 0 | 1 | 1 |
| | LT-8 | 6 | 1 | 3 | 0 | 10 | 0 | 0 | 0 | 8 | 0 | 2 | 0 | 6 | 2 | 1 | 1 | 8 | 0 | 2 | 0 | 8 | 0 | 2 | 0 |
| | LT-9 | 6 | 1 | 3 | 0 | 9 | 0 | 1 | 0 | 8 | 0 | 1 | 1 | 9 | 1 | 0 | 0 | 7 | 1 | 2 | 0 | 9 | 0 | 1 | 0 |
| | LT- | 7 | 1 | 2 | 0 | 8 | 1 | 0 | 1 | 10 | 0 | 0 | 0 | 7 | 0 | 3 | 0 | 8 | 1 | 1 | 0 | 9 | 1 | 0 | 0 |
| | 10 | | | | | | | | | | | | | | | | | | | | | | | | |
| | LT- | 9 | 1 | 0 | 0 | 6 | 2 | 2 | 0 | 9 | 1 | 0 | 0 | 9 | 1 | 0 | 0 | 4 | 3 | 3 | 0 | 7 | 0 | 3 | 0 |
| | 11 | | | | | | | | | | | | | | | | | | | | | | | | |
| | LT- | 9 | 0 | 1 | 0 | 9 | 1 | 0 | 0 | 9 | 0 | 1 | 0 | 6 | 2 | 1 | 1 | 9 | 0 | 1 | 0 | 5 | 2 | 0 | 3 |
| | 12 | | | | | | | | | | | | | | | | | | | | | | | | |
| AVG | | 0.75 | 0.08 | 0.17 | 0.01 | 0.83 | 0.09 | 0.08 | 0.01 | 0.78 | 0.10 | 0.09 | 0.02 | 0.78 | 0.09 | 0.10 | 0.03 | 0.71 | 0.12 | 0.18 | 0.00 | 0.75 | 0.05 | 0.15 | 0.05 |
| SEM | | 0.38 | 0.18 | 0.31 | 0.08 | 0.39 | 0.29 | 0.25 | 0.08 | 0.39 | 0.25 | 0.19 | 0.11 | 0.41 | 0.23 | 0.37 | 0.14 | 0.48 | 0.30 | 0.43 | 0.00 | 0.70 | 0.19 | 0.57 | 0.26 |
| Com- | LT-1 | 10 | 0 | 0 | 0 | 9 | 0 | 1 | 0 | 9 | 0 | 1 | 0 | 5 | 0 | 5 | 0 | 9 | 0 | 1 | 0 | 5 | 0 | 3 | 2 |
| pound | LT-2 | 8 | 1 | 1 | 0 | 10 | 0 | 0 | 0 | 9 | 1 | 0 | 0 | 10 | 0 | 0 | 0 | 10 | 0 | 0 | 0 | 8 | 1 | 1 | 0 |
| I-19 | LT-3 | 8 | 0 | 2 | 0 | 9 | 0 | 1 | 0 | 9 | 0 | 1 | 0 | 9 | 0 | 0 | 1 | 10 | 0 | 0 | 0 | 8 | 1 | 1 | 0 |
| (0.1 | LT-4 | 8 | 0 | 2 | 0 | 10 | 0 | 0 | 0 | 10 | 0 | 0 | 0 | 10 | 0 | 0 | 0 | 6 | 0 | 3 | 1 | 9 | 0 | 1 | 0 |
| mg/ | LT-5 | 10 | 0 | 0 | 0 | 10 | 0 | 0 | 0 | 10 | 0 | 0 | 0 | 4 | 1 | 4 | 1 | 9 | 0 | 0 | 1 | 10 | 0 | 0 | 0 |
| kg) | LT-6 | 9 | 0 | 1 | 0 | 10 | 0 | 0 | 0 | 10 | 0 | 0 | 0 | 9 | 1 | 0 | 0 | 10 | 0 | 0 | 0 | 7 | 0 | 3 | 0 |
| | LT-7 | 9 | 0 | 1 | 0 | 10 | 0 | 0 | 0 | 9 | 1 | 0 | 0 | 10 | 0 | 0 | 0 | 10 | 0 | 0 | 0 | 8 | 1 | 1 | 0 |
| | LT-8 | 9 | 0 | 1 | 0 | 10 | 0 | 0 | 0 | 10 | 0 | 0 | 0 | 9 | 1 | 0 | 0 | 7 | 0 | 1 | 2 | 10 | 0 | 0 | 0 |
| | LT-9 | 10 | 0 | 0 | 0 | 9 | 0 | 1 | 0 | 10 | 0 | 0 | 0 | 9 | 0 | 1 | 0 | 10 | 0 | 0 | 0 | 8 | 1 | 1 | 0 |
| | LT- | 9 | 0 | 1 | 0 | 9 | 0 | 1 | 0 | 9 | 1 | 0 | 0 | 10 | 0 | 0 | 0 | 7 | 0 | 3 | 0 | 5 | 1 | 4 | 0 |
| | 10 | | | | | | | | | | | | | | | | | | | | | | | | |
| | LT- | 8 | 1 | 1 | 0 | 9 | 0 | 1 | 0 | 10 | 0 | 0 | 0 | 9 | 0 | 1 | 0 | 10 | 0 | 0 | 0 | 9 | 0 | 1 | 0 |
| | 11 | | | | | | | | | | | | | | | | | | | | | | | | |
| | LT- | 9 | 0 | 1 | 0 | 10 | 0 | 0 | 0 | 9 | 0 | 1 | 0 | 10 | 0 | 0 | 0 | 9 | 0 | 1 | 0 | 10 | 0 | 0 | 0 |
| | 12 | | | | | | | | | | | | | | | | | | | | | | | | |
| AVG | | 0.89 | 0.02 | 0.09 | 0.00 | 0.96 | 0.00 | 0.04 | 0.00 | 0.95 | 0.03 | 0.03 | 0.00 | 0.87 | 0.03 | 0.09 | 0.02 | 0.89 | 0.00 | 0.08 | 0.03 | 0.81 | 0.04 | 0.13 | 0.02 |
| SEM | | 0.23 | 0.11 | 0.19 | 0.00 | 0.15 | 0.00 | 0.15 | 0.00 | 0.15 | 0.13 | 0.13 | 0.00 | 0.58 | 0.13 | 0.50 | 0.11 | 0.42 | 0.00 | 0.33 | 0.19 | 0.50 | 0.15 | 0.38 | 0.17 |
| Com- | LT-1 | 9 | 0 | 1 | 0 | 9 | 1 | 0 | 0 | 10 | 0 | 0 | 0 | 10 | 0 | 0 | 0 | 6 | 1 | 3 | 0 | 9 | 0 | 1 | 0 |
| pound | LT-2 | 9 | 1 | 0 | 0 | 9 | 1 | 0 | 0 | 10 | 0 | 0 | 0 | 10 | 0 | 0 | 0 | 9 | 1 | 0 | 0 | 10 | 0 | 0 | 0 |
| I-19 | LT-3 | 7 | 0 | 2 | 1 | 9 | 1 | 0 | 0 | 10 | 0 | 0 | 0 | 9 | 0 | 1 | 0 | 9 | 0 | 0 | 1 | 7 | 0 | 0 | 3 |
| (0.3 | LT-4 | 8 | 0 | 2 | 0 | 10 | 0 | 0 | 0 | 9 | 1 | 0 | 0 | 10 | 0 | 0 | 0 | 10 | 0 | 0 | 0 | 8 | 0 | 2 | 0 |
| mg/ | LT-5 | 8 | 1 | 1 | 0 | 10 | 0 | 0 | 0 | 10 | 0 | 0 | 0 | 8 | 0 | 2 | 0 | 10 | 0 | 0 | 0 | 8 | 1 | 1 | 0 |
| kg) | LT-6 | 10 | 0 | 0 | 0 | 10 | 0 | 0 | 0 | 9 | 1 | 0 | 0 | 10 | 0 | 0 | 0 | 9 | 1 | 0 | 0 | 10 | 0 | 0 | 0 |
| | LT-7 | 9 | 0 | 1 | 0 | 10 | 0 | 0 | 0 | 8 | 0 | 2 | 0 | 9 | 0 | 0 | 1 | 9 | 1 | 0 | 0 | 10 | 0 | 0 | 0 |
| | LT-8 | 8 | 0 | 2 | 0 | 10 | 0 | 0 | 0 | 10 | 0 | 0 | 0 | 9 | 0 | 1 | 0 | 9 | 0 | 1 | 0 | 8 | 0 | 2 | 0 |
| | LT-9 | 10 | 0 | 0 | 0 | 10 | 0 | 0 | 0 | 10 | 0 | 0 | 0 | 9 | 1 | 0 | 0 | 10 | 0 | 0 | 0 | 10 | 0 | 0 | 0 |
| | LT- | 9 | 0 | 1 | 0 | 9 | 0 | 1 | 0 | 9 | 0 | 1 | 0 | 9 | 0 | 1 | 0 | 9 | 0 | 1 | 0 | 10 | 0 | 0 | 0 |
| | 10 | | | | | | | | | | | | | | | | | | | | | | | | |
| | LT- | 9 | 1 | 0 | 0 | 9 | 0 | 1 | 0 | 9 | 0 | 1 | 0 | 6 | 0 | 4 | 0 | 8 | 0 | 2 | 0 | 6 | 1 | 2 | 1 |
| | 11 | | | | | | | | | | | | | | | | | | | | | | | | |
| | LT- | 9 | 0 | 1 | 0 | 9 | 1 | 0 | 0 | 9 | 1 | 0 | 0 | 9 | 0 | 0 | 1 | 7 | 0 | 1 | 2 | 9 | 0 | 1 | 0 |
| | 12 | | | | | | | | | | | | | | | | | | | | | | | | |
| AVG | | 0.88 | 0.03 | 0.09 | 0.01 | 0.95 | 0.03 | 0.02 | 0.00 | 0.94 | 0.03 | 0.03 | 0.00 | 0.90 | 0.01 | 0.08 | 0.02 | 0.88 | 0.03 | 0.07 | 0.03 | 0.88 | 0.02 | 0.08 | 0.03 |
| SEM | | 0.25 | 0.13 | 0.23 | 0.08 | 0.15 | 0.14 | 0.11 | 0.00 | 0.19 | 0.13 | 0.19 | 0.00 | 0.33 | 0.08 | 0.35 | 0.11 | 0.35 | 0.14 | 0.28 | 0.18 | 0.39 | 0.11 | 0.25 | 0.26 |
| Com- | LT-1 | 10 | 0 | 0 | 0 | 10 | 0 | 0 | 0 | 10 | 0 | 0 | 0 | 10 | 0 | 0 | 0 | 7 | 1 | 2 | 0 | 6 | 0 | 2 | 2 |
| pound | LT-2 | 9 | 0 | 1 | 0 | 8 | 2 | 0 | 0 | 10 | 0 | 0 | 0 | 10 | 0 | 0 | 0 | 10 | 0 | 0 | 0 | 10 | 0 | 0 | 0 |
| I-19 | LT-3 | 7 | 1 | 2 | 0 | 10 | 0 | 0 | 0 | 9 | 0 | 1 | 0 | 9 | 0 | 1 | 0 | 9 | 0 | 0 | 1 | 10 | 0 | 0 | 0 |
| (1 | LT-4 | 8 | 0 | 2 | 0 | 8 | 0 | 2 | 0 | 9 | 0 | 1 | 0 | 9 | 1 | 0 | 0 | 8 | 1 | 1 | 0 | 10 | 0 | 0 | 0 |
| mg/ | LT-5 | 9 | 0 | 1 | 0 | 9 | 0 | 0 | 1 | 8 | 0 | 1 | 1 | 9 | 0 | 0 | 1 | 10 | 0 | 0 | 0 | 1 | 1 | 7 | 1 |
| kg) | LT-6 | 9 | 0 | 1 | 0 | 10 | 0 | 0 | 0 | 8 | 1 | 1 | 0 | 10 | 0 | 0 | 0 | 10 | 0 | 0 | 0 | 10 | 0 | 0 | 0 |
| | LT-7 | 8 | 0 | 2 | 0 | 8 | 1 | 1 | 0 | 10 | 0 | 0 | 0 | 8 | 1 | 0 | 1 | 9 | 0 | 1 | 0 | 7 | 0 | 3 | 0 |
| | LT-8 | 8 | 0 | 2 | 0 | 9 | 1 | 0 | 0 | 9 | 0 | 1 | 0 | 9 | 0 | 0 | 1 | 9 | 0 | 1 | 0 | 9 | 0 | 1 | 0 |
| | LT-9 | 10 | 0 | 0 | 0 | 10 | 0 | 0 | 0 | 9 | 0 | 1 | 0 | 9 | 1 | 0 | 0 | 9 | 0 | 1 | 0 | 8 | 0 | 2 | 0 |
| | LT- | 9 | 0 | 1 | 0 | 7 | 2 | 1 | 0 | 8 | 1 | 1 | 0 | 10 | 0 | 0 | 0 | 7 | 1 | 2 | 0 | 8 | 0 | 2 | 0 |
| | 10 | | | | | | | | | | | | | | | | | | | | | | | | |
| | LT- | 8 | 1 | 1 | 0 | 8 | 1 | 1 | 0 | 7 | 2 | 1 | 0 | 10 | 0 | 0 | 0 | 4 | 1 | 4 | 1 | 10 | 0 | 0 | 0 |
| | 11 | | | | | | | | | | | | | | | | | | | | | | | | |
| | LT- | 10 | 0 | 0 | 0 | 9 | 1 | 0 | 0 | 9 | 0 | 0 | 1 | 9 | 0 | 1 | 0 | 8 | 0 | 0 | 2 | 7 | 0 | 3 | 0 |
| | 12 | | | | | | | | | | | | | | | | | | | | | | | | |
| AVG | | 0.88 | 0.02 | 0.11 | 0.00 | 0.88 | 0.07 | 0.04 | 0.01 | 0.88 | 0.03 | 0.07 | 0.02 | 0.93 | 0.03 | 0.02 | 0.03 | 0.83 | 0.03 | 0.10 | 0.03 | 0.80 | 0.01 | 0.17 | 0.03 |
| SEM | | 0.28 | 0.11 | 0.23 | 0.00 | 0.30 | 0.22 | 0.19 | 0.08 | 0.27 | 0.19 | 0.14 | 0.11 | 0.19 | 0.13 | 0.11 | 0.13 | 0.50 | 0.14 | 0.35 | 0.19 | 0.76 | 0.08 | 0.59 | 0.18 |
| Com- | LT-1 | 9 | 0 | 1 | 0 | 9 | 0 | 1 | 0 | 9 | 0 | 1 | 0 | 10 | 0 | 0 | 0 | 8 | 1 | 1 | 0 | 9 | 0 | 1 | 0 |
| pound | LT-2 | 9 | 0 | 1 | 0 | 8 | 2 | 0 | 0 | 10 | 0 | 0 | 0 | 8 | 1 | 1 | 0 | 10 | 0 | 0 | 0 | 10 | 0 | 0 | 0 |
| I-19 | LT-3 | 8 | 1 | 1 | 0 | 10 | 0 | 0 | 0 | 8 | 0 | 2 | 0 | 8 | 1 | 1 | 0 | 10 | 0 | 0 | 0 | 7 | 0 | 1 | 2 |
| (3 | LT-4 | 8 | 2 | 0 | 0 | 10 | 0 | 0 | 0 | 8 | 0 | 2 | 0 | 10 | 0 | 0 | 0 | 8 | 1 | 1 | 0 | 9 | 0 | 1 | 0 |
| mg/ | LT-5 | 9 | 0 | 1 | 0 | 10 | 0 | 0 | 0 | 7 | 1 | 0 | 2 | 9 | 0 | 0 | 1 | 9 | 0 | 1 | 0 | 6 | 0 | 2 | 2 |
| kg) | LT-6 | 9 | 0 | 1 | 0 | 10 | 0 | 0 | 0 | 8 | 0 | 2 | 0 | 10 | 0 | 0 | 0 | 7 | 1 | 1 | 1 | 10 | 0 | 0 | 0 |
| | LT-7 | 8 | 0 | 2 | 0 | 8 | 1 | 1 | 0 | 8 | 0 | 2 | 0 | 9 | 0 | 0 | 1 | 9 | 0 | 1 | 0 | 8 | 0 | 2 | 0 |
| | LT-8 | 9 | 0 | 1 | 0 | 10 | 0 | 0 | 0 | 9 | 0 | 1 | 0 | 7 | 0 | 3 | 0 | 9 | 0 | 1 | 0 | 9 | 0 | 1 | 0 |
| | LT-9 | 7 | 0 | 3 | 0 | 10 | 0 | 0 | 0 | 10 | 0 | 0 | 0 | 10 | 0 | 0 | 0 | 9 | 1 | 0 | 0 | 10 | 0 | 0 | 0 |
| | LT- | 10 | 0 | 0 | 0 | 8 | 1 | 0 | 1 | 10 | 0 | 0 | 0 | 8 | 0 | 2 | 0 | 10 | 0 | 0 | 0 | 5 | 1 | 4 | 0 |
| | 10 | | | | | | | | | | | | | | | | | | | | | | | | |
| | LT- | 9 | 0 | 1 | 0 | 9 | 0 | 0 | 1 | 10 | 0 | 0 | 0 | 4 | 0 | 6 | 0 | 8 | 1 | 0 | 1 | 8 | 0 | 1 | 1 |
| | 11 | | | | | | | | | | | | | | | | | | | | | | | | |
| | LT- | 8 | 1 | 1 | 0 | 10 | 0 | 0 | 0 | 7 | 0 | 3 | 0 | 7 | 0 | 3 | 0 | 8 | 1 | 1 | 0 | 10 | 0 | 0 | 0 |
| | 12 | | | | | | | | | | | | | | | | | | | | | | | | |
| AVG | | 0.86 | 0.03 | 0.11 | 0.00 | 0.93 | 0.03 | 0.02 | 0.02 | 0.87 | 0.01 | 0.11 | 0.02 | 0.83 | 0.02 | 0.13 | 0.02 | 0.88 | 0.05 | 0.06 | 0.02 | 0.84 | 0.01 | 0.11 | 0.04 |
| SEM | | 0.23 | 0.19 | 0.23 | 0.00 | 0.26 | 0.19 | 0.11 | 0.11 | 0.33 | 0.08 | 0.31 | 0.17 | 0.51 | 0.11 | 0.54 | 0.11 | 0.28 | 0.15 | 0.15 | 0.11 | 0.48 | 0.08 | 0.34 | 0.23 |
| | Ani- | Time Bin 7 (30- | | | | Time Bin 8 (35- | | | | Time Bin 9 (40- | | | | Time Bin 10 (45- | | | | Time Bin 11 (50- | | | | Time Bin 12 (55- | | | |
| Treat- | mal | 35 TOTAL) | | | | 40 TOTAL) | | | | 45 TOTAL) | | | | 50 TOTAL) | | | | 55 TOTAL) | | | | 60 TOTAL) | | | |
| ment | ID | E | G | A | R | E | G | A | R | E | G | A | R | E | G | A | R | E | G | A | R | E | G | A | R |
| Ve- | LT-1 | 8 | 0 | 2 | 0 | 8 | 0 | 2 | 0 | 8 | 0 | 0 | 2 | 8 | 0 | 0 | 2 | 9 | 0 | 0 | 1 | 5 | 2 | 3 | 0 |
| hicle | LT-2 | 10 | 0 | 0 | 0 | 8 | 0 | 2 | 0 | 9 | 0 | 1 | 0 | 8 | 1 | 1 | 0 | 8 | 0 | 2 | 0 | 10 | 0 | 0 | 0 |
| | LT-3 | 10 | 0 | 0 | 0 | 8 | 1 | 1 | 0 | 9 | 0 | 1 | 0 | 10 | 0 | 0 | 0 | 10 | 0 | 0 | 0 | 10 | 0 | 0 | 0 |
| | LT-4 | 9 | 0 | 1 | 0 | 9 | 1 | 0 | 0 | 10 | 0 | 0 | 0 | 10 | 0 | 0 | 0 | 9 | 0 | 1 | 0 | 7 | 0 | 3 | 0 |
| | LT-5 | 8 | 0 | 0 | 2 | 4 | 0 | 5 | 1 | 8 | 0 | 2 | 0 | 10 | 0 | 0 | 0 | 6 | 0 | 1 | 3 | 8 | 0 | 0 | 2 |
| | LT-6 | 9 | 0 | 1 | 0 | 9 | 0 | 1 | 0 | 8 | 1 | 1 | 0 | 10 | 0 | 0 | 0 | 8 | 0 | 1 | 1 | 10 | 0 | 0 | 0 |
| | LT-7 | 8 | 0 | 2 | 0 | 10 | 0 | 0 | 0 | 10 | 0 | 0 | 0 | 5 | 0 | 5 | 0 | 9 | 1 | 0 | 0 | 8 | 0 | 2 | 0 |
| | LT-8 | 9 | 0 | 1 | 0 | 9 | 0 | 1 | 0 | 6 | 0 | 3 | 1 | 8 | 0 | 2 | 0 | 8 | 0 | 2 | 1 | 6 | 0 | 2 | 2 |
| | LT-9 | 9 | 1 | 0 | 0 | 10 | 0 | 0 | 0 | 8 | 1 | 1 | 0 | 7 | 0 | 3 | 0 | 10 | 0 | 0 | 0 | 8 | 2 | 0 | 0 |
| | LT- | 9 | 0 | 0 | 1 | 7 | 2 | 1 | 0 | 8 | 0 | 2 | 0 | 10 | 0 | 0 | 0 | 9 | 0 | 1 | 0 | 8 | 1 | 1 | 0 |
| | 10 | | | | | | | | | | | | | | | | | | | | | | | | |
| | LT- | 9 | 1 | 0 | 0 | 10 | 0 | 0 | 0 | 5 | 0 | 4 | 1 | 6 | 1 | 3 | 0 | 10 | 0 | 0 | 0 | 6 | 1 | 3 | 0 |
| | 11 | | | | | | | | | | | | | | | | | | | | | | | | |
| | LT- | 9 | 1 | 0 | 0 | 8 | 1 | 0 | 1 | 9 | 0 | 1 | 0 | 7 | 0 | 3 | 0 | 7 | 0 | 3 | 0 | 5 | 2 | 3 | 0 |
| | 12 | | | | | | | | | | | | | | | | | | | | | | | | |
| AVG | | 0.89 | 0.03 | 0.06 | 0.03 | 0.83 | 0.04 | 0.11 | 0.02 | 0.82 | 0.02 | 0.13 | 0.03 | 0.83 | 0.02 | 0.14 | 0.02 | 0.86 | 0.01 | 0.09 | 0.05 | 0.76 | 0.07 | 0.14 | 0.03 |
| SEM | | 0.19 | 0.13 | 0.23 | 0.18 | 0.48 | 0.19 | 0.42 | 0.11 | 0.42 | 0.11 | 0.36 | 0.19 | 0.51 | 0.11 | 0.50 | 0.17 | 0.36 | 0.08 | 0.29 | 0.26 | 0.53 | 0.26 | 0.40 | 0.22 |
| Lor- | LT-1 | 6 | 3 | 0 | 1 | 5 | 1 | 4 | 0 | 10 | 0 | 0 | 0 | 8 | 0 | 2 | 0 | 10 | 0 | 0 | 0 | 6 | 2 | 2 | 0 |
| caserin | LT-2 | 6 | 1 | 3 | 0 | 7 | 1 | 2 | 0 | 7 | 1 | 2 | 0 | 6 | 0 | 4 | 0 | 7 | 1 | 2 | 0 | 7 | 2 | 0 | 1 |
| (1 | LT-3 | 7 | 2 | 0 | 1 | 5 | 1 | 4 | 0 | 9 | 1 | 0 | 0 | 6 | 0 | 0 | 4 | 7 | 1 | 0 | 2 | 9 | 0 | 1 | 0 |
| mg/ | LT-4 | 8 | 1 | 1 | 0 | 9 | 0 | 1 | 0 | 8 | 1 | 1 | 0 | 8 | 0 | 2 | 0 | 1 | 1 | 1 | 7 | 9 | 0 | 1 | 0 |
| kg) | LT-5 | 7 | 2 | 1 | 0 | 8 | 1 | 1 | 0 | 8 | 2 | 0 | 0 | 10 | 0 | 0 | 0 | 7 | 1 | 0 | 2 | 9 | 1 | 0 | 0 |
| | LT-6 | 5 | 1 | 4 | 0 | 4 | 2 | 3 | 1 | 2 | 0 | 7 | 1 | 0 | 0 | 10 | 0 | 8 | 0 | 1 | 1 | 10 | 0 | 0 | 0 |
| | LT-7 | 6 | 2 | 1 | 1 | 8 | 0 | 1 | 1 | 9 | 0 | 1 | 0 | 10 | 0 | 0 | 0 | 10 | 0 | 0 | 0 | 9 | 0 | 0 | 1 |
| | LT-8 | 8 | 0 | 2 | 0 | 6 | 1 | 3 | 0 | 6 | 1 | 0 | 3 | 10 | 0 | 0 | 0 | 8 | 0 | 2 | 0 | 5 | 0 | 5 | 0 |
| | LT-9 | 3 | 0 | 7 | 0 | 10 | 0 | 0 | 0 | 8 | 1 | 1 | 0 | 9 | 0 | 0 | 1 | 10 | 0 | 0 | 0 | 7 | 1 | 2 | 0 |
| | LT- | 10 | 0 | 0 | 0 | 8 | 1 | 1 | 0 | 10 | 0 | 0 | 0 | 10 | 0 | 0 | 0 | 8 | 1 | 1 | 0 | 10 | 0 | 0 | 0 |
| | 10 | | | | | | | | | | | | | | | | | | | | | | | | |
| | LT- | 6 | 2 | 2 | 0 | 6 | 2 | 2 | 0 | 7 | 3 | 0 | 0 | 7 | 1 | 1 | 1 | 6 | 0 | 4 | 0 | 9 | 0 | 1 | 0 |
| | 11 | | | | | | | | | | | | | | | | | | | | | | | | |
| | LT- | 10 | 0 | 0 | 0 | 5 | 1 | 3 | 1 | 5 | 4 | 0 | 1 | 9 | 0 | 0 | 1 | 8 | 0 | 0 | 2 | 10 | 0 | 0 | 0 |
| | 12 | | | | | | | | | | | | | | | | | | | | | | | | |
| AVG | | 0.68 | 0.12 | 0.18 | 0.03 | 0.68 | 0.09 | 0.21 | 0.03 | 0.74 | 0.12 | 0.10 | 0.04 | 0.78 | 0.01 | 0.16 | 0.06 | 0.75 | 0.04 | 0.09 | 0.12 | 0.83 | 0.05 | 0.10 | 0.02 |
| SEM | | 0.58 | 0.30 | 0.60 | 0.13 | 0.54 | 0.19 | 0.38 | 0.13 | 0.66 | 0.37 | 0.58 | 0.26 | 0.83 | 0.08 | 0.85 | 0.34 | 0.70 | 0.15 | 0.36 | 0.59 | 0.48 | 0.23 | 0.43 | 0.11 |
| Com- | LT-1 | 6 | 0 | 2 | 2 | 6 | 1 | 2 | 1 | 9 | 0 | 1 | 0 | 7 | 1 | 1 | 1 | 8 | 0 | 1 | 1 | 5 | 1 | 1 | 3 |
| pound | LT-2 | 9 | 1 | 0 | 0 | 9 | 1 | 0 | 0 | 6 | 1 | 2 | 1 | 7 | 1 | 1 | 1 | 10 | 0 | 0 | 0 | 8 | 0 | 2 | 0 |
| I-19 | LT-3 | 9 | 0 | 0 | 1 | 7 | 1 | 0 | 2 | 9 | 0 | 1 | 0 | 7 | 1 | 0 | 2 | 8 | 0 | 2 | 0 | 10 | 0 | 0 | 0 |
| (0.1 | LT-4 | 10 | 0 | 0 | 0 | 10 | 0 | 0 | 0 | 8 | 0 | 2 | 0 | 10 | 0 | 0 | 0 | 9 | 1 | 0 | 0 | 9 | 0 | 1 | 0 |
| mg/ | LT-5 | 10 | 0 | 0 | 0 | 7 | 0 | 2 | 1 | 5 | 0 | 5 | 0 | 4 | 2 | 4 | 0 | 7 | 0 | 2 | 1 | 10 | 0 | 0 | 0 |
| kg) | LT-6 | 10 | 0 | 0 | 0 | 10 | 0 | 0 | 0 | 9 | 0 | 1 | 0 | 8 | 1 | 1 | 0 | 9 | 0 | 0 | 1 | 10 | 0 | 0 | 0 |
| | LT-7 | 10 | 0 | 0 | 0 | 8 | 0 | 2 | 0 | 8 | 0 | 2 | 0 | 8 | 0 | 0 | 2 | 6 | 0 | 0 | 4 | 7 | 1 | 2 | 0 |
| | LT-8 | 10 | 0 | 0 | 0 | 7 | 0 | 2 | 1 | 6 | 0 | 1 | 3 | 7 | 0 | 3 | 0 | 9 | 0 | 1 | 0 | 6 | 0 | 4 | 0 |
| | LT-9 | 10 | 0 | 0 | 0 | 8 | 1 | 1 | 0 | 10 | 0 | 0 | 0 | 10 | 0 | 0 | 0 | 6 | 0 | 4 | 0 | 10 | 0 | 0 | 0 |
| | LT- | 7 | 0 | 3 | 0 | 8 | 0 | 2 | 0 | 10 | 0 | 0 | 0 | 7 | 1 | 2 | 0 | 10 | 0 | 0 | 0 | 9 | 0 | 1 | 0 |
| | 10 | | | | | | | | | | | | | | | | | | | | | | | | |
| | LT- | 7 | 0 | 3 | 0 | 7 | 1 | 1 | 1 | 9 | 0 | 0 | 1 | 5 | 1 | 4 | 0 | 8 | 1 | 1 | 0 | 6 | 1 | 3 | 0 |
| | 11 | | | | | | | | | | | | | | | | | | | | | | | | |
| | LT- | 8 | 1 | 1 | 0 | 9 | 0 | 1 | 0 | 9 | 0 | 1 | 0 | 8 | 1 | 1 | 0 | 10 | 0 | 0 | 0 | 8 | 0 | 2 | 0 |
| | 12 | | | | | | | | | | | | | | | | | | | | | | | | |
| AVG | | 0.88 | 0.02 | 0.08 | 0.03 | 0.80 | 0.04 | 0.11 | 0.05 | 0.82 | 0.01 | 0.13 | 0.04 | 0.73 | 0.08 | 0.14 | 0.05 | 0.83 | 0.02 | 0.09 | 0.06 | 0.82 | 0.03 | 0.13 | 0.03 |
| SEM | | 0.42 | 0.11 | 0.35 | 0.18 | 0.37 | 0.15 | 0.26 | 0.19 | 0.47 | 0.08 | 0.40 | 0.26 | 0.50 | 0.18 | 0.43 | 0.23 | 0.41 | 0.11 | 0.36 | 0.34 | 0.52 | 0.13 | 0.38 | 0.25 |
| Com- | LT-1 | 9 | 1 | 0 | 0 | 8 | 1 | 1 | 0 | 10 | 0 | 0 | 0 | 4 | 2 | 4 | 0 | 6 | 0 | 2 | 2 | 10 | 0 | 0 | 0 |
| pound | LT-2 | 9 | 1 | 0 | 0 | 6 | 0 | 4 | 0 | 9 | 1 | 0 | 0 | 9 | 0 | 1 | 0 | 10 | 0 | 0 | 0 | 10 | 0 | 0 | 0 |
| I-19 | LT-3 | 10 | 0 | 0 | 0 | 10 | 0 | 0 | 0 | 8 | 0 | 2 | 0 | 10 | 0 | 0 | 0 | 5 | 0 | 5 | 0 | 9 | 0 | 1 | 0 |
| (0.3 | LT-4 | 10 | 0 | 0 | 0 | 10 | 0 | 0 | 0 | 8 | 0 | 2 | 0 | 9 | 0 | 1 | 0 | 9 | 0 | 0 | 1 | 10 | 0 | 0 | 0 |
| mg/ | LT-5 | 10 | 0 | 0 | 0 | 6 | 0 | 4 | 0 | 4 | 1 | 4 | 1 | 5 | 1 | 2 | 2 | 9 | 0 | 0 | 1 | 9 | 0 | 0 | 1 |
| kg) | LT-6 | 6 | 0 | 4 | 0 | 10 | 0 | 0 | 0 | 10 | 0 | 0 | 0 | 7 | 1 | 2 | 0 | 10 | 0 | 0 | 0 | 7 | 0 | 3 | 0 |
| | LT-7 | 9 | 0 | 0 | 1 | 5 | 0 | 5 | 0 | 9 | 1 | 0 | 0 | 8 | 0 | 0 | 2 | 10 | 0 | 0 | 0 | 6 | 0 | 4 | 0 |
| | LT-8 | 8 | 0 | 2 | 0 | 10 | 0 | 0 | 0 | 7 | 0 | 3 | 0 | 3 | 1 | 4 | 2 | 7 | 0 | 1 | 2 | 10 | 0 | 0 | 0 |
| | LT-9 | 8 | 0 | 2 | 0 | 10 | 0 | 0 | 0 | 4 | 0 | 3 | 3 | 10 | 0 | 0 | 0 | 10 | 0 | 0 | 0 | 9 | 1 | 0 | 0 |
| | LT- | 8 | 1 | 1 | 0 | 9 | 0 | 1 | 0 | 10 | 0 | 0 | 0 | 4 | 1 | 5 | 0 | 10 | 0 | 0 | 0 | 10 | 0 | 0 | 0 |
| | 10 | | | | | | | | | | | | | | | | | | | | | | | | |
| | LT- | 7 | 0 | 3 | 0 | 7 | 0 | 2 | 1 | 9 | 0 | 0 | 1 | 5 | 0 | 3 | 2 | 7 | 1 | 2 | 0 | 3 | 0 | 7 | 0 |
| | 11 | | | | | | | | | | | | | | | | | | | | | | | | |
| | LT- | 9 | 0 | 1 | 0 | 8 | 1 | 0 | 1 | 8 | 0 | 2 | 0 | 9 | 0 | 1 | 0 | 8 | 1 | 1 | 0 | 8 | 1 | 1 | 0 |
| | 12 | | | | | | | | | | | | | | | | | | | | | | | | |
| AVG | | 0.86 | 0.03 | 0.11 | 0.01 | 0.83 | 0.02 | 0.14 | 0.02 | 0.80 | 0.03 | 0.13 | 0.04 | 0.69 | 0.05 | 0.19 | 0.07 | 0.84 | 0.02 | 0.09 | 0.05 | 0.84 | 0.02 | 0.13 | 0.01 |
| SEM | | 0.36 | 0.13 | 0.40 | 0.08 | 0.54 | 0.11 | 0.54 | 0.11 | 0.60 | 0.13 | 0.43 | 0.26 | 0.74 | 0.19 | 0.50 | 0.28 | 0.51 | 0.11 | 0.43 | 0.23 | 0.62 | 0.11 | 0.64 | 0.08 |
| Com- | LT-1 | 7 | 0 | 3 | 0 | 10 | 0 | 0 | 0 | 10 | 0 | 0 | 0 | 1 | 2 | 7 | 0 | 7 | 0 | 3 | 0 | 4 | 0 | 6 | 0 |
| pound | LT-2 | 8 | 1 | 1 | 0 | 10 | 0 | 0 | 0 | 9 | 0 | 1 | 0 | 5 | 0 | 4 | 1 | 6 | 2 | 2 | 0 | 9 | 0 | 1 | 0 |
| I-19 | LT-3 | 9 | 0 | 1 | 0 | 9 | 0 | 1 | 0 | 6 | 0 | 4 | 0 | 9 | 1 | 0 | 0 | 9 | 0 | 0 | 1 | 10 | 0 | 0 | 0 |
| (1 | LT-4 | 9 | 0 | 1 | 0 | 9 | 0 | 0 | 1 | 7 | 1 | 2 | 0 | 7 | 0 | 3 | 0 | 8 | 1 | 1 | 0 | 10 | 0 | 0 | 0 |
| mg/ | LT-5 | 3 | 0 | 5 | 2 | 7 | 0 | 0 | 3 | 10 | 0 | 0 | 0 | 9 | 0 | 1 | 0 | 5 | 0 | 3 | 2 | 0 | 0 | 4 | 6 |
| kg) | LT-6 | 10 | 0 | 0 | 0 | 4 | 0 | 6 | 0 | 10 | 0 | 0 | 0 | 4 | 1 | 5 | 0 | 8 | 0 | 1 | 1 | 10 | 0 | 0 | 0 |
| | LT-7 | 7 | 1 | 2 | 0 | 2 | 1 | 3 | 4 | 0 | 0 | 2 | 8 | 5 | 0 | 2 | 3 | 10 | 0 | 0 | 0 | 6 | 0 | 4 | 0 |
| | LT-8 | 9 | 0 | 1 | 0 | 9 | 0 | 1 | 0 | 7 | 1 | 2 | 0 | 7 | 1 | 2 | 0 | 7 | 0 | 3 | 0 | 6 | 1 | 2 | 1 |
| | LT-9 | 9 | 0 | 1 | 0 | 7 | 0 | 3 | 0 | 10 | 0 | 0 | 0 | 10 | 0 | 0 | 0 | 8 | 0 | 2 | 0 | 10 | 0 | 0 | 0 |
| | LT- | 5 | 2 | 3 | 0 | 7 | 0 | 3 | 0 | 5 | 0 | 5 | 0 | 3 | 0 | 4 | 2 | 3 | 2 | 4 | 1 | 8 | 0 | 2 | 0 |
| | 10 | | | | | | | | | | | | | | | | | | | | | | | | |
| | LT- | 8 | 1 | 0 | 1 | 9 | 0 | 1 | 0 | 5 | 0 | 2 | 3 | 7 | 0 | 2 | 1 | 4 | 1 | 2 | 3 | 2 | 0 | 5 | 3 |
| | 11 | | | | | | | | | | | | | | | | | | | | | | | | |
| | LT- | 9 | 1 | 0 | 0 | 9 | 1 | 0 | 0 | 9 | 0 | 1 | 0 | 5 | 0 | 5 | 0 | 8 | 0 | 2 | 0 | 7 | 0 | 3 | 0 |
| | 12 | | | | | | | | | | | | | | | | | | | | | | | | |
| AVG | | 0.78 | 0.05 | 0.15 | 0.03 | 0.77 | 0.02 | 0.15 | 0.07 | 0.73 | 0.02 | 0.16 | 0.09 | 0.60 | 0.04 | 0.29 | 0.06 | 0.69 | 0.05 | 0.19 | 0.07 | 0.68 | 0.01 | 0.23 | 0.08 |
| SEM | | 0.58 | 0.19 | 0.44 | 0.18 | 0.71 | 0.11 | 0.54 | 0.40 | 0.87 | 0.11 | 0.47 | 0.69 | 0.77 | 0.19 | 0.62 | 0.29 | 0.60 | 0.23 | 0.36 | 0.28 | 0.98 | 0.08 | 0.62 | 0.53 |
| Com- | LT-1 | 5 | 0 | 5 | 0 | 6 | 0 | 4 | 0 | 10 | 0 | 0 | 0 | 8 | 0 | 0 | 2 | 6 | 1 | 3 | 0 | 5 | 0 | 5 | 0 |
| pound | LT-2 | 10 | 0 | 0 | 0 | 9 | 0 | 1 | 0 | 6 | 0 | 3 | 1 | 9 | 0 | 1 | 0 | 10 | 0 | 0 | 0 | 4 | 2 | 4 | 0 |
| I-19 | LT-3 | 9 | 0 | 1 | 0 | 10 | 0 | 0 | 0 | 9 | 1 | 0 | 0 | 6 | 0 | 0 | 4 | 9 | 0 | 1 | 0 | 8 | 0 | 2 | 0 |
| (3 | LT-4 | 8 | 0 | 2 | 0 | 8 | 0 | 2 | 0 | 9 | 1 | 0 | 0 | 10 | 0 | 0 | 0 | 7 | 0 | 3 | 0 | 10 | 0 | 0 | 1 |
| mg/ | LT-5 | 7 | 0 | 2 | 1 | 8 | 0 | 1 | 1 | 8 | 0 | 1 | 1 | 9 | 0 | 0 | 1 | 7 | 1 | 0 | 2 | 9 | 0 | 0 | 1 |
| kg) | LT-6 | 8 | 0 | 2 | 0 | 9 | 1 | 0 | 0 | 10 | 0 | 0 | 0 | 10 | 0 | 0 | 0 | 9 | 1 | 0 | 0 | 5 | 0 | 5 | 0 |
| | LT-7 | 7 | 0 | 3 | 0 | 8 | 0 | 0 | 2 | 4 | 1 | 4 | 1 | 1 | 0 | 2 | 7 | 7 | 0 | 1 | 2 | 3 | 0 | 5 | 2 |
| | LT-8 | 10 | 0 | 0 | 0 | 9 | 0 | 1 | 0 | 10 | 0 | 0 | 0 | 6 | 0 | 4 | 0 | 8 | 0 | 0 | 2 | 9 | 0 | 0 | 1 |
| | LT-9 | 8 | 0 | 2 | 0 | 10 | 0 | 0 | 0 | 9 | 0 | 0 | 0 | 9 | 1 | 0 | 0 | 3 | 1 | 5 | 1 | 10 | 0 | 0 | 0 |
| | LT- | 10 | 0 | 0 | 0 | 10 | 0 | 0 | 0 | 2 | 2 | 4 | 2 | 10 | 0 | 0 | 0 | 8 | 1 | 0 | 1 | 6 | 0 | 0 | 4 |
| | 10 | | | | | | | | | | | | | | | | | | | | | | | | |
| | LT- | 8 | 0 | 2 | 0 | 9 | 0 | 0 | 1 | 0 | 0 | 9 | 1 | 10 | 0 | 0 | 0 | 8 | 0 | 0 | 2 | 2 | 2 | 6 | 0 |
| | 11 | | | | | | | | | | | | | | | | | | | | | | | | |
| | LT- | 9 | 0 | 0 | 1 | 7 | 1 | 2 | 0 | 9 | 0 | 1 | 0 | 7 | 0 | 2 | 1 | 7 | 1 | 1 | 1 | 7 | 0 | 0 | 3 |
| | 12 | | | | | | | | | | | | | | | | | | | | | | | | |

| | | | | | | | | | | | | | | | | | | | | | | | | |
|---|---|---|---|---|---|---|---|---|---|---|---|---|---|---|---|---|---|---|---|---|---|---|---|---|
| AVG | 0.83 | 0.00 | 0.16 | 0.02 | 0.86 | 0.02 | 0.09 | 0.03 | 0.72 | 0.04 | 0.18 | 0.05 | 0.79 | 0.01 | 0.08 | 0.13 | 0.74 | 0.05 | 0.12 | 0.09 | 0.65 | 0.03 | 0.23 | 0.10 |
| SEM | 0.43 | 0.00 | 0.43 | 0.11 | 0.36 | 0.11 | 0.36 | 0.19 | 0.98 | 0.19 | 0.80 | 0.19 | 0.76 | 0.08 | 0.37 | 0.63 | 0.51 | 0.15 | 0.47 | 0.26 | 0.79 | 0.22 | 0.73 | 0.39 |

[symbol]

Table 139. BSS (Behavioral Satiety Sequence) Scoring—Raw Data 0.5-1 hours. E is eating/drinking; G is grooming; A is active; and R is resting.

TABLE 140
BSS (Behavioral Satiety Sequence) Scoring - Raw Data 1-1.5 hours. E is
eating/drinking; G is grooming; A is active; and R is resting.

| | Ani- | Time Bin 13 (60- | | | | Time Bin 14 (65- | | | | Time Bin 15 (70- | | | | Time Bin 16 (75- | | | | Time Bin 17 (80- | | | | Time Bin 18 (85- | | | |
|---|---|---|---|---|---|---|---|---|---|---|---|---|---|---|---|---|---|---|---|---|---|---|---|---|---|
| Treat- | mal | 65 TOTAL) | | | | 70 TOTAL) | | | | 75 TOTAL) | | | | 80 TOTAL) | | | | 85 TOTAL) | | | | 90 TOTAL) | | | |
| ment | ID | E | G | A | R | E | G | A | R | E | G | A | R | E | G | A | R | E | G | A | R | E | G | A | R |
| Ve- | LT-1 | 9 | 0 | 0 | 1 | 5 | 0 | 0 | 5 | 5 | 1 | 0 | 4 | 7 | 0 | 0 | 3 | 5 | 1 | 4 | 0 | 7 | 0 | 0 | 3 |
| hicle | LT-2 | 8 | 1 | 1 | 0 | 7 | 0 | 3 | 0 | 9 | 0 | 0 | 1 | 6 | 1 | 3 | 0 | 7 | 1 | 2 | 0 | 8 | 0 | 2 | 0 |
| | LT-3 | 9 | 0 | 0 | 1 | 1 | 1 | 7 | 1 | 7 | 0 | 3 | 0 | 9 | 0 | 1 | 0 | 10 | 0 | 0 | 0 | 10 | 0 | 0 | 0 |
| | LT-4 | 10 | 0 | 0 | 0 | 9 | 1 | 0 | 0 | 7 | 0 | 3 | 0 | 10 | 0 | 0 | 0 | 10 | 0 | 0 | 0 | 8 | 0 | 0 | 2 |
| | LT-5 | 7 | 0 | 3 | 0 | 8 | 0 | 0 | 2 | 7 | 0 | 0 | 3 | 5 | 0 | 2 | 3 | 5 | 0 | 0 | 5 | 7 | 0 | 0 | 3 |
| | LT-6 | 8 | 1 | 0 | 1 | 8 | 0 | 0 | 2 | 4 | 1 | 5 | 0 | 9 | 0 | 0 | 1 | 6 | 2 | 1 | 1 | 9 | 0 | 1 | 0 |
| | LT-7 | 7 | 0 | 2 | 1 | 7 | 0 | 2 | 1 | 9 | 1 | 0 | 0 | 8 | 0 | 1 | 1 | 9 | 0 | 0 | 1 | 9 | 0 | 0 | 1 |
| | LT-8 | 8 | 0 | 1 | 1 | 3 | 1 | 5 | 1 | 9 | 1 | 0 | 0 | 4 | 1 | 5 | 0 | 5 | 0 | 4 | 1 | 3 | 2 | 5 | 0 |
| | LT-9 | 10 | 0 | 0 | 0 | 7 | 1 | 1 | 1 | 7 | 0 | 3 | 0 | 8 | 0 | 1 | 1 | 10 | 0 | 0 | 0 | 8 | 0 | 0 | 2 |
| | LT-10 | 9 | 0 | 1 | 0 | 10 | 0 | 0 | 0 | 8 | 0 | 1 | 1 | 10 | 0 | 0 | 0 | 6 | 1 | 3 | 0 | 8 | 0 | 2 | 0 |
| | LT-11 | 9 | 0 | 1 | 0 | 7 | 1 | 2 | 0 | 9 | 0 | 0 | 1 | 5 | 2 | 3 | 0 | 10 | 0 | 0 | 0 | 10 | 0 | 0 | 0 |
| | LT-12 | 7 | 0 | 2 | 1 | 9 | 0 | 0 | 1 | 6 | 0 | 3 | 1 | 4 | 3 | 2 | 1 | 9 | 0 | 1 | 0 | 7 | 0 | 3 | 0 |
| AVG | | 0.84 | 0.02 | 0.09 | 0.05 | 0.68 | 0.04 | 0.17 | 0.12 | 0.73 | 0.03 | 0.15 | 0.09 | 0.71 | 0.06 | 0.15 | 0.08 | 0.77 | 0.04 | 0.13 | 0.07 | 0.78 | 0.02 | 0.11 | 0.09 |
| SEM | | 0.31 | 0.11 | 0.29 | 0.15 | 0.75 | 0.15 | 0.67 | 0.41 | 0.48 | 0.14 | 0.51 | 0.38 | 0.65 | 0.29 | 0.45 | 0.32 | 0.63 | 0.19 | 0.46 | 0.41 | 0.53 | 0.17 | 0.47 | 0.36 |
| Lor- | LT-1 | 4 | 0 | 6 | 0 | 6 | 0 | 4 | 0 | 6 | 0 | 0 | 4 | 10 | 0 | 0 | 0 | 8 | 0 | 0 | 2 | 4 | 2 | 4 | 0 |
| caserin | LT-2 | 3 | 2 | 5 | 0 | 5 | 0 | 4 | 1 | 6 | 3 | 0 | 1 | 1 | 6 | 2 | 1 | 9 | 0 | 0 | 1 | 9 | 1 | 0 | 0 |
| (1 mg/ | LT-3 | 6 | 0 | 2 | 2 | 7 | 1 | 2 | 0 | 9 | 1 | 0 | 0 | 10 | 0 | 0 | 0 | 10 | 0 | 0 | 0 | 6 | 1 | 1 | 2 |
| kg) | LT-4 | 5 | 0 | 1 | 4 | 10 | 0 | 0 | 0 | 2 | 7 | 1 | 0 | 9 | 0 | 1 | 0 | 7 | 1 | 2 | 0 | 10 | 0 | 0 | 0 |
| | LT-5 | 7 | 3 | 0 | 0 | 4 | 0 | 2 | 4 | 9 | 0 | 1 | 0 | 5 | 3 | 0 | 2 | 9 | 0 | 0 | 1 | 10 | 0 | 0 | 0 |
| | LT-6 | 8 | 2 | 0 | 0 | 8 | 0 | 2 | 0 | 9 | 0 | 1 | 0 | 10 | 0 | 0 | 0 | 8 | 0 | 0 | 2 | 10 | 0 | 0 | 0 |
| | LT-7 | 5 | 0 | 4 | 1 | 4 | 1 | 1 | 4 | 10 | 0 | 0 | 0 | 10 | 0 | 0 | 0 | 10 | 0 | 0 | 0 | 7 | 1 | 1 | 1 |
| | LT-8 | 8 | 0 | 2 | 0 | 3 | 0 | 4 | 3 | 3 | 1 | 4 | 2 | 6 | 0 | 4 | 0 | 8 | 0 | 2 | 0 | 7 | 2 | 1 | 0 |
| | LT-9 | 9 | 0 | 0 | 1 | 9 | 0 | 1 | 0 | 8 | 1 | 1 | 0 | 9 | 0 | 0 | 1 | 10 | 0 | 0 | 0 | 3 | 0 | 6 | 1 |
| | LT-10 | 9 | 0 | 1 | 0 | 8 | 1 | 1 | 0 | 9 | 0 | 0 | 1 | 10 | 0 | 0 | 0 | 7 | 1 | 2 | 0 | 6 | 2 | 2 | 0 |
| | LT-11 | 7 | 0 | 3 | 0 | 6 | 2 | 1 | 1 | 6 | 0 | 4 | 0 | 10 | 0 | 0 | 0 | 9 | 1 | 0 | 0 | 7 | 3 | 0 | 0 |
| | LT-12 | 8 | 1 | 0 | 1 | 8 | 0 | 2 | 0 | 7 | 1 | 2 | 0 | 10 | 0 | 0 | 0 | 6 | 2 | 2 | 0 | 9 | 0 | 1 | 0 |
| AVG | | 0.66 | 0.07 | 0.20 | 0.08 | 0.65 | 0.04 | 0.20 | 0.11 | 0.70 | 0.12 | 0.12 | 0.07 | 0.83 | 0.08 | 0.06 | 0.03 | 0.84 | 0.04 | 0.07 | 0.05 | 0.73 | 0.10 | 0.13 | 0.03 |
| SEM | | 0.57 | 0.31 | 0.60 | 0.35 | 0.63 | 0.19 | 0.39 | 0.47 | 0.73 | 0.59 | 0.42 | 0.36 | 0.83 | 0.54 | 0.36 | 0.19 | 0.38 | 0.19 | 0.28 | 0.23 | 0.68 | 0.30 | 0.54 | 0.19 |
| Com- | LT-1 | 8 | 0 | 0 | 2 | 10 | 0 | 0 | 0 | 5 | 0 | 5 | 0 | 6 | 0 | 4 | 0 | 10 | 0 | 0 | 0 | 10 | 0 | 0 | 0 |
| pound | LT-2 | 5 | 1 | 4 | 0 | 8 | 1 | 1 | 0 | 7 | 0 | 3 | 0 | 3 | 4 | 3 | 0 | 7 | 0 | 3 | 0 | 10 | 0 | 0 | 0 |
| I-19 | LT-3 | 8 | 1 | 0 | 1 | 10 | 0 | 0 | 0 | 10 | 0 | 0 | 0 | 3 | 0 | 5 | 2 | 0 | 0 | 9 | 1 | 8 | 0 | 1 | 1 |
| (0.1 | LT-4 | 6 | 0 | 4 | 0 | 9 | 1 | 0 | 0 | 10 | 0 | 0 | 0 | 10 | 0 | 0 | 0 | 9 | 0 | 1 | 0 | 7 | 1 | 1 | 1 |
| mg/ | LT-5 | 10 | 0 | 0 | 0 | 9 | 0 | 0 | 1 | 9 | 0 | 0 | 1 | 3 | 0 | 4 | 3 | 3 | 1 | 6 | 0 | 0 | 0 | 7 | 3 |
| (kg) | LT-6 | 5 | 0 | 5 | 0 | 9 | 1 | 0 | 0 | 9 | 0 | 0 | 1 | 9 | 0 | 0 | 0 | 4 | 1 | 5 | 0 | 10 | 0 | 0 | 0 |
| | LT-7 | 8 | 0 | 1 | 1 | 5 | 1 | 4 | 0 | 9 | 0 | 0 | 1 | 8 | 1 | 0 | 1 | 5 | 1 | 4 | 0 | 7 | 0 | 3 | 0 |
| | LT-8 | 10 | 0 | 0 | 0 | 8 | 0 | 2 | 0 | 7 | 0 | 3 | 0 | 6 | 1 | 2 | 1 | 5 | 0 | 4 | 1 | 5 | 0 | 5 | 0 |
| | LT-9 | 10 | 0 | 0 | 0 | 10 | 0 | 0 | 0 | 6 | 1 | 3 | 0 | 8 | 0 | 1 | 1 | 10 | 0 | 0 | 0 | 10 | 0 | 0 | 0 |
| | LT-10 | 9 | 0 | 1 | 0 | 7 | 1 | 2 | 0 | 10 | 0 | 0 | 0 | 9 | 0 | 0 | 1 | 6 | 2 | 2 | 0 | 8 | 0 | 2 | 0 |
| | LT-11 | 2 | 0 | 8 | 0 | 8 | 1 | 1 | 0 | 8 | 0 | 2 | 0 | 4 | 0 | 5 | 1 | 2 | 0 | 9 | 0 | 7 | 0 | 2 | 1 |
| | LT-12 | 8 | 1 | 1 | 0 | 7 | 0 | 2 | 1 | 10 | 0 | 0 | 0 | 6 | 0 | 4 | 0 | 9 | 1 | 0 | 0 | 3 | 1 | 5 | 1 |
| AVG | | 0.74 | 0.03 | 0.20 | 0.03 | 0.83 | 0.05 | 0.10 | 0.02 | 0.83 | 0.01 | 0.13 | 0.03 | 0.63 | 0.05 | 0.23 | 0.08 | 0.58 | 0.05 | 0.36 | 0.02 | 0.71 | 0.02 | 0.22 | 0.06 |
| SEM | | 0.71 | 0.13 | 0.76 | 0.19 | 0.43 | 0.15 | 0.37 | 0.11 | 0.50 | 0.08 | 0.51 | 0.13 | 0.74 | 0.34 | 0.59 | 0.27 | 0.94 | 0.19 | 0.93 | 0.11 | 0.90 | 0.11 | 0.68 | 0.26 |
| Com- | LT-1 | 10 | 0 | 0 | 0 | 9 | 0 | 1 | 0 | 9 | 0 | 0 | 1 | 10 | 0 | 0 | 0 | 10 | 0 | 0 | 0 | 4 | 2 | 4 | 0 |
| pound | LT-2 | 10 | 0 | 0 | 0 | 6 | 1 | 3 | 0 | 6 | 0 | 4 | 0 | 6 | 0 | 2 | 2 | 5 | 2 | 2 | 1 | 9 | 1 | 0 | 0 |
| I-19 | LT-3 | 7 | 1 | 2 | 0 | 10 | 0 | 0 | 0 | 4 | 0 | 6 | 0 | 9 | 0 | 0 | 1 | 10 | 0 | 0 | 0 | 9 | 0 | 0 | 1 |
| (0.3 | LT-4 | 8 | 0 | 0 | 2 | 9 | 0 | 0 | 1 | 1 | 1 | 8 | 0 | 6 | 1 | 3 | 0 | 4 | 1 | 1 | 4 | 8 | 1 | 0 | 1 |
| mg/ | LT-5 | 8 | 0 | 0 | 2 | 9 | 0 | 0 | 1 | 7 | 0 | 0 | 3 | 8 | 0 | 0 | 2 | 10 | 0 | 0 | 0 | 0 | 1 | 8 | 1 |
| kg) | LT-6 | 10 | 0 | 0 | 0 | 7 | 1 | 0 | 2 | 6 | 1 | 1 | 2 | 7 | 0 | 2 | 1 | 7 | 2 | 1 | 0 | 6 | 2 | 2 | 0 |
| | LT-7 | 9 | 1 | 0 | 0 | 9 | 0 | 0 | 1 | 5 | 0 | 4 | 1 | 7 | 0 | 2 | 1 | 4 | 0 | 3 | 3 | 9 | 1 | 0 | 0 |
| | LT-8 | 8 | 1 | 0 | 1 | 9 | 0 | 1 | 0 | 6 | 1 | 3 | 0 | 5 | 0 | 3 | 2 | 4 | 1 | 5 | 0 | 6 | 1 | 1 | 2 |
| | LT-9 | 7 | 0 | 3 | 0 | 10 | 0 | 0 | 0 | 7 | 0 | 3 | 0 | 10 | 0 | 0 | 0 | 9 | 0 | 1 | 0 | 10 | 0 | 0 | 0 |
| | LT-10 | 7 | 1 | 1 | 1 | 8 | 1 | 1 | 0 | 3 | 1 | 5 | 1 | 10 | 0 | 0 | 0 | 9 | 0 | 1 | 0 | 3 | 1 | 5 | 1 |
| | LT-11 | 7 | 0 | 1 | 2 | 2 | 0 | 8 | 0 | 7 | 0 | 3 | 0 | 10 | 0 | 0 | 0 | 7 | 0 | 1 | 2 | 3 | 1 | 5 | 1 |
| | LT-12 | 6 | 0 | 4 | 0 | 8 | 0 | 1 | 1 | 6 | 1 | 1 | 2 | 7 | 0 | 3 | 0 | 4 | 0 | 5 | 1 | 7 | 1 | 2 | 0 |
| AVG | | 0.81 | 0.03 | 0.09 | 0.07 | 0.80 | 0.03 | 0.13 | 0.05 | 0.56 | 0.04 | 0.32 | 0.08 | 0.79 | 0.01 | 0.13 | 0.08 | 0.69 | 0.05 | 0.17 | 0.09 | 0.62 | 0.10 | 0.23 | 0.06 |
| SEM | | 0.40 | 0.14 | 0.40 | 0.26 | 0.64 | 0.13 | 0.66 | 0.19 | 0.61 | 0.15 | 0.71 | 0.30 | 0.53 | 0.08 | 0.39 | 0.25 | 0.75 | 0.23 | 0.51 | 0.40 | 0.89 | 0.17 | 0.77 | 0.19 |
| Com- | LT-1 | 8 | 0 | 2 | 0 | 10 | 0 | 0 | 0 | 10 | 0 | 0 | 0 | 7 | 2 | 1 | 0 | 1 | 0 | 9 | 0 | 9 | 0 | 1 | 0 |
| pound | LT-2 | 6 | 2 | 1 | 1 | 8 | 1 | 1 | 0 | 8 | 0 | 0 | 2 | 7 | 1 | 2 | 0 | 10 | 0 | 0 | 0 | 9 | 0 | 0 | 1 |
| I-19 | LT-3 | 10 | 0 | 0 | 0 | 10 | 0 | 0 | 0 | 8 | 0 | 2 | 0 | 9 | 0 | 1 | 0 | 10 | 0 | 0 | 0 | 6 | 0 | 4 | 0 |
| (1 | LT-4 | 6 | 1 | 3 | 0 | 9 | 0 | 1 | 0 | 7 | 0 | 2 | 1 | 10 | 0 | 0 | 0 | 6 | 0 | 4 | 0 | 7 | 1 | 0 | 2 |
| mg/ | LT-5 | 0 | 0 | 0 | 10 | 1 | 0 | 1 | 8 | 6 | 0 | 0 | 4 | 10 | 0 | 0 | 0 | 8 | 0 | 0 | 2 | 7 | 1 | 0 | 1 |
| kg) | LT-6 | 6 | 1 | 1 | 2 | 7 | 0 | 1 | 2 | 8 | 0 | 2 | 0 | 7 | 0 | 1 | 2 | 8 | 0 | 2 | 0 | 7 | 0 | 3 | 0 |
| | LT-7 | 8 | 1 | 0 | 1 | 7 | 1 | 2 | 0 | 3 | 1 | 1 | 5 | 0 | 0 | 0 | 10 | 1 | 1 | 3 | 5 | 1 | 0 | 2 | 7 |
| | LT-8 | 9 | 0 | 1 | 0 | 8 | 1 | 0 | 1 | 10 | 0 | 0 | 0 | 1 | 0 | 4 | 5 | 0 | 0 | 0 | 10 | 0 | 1 | 1 | 8 |
| | LT-9 | 10 | 0 | 0 | 0 | 7 | 0 | 3 | 0 | 4 | 0 | 5 | 1 | 9 | 0 | 0 | 1 | 10 | 0 | 0 | 0 | 8 | 1 | 1 | 0 |
| | LT-10 | 10 | 0 | 0 | 0 | 10 | 0 | 0 | 0 | 4 | 3 | 1 | 2 | 8 | 0 | 0 | 2 | 9 | 0 | 1 | 0 | 10 | 0 | 0 | 0 |
| | LT-11 | 9 | 0 | 0 | 1 | 5 | 1 | 3 | 1 | 7 | 0 | 2 | 1 | 6 | 0 | 4 | 0 | 9 | 0 | 1 | 0 | 1 | 0 | 2 | 7 |
| | LT-12 | 5 | 2 | 3 | 0 | 6 | 0 | 4 | 0 | 3 | 2 | 5 | 0 | 3 | 0 | 6 | 1 | 9 | 0 | 1 | 0 | 0 | 0 | 0 | 10 |
| AVG | | 0.73 | 0.06 | 0.09 | 0.13 | 0.73 | 0.03 | 0.13 | 0.10 | 0.65 | 0.05 | 0.17 | 0.13 | 0.64 | 0.03 | 0.16 | 0.18 | 0.68 | 0.01 | 0.18 | 0.14 | 0.54 | 0.03 | 0.12 | 0.30 |
| SEM | | 0.84 | 0.23 | 0.34 | 0.82 | 0.74 | 0.14 | 0.40 | 0.66 | 0.72 | 0.29 | 0.51 | 0.48 | 0.97 | 0.18 | 0.58 | 0.86 | 1.11 | 0.08 | 0.76 | 0.89 | 1.10 | 0.14 | 0.39 | 1.10 |
| Com- | LT-1 | 6 | 1 | 3 | 0 | 2 | 0 | 8 | 0 | 7 | 0 | 3 | 0 | 10 | 0 | 0 | 0 | 7 | 0 | 0 | 3 | 10 | 0 | 0 | 0 |
| pound | LT-2 | 8 | 0 | 0 | 2 | 8 | 0 | 0 | 2 | 9 | 1 | 0 | 0 | 10 | 0 | 0 | 0 | 2 | 0 | 8 | 0 | 8 | 0 | 2 | 0 |
| I-19 | LT-3 | 8 | 0 | 2 | 0 | 9 | 0 | 1 | 0 | 8 | 0 | 0 | 2 | 8 | 1 | 0 | 1 | 8 | 1 | 0 | 1 | 8 | 0 | 0 | 2 |
| (3 | LT-4 | 7 | 1 | 2 | 0 | 9 | 0 | 0 | 1 | 5 | 0 | 5 | 0 | 3 | 3 | 1 | 3 | 10 | 0 | 0 | 0 | 10 | 0 | 0 | 0 |
| mg/ | LT-5 | 6 | 0 | 2 | 2 | 8 | 0 | 0 | 2 | 0 | 1 | 4 | 5 | 0 | 1 | 0 | 9 | 0 | 0 | 2 | 8 | 5 | 0 | 3 | 2 |
| kg) | LT-6 | 8 | 0 | 1 | 1 | 10 | 0 | 0 | 0 | 5 | 0 | 5 | 0 | 8 | 0 | 0 | 2 | 10 | 0 | 0 | 0 | 10 | 0 | 0 | 0 |
| | LT-7 | 8 | 0 | 2 | 0 | 3 | 0 | 5 | 2 | 0 | 0 | 5 | 5 | 2 | 0 | 0 | 8 | 0 | 2 | 3 | 5 | 0 | 4 | 0 | 6 |
| | LT-8 | 9 | 0 | 1 | 0 | 9 | 0 | 1 | 0 | 6 | 4 | 0 | 0 | 10 | 0 | 0 | 0 | 3 | 0 | 4 | 3 | 0 | 1 | 0 | 9 |
| | LT-9 | 10 | 0 | 0 | 0 | 3 | 0 | 1 | 6 | 10 | 0 | 0 | 0 | 9 | 0 | 0 | 1 | 6 | 0 | 4 | 0 | 10 | 0 | 0 | 0 |
| | LT-10 | 10 | 0 | 0 | 0 | 7 | 2 | 0 | 1 | 7 | 0 | 3 | 0 | 7 | 0 | 2 | 1 | 10 | 0 | 0 | 0 | 9 | 0 | 1 | 0 |
| | LT-11 | 0 | 0 | 4 | 6 | 2 | 0 | 1 | 7 | 7 | 0 | 0 | 3 | 8 | 1 | 0 | 1 | 10 | 0 | 0 | 0 | 5 | 1 | 3 | 1 |
| | LT-12 | 6 | 0 | 4 | 0 | 5 | 0 | 1 | 4 | 0 | 1 | 9 | 0 | 10 | 0 | 0 | 0 | 9 | 0 | 1 | 0 | 3 | 2 | 5 | 0 |

| | | | | | | | | | | | | | | | | | | | | | | | | |
|---|---|---|---|---|---|---|---|---|---|---|---|---|---|---|---|---|---|---|---|---|---|---|---|---|
| AVG | 0.72 | 0.02 | 0.18 | 0.09 | 0.63 | 0.02 | 0.15 | 0.21 | 0.53 | 0.06 | 0.28 | 0.13 | 0.71 | 0.05 | 0.03 | 0.22 | 0.63 | 0.03 | 0.18 | 0.17 | 0.65 | 0.07 | 0.12 | 0.17 |
| SEM | 0.77 | 0.11 | 0.41 | 0.51 | 0.88 | 0.17 | 0.71 | 0.69 | 1.02 | 0.34 | 0.84 | 0.58 | 1.00 | 0.26 | 0.18 | 0.89 | 1.15 | 0.18 | 0.73 | 0.75 | 1.10 | 0.36 | 0.49 | 0.84 |

TABLE 141
BSS (Behavioral Satiety Sequence) Scoring - Raw Data 1.5-2 hours. E is
eating/drinking; G is grooming; A is active; and R is resting.

| | Ani- | Time Bin 19 (90- | | | | Time Bin 20 (95- | | | | Time Bin 21 (100- | | | | Time Bin 22 (105- | | | | Time Bin 23 (110- | | | | Time Bin 24 (115- | | | |
|---|---|---|---|---|---|---|---|---|---|---|---|---|---|---|---|---|---|---|---|---|---|---|---|---|---|
| Treat- | mal | 95 TOTAL) | | | | 100 TOTAL) | | | | 105 TOTAL) | | | | 110 TOTAL) | | | | 115 TOTAL) | | | | 120 TOTAL) | | | |
| ment | ID | E | G | A | R | E | G | A | R | E | G | A | R | E | G | A | R | E | G | A | R | E | G | A | R |
| Ve- | LT-1 | 7 | 0 | 0 | 3 | 7 | 1 | 2 | 0 | 6 | 0 | 4 | 0 | 10 | 0 | 0 | 0 | 10 | 0 | 0 | 0 | 10 | 0 | 0 | 0 |
| hicle | LT-2 | 7 | 1 | 2 | 0 | 7 | 1 | 2 | 0 | 6 | 2 | 2 | 0 | 8 | 1 | 1 | 0 | 8 | 0 | 1 | 1 | 9 | 0 | 1 | 0 |
| | LT-3 | 6 | 0 | 4 | 0 | 9 | 0 | 0 | 1 | 4 | 0 | 6 | 0 | 7 | 1 | 1 | 1 | 7 | 0 | 0 | 3 | 10 | 0 | 0 | 0 |
| | LT-4 | 4 | 0 | 3 | 3 | 1 | 2 | 6 | 1 | 5 | 0 | 4 | 1 | 10 | 0 | 0 | 0 | 5 | 1 | 4 | 0 | 7 | 0 | 3 | 0 |
| | LT-5 | 5 | 1 | 1 | 3 | 3 | 0 | 3 | 4 | 8 | 0 | 0 | 2 | 7 | 0 | 0 | 3 | 7 | 0 | 0 | 3 | 0 | 0 | 0 | 0 |
| | LT-6 | 9 | 1 | 0 | 0 | 6 | 1 | 3 | 0 | 10 | 0 | 0 | 0 | 9 | 0 | 0 | 1 | 6 | 0 | 4 | 0 | 9 | 0 | 1 | 0 |
| | LT-7 | 3 | 1 | 5 | 1 | 4 | 0 | 5 | 1 | 8 | 2 | 0 | 0 | 9 | 0 | 1 | 0 | 3 | 1 | 2 | 4 | 1 | 0 | 2 | 7 |
| | LT-8 | 8 | 0 | 2 | 0 | 9 | 0 | 1 | 0 | 1 | 0 | 9 | 0 | 2 | 2 | 6 | 0 | 10 | 0 | 0 | 0 | 10 | 0 | 0 | 0 |
| | LT-9 | 6 | 0 | 4 | 0 | 10 | 0 | 0 | 0 | 8 | 0 | 2 | 0 | 8 | 0 | 1 | 1 | 10 | 0 | 0 | 0 | 7 | 0 | 2 | 1 |
| | LT-10 | 10 | 0 | 0 | 0 | 6 | 1 | 3 | 0 | 8 | 0 | 2 | 0 | 8 | 0 | 2 | 0 | 5 | 0 | 4 | 1 | 9 | 0 | 1 | 0 |
| | LT-11 | 3 | 1 | 6 | 0 | 9 | 0 | 1 | 0 | 8 | 0 | 2 | 0 | 0 | 1 | 9 | 0 | 7 | 0 | 3 | 0 | 0 | 0 | 0 | 0 |
| | LT-12 | 7 | 0 | 3 | 0 | 5 | 1 | 2 | 2 | 9 | 0 | 0 | 1 | 6 | 1 | 3 | 0 | 4 | 2 | 4 | 0 | 5 | 2 | 3 | 0 |
| AVG | | 0.63 | 0.04 | 0.25 | 0.08 | 0.63 | 0.06 | 0.23 | 0.08 | 0.68 | 0.03 | 0.26 | 0.03 | 0.70 | 0.05 | 0.20 | 0.05 | 0.68 | 0.03 | 0.18 | 0.10 | 0.64 | 0.02 | 0.11 | 0.07 |
| SEM | | 0.64 | 0.15 | 0.58 | 0.39 | 0.79 | 0.19 | 0.53 | 0.35 | 0.72 | 0.22 | 0.80 | 0.19 | 0.89 | 0.19 | 0.81 | 0.26 | 0.68 | 0.19 | 0.53 | 0.43 | 1.14 | 0.17 | 0.34 | 0.58 |
| Lor- | LT-1 | 8 | 0 | 2 | 0 | 9 | 0 | 0 | 1 | 8 | 0 | 0 | 2 | 7 | 0 | 0 | 3 | 6 | 4 | 0 | 0 | 10 | 0 | 0 | 0 |
| caserin | LT-2 | 9 | 0 | 0 | 1 | 8 | 1 | 0 | 1 | 9 | 1 | 0 | 0 | 8 | 0 | 0 | 2 | 6 | 2 | 1 | 1 | 10 | 0 | 0 | 0 |
| (1 | LT-3 | 5 | 0 | 2 | 3 | 9 | 0 | 1 | 0 | 7 | 0 | 0 | 3 | 9 | 1 | 0 | 0 | 8 | 0 | 0 | 2 | 9 | 0 | 0 | 1 |
| mg/ | LT-4 | 10 | 0 | 0 | 0 | 9 | 1 | 0 | 0 | 7 | 2 | 1 | 0 | 10 | 0 | 0 | 0 | 9 | 0 | 1 | 0 | 0 | 0 | 0 | 0 |
| kg) | LT-5 | 7 | 0 | 0 | 3 | 0 | 2 | 1 | 7 | 8 | 1 | 1 | 0 | 8 | 0 | 0 | 2 | 6 | 0 | 1 | 3 | 7 | 2 | 0 | 1 |
| | LT-6 | 7 | 3 | 0 | 0 | 10 | 0 | 0 | 0 | 8 | 1 | 1 | 0 | 5 | 0 | 2 | 3 | 8 | 0 | 0 | 2 | 4 | 2 | 0 | 4 |
| | LT-7 | 6 | 1 | 3 | 0 | 9 | 0 | 0 | 1 | 9 | 0 | 0 | 1 | 10 | 0 | 0 | 0 | 8 | 1 | 1 | 0 | 4 | 0 | 6 | 0 |
| | LT-8 | 5 | 1 | 2 | 2 | 4 | 0 | 4 | 2 | 7 | 0 | 3 | 0 | 9 | 0 | 0 | 1 | 6 | 1 | 2 | 1 | 5 | 2 | 1 | 2 |
| | LT-9 | 9 | 0 | 0 | 1 | 9 | 0 | 0 | 1 | 10 | 0 | 0 | 0 | 8 | 0 | 0 | 2 | 9 | 0 | 0 | 1 | 6 | 1 | 3 | 0 |
| | LT-10 | 7 | 0 | 3 | 0 | 10 | 0 | 0 | 0 | 10 | 0 | 0 | 0 | 10 | 0 | 0 | 0 | 6 | 2 | 1 | 1 | 0 | 0 | 0 | 0 |
| | LT-11 | 9 | 0 | 0 | 1 | 6 | 1 | 2 | 1 | 8 | 1 | 1 | 0 | 8 | 0 | 0 | 2 | 9 | 0 | 0 | 1 | 8 | 0 | 0 | 2 |
| | LT-12 | 10 | 0 | 0 | 0 | 7 | 1 | 2 | 0 | 10 | 0 | 0 | 0 | 9 | 0 | 0 | 1 | 9 | 0 | 0 | 1 | 7 | 1 | 2 | 0 |
| AVG | | 0.77 | 0.04 | 0.10 | 0.09 | 0.75 | 0.05 | 0.08 | 0.12 | 0.84 | 0.05 | 0.06 | 0.05 | 0.84 | 0.01 | 0.02 | 0.13 | 0.75 | 0.08 | 0.06 | 0.11 | 0.58 | 0.07 | 0.10 | 0.08 |
| SEM | | 0.51 | 0.26 | 0.37 | 0.34 | 0.85 | 0.19 | 0.37 | 0.56 | 0.34 | 0.19 | 0.26 | 0.29 | 0.42 | 0.08 | 0.17 | 0.33 | 0.40 | 0.37 | 0.19 | 0.26 | 0.98 | 0.26 | 0.54 | 0.37 |
| Com- | LT-1 | 2 | 0 | 8 | 0 | 9 | 0 | 1 | 0 | 10 | 0 | 0 | 0 | 10 | 0 | 0 | 0 | 9 | 0 | 1 | 0 | 10 | 0 | 0 | 0 |
| pound | LT-2 | 4 | 0 | 2 | 4 | 5 | 2 | 1 | 2 | 3 | 0 | 3 | 4 | 3 | 2 | 4 | 1 | 10 | 0 | 0 | 0 | 5 | 1 | 4 | 0 |
| I-19 | LT-3 | 6 | 1 | 2 | 1 | 10 | 0 | 0 | 0 | 9 | 0 | 0 | 1 | 10 | 0 | 0 | 0 | 3 | 1 | 4 | 2 | 10 | 0 | 0 | 0 |
| (0.1 | LT-4 | 4 | 1 | 5 | 0 | 7 | 1 | 2 | 0 | 9 | 1 | 0 | 0 | 9 | 0 | 0 | 1 | 9 | 0 | 0 | 1 | 8 | 0 | 1 | 1 |
| mg/ | LT-5 | 0 | 0 | 1 | 9 | 0 | 0 | 0 | 10 | 0 | 0 | 0 | 10 | 0 | 1 | 1 | 8 | 3 | 1 | 2 | 4 | 2 | 0 | 0 | 8 |
| kg) | LT-6 | 7 | 1 | 2 | 0 | 8 | 0 | 1 | 1 | 9 | 1 | 0 | 0 | 9 | 0 | 0 | 1 | 9 | 0 | 0 | 1 | 0 | 0 | 0 | 0 |
| | LT-7 | 3 | 0 | 7 | 0 | 6 | 0 | 2 | 2 | 8 | 0 | 1 | 1 | 6 | 1 | 0 | 3 | 0 | 1 | 8 | 1 | 6 | 2 | 2 | 0 |
| | LT-8 | 4 | 1 | 2 | 3 | 6 | 2 | 1 | 1 | 10 | 0 | 0 | 0 | 8 | 0 | 0 | 2 | 9 | 0 | 0 | 1 | 1 | 1 | 3 | 5 |
| | LT-9 | 10 | 0 | 0 | 0 | 3 | 1 | 6 | 0 | 8 | 0 | 0 | 2 | 8 | 0 | 1 | 1 | 9 | 0 | 0 | 1 | 8 | 0 | 1 | 1 |
| | LT-10 | 8 | 0 | 2 | 0 | 10 | 0 | 0 | 0 | 5 | 1 | 4 | 0 | 8 | 0 | 2 | 0 | 4 | 1 | 5 | 0 | 8 | 0 | 2 | 0 |
| | LT-11 | 7 | 0 | 2 | 1 | 10 | 0 | 0 | 0 | 10 | 0 | 0 | 0 | 4 | 1 | 5 | 0 | 5 | 0 | 3 | 2 | 4 | 0 | 6 | 0 |
| | LT-12 | 7 | 0 | 3 | 0 | 7 | 0 | 2 | 1 | 6 | 2 | 2 | 0 | 6 | 0 | 3 | 1 | 7 | 0 | 2 | 1 | 0 | 0 | 0 | 0 |
| AVG | | 0.52 | 0.03 | 0.30 | 0.15 | 0.68 | 0.05 | 0.13 | 0.14 | 0.73 | 0.04 | 0.08 | 0.15 | 0.68 | 0.04 | 0.13 | 0.15 | 0.64 | 0.03 | 0.21 | 0.12 | 0.52 | 0.03 | 0.16 | 0.13 |
| SEM | | 0.81 | 0.14 | 0.70 | 0.78 | 0.88 | 0.23 | 0.48 | 0.81 | 0.91 | 0.19 | 0.41 | 0.85 | 0.89 | 0.19 | 0.51 | 0.65 | 0.95 | 0.14 | 0.73 | 0.32 | 1.08 | 0.19 | 0.56 | 0.74 |
| Com- | LT-1 | 9 | 0 | 0 | 1 | 7 | 0 | 3 | 0 | 10 | 0 | 0 | 0 | 10 | 0 | 0 | 0 | 5 | 1 | 4 | 0 | 0 | 0 | 0 | 0 |
| pound | LT-2 | 9 | 1 | 0 | 0 | 6 | 1 | 2 | 1 | 10 | 0 | 0 | 0 | 10 | 0 | 0 | 0 | 6 | 1 | 3 | 0 | 6 | 1 | 3 | 0 |
| I-19 | LT-3 | 4 | 0 | 5 | 1 | 10 | 0 | 0 | 0 | 9 | 1 | 0 | 0 | 10 | 0 | 0 | 0 | 9 | 0 | 1 | 0 | 10 | 0 | 0 | 0 |
| (0.3 | LT-4 | 9 | 1 | 0 | 0 | 10 | 0 | 0 | 0 | 9 | 0 | 0 | 1 | 10 | 0 | 0 | 0 | 9 | 1 | 0 | 0 | 4 | 1 | 5 | 0 |
| mg/ | LT-5 | 4 | 3 | 2 | 1 | 9 | 0 | 1 | 0 | 6 | 0 | 1 | 3 | 0 | 0 | 4 | 4 | 1 | 0 | 6 | 3 | 0 | 0 | 0 | 10 |
| kg) | LT-6 | 9 | 0 | 0 | 1 | 8 | 1 | 0 | 1 | 7 | 0 | 1 | 2 | 10 | 0 | 0 | 0 | 4 | 0 | 0 | 6 | 4 | 0 | 0 | 6 |
| | LT-7 | 0 | 1 | 6 | 2 | 1 | 2 | 3 | 4 | 0 | 0 | 0 | 10 | 0 | 0 | 0 | 10 | 0 | 2 | 2 | 6 | 0 | 0 | 0 | 0 |
| | LT-8 | 2 | 0 | 2 | 6 | 4 | 0 | 4 | 2 | 10 | 0 | 0 | 0 | 3 | 0 | 1 | 6 | 8 | 0 | 0 | 2 | 2 | 1 | 0 | 7 |
| | LT-9 | 7 | 0 | 3 | 0 | 10 | 0 | 0 | 0 | 10 | 0 | 0 | 0 | 10 | 0 | 0 | 0 | 10 | 0 | 0 | 0 | 8 | 0 | 1 | 1 |
| | LT-10 | 9 | 0 | 1 | 0 | 8 | 0 | 1 | 1 | 8 | 0 | 1 | 1 | 7 | 0 | 2 | 1 | 5 | 1 | 4 | 0 | 8 | 1 | 1 | 0 |
| | LT-11 | 8 | 0 | 1 | 1 | 6 | 2 | 2 | 0 | 5 | 0 | 2 | 3 | 8 | 0 | 0 | 2 | 1 | 0 | 7 | 2 | 5 | 0 | 4 | 1 |
| | LT-12 | 5 | 0 | 5 | 0 | 4 | 0 | 4 | 2 | 4 | 1 | 2 | 3 | 5 | 1 | 2 | 2 | 4 | 0 | 6 | 0 | 6 | 1 | 3 | 0 |
| AVG | | 0.63 | 0.05 | 0.21 | 0.11 | 0.69 | 0.05 | 0.17 | 0.09 | 0.73 | 0.02 | 0.06 | 0.19 | 0.69 | 0.01 | 0.08 | 0.21 | 0.52 | 0.05 | 0.28 | 0.16 | 0.44 | 0.04 | 0.14 | 0.21 |
| SEM | | 0.91 | 0.26 | 0.63 | 0.48 | 0.82 | 0.23 | 0.45 | 0.36 | 0.90 | 0.11 | 0.23 | 0.82 | 1.14 | 0.08 | 0.37 | 0.91 | 0.98 | 0.19 | 0.76 | 0.67 | 0.98 | 0.15 | 0.53 | 1.01 |
| Com- | LT-1 | 10 | 0 | 0 | 0 | 10 | 0 | 0 | 0 | 10 | 0 | 0 | 0 | 9 | 0 | 1 | 0 | 0 | 1 | 8 | 1 | 0 | 0 | 6 | 4 |
| pound | LT-2 | 8 | 0 | 0 | 2 | 10 | 0 | 0 | 0 | 2 | 0 | 5 | 3 | 6 | 1 | 3 | 0 | 7 | 0 | 0 | 3 | 2 | 1 | 0 | 7 |
| I-19 | LT-3 | 5 | 0 | 4 | 1 | 10 | 0 | 0 | 0 | 10 | 0 | 0 | 0 | 10 | 0 | 0 | 0 | 10 | 0 | 0 | 0 | 0 | 0 | 0 | 0 |
| (1 | LT-4 | 10 | 0 | 0 | 0 | 6 | 0 | 0 | 4 | 5 | 1 | 1 | 3 | 5 | 0 | 4 | 1 | 3 | 2 | 4 | 1 | 10 | 0 | 0 | 0 |
| mg/ | LT-5 | 9 | 0 | 1 | 0 | 3 | 0 | 6 | 1 | 3 | 0 | 5 | 2 | 6 | 0 | 0 | 4 | 7 | 0 | 0 | 3 | 5 | 0 | 1 | 4 |
| kg) | LT-6 | 8 | 0 | 0 | 2 | 8 | 0 | 0 | 2 | 10 | 0 | 0 | 0 | 7 | 0 | 2 | 1 | 10 | 0 | 0 | 0 | 5 | 0 | 3 | 2 |
| | LT-7 | 0 | 0 | 1 | 9 | 2 | 0 | 3 | 5 | 10 | 0 | 0 | 0 | 6 | 0 | 0 | 4 | 5 | 0 | 2 | 3 | 3 | 0 | 2 | 5 |
| | LT-8 | 8 | 0 | 0 | 2 | 10 | 0 | 0 | 0 | 2 | 0 | 5 | 3 | 6 | 1 | 3 | 0 | 7 | 0 | 0 | 3 | 2 | 1 | 0 | 7 |
| | LT-9 | 2 | 1 | 7 | 0 | 10 | 0 | 0 | 0 | 10 | 0 | 0 | 0 | 7 | 2 | 1 | 0 | 7 | 0 | 1 | 2 | 0 | 0 | 0 | 0 |
| | LT-10 | 10 | 0 | 0 | 0 | 9 | 0 | 0 | 1 | 8 | 0 | 0 | 2 | 8 | 0 | 0 | 2 | 6 | 2 | 1 | 1 | 9 | 0 | 0 | 1 |
| | LT-11 | 0 | 1 | 1 | 8 | 9 | 0 | 1 | 0 | 3 | 0 | 1 | 6 | 3 | 3 | 1 | 3 | 4 | 1 | 1 | 4 | 8 | 0 | 2 | 0 |
| | LT-12 | 0 | 0 | 3 | 7 | 0 | 1 | 2 | 7 | 0 | 0 | 0 | 10 | 3 | 2 | 3 | 2 | 5 | 1 | 4 | 0 | 5 | 0 | 4 | 1 |
| AVG | | 0.58 | 0.02 | 0.14 | 0.26 | 0.73 | 0.01 | 0.10 | 0.17 | 0.61 | 0.01 | 0.14 | 0.24 | 0.63 | 0.08 | 0.15 | 0.14 | 0.59 | 0.06 | 0.18 | 0.18 | 0.41 | 0.02 | 0.15 | 0.26 |
| SEM | | 1.21 | 0.11 | 0.63 | 0.98 | 1.05 | 0.08 | 0.54 | 0.69 | 1.14 | 0.08 | 0.63 | 0.87 | 0.61 | 0.30 | 0.42 | 0.45 | 0.81 | 0.23 | 0.71 | 0.41 | 1.02 | 0.11 | 0.57 | 0.78 |
| Com- | LT-1 | 7 | 0 | 0 | 3 | 4 | 0 | 5 | 1 | 3 | 0 | 7 | 0 | 8 | 0 | 0 | 2 | 10 | 0 | 0 | 0 | 6 | 0 | 3 | 1 |
| pound | LT-2 | 7 | 2 | 1 | 0 | 10 | 0 | 0 | 0 | 10 | 0 | 0 | 0 | 5 | 2 | 1 | 2 | 10 | 0 | 0 | 0 | 0 | 0 | 0 | 0 |
| I-19 | LT-3 | 8 | 0 | 2 | 0 | 5 | 0 | 3 | 2 | 9 | 1 | 0 | 0 | 7 | 0 | 3 | 0 | 10 | 0 | 0 | 0 | 8 | 0 | 2 | 0 |
| (3 | LT-4 | 9 | 0 | 1 | 0 | 4 | 1 | 4 | 1 | 7 | 0 | 0 | 3 | 9 | 0 | 0 | 1 | 6 | 1 | 3 | 0 | 0 | 1 | 7 | 2 |
| mg/ | LT-5 | 8 | 0 | 0 | 2 | 8 | 0 | 0 | 2 | 0 | 2 | 1 | 6 | 0 | 0 | 0 | 10 | 0 | 0 | 0 | 10 | 4 | 0 | 2 | 4 |
| kg) | LT-6 | 6 | 1 | 2 | 1 | 9 | 0 | 0 | 1 | 9 | 0 | 0 | 1 | 3 | 1 | 5 | 1 | 8 | 1 | 0 | 1 | 7 | 0 | 1 | 2 |
| | LT-7 | 0 | 0 | 2 | 8 | 0 | 1 | 1 | 8 | 1 | 0 | 5 | 4 | 3 | 2 | 0 | 5 | 0 | 0 | 5 | 5 | 2 | 0 | 4 | 4 |
| | LT-8 | 1 | 1 | 2 | 6 | 7 | 1 | 0 | 2 | 9 | 0 | 0 | 1 | 9 | 0 | 0 | 1 | 4 | 0 | 1 | 5 | 0 | 0 | 0 | 0 |
| | LT-9 | 5 | 0 | 4 | 1 | 8 | 0 | 2 | 0 | 7 | 0 | 3 | 0 | 7 | 0 | 2 | 1 | 3 | 0 | 6 | 1 | 1 | 0 | 2 | 7 |
| | LT-10 | 10 | 0 | 0 | 0 | 4 | 2 | 4 | 0 | 10 | 0 | 0 | 0 | 4 | 3 | 2 | 1 | 5 | 0 | 5 | 0 | 10 | 0 | 0 | 0 |
| | LT-11 | 5 | 0 | 5 | 0 | 9 | 0 | 0 | 1 | 0 | 2 | 5 | 3 | 0 | 0 | 8 | 2 | 8 | 0 | 0 | 2 | 4 | 1 | 4 | 1 |
| | LT-12 | 5 | 2 | 2 | 1 | 6 | 0 | 4 | 0 | 2 | 2 | 5 | 1 | 7 | 0 | 3 | 0 | 5 | 0 | 3 | 2 | 2 | 0 | 7 | 1 |

| | | | | | | | | | | | | | | | | | | | | | | | | |
|---|---|---|---|---|---|---|---|---|---|---|---|---|---|---|---|---|---|---|---|---|---|---|---|---|
| AVG | 0.59 | 0.05 | 0.18 | 0.18 | 0.62 | 0.04 | 0.19 | 0.15 | 0.56 | 0.06 | 0.22 | 0.16 | 0.52 | 0.07 | 0.20 | 0.22 | 0.58 | 0.02 | 0.19 | 0.22 | 0.37 | 0.02 | 0.27 | 0.18 |
| SEM | 0.87 | 0.23 | 0.45 | 0.76 | 0.83 | 0.19 | 0.57 | 0.63 | 1.17 | 0.26 | 0.77 | 0.57 | 0.92 | 0.31 | 0.72 | 0.81 | 1.04 | 0.11 | 0.68 | 0.89 | 0.99 | 0.11 | 0.71 | 0.63 |

In summary, acute treatment of Compound I-19 significantly decreased food intake at 1 and 3 mg/kg, without impacting BSS. Based on these observations, it is posited that Compound I-19 facilitated this reduction in food intake by increasing satiety. Moreover, the positive control, lorcaserin, induced a similar feeding pattern, in which food intake decreased as a function of satiety.

### Example 12. Evaluation of Compound I-4 and Compound I-19 in the Rat Five Choice Serial Reaction Time Task

The purpose of this study was to evaluate Compounds I-4 and I-19 for effects on sustained attention and inhibitory response control in an experimental animal model. The behavioral tasks utilized were two versions of the rat Five Choice Serial Reaction Time Task (5C-SRTT): variable stimulus duration (vSD) and variable intertrial interval (vITI).

### Materials and Methods

### Study Subjects:

Experimentally naive, male Wistar rats (7 to 8 weeks old, Envigo, Inc, Indianapolis, IN) were double housed in polycarbonate cages (45×30×18 cm) with corncob bedding in a vivarium of constant temperature (21-23° C.) and humidity (40-50%). Lighting was maintained on a 12-hr light-dark cycle (7:00 a.m.-7:00 p.m.) with free access to water and food during the first week (see subsequent food restriction procedures below). All behavioral testing was performed during the light portion (9 a.m.-5 p.m.) of the light/dark cycle (Monday thru Friday). Animals were cared for in accordance with the Guide for the Care and Use of Laboratory Animals (U.S. National Institutes of Health) and all experimental protocols were approved by the Institutional Animal Care and Use Committee at Augusta University.

### 5-Choice Serial Reaction Time (5C-SRTT) Procedures

The 5C-SRTT procedure (reviewed, Robbins, 2002, Psychopharmacology (Berl); 163(3-4):362-80) was conducted as we have described previously (Terry et al., 2014, Neurotoxicology and Teratology 44:18-29, 2014; Callahan et al., 2020, Neuropharmacology, August 15; 173:107994). One week prior to 5C-SRTT training and throughout testing, rats were food restricted to approximately 85% of their age-dependent, free-feeding weights based upon Harlan Laboratories growth rate curves. Animals were trained in eight automated 5C-SRTT operant chambers (Med Associates, St. Albans, VT, USA), controlled by MedPC software (Med Associates). Briefly, each operant chamber was equipped with 5 apertures containing a photocell beam to detect nose pokes and a lamp (2.8 W) that could be illuminated randomly at varying durations. Food pellets (45 mg chow pellet, BioServ, Frenchtown, NJ, USA) were delivered automatically to a magazine, located on the opposite wall to the nose pokes, that was also equipped with a light that turned on to indicate that a pellet had been dispensed. The house-light remained on for the entire session unless an error or omission occurred. Training sessions began with the delivery of a food reward and retrieval triggered the first trial. After a 5 sec inter-trial interval (ITI), a stimulus light within one of the five apertures was illuminated for a fixed duration (see below) and a single nose-poke into this opening during the signal illumination period or during the 5 sec limited hold period delivered a reward (correct response); a nose-poke into a non-illuminated aperture (incorrect response) resulted in a 5 sec time-out period and no food reward. Failure to respond within the 5 sec limited hold period (omission) also resulted in a time-out. Training began with the stimulus duration set at 10 sec, a limited hold period of 5 sec and an ITI of 5 sec. The stimulus duration (e.g., 5, 2.5, 2.0, 1.5, and 1.25 sec) was gradually reduced, maintaining stable performance, until a final duration of 1.0 sec was achieved. Sessions ended when 40 minutes had lapsed or 100 trials had been completed. Twenty animals were trained 5 days per week until they reached stable performance criteria with a fixed (1.0 sec) stimulus duration and a fixed ITI (5 sec) of 70-75% accuracy, <20% omissions and completion of all 100 trials for 5 consecutive days.

Upon meeting the performance criteria, animals were assessed in the vSD version of the task where the following stimulus durations were presented in a pseudorandom manner: 0.3, 0.6 and 0.9 sec. Later, subjects were assessed in the vITI version where 2.5, 5.0 and 10.0 sec ITIs were employed in a pseudorandom manner. Performance parameters measured were: % hit ((# correct/(# correct+# incorrect including omissions))×100); % correct ((# correct/(# correct+# incorrect excluding omissions))×100); premature responses (# of nose-pokes into any aperture after trial initiation but before onset of the stimulus light); timeout responses (# of nose pokes into any aperture during a timeout period), perseverative responses (# of nose pokes occurring after the correct response had been made but before reward collection); # of food magazine head entries, omissions and total trials completed. The latency to correct response (time taken from the onset of the nose poke light stimulus to making the correct nose poke response), latency to incorrect response (time taken from onset of nose poke light stimulus to making the incorrect nose poke response), and latency to reward (i.e., the magazine latency, time taken from making a correct nose poke response to retrieving the reward from the magazine) was also recorded.

### Drug Preparation and Administration:

Nicotine was dissolved in normal (0.9%) saline and administered by subcutaneous (sc) injection 15 min. prior to 5C-SRTT testing. Normal saline was used as the vehicle control for the nicotine experiments. Test compounds were initially dissolved in 4% DMSO+30% PEG400+66% HβCD (30%). They were subsequently diluted to achieve the proper drug concentration for the injections, and the final concentrations of the diluents were as follows: 0.8% DMSO, 6% PEG400, 93.2% HβCD (6%). Compound I-4 (dose range 0.1-3.0 mg/kg), Compound I-19 (dose range 0.03-1.0 mg/kg) and the vehicle described above were administered by intraperitoneal (ip) injection, 30 min. before behavioral testing. With the exception of the 3.0 mg/kg dose of Compound I-4, which was administered in a volume of 2.0 ml/kg, all of the other doses of Compound I-4 and Compound I-19 were administered in a volume of 1.0 ml/kg. Drug doses and vehicle were administered in a pseudorandom fashion. Drug or vehicle sessions were performed 1-2 times per week with maintenance training (behavioral testing without drug or vehicle injections) interspersed.

### Statistical Analysis

All data were collated and entered into Microsoft Excel spreadsheets. The data were subsequently imported into StatView 5.0 for statistical analyses. One and two factor analysis of variance (ANOVA) tests were used with repeated measures when indicated, followed by Fisher LSD or Student Newman Keuls as post-hoc tests. All results were expressed as the mean (±S.E.M.). Differences between means from experimental groups were considered significant at the p<0.05 level. The statistical F values and degrees of freedom associated with the two factor ANOVA analyses are provided in the text of this report. Please refer to the statistical reports in the appendices for the values associated with one factor ANOVAs for the overall means and overall total values.

### Results

### Effects of Nicotine on Performance of the vSD Version of the 5C-SRTT (Tables 142 and 143)

In the initial round of experiments, two groups of rats (N=10) were treated with nicotine (0.05-0.2 mg/kg, sc) and evaluated in the vSD version of the 5C-SRTT. Due to the modest effects on accuracy (specifically the % hit measure), the groups were combined and the data reanalyzed with all 20 rats included. In the larger cohort of animals, nicotine was associated with an increase in the % hit rate, showing a dose (treatment) effect [F(3,57)=4.7, p<0.001] and effect of stimulus duration [F(2,171)=41.9, p=0.004], without a significant dose×stimulus duration interaction. Post hoc analyses indicated that all 3 doses of nicotine slightly improved the % hit rate at the 0.3 sec SD, while two of the doses (0.05 and 0.2 mg/kg) improved performance at both the 0.6 and 0.9 sec SD. The later observation was also evident when the overall % hit rate (average across the 3 SDs) was analyzed. There were no statistically significant effects observed in the % correct assessment. While the number of omissions was relatively low in this study, all 3 doses of nicotine were associated with a decrease in omissions, observed as an effect of dose [F(3,57)=10.7, p<0.001] and effect of stimulus duration [F(2,171)=2.7, p=0.08], with no significant dose×stimulus duration interaction. In the premature response analysis, the higher 2 doses of nicotine were associated with an increase, noted as an effect of dose [F(3,57)=37.0, p<0.001], while the effect of stimulus duration and the dose×stimulus duration interactions were insignificant. A similar effect of nicotine was observed when the number of timeout responses [i.e., effect of dose [F(3,57)=7.8, p<0.001] and magazine head entries [i.e., effect of dose [F(3,57)=17.8, p<0.001] were analyzed. There were no statistically significant effects on perseverative responses or reward latencies. Nicotine, however, was associated with a modest decrease in the response latencies associated with correct responses at the 0.9 sec SD (and overall), observed as a significant effect of dose [F(3,57)=3.2, p=0.02], as well as incorrect response latencies [i.e., effect of dose, [F(3,57)=19.2 p<0.001]. Nicotine had no effect on the total number of trials completed with all subjects completing all 100 trials (data not shown).

In summary, in the vSD version of the 5C-SRTT, nicotine was associated with modest improvements in sustained attention, which may in part be explained by its tendency to decrease the number of omissions. Here it is important to note that omissions are considered as errors in the % hit analysis, but not in the % accuracy assessment. While nicotine was not associated with compulsivity-like behaviors (as would be evident if the number of perseverative responses increased), it was associated with impulsivity-like responding as evident by an increase in the number of premature responses, timeout responses, magazine head entries, and the decreases in the response latencies.

TABLE 142
Statistical analyses of nicotine, Compound I-4, and Compound I-19 on the primary
measures of attention and impulsivity in the vSD condition of 5CSRTT.

| | | Stimulation | Dose × Stimulation |
|---|---|---|---|
| vSD | Dose | Duration | Duration |
| % Hit | | | |
| Nicotine (ANOVA) | F(3, 57) = 4.7, p < 0.001 | F(2, 171) = 41.9, p = 0.004 | n.s. |
| Compound I-4 | F(4, 57) = 24.5, p < 0.001 | F(2, 228) = 24.2, p < 0.001 | n.s. |
| Compound I-19 | F(4, 57) = 12.8, p < 0.001 | F(2, 228) = 72.8, p < 0.001 | n.s. |
| % Omissions | | | |
| Nicotine (ANOVA) | F(3, 57) = 10.7, p < 0.001 | F(2, 171) = 2.7, p = 0.08 | n.s. |
| Compound I-4 | F(4, 57) = 40.0 p < 0.001 | n.s. | n.s. |
| Compound I-19 | F(4, 57) = 24.1 p < 0.001 | n.s. | n.s. |
| Premature | | | |
| Responses | | | |
| Nicotine (ANOVA) | F(3, 57) = 37.0, p < 0.001 | n.s. | n.s. |
| Compound I-4 | F(4, 57) = 13.6, p < 0.001 | n.s. | n.s. |
| Compound I-19 | F(4, 57) = 17.4, p < 0.001 | n.s. | F(8, 228) = 2.1, p = 0.03 |
| Time Out | | | |
| Nicotine (ANOVA) | F(3, 57) = 7.8, p < 0.001 | n.s. | n.s. |
| Compound I-4 | F(4, 57) = 6.3 p < 0.001 | n.s. | n.s. |
| Compound I-19 | F(4, 57) = 10.9, p < 0.001 | F(2, 228) = 4.4, p = 0.016 | n.s. |
| Head Entries | | | |
| Nicotine (ANOVA) | F(3, 57) = 17.8, p < 0.001 | n.s. | n.s. |
| Compound I-4 | F(4, 57) = 32.7 p < 0.001 | n.s. | n.s. |
| Compound I-19 | F(4, 57) = 15.4 p < 0.001 | n.s. | n.s. |
| Perseverative | | | |
| Response | | | |
| Nicotine (ANOVA) | n.s. | n.s. | n.s. |
| Compound I-4 | n.s. | n.s. | n.s. |
| Compound I-19 | F(4, 57) = 2.9, p = 0.02 | n.s. | n.s. |
| Trials Completed | | | |
| Nicotine (ANOVA) | n.s. | n.s. | n.s. |
| Compound I-4 | [F(4, 57) − 27.9 p < 0.01 | n.s. | n.s. |
| Compound I-19 | F(4, 57) = 7.8 p < 0.001 | n.s. | n.s. |

TABLE 143
Statistical analyses of response latencies following nicotine,
Compound I-4, and Compound I-19 in the vSD condition of 5CSRTT.

| | | Stimulation | Dose × Stimulation |
|---|---|---|---|
| vSD | Dose | Duration | Duration |
| Correct Response | | | |
| Latency | | | |
| Nicotine (ANOVA) | F(3, 57) = 3.2, p = 0.02 | n.s. | n.s. |
| Compound I-4 | F(4, 57) = 5.8, p < 0.001 | n.s. | n.s. |
| Compound I-19 | F(4, 57) = 3.8, p = 0.005 | n.s. | n.s. |
| Incorrect | | | |
| Response Latency | | | |
| Nicotine (ANOVA) | F(3, 57) = 19.2 p < 0.001 | n.s. | n.s. |
| Compound I-4 | F(4, 57) = 9.9 p < 0.001 | n.s. | n.s. |
| Compound I-19 | F(4, 57) = 4.5 p = 0.002 | n.s. | n.s. |
| Reward Latency | | | |
| Nicotine (ANOVA) | n.s. | n.s. | n.s. |
| Compound I-4 | n.s. | n.s. | n.s. |
| Compound I-19 | n.s. | n.s. | n.s. |

### Effects of Compound I-4 on Performance of the vSD Version of the 5C-SRTT (Tables 142 and 143)

In the vSD version of the 5C-SRTT, Compound I-4 was associated with a decrease in the % hit rate, due to significant dose (treatment) [F(4,57)=24.5, p<0.001] and stimulus duration effects [F(2,228)=24.2, p<0.001], without a significant dose×stimulus duration interaction. Post hoc analyses indicated that the 3 higher doses of Compound I-4 (0.3-3.0 mg/kg) decreased the % hit rate across all 3 SDs as well as the overall % hit rate (average across the 3SDs). There were no statistically significant dose-related effects observed in the % correct assessment. The three higher doses of Compound I-4 were also associated with an increase in the number of omissions, as seen by a dose effect [F(4,57)=40.0 p<0.001], while the effect of stimulus duration and the dose×stimulus duration interactions were insignificant. In the premature response analysis, the higher 3 doses of Compound I-4 were associated with a decrease [i.e., effect of dose [F(4,57)=13.6, p<0.001]] at the two higher SDs and overall, while the effect of stimulus duration and the dose×stimulus duration interactions were insignificant. A similar effect of Compound I-4 was observed when the number of timeout responses [effect of dose [F(4,57)=6.3 p<0.001] and magazine head entries [effect of dose [F(4,57)=32.7 p<0.001] were analyzed. There were no statistically significant effects on perseverative responses. Depending on the dose and stimulus duration, Compound I-4 was associated with increases in the response latencies and decreases in the number of trials completed. Significant effects of dose were observed for the following: correct response latencies [F(4,57)=5.8, p=0.0002], incorrect response latencies [F(4,57)=9.9 p<0.001], and number of trials completed [F(4,57)=27.9 p<0.001]. There were some increases in the reward latencies, but they were not statistically significant.

In summary, in the vSD version of the 5C-SRTT, Compound I-4 was associated with impairments in sustained attention, which are likely explained by its tendency to increase the number of omissions, as well as to increase the response latencies and to decrease the total number of trials completed. These observations are indications of sedation, locomotor impairment, or decreases in motivation. Visual observations and handling after 5C-SRTT testing indicated that the animals appeared to be sluggish and/or possibly sedated. While Compound I-4 was also associated with decreases in impulsivity-like responding as evident by decreases in the number of premature responses, timeout responses, and magazine head entries, these observations should be viewed with caution given the negative effects of Compound I-4 described above.

Effects of Compound I-4 on Performance of the vITI Version of the 5C-SRTT (Tables 143 and 144)

In the vITI version of the 5C-SRTT, Compound I-4 was associated with a modest decrease in the % hit rate, displaying a dose (treatment) effect [F(3,54)=5.4, p=0.015] and effect of ITI [F(2,162)=10.1, p=0.002], without a significant dose×ITI interaction. Post hoc analyses indicated that the 0.3 mg/kg dose of Compound I-4 decreased the % hit rate at the 2.5 sec ITI, as well as the overall % hit rate (average across the 3 ITIs). There were no statistically significant dose-related effects observed in the % correct assessment. Depending on the dose and ITI, Compound I-4 was also associated with an increase in the number of omissions, showing a dose effect [F(3,54)=12.3 p<0.001], effect of ITI [F(2,162)=9.1, p=0.004], and dose×ITI interaction [F(6,162)=4.8, p=0.002]. In the premature response analysis, the higher 2 doses of Compound I-4 (0.3 and 1.0 mg/kg) were associated with a decrease in the number of premature responses, demonstrating a significant dose effect [F(3,54)=53.2 p<0.001], effect of ITI [F(2,162)=259.2, p<0.001], and dose×ITI interaction [F(6,162)=38.2, p<0.001]. A similar effect of Compound I-4 was observed when the number of timeout responses and magazine head entries were analyzed. Statistical analysis revealed the following: timeout responses: dose effect [F(3,54)=11.7 p<0.001], effect of ITI [F(2,162)=19.7, p<0.001], dose×ITI interaction [F(6,162)=7.7, p<0.001]; magazine head entries: dose effect [F(3,54)=39.5 p<0.001], effect of ITI [F(2,162)=54.9, p<0.001], dose×ITI interaction [F(6,162)=26.7, p<0.001]. There were no statistically significant effects on perseverative responses. Depending on the dose and ITI, Compound I-4 was also associated with increases in the response and reward latencies, although the number of trials completed was not affected. The following statistical results for latencies were obtained: correct response latencies: dose effect [F(3,54)=11.9 p<0.001], effect of ITI [F(2,162)=9.9, p=0.002], dose×ITI interaction [F(6,162)=2.4, p<0.03]; incorrect response latencies: dose effect [F(3,54)=4.0 p=0.0085], effect of ITI [F(2,162)=66.7, p<0.001], dose×ITI interaction [F(6,162)=4.0, p=0.009]; reward latencies: dose effect [F(3,54)=2.7 p=0.049], nonsignificant effect of ITI, dose×ITI interaction [F(6,162)=2.8, p=0.012].

In summary, in the vITI version of the 5C-SRTT, Compound I-4 was associated with very modest impairments in sustained attention, which are likely explained by its tendency to increase the number of omissions as well as to increase the response latencies. These observations may be indicative of mild sedation, locomotor impairment, or decreases in motivation. As noted in the vSD-5C-SRTT studies, visual observations and handling after testing indicated that the animals appeared to be somewhat sluggish and/or possibly sedated. As observed in the vSD studies, Compound I-4 was also associated with decreases in impulsivity-like responding as evident by decreases in the number of premature responses, timeout responses, and magazine head entries. However, these observations should be viewed with caution given the negative effects of Compound I-4 described above.

TABLE 143
Statistical analyses of Compound I-4 and Compound I-19 on the primary
measures of attention and impulsivity in the vITI condition of 5CSRTT.

| vITI | Dose | ITI | Dose × ITI |
|---|---|---|---|
| % Hit | | | |
| Compound I-4 | F(3, 54) = 5.4, p = 0.015 | F(2, 162) = 10.1, p = 0.002 | n.s. |
| Compound I-19 | F(4, 54) = 5.3, p < 0.001 | F(2, 216) = 13.6, p < 0.001 | n.s. |
| % Omissions | | | |
| Compound I-4 | F(3, 54) = 12.3 p < 0.001 | F(2, 162) = 9.1, p = 0.004 | F(6, 162) = 4.8, p = 0.002 |
| Compound I-19 | F(4, 54) = 8.8 p < 0.001 | F(2, 216) = 15.1, p < 0.001 | n.s. |
| Premature | | | |
| Responses | | | |
| Compound I-4 | F(3, 54) = 53.2 p < 0.001 | F(2, 162) = 259.2, p < 0.001 | F(6, 162) = 38.2, p < 0.001 |
| Compound I-19 | F(4, 54) = 33.1, p < 0.001 | F(2, 216) = 225.8, p < 0.001 | F(8, 216) = 24.7 p < 0.001 |
| Time Out | | | |
| Compound I-4 | F(3, 54) = 11.7 p < 0.001 | F(2, 162) = 19.7, p < 0.001 | F(6, 162) = 7.7, p < 0.001 |
| Compound I-19 | F(4, 54) = 11.8 p < 0.001 | F(2, 216) = 27.4, p < 0.001 | F(8, 216) = 10.0, p < 0.001 |
| Head Entries | | | |
| Compound I-4 | F(3, 54) = 39.5 p < 0.001 | F(2, 162) = 54.9, p < 0.001 | F(6, 162) = 26.7, p < 0.001 |
| Compound I-19 | F(4, 54) = 26.8 p < 0.001 | F(2, 216) = 55.3, p < 0.001 | F(8, 216) = 18.5, p < 0.001 |
| Perseverative | | | |
| Response | | | |
| Compound I-4 | n.s. | n.s. | n.s. |
| Compound I-19 | n.s. | n.s. | n.s. |
| Trials Completed | | | |
| Compound I-4 | n.s. | n.s. | n.s. |
| Compound I-19 | n.s. | n.s. | n.s. |

TABLE 144
Statistical analyses of response latencies following Compound
I-4 and Compound I-19 in the vITI condition of 5CSRTT.

| vSD | Dose | ITI | Dose × ITI |
|---|---|---|---|
| Correct Response | | | |
| Latency | | | |
| Compound I-4 | F(3, 54) = 11.9 p < 0.001 | F(2, 162) = 9.9, p = 0.002 | F(6, 162) = 2.4, p < 0.03 |
| Compound I-19 | F(4, 54) = 4.1 p = 0.003 | F(2, 216) = 13.1, p < 0.001 | n.s. |
| Incorrect | | | |
| Response Latency | | | |
| Compound I-4 | [F(3, 54) = 4.0 p = 0.009 | F(2, 162) = 66.7, p < 0.001 | F(6, 162) = 4.0, p = 0.009 |
| Compound I-19 | F(4, 54) = 5.5 p = 0.003 | F(2, 216) = 41.6, p < 0.001 | n.s. |
| Reward Latency | | | |
| Compound I-4 | F(3, 54) = 2.7 p = 0.049 | n.s. | F(6, 162) = 2.8, p = 0.012 |
| Compound I-19 | n.s. | n.s. | n.s. |

### Effects of Compound I-19 on Performance of the vSD Version of the 5C-SRTT (Tables 141 and 142)

In the vSD version of the 5C-SRTT, Compound I-19 was associated with a dose-dependent decrease in the % hit rate, showing dose (treatment) [F(4,57)=12.8, p<0.001] and stimulus duration effects [F(2,228)=72.8, p<0.001], without a significant dose×ITI interaction. Post hoc analyses indicated that the 3 higher doses of Compound I-19 (0.1-1.0 mg/kg) decreased the % hit rate across all 3 SDs, as well as the overall % hit rate (average across the 3 SDs). There were no statistically significant dose-related effects observed in the % correct assessment. The three higher doses of Compound I-19 were also associated with an increase in the number of omissions, as observed by a dose effect [F(4,57)=24.1 p<0.001], while the effect of stimulus duration and the dose×stimulus duration interactions were insignificant. In the premature response analysis, the higher 3 doses of Compound I-19 were associated with a decrease in the number of premature responses, demonstrating a significant effect of dose [F(4,57)=17.4, p<0.001], without a significant effect of stimulus duration, or dose×stimulus duration interaction]. A similar effect of Compound I-19 was observed when the number of timeout responses were analyzed [i.e., effect of dose [F(4,57)=10.9, p<0.001], stimulus duration [F(2,228)=4.4, p=0.016], without a significant dose×stimulus duration interaction]. Likewise, the number magazine head entries were decreased in a dose-dependent manner, exhibited by a significant effect of dose [F(4,57)=15.4 p<0.001], without a significant effect of stimulus duration or dose×stimulus duration interaction. There was also a very modest reduction in the number of perseverative responses, most notable at the 0.3 mg/kg dose [i.e., effect of dose [F(4,57)=2.9, p=0.02]], without a significant stimulus duration effect or dose×stimulus duration interaction. The highest dose of Compound I-19 (1.0 mg/kg) was also associated with very modest increases in the response latencies and decreases in the number of trials completed. The following significant dose-related effects were observed: correct response latencies: [F(4,57)=3.8, p=0.005], incorrect response latencies: [F(4,57)=4.5 p=0.002], and number of trials completed [F(4,57)=7.8 p<0.001]. There were some increases in the reward latencies, but they were not statistically significant.

In summary, in the vSD version of the 5C-SRTT, Compound I-19 was associated with dose-related impairments in sustained attention, which are likely explained by its tendency to increase the number of omissions, increase response latencies, and decrease the total number of trials completed. These observations were most notable with the highest dose of Compound I-19 (1.0 mg/kg) and are indications of sedation, locomotor impairment, or decreases in motivation. Like the case of Compound I-4, visual observation and handling after 5C-SRTT testing indicated that the animals (especially those administered the 1.0 mg/kg dose of Compound I-19) appeared to be sluggish and/or possibly sedated. While Compound I-19 was associated with decreases in impulsivity and compulsivity-related responding, as made evident by the observed decrease in the number of premature responses, timeout responses, magazine head entries, and perseverative responses. However, these observations should be viewed with caution given the negative effects of Compound I-19 described above.

Effects of Compound I-19 on Performance of the vITI Version of the 5C-SRTT (Tables 143 and 144)

In the vITI version of the 5C-SRTT, Compound I-19 was associated with a modest decrease in the % hit rate, as demonstrated by a dose (treatment) effect [F(4,54)=5.3, p=0.0004] and effect of ITI [F(2,216)=13.6, p<0.001], without a significant dose×ITI interaction. Post hoc analyses indicated that the 0.3 and 1.0 mg/kg dose of Compound I-19 decreased the % hit rate at the 2.5 sec ITI as well as the overall % hit rate (average across the 3ITIs), while the 0.3 mg/kg dose also decreased the % hit rate at the 5.0 sec ITI. There were no statistically significant dose-related effects observed in the % correct assessment. Depending on the dose and ITI, Compound I-19 was also associated with an increase in the number of omissions, exhibiting both a dose effect [F(4,54)=8.8 p<0.001] and ITI effect [F(2,216)=15.1, p<0.001], without a significant dose×ITI interaction. In the premature response analysis, the higher 3 doses of Compound I-19 (0.1, 0.3 and 1.0 mg/kg) were associated with decreases in the number of premature responses that were most notable at the 10 sec ITI [i.e., dose effect [F(4,54)=33.1, p<0.001], effect of ITI [F(2,216)=225.8, p<0.001], dose×ITI interaction [F(8,216)=24.7 p<0.001]. A similar effect of Compound I-19 was observed when the number of timeout responses and magazine head entries were analyzed. Statistical analysis revealed the following: timeout responses: dose effect [F(4,54)=11.8 p<0.001], effect of ITI [F(2,216)=27.4, p<0.001], dose×ITI interaction [F(8,216)=10.0, p<0.001]; magazine head entries: dose effect [F(4,54)=26.8 p<0.001], effect of ITI [F(2,216)=55.3, p<0.001], dose×ITI interaction [F(8,216)=18.5, p<0.001]. There were no statistically significant effects on perseverative responses. Depending on the dose and ITI, Compound I-19 was also associated with modest increases in the response latencies, although the reward latencies and the number of trials completed were not significantly affected. The following statistical results for latencies were obtained: correct response latencies: dose effect [F(4,54)=4.1 p=0.003], effect of ITI [F(2,216)=13.1, p<0.001], dose×ITI interaction [F(8,216)=1.0, p=0.41]; incorrect response latencies: dose effect [F(4,54)=5.5 p=0.003] effect of ITI [F(2,216)=41.6, p<0.001], without a significant dose×ITI interaction

In summary, in the vITI version of the 5C-SRTT, Compound I-19 was associated with very modest, dose-dependent impairments in sustained attention, which are likely explained by its tendency to increase the number of omissions and response latencies. These observations may be indicative of mild sedation, locomotor impairment, or decreases in motivation. As observed in the vSD studies, Compound I-19 was also associated with decreases in impulsivity-like responding as evident by a decrease in the number of premature responses, timeout responses, and magazine head entries. These observations should be viewed with caution given the negative effects of Compound I-19 described above. It should be noted, however, that the 0.1 mg/kg dose of Compound I-19 did slightly reduce the number of premature responses and magazine head entries (most notably at the 10 sec ITI) without increasing the number of omissions or increasing the response latencies associated with correct responses. This observation may indicate that a narrow range of lower doses of Compound I-19 (˜0.1 mg/kg) may have therapeutic potential for reducing impulsivity-like behaviors. Since the 0.1 mg/kg dose of Compound I-19 was associated with modest increases in omissions in the vSD study, it is possible that the negative effects of Compound I-19 on motivation or locomotor activity may abate with repeated exposures.

### Conclusions

Nicotine produced modest but statistically significant improvements in an attentional measure (% hit) in the vSD version of the 5C-SRTT when a cohort of 20 rats were assessed.

Nicotine, however, was also associated with impairments of inhibitory response control as evident by an increase in the number of premature responses, timeout responses, magazine head entries, and decreases in response latencies.

Based on the modest nicotine effects on attention, the decision was made to include all 20 rats in the assessments of Compound I-4 and Compound I-19 in a sequential manner to improve statistical power.

Neither Compound I-4 nor Compound I-19 was associated with improvements in sustained attention (% hit or % accuracy) in the vSD or the vITI version of the 5C-SRTT, and doses of 0.3 mg/kg and above of both compounds were associated with increases in omissions and response latencies, which are indications of sedation, locomotor impairment, or decreased motivation.

Both Compound I-4 and Compound I-19 reduced premature responses, timeout responses, and magazine head entries in a dose-dependent manner, which is indicative of decreased impulsivity and compulsivity-like behavior. These observations should be viewed with caution, however, given the concomitant increases in omissions and response latencies noted above with most of the doses of the compounds, with the exception of the 0.1 mg/kg dose of Compound I-19 in the vITI study.

Conclusion—The doses of Compound I-4 and Compound I-19 that were evaluated did not appear to improve sustained attention in rats, but there was some evidence of dose-dependent improvements in inhibitory response control, especially with the 0.1 mg/kg dose of Compound I-19.

### Example 13. Dose-Response Evaluation of Compound I-19 in Mice Using SmartCage and Tail Suspension Test

### Introduction

The Locomotor Activity assessment is a simple means of establishing spontaneous locomotor activity, arousal, and willingness to explore in rodents. It is one of the most common rodent tests which can be used to test the effects of various medications on animal behavior. This study used a SmartCage™ system which is an automated non-invasive rodent behavioral monitoring system to monitor rodent home cage activity and behavior. (Xie et al, 2012, Animal Models of Acute Neurological Injuries II. Springer Protocols Handbooks).

The Tail Suspension Test is a mouse behavioral test useful for screening for potential antidepressant drugs. The tail suspension test is an experimental method used in scientific research to measure a state of being helpless in rodents, especially in mice. It is based on the observation that if a mouse is subjected to short term inescapable stress, then it will stop struggling. Immobility is quantified by measuring the amount of time lacking whole-body activity. A decrease in immobilized time (sec) following a treatment indicates that a drug might have antidepressant effects.

### Objective

The objective of this study was to evaluate the behavioral effects of acute treatment of the Compound A derivative, Compound I-19, on locomotor activity over 24 hr. using the Smart Cage and evaluate potential anti-depressant effects through the Tail Suspension Test. Results will be used for the selection of the dose range in a follow-up EEG sleep/wake study.

### Materials and Methods

### Animals

A cohort of 32 adult male mice was used for locomotor activity and a separate cohort of 40 male mice were used for tail suspension (C57BL/6, 7-8 weeks old; body weights 18-25 g, Charles River Laboratories). Upon arrival at the facility, animals were group-housed (5/cage) with access to food and water ad libitum. Animals were maintained on a 12/12-hour light/dark cycle in a temperature—(22±2° C.) and humidity—(approx. 50%) controlled room. Animals were numbered consecutively by tail ID marker in each home cage. Each home cage was identified by a colored ID card indicating the study number, sex, animal numbers and date of birth.

Experiments were conducted in accordance with the protocols approved by the Institutional Animal Care and Use Committee of AFASCI (Protocol #0220).

### Substances and Formulation

Test article Compound I-19 (Batch No. 115-108). Test article formulation was prepared fresh based on weight-to-volume in the vehicle on each dosing day. Compounds were weighed and then vehicle was added. The appearance of the formulation was slight pinky shade color without suspension. The test articles were formulated according to Table 145.

TABLE 145
Compound and Dosing Example 13. Administration of i.p. dosing at around 12-
1 pm circadian time.

| | | | Dose | Concentration | Volume | | |
|---|---|---|---|---|---|---|---|
| Substance | MW | FW | (mg/kg) | (mg/ml) | (mL/kg) | Route | Formulation |
| Vehicle | N/A | | N/A | N/A | 10 | IP | 4% DMSO, |
| Compound | 370.83 | 370.83 | 0.3 | 0.03 | 10 | IP | 30% |
| I-19 | | | | | | | PEG400, |
| | | | 1 | 0.1 | 10 | IP | 66% |
| | | | | | | | HPbCD |
| | | | 3 | 0.3 | 10 | IP | (30% in |
| | | | 10 | 1 | 10 | IP | H₂O) |

Animals (8/group) were injected (10 mL/kg, ip) 30 min prior to the TST. For locomotor activity monitoring, mice were injected (10 mL/kg, ip; around 12-1 pm) after a full 24 hr period of recordings were collected for baseline activity. For both locomotor activity recordings and TST, PV-03446 doses included 0.3, 1, 3, and 10 mg/kg.

Vehicle Selection: Based in the PK study this formulation results in a clear solution for 811 derivatives.

Dose Selection Rationale: 1) Compound I-19 is a Compound A derivative with similar in vitro binding profile as Compound A. 2) Compound A in sleep-EEG-EMG Sleep/Wake Studies in rats (Gruner et al, 2009) have a minimal effective dose of 0.3 mg/kg (i.p.) to change the wake and REM state. 3) According to the PK profile in mice (p.o. dosing), Compound I-19 has higher exposure of released Compound A than the equivalent Compound A dose. 4) The dog PK profile of Compound I-19 showed a significant amount of Compound I-19 in the dog plasma after p.o. dosing. 5) In this study Applicants use i.p. dosing to avoid the conversion to Compound A and evaluate the effect of the intact derivative in behavior. 6) In mice after p.o. dosing the Tₘₐₓ for the released Compound A is 20 min. and T_{1/2} of 52 minutes.

### Behavioral Methods

Locomotor Activity Monitoring: Mice underwent Smart Cage monitoring for 48 h. Baseline activities were measured during the first 24 h. After the first day, animals were administered one of three doses (PO) of test article and monitored for the next 24 hours. The animal's activity during the monitored hours was quantified using the parameters of distance travelled, velocity, time active, and rearing activity.

SmartCage: Individual mice were placed into freshly prepared home cages and recorded simultaneously using the SmartCage™ system and a video camera placed above the SmartCage™ system. The activity over a defined time block was automatically calculated by the CageScore™ program, which is the program associated with the SmartCage™ system (AfaSci, Inc) (Xie et al, 2012, Animal Models of Acute Neurological Injuries II. Springer Protocols Handbooks.).

Active wakefulness is defined as actively moving, rearing, and exploratory behaviors. Home cage activity variables include activity counts (i.e., the photocell beam breaks), locomotion (distance traveled and speed), and rearing counts. Activity counts are obtained from the lower horizontal infrared (IR) sensors (along the X and Y axis). Likewise, distance traveled in centimeters is obtained from the lower horizontal IR and calculated taking into account the animal's path. Locomotion is defined as moving a distance longer than the body-length of the test animal. The calculated distance traveled (at any given time period, called ‘block’, or ‘total measuring period’) and speed are the two main parameters for locomotor activity. The z-axis photocell beam break counts reflect the number of beam interruptions in the upper row of IR sensors and indicate rearing or climbing activity, which is considered to be part of exploratory behavior parameters. All IR data (beam break activity counts, locomotion, rearing, and rotations) are continuously recorded at a 4 Hz sampling rate. Absolute and percent time in a chosen block (or ‘time bin’) spent in this arousal state were also collected. Mice are most active within the first 1 hour when transferred to a fresh or new cage, after which their activity levels gradually decrease. Once their activity was stable, treatment with the test article was started.

Endpoints/parameters: SmartCage behavioral evaluation for a 48 hr. period, measured in 2-hr blocks: Activity Time; Distance traveled (and travel velocity); Rearing.

Scoring method: The SmartCage system uses an automatic scoring algorithm to calculate traveling distance and speed by integrating X and Y coordinates and time elapsed. The upper row of IR sensors detects the Z photobeam break account to indicate rearing activity.

Data analysis included calculating light (diurnal) and dark (nocturnal) time ratios. The nocturnal time ratio was calculated using the full 12 h of dark period (T34-46) following drug administration, which occurred at T28, and compared IR data with the identical time before drug treatment (i.e., T10-T22). The diurnal time ratio was calculated using a 2 h post-drug period (T30-32) and comparing it with the identical time before drug treatment (T6-8).

At the end of the experiment, mice were euthanized using CO2, followed by a secondary method. No tissue was collected.

Tail Suspension Test: A piezoelectric sensor operated by the SmartCage system was utilized and video recording was conducted for scoring. Once animals were acclimated to the testing room, the TST was initiated by placing the tail of the test mouse onto the piezoelectric sensor-board and hanging the mouse upside down. The recording period was initiated once the mouse was upside down and lasted for 6 min. At the end of the recording period, the mouse was removed from the recording device and returned to its home cage. The immobilized time, indicating depression-like behavior, was quantified using manual scoring of the video recording from 120 seconds to 360 seconds during the TST. The percent immobility time was calculated as (immobility time (seconds)/240 seconds)*100.

### Statistical Analysis

Basic statistical analysis was performed using GraphPad Prism and presented as mean±standard error of mean (SEM). The data were evaluated for normal distribution and homogeneous variances. Data that were not normally distributed were analyzed using alternative non-parametric tests.

Group differences found to have normal distribution were evaluated using a 1-way ANOVA followed by a Dunnett's Multiple Comparison Test to compare all treatment groups to the vehicle control. Statistical significance was set at p≤0.05.

### Results

There were no obvious increases in active time, nor enhancement in locomotion parameters of travel distance, velocity or rearing counts after dosing of Compound I-19. By averaging and comparing the nocturnaland diurnal activity ratios, there was no difference between pre- and post-treatment with Compound I-19 in any of the activity parameters measured (data not shown).

Tail Suspension Test: Compound I-19 produced a significant reduction in immobility (%) at all doses tested (0.3, 1.0, 3.0 and 10.0 mg/kg, IP) compared to the vehicle-treated animals. Compound I-19 resulted in a significant reduction in immobility (F4,35=4.71, p=0.004; Table 146). A post-hoc test revealed that mice were significantly more mobile across all doses of Compound I-19, with greater effects at 1.0 (p≤0.01) and 3.0 mg/kg (p≤0.01) when compared to the vehicle-treated group.

TABLE 146
Statistical Output for PV-03443 effects on immobility on the
TST. Control had a percent mobility of approximately 80%.

| | |
|---|---|
| One-way analysis of variance | |
| P value | 0.0038 |
| P value summary | ** |
| Are means signif. different? | Yes |
| (P < 0.05) | |
| Number of groups | 5 |
| F | 4.71 |
| R squared | 0.3499 |
| Bartlett's test for equal variances | |
| Bartlett's statistic (corrected) | 2.697 |
| P value | 0.6097 |
| P value summary | ns |
| Do the variances differ signif. | No |
| (P < 0.05) | |

| ANOVA Table | SS | df | MS |
|---|---|---|---|
| Treatment (between columns) | 4439 | 4 | 1110 |
| Residual (within columns) | 8247 | 35 | 235.6 |
| Total | 12690 | 39 | |

| Dunnett's Multiple | Mean | | Significant? | | 95% CI of |
|---|---|---|---|---|---|
| Comparison Test | Diff. | q | P < 0.05? | Summary | diff |
| Control | 19.9 | 2.592 | Yes | * | 0.2430 to |
| vs 0.3 | | | | | 39.55 |
| Control | 28.7 | 3.739 | Yes | ** | 9.045 to |
| vs 1.0 | | | | | 48.35 |
| Control | 28.96 | 3.773 | Yes | ** | 9.306 to |
| vs 3.0 | | | | | 48.61 |
| Control | 20.16 | 2.626 | Yes | * | 0.5035 to |
| vs 10.0 | | | | | 39.81 |

The differences between control and the treatments are found in the table.
*p ≤ 0.5;
**p ≤ 0.1.

### Conclusion

(Home cage activity exhibited a typical circadian rhythm, and Compound I-19 (0.3, 1.0, 3.0 and 10 mg/kg, IP) had no significant effect on the diurnal or nocturnal activity measures, including activity, rearing, velocity, and distance. On the TST, however, Compound I-19 significantly decreased immobility in mice by 26-38% depending on the dose when compared to vehicle, suggesting an anti-depressive effect.

### Example 14. Evaluation of the Effects of Compound I-19 on Sleep/Wake and EEG Parameters During the Active Period in the Orexin/tTA; TET-O/Diphtheria Toxin a Mouse Model of Narcolepsy

This report describes the results of a study on the effects of Compound 1-19 in a novel mouse model of narcolepsy. Using a repeated-measures, counter-balanced design, Compound 1-19 (0.3, 1 and 3 mg/kg, i.p.) and desipramine (Des; 5 mg/kg, p.o.) were tested for their effects on cataplexy, sleep/wake parameters, core body temperature (Tb), and locomotor activity (LMA) compared to a vehicle control (Veh; 4% DMSO, 30% PEG400, 66% HPBCD [30% in H₂O]) in orexin/tTA; Tet-O diphtheria toxin A mice (“DTA mice”). DTA mice are a conditional model of hypocretin/orexin neuron ablation and, as such, a novel mouse model of narcolepsy. EEG, EMG, Tb, and LMA were recorded via telemetry along with video recordings. Latency to sleep onset, hourly and cumulative sleep/wake amounts, and sleep/wake consolidation measures (bout duration and number of bouts per hour) were assessed for the 6 h period after injections that occurred just before onset of the dark period (at the start of Zeitgeber Hour [ZT]12). The EEG and EMG recordings were scored in 10 s epochs for waking (W), rapid eye movement sleep (REM), non-rapid eye movement sleep (NREM) and cataplexy (C). The EEG power spectrum (0.3-100 Hz, normalized) was calculated within state (W, NREM, REM and C)

Administration of Compound I-19 at the start of the active phase was followed by very strong suppression of C by all concentrations tested. REM was decreased in a dose-related manner with significant reductions observed following Compound I-19 at 1 and 3 mg/kg. Almost no effects on W or NREM time were found. However, some changes in EEG power during W and NREM were observed with increased low gamma during W and decreased alpha, beta and low gamma during NREM.

Strong suppression of C following Compound I-19 administration is an encouraging result for a possible narcolepsy therapeutic. Further studies are required to determine if the positive effects of Compound I-19 administration continue during the second half of the active phase or whether a “rebound” increase in C occurs.

As expected, Desipramine significantly increased the latency to REM, thereby validating the EEG biobehavioral assay used here. Desipramine also transiently decreased C for the first 2 h post-administration although this effect was not significant overall across the 6 h recording period; few other effects were observed on the parameters evaluated in this study.

### Introduction

The aim of this study was to investigate the dose-related effects of the test compound Compound I-19 in a novel, inducible mouse model of narcolepsy. Telemetry-based electroencephalography (EEG) was employed to determine whether Compound I-19 had a therapeutic effect on symptoms following the induction of the narcolepsy phenotype. EEG patterns, electromyograph (EMG), core body temperature (T_{b}), and gross locomotor activity (LMA) were collected and analyzed.

### Materials and Methods

### General Procedures

Animals were housed in a temperature-controlled recording room under a 12/12 light/dark cycle and had food and water available ad libitum. Room temperature (24±2° C.), humidity (50±20% relative humidity), and lighting conditions were monitored and recorded daily. Animals were inspected daily in accordance with AAALAC and SRI guidelines. All experimental procedures involving animals were approved by SRI International's Institutional Animal Care and Use Committee (IACUC, protocol 01026) and were in accordance with National Institutes of Health (NIH) guidelines.

Breeding of Orexin tTA; Tet-O Diphtheria Toxin A (“DTA”) Mice

A conditional model of hypocretin neuron ablation (orexin tTA; Tet-O diphtheria toxin A or “DTA mice”) was used in this study. In this model of narcolepsy, degeneration of hypocretin/orexin neurons occurs when the neurotoxic diphtheria toxin subunit A (DTA) protein is synthesized in these cells. Expression of the DTA transgene is controlled through the tetracycline transactivator (Tet-off) system. When doxycycline (Dox) is in the diet, it binds the tetracycline transactivator (tTA) which prevents tTA from binding to the Tet-O regulatory site upstream of the prepro-hypocretin DTA transgene. Removal of Dox from the diet enables tTA to bind Tet-O, thereby initiating transgene transcription. Because the Tet-O binding site is located exclusively in hypocretin/orexin (Hcrt) neurons, removal of dietary Dox (Dox(−)) results in accumulation of the neurotoxic DTA protein within these cells and degeneration of the Hcrt neurons occurs. After 6 weeks of Dox(−), >97% of Hcrt cells have degenerated and key features of narcolepsy, including wakefulness fragmentation and cataplexy, are readily evident.

Male DTA mice used in this study were bred at SRI and confirmed via genotyping. Mice were maintained on Dox+chow until approximately 14 weeks of age before entering a 6-week period of degeneration by removal of dietary Dox. Therefore, mice were approximately 20 weeks of age at the start of the experimental period. SRI staff was responsible for colony management, including daily monitoring, pairing, weaning, culling, and genotyping.

### Surgical Procedures

For this study, 8 male DTA mice were implanted with chronic recording devices for continuous recordings of EEG, EMG, Tb, and LMA via telemetry. Under isoflurane anesthesia (1-4%), the fur was shaved from the top of the head and from the midabdominal region. After the skin had been disinfected with chlorhexidine and sterile water, a ˜2.5 cm dorsal midline incision on top of the head was made. A subcutaneous pocket was blunt dissected along the left dorsal flank and then irrigated with 1.5-3.0 ml of sterile saline. A sterile miniature transmitter (HD-X02, Data Sciences Inc., St Paul, MN) was then inserted through the incision and placed into the subcutaneous pocket. The temporalis muscle was then retracted, and the skull was cauterized and thoroughly cleaned with a 3% hydrogen peroxide solution. Holes were drilled through the skull at the coordinates −2.0 mm AP from bregma and 2.0 mm ML and at −1 mm AP from lambda on the midline. The two biopotential leads that were used as EEG electrodes were inserted into the holes and affixed to the skull with dental acrylic. The two biopotential leads that were used as EMG electrodes were sutured into the neck musculature. The incision was closed with absorbable suture.

Animals were administered an anti-inflammatory (NSAID, e.g., meloxicam), an analgesic (opioid, e.g., buprenorphine), and saline during anesthetic recovery as recommended by LAMD veterinary staff. Animals were closely monitored during anesthetic recovery until they were ambulatory. Subsequently, they were carefully observed daily (˜5 min/day) until the incision was healed and the sutures were removed (1-2 wk post-surgery). NSAIDs were then administered once per day for 72 h and opioids once per day for 24 hours following surgery, or as needed for signs of pain. Signs of pain included decreased activity, decreased food/water consumption, weight loss, hunched posture, abnormal respiratory rate or character, chattering/grinding teeth, piloerection, changes in facial expression (e.g., position/status of ears, eyes, whiskers), failure to groom, or overgrooming.

Note: The data reported in this report were collected as part of a larger study in which another test compound was investigated. Three concentrations of the second compound were tested along with three concentrations of Compound I-19, Des and Veh (a total of 8 treatment conditions, of which 5 conditions are reported in this example).

### Experimental Design

Using a repeated-measures, counter-balanced design, Compound I-19 (0.3, 1 and 3 mg/kg, i.p.) and desipramine (Des; 5 mg/kg, p.o.) were administered at 10 ml/kg and tested for their effects on cataplexy, sleep/wake parameters, Tb, and LMA compared to a vehicle control (Veh; 4% DMSO, 30% PEG400, 66% HPBCD [30% in H₂O]) in DTA mice. Injections occurred just prior to the start of the dark period (before the start of Zeitgeber Hour [ZT]12). EEG, EMG, T_{b}, and LMA were recorded via telemetry along with video recordings using Ponemah 6.41 software (Data Sciences Inc., St Paul, MN). A minimum of 3 days elapsed between treatments and the 8 dosings per animal were completed over a 4-week period (only 5 treatments reported here, see Note above). Animals were acclimated to the handling procedures and were administered multiple 0.2 ml doses of water (p.o.) during the week before the first experimental day.

### Data Analysis

Following completion of the data collection, expert scorers (blinded to experimental condition) determined states of sleep and wakefulness for eight mice per group (N=8) by examining the recordings visually using NeuroScore software (Data Sciences Inc., St Paul, MN). The EEG and EMG recordings for the 6 h following dosing were scored in 10 s epochs for waking (W), rapid eye movement sleep (REM), non-rapid eye movement sleep (NREM) and cataplexy (C). Scored data were analyzed and expressed as time spent in each state per time bin. To determine whether any of the treatments affected behavioral state consolidation, the duration and number of bouts for each state were calculated in hourly bins. For W, NREM and REM, a “bout” consisted of a minimum of two consecutive 10 s epochs of a given state and ended with any single state change epoch. Latency to sleep onset was calculated from the time of each dosing to the first consecutive 60 s of sleep. Latency to REM onset was calculated from the time of each dosing to the first consecutive 30 s of REM. Cataplexy was scored according to the consensus criteria published by the International Working Group on Rodent Models of Narcolepsy (Scammell et al., 2009, SLEEP 2009; 32(1):111-116.). The EEG power spectrum (0.3-100 Hz) during W, NREM, REM and C were obtained offline with a fast Fourier transform algorithm on epochs without artifact. The EEG spectra were analyzed in 1 Hz bins and in standard frequency bands (delta: 0.5-4 Hz, theta: 4-9 Hz, alpha: 9-12 Hz, beta: 12-30 Hz, low gamma: 30-60 Hz, and high gamma: 60-100 Hz). For each individual animal, power was normalized to the average power per bin during the 6 h vehicle recording period. Hourly averages of T_{b} and LMA data were also analyzed.

Latency to NREM and REM, REM:NR ratios, and cumulative state data were analyzed using one-way repeated-measures analysis of variance (ANOVA). The remaining data analyses were by two-way repeated-measures ANOVA. For two-way repeated-measures ANOVA, we expected both a treatment effect and an effect that changed over time. Therefore, we analyzed for both a treatment effect (factor A) and a time (factor B) by treatment effect within each animal. When ANOVA indicated statistical significance, paired two-tailed t-tests were performed to determine specific differences. All statistics were performed in MATLAB using functions from mathworks.com/matlabcentral/fileexchange. For the EEG frequency data, statistical comparisons were performed only on the standard frequency bands.

### Results

### Compound I-19:

As used in this section, significant changes refer to a p-value of <0.05.

Administration of Compound I-19 was followed by significant dose-related decreases in REM and Compound I-19 at 1 and 3 mg/kg significantly increased the latency to REM and decreased REM:NR ratios. In the vehicle, 1 mg/kg and 3 mg/kg doses, latency to REM was about 100 min, 275 min, and 325 min, respectively. In the vehicle, 1 mg/kg, and 3 mg/kg REM:NR ratio was about 0.08, 0.2, and 0.1 respectively. Total C time significantly decreased following all three concentrations of Compound I-19 and REM time significantly decreased following Compound I-19 at the two highest doses (1 and 3 mg/kg). No significant effects on total W or NREM were observed.

REM and C significantly decreased overall following Compound I-19 at 1 and 3 mg/kg and C significantly decreased overall following all concentrations of Compound I-19. C also decreased significantly during ZT13-ZT14 and ZT16-ZT17 following Compound I-19 at 1 and 3 mg/kg and during ZT13 and ZT16-ZT17 following Compound I-19 at 0.3 mg/kg. No significant effects on hourly W or NREM were observed.

Cumulative REM significantly decreased overall and during every hour of the recording following PV-03443 at 1 and 3 mg/kg. Cumulative C decreased overall and during ZT13-ZT17 following every concentration of Compound I-19. Cumulative NREM increased during ZT16 following Compound I-19 at 3 mg/kg.

Both REM bout duration and the number of REM bouts significantly decreased overall following PV—03443 at 1 and 3 mg/kg. C bout duration significantly deceased overall following Compound I-19 at 0.3 mg/kg and the number of C bouts significantly decreased overall following all concentrations of PV—03443. No significant effects on bout durations or the number of bouts for W or NREM were found.

Some significant changes in EEG spectra were observed. W beta significantly decreased during ZT 16-ZT17 following Compound I-19 at 1 mg/kg, while W low gamma significantly increased during ZT13-ZT14 following all three concentrations of Compound I-19. NREM alpha and beta significantly decreased overall following Compound I-19 at 1 and 3 mg/kg. NREM alpha significantly decreased during ZT12-ZT17 following Compound I-19 at 3 mg/kg, during ZT13-ZT14 following PV—03443 at 1 mg/kg, and during ZT15 and ZT17 following Compound I-19 at 0.3 mg/kg. NREM low gamma significantly decreased during ZT13 and ZT16-ZT17 following Compound I-19 at 3 mg/kg, during ZT17 following Compound I-19 at 1 mg/kg, and during ZT16-ZT17 following Compound I-19 at 0.3 mg/kg. NREM low gamma significantly increased during ZT14 following Compound I-19 at 0.3 mg/kg. NREM high gamma significantly decreased during ZT16 following Compound I-19 at 0.3 mg/kg. Too little REM and C occurred during some hours of some conditions to perform statistical analyses of the EEG spectra during these states.

No significant effects on LMA or T_{b} were found.

### Desipramine:

As expected for the positive control in this assay, Des increased the latency to REM onset although the overall reduction in REM sleep did not reach significance (p=0.12). C decreased for the first 2 h post administration but was not significant overall. NREM bout duration significantly decreased during ZT14. No other significant effects were observed with Des treatment.

### Conclusions

Administration of Compound I-19 at the start of the active phase was followed by very strong suppression of C by all three concentrations tested. REM was also suppressed in a dose-related manner with significant reductions observed following Compound I-19 at 1 and 3 mg/kg. Almost no effects on W or NREM time were found. However, some changes in EEG power during W and NREM were observed. The primary EEG spectra effects observed were increased low gamma during W and decreased alpha, beta and low gamma during NREM. The physiological consequence of these changes are unclear at the present time.

Strong suppression of C following Compound I-19 even at the lowest concentration tested is an encouraging result for a possible narcolepsy therapeutic. Further studies are required to determine if the positive effects of Compound I-19 administration continue during the second half of the active phase or whether a “rebound” increase in C occurs.

### Example 15. Additional Analyses on the In Vivo Effect of Compound I-19 in Orexin-DTA Transgenic Mice

Applicant has performed a study using a mouse model of narcolepsy where a repeated measure, counter-balanced design was employed to administer eight (8) dosing conditions: one (1) positive control d-desipramine (5 mg/kg), 3 doses of the test compound Compound I-19 (0.3, 1 and 3 mg/kg), one (1) vehicle control (Veh; 4% DMSO, 30% PEG400, 66% HPßCD [30% in H₂O]) and three (3) concentrations of another test compound not described here. EEG, electromyography (EMG), subcutaneous body temperature (s.c. Tb) and gross locomotor activity (LMA) were collected via telemetry using a DSI data collection system (N=9 mice). Dose administration occurred just prior to light offset, the major activity period for nocturnal rodents such as mice. Only the first six (6) hours just following dose administration were scored and analyzed initially in Example 14. Based on the results, further analyses were warranted to determine the time course for the suppression of cataplexy. Since cataplexy was at baseline levels following the administration of desipramine at the end of the first 6 hours, no further analyses of this compound were performed. Therefore, for the second 6-h period further analyses were performed on four (4) conditions: Compound I-19 at 0.3, 1 and 3 mg/kg and Veh as a control.

Hourly and cumulative sleep/wake amounts, sleep/wake/cataplexy consolidation measures (bout duration and number of bouts per hour), Tb and LMA were assessed for the last 6 h of the dark period (Zeitgeber Hour [ZT]19-ZT24). Latency to sleep onset is not reported here since this measure is not informative for this period of the experiment. No experimental manipulation occurred at the start of the recording period report on in this report so sleep latency is not relevant. The EEG and EMG recordings were scored in 10 s epochs for wake (W), rapid eye movement sleep (REM), non-rapid eye movement sleep (NREM), and cataplexy (C). This summary report describes our findings.

Hourly, cumulative and total time in W, NREM or REM were not different from Veh following any concentration of Compound I-19. For C, levels remained decreased following Compound I-19 at 3 mg/kg. Hourly, cumulative and total time in C were significantly less following the high concentration of Compound I-19 (3 mg/kg) while C following the other two concentrations of Compound I-19 were not different from Veh. No differences in REM:NR ratios were observed.

No significant differences from Veh were found for W, NREM or REM bout durations or number of bouts. The significant decrease in C observed following Compound I-19 at 3 mg/kg occurred primarily via a decrease in the number of C bouts (at every timepoint 3 mg/kg Compound I-19 had fewer C bouts than the control. Some hourly time points for C bout duration were also significantly different following the high concentration of Compound I-19 with decreased C bout duration for ZT21 and increased C bout duration for ZT23 and ZT24.

No significant effects on s.c. T_{b} or gross LMA were observed.

### Summary Conclusions

Most of the effects on sleep/wake parameters described in the first report were absent during the second half of the dark period. The exception was for C following Compound I-19 at 3 mg/kg. C remained reduced compared to Veh primarily due to a reduced number of C bouts. However, C following Compound I-19 at 3 mg/kg was considerably greater during this period compared to ZT13-ZT18 suggesting levels were returning to baseline (see Example 14).

### Example 16. Evaluation of the Chronic Effects of Two Doses of Supernus Compound Compound I-19 in the Rat Five Choice Serial Reaction Time Task

The purpose of this study was to evaluate the effects of repeated administration of two lower doses of Compound I-19 (0.1 and 0.3 mg/kg) on sustained attention and inhibitory response control in an experimental animal model. The behavioral tasks utilized were three versions of the rat Five Choice Serial Reaction Time Task (5C-SRTT), the standard version of the task (STD) with a fixed stimulus duration and a fixed intertrial interval, the variable stimulus duration (vSD) and the variable intertrial interval (vITI) version. The principal readout parameters in each task were the % Hit and the % correct choices (measures of sustained attention) and three measures of inhibitory response control (premature, perseverative, and timeout responses), although a number of additional outcome measures were obtained (see under Methods and Results below).

### Materials and Methods

### Study Subjects

Twenty-four adult (approx. 10 months old) male Wistar rats (Envigo, Inc, Indianapolis, IN) were used in the study. These subjects were previously employed for evaluating the acute effects of Compound I-4 (i.e., Compound I-4) and Compound I-19 (i.e., Compound I-19) in the 5C-SRTT but were given a long drug-free washout period (approximately 2 months) before the chronic studies were initiated. Subjects were double housed in polycarbonate cages (45×30×18 cm) with corncob bedding in a vivarium of constant temperature (21-23° C.) and humidity (40-50%). Lighting was maintained on a 12-hr light-dark cycle (7:00 a.m.-7:00 p.m.) with free access to water and food up until two weeks before the initiation of the chronic Compound I-19 studies (see subsequent food restriction procedures below). All behavioral testing was performed during the light portion (9 a.m.-5 p.m.) of the light/dark cycle (Monday thru Friday). Animals were cared for in accordance with the Guide for the Care and Use of Laboratory Animals (U.S. National Institutes of Health) and the experimental protocols were approved by the Institutional Animal Care and Use Committee at Augusta University (Protocol #2010-0044).

### 5-Choice Serial Reaction Time (5C-SRTT) Procedures

The 5C-SRTT procedure (reviewed, Robbins, 2002. Psychopharmacology (Berl); 163(3-4):362-80) was conducted as we have described previously (Terry et al., 2014, Neurotoxicology and Teratology 44:18-29, 2014; Callahan et al., 2020, Neuropharmacology. August 15; 173:107994). One week prior to 5C-SRTT training and throughout testing rats were food restricted to approximately 85% of their age-dependent, free-feeding weights based upon Harlan Laboratories growth rate curves. Animals were trained in eight automated 5C-SRTT operant chambers (Med Associates, St. Albans, VT, USA), controlled by MedPC software (Med Associates). Briefly, each operant chamber was equipped with 5 apertures containing a photocell beam to detect nose pokes and a lamp (2.8 W) that could be illuminated randomly at varying durations. Food pellets (45 mg chow pellet, BioServ, Frenchtown, NJ, USA) were delivered automatically to a magazine, located on the opposite wall to the nose pokes, that was also equipped with a light that turned on to indicate that a pellet had been dispensed. The house-light remained on for the entire session unless an error or omission occurred. Training sessions began with the delivery of a food reward and retrieval triggered the first trial. After a 5 sec inter-trial-interval (ITI), a stimulus light within one of the five apertures was illuminated for a fixed duration (see below) and a single nose-poke into this opening during the signal illumination period or during the 5 sec limited hold period delivered a reward (correct response); a nose-poke into a non-illuminated aperture (incorrect response) resulted in a 5 sec time-out period and no food reward. Failure to respond within the 5 sec limited hold period (omission) also resulted in a time-out. Sessions ended when 40 minutes had lapsed or 100 trials had been completed.

For this study, subjects were re-trained 5 days per week until they re-established a stable performance criteria with a fixed (1.0 sec) stimulus duration and a fixed ITI (5 sec) of 70-75% accuracy, <20% omissions and completion of all 100 trials for 5 consecutive days. Upon meeting these performance criteria animals were trained for one additional week and two groups of subjects (N=12) were assembled and balanced according to their performance accuracy, specifically the % Hit evaluation, which takes into account correct responses as well as all incorrect responses including the number of omissions. Compound I-19 was subsequently evaluated in the STD version of the 5C-SRTT as well as in two other versions of the task, a randomized vSD version of the task and a randomized vITI version of the task. The vSD version of the task increases the attentional load while the vITI increases impulsivity-like behavior. The following outcome measures and behavioral domains were assessed: % Hit and % correct=sustained attention; omissions=attention/motivation; premature responses=impulsivity; perseverative responses=compulsivity; timeout responses=cognitive inflexibility; magazine head entries=appetitive motivation and compulsivity-like behavior; response latencies=information processing speed and/or motivation. For the STD version of the task, the stimulus duration (SD) was fixed at 1.0 sec and the ITI was fixed at 5 sec. In the vSD version of the task the following stimulus durations were presented in a pseudorandom manner: 0.3, 0.6, and 0.9 sec. In the vITI version, 2.5, 5.0, and 10.0 sec ITIs were employed in a pseudorandom manner. Performance parameters measured were: % Hit ((# correct/(# correct+# incorrect including omissions))×100) correct; % correct ((# correct/(# correct+# incorrect excluding omissions))×100); premature responses (# of nose-pokes into any aperture after trial initiation but before onset of the stimulus light); timeout responses (# of nose pokes into any aperture during a timeout period), perseverative responses (# of nose pokes occurring after the correct response had been made but before reward collection); # of food magazine head entries, omissions, and total trials completed. The latency to correct response (time taken from the onset of the nose poke light stimulus to making the correct nose poke response), latency to incorrect response (time taken from onset of nose poke light stimulus to making the incorrect nose poke response), and latency to reward (i.e., the magazine latency, time taken from making a correct nose poke response to retrieving the reward from the magazine) were also recorded.

### Drug Preparation and Administration

Compound I-19-Vehicle Formulation: Compound I-19 was dissolved in 4% DMSO+30% PEG400+66% HβCD (30%), then subsequently diluted to achieve the proper drug concentration for the injections, and the final concentrations of the diluents were: 0.8% DMSO, 6% PEG400, 93.2% HβCD (6%). The doses of Compound I-19 (0.1 and 0.3 mg/kg) were calculated based on the free base of each compound.

### Rationale for Doses

In a previous study, acute administration of Compound I-19 was associated with dose-dependent increases in task omissions and response latencies, which were indicators of locomotor impairments, sedative effects, or decreases in motivation. These effects were less notable with the lower doses, but not absent. Thus, this study sought to determine if the untoward effects of Compound I-19 abate with repeated administration and if positive effects on 5C-SRTT performance could be separated from the untoward effects. The route of administration selected was IP to avoid the conversion to Compound A and evaluate the effect of the intact derivative in behavior.

Table 147 below provides an outline of the weekly vehicle or drug administration protocol.

TABLE 147
Compound I-19 weekly vehicle or drug administration protocol.

| | Group 1 (N = 12) | | 5C | | Group 2 (N = 12) | | 5C |
|---|---|---|---|---|---|---|---|
| Week | Day | Drug | Task | Week | Day | Drug | Task |
| 1 | Re-Establish Baselines | | | 1 | Re-Establish Baselines | | |
| 2 | 1 | VEH | STD | 2 | 1 | VEH | STD |
| | 2 | VEH | vSD | | 2 | VEH | vSD |
| | 3 | VEH | STD | | 3 | VEH | STD |
| | 4 | VEH | vITI | | 4 | VEH | vITI |
| | 5 | VEH | STD | | 5 | VEH | STD |
| 3 | 1 | VEH | STD | 3 | 1 | VEH | STD |
| | 2 | VEH | vSD | | 2 | VEH | vSD |
| | 3 | VEH | STD | | 3 | VEH | STD |
| | 4 | VEH | vITI | | 4 | VEH | vITI |
| | 5 | VEH | STD | | 5 | VEH | STD |
| 4 | 1 | PV 0.1 mg/kg | STD | 4 | 1 | PV 0.3 mg/kg | STD |
| | 2 | PV 0.1 mg/kg | vSD | | 2 | PV 0.3 mg/kg | vSD |
| | 3 | PV 0.1 mg/kg | STD | | 3 | PV 0.3 mg/kg | STD |
| | 4 | PV 0.1 mg/kg | vITI | | 4 | PV 0.3 mg/kg | vITI |
| | 5 | PV 0.1 mg/kg | STD | | 5 | PV 0.3 mg/kg | STD |
| 5 | 1 | PV 0.1 mg/kg | STD | 5 | 1 | PV 0.3 mg/kg | STD |
| | 2 | PV 0.1 mg/kg | vSD | | 2 | PV 0.3 mg/kg | vSD |
| | 3 | PV 0.1 mg/kg | STD | | 3 | PV 0.3 mg/kg | STD |
| | 4 | PV 0.1 mg/kg | vITI | | 4 | PV 0.3 mg/kg | vITI |
| | 5 | PV 0.1 mg/kg | STD | | 5 | PV 0.3 mg/kg | STD |
| 6 | 1 | VEH | STD | 6 | 1 | VEH | STD |
| | 2 | VEH | vSD | | 2 | VEH | vSD |
| | 3 | VEH | STD | | 3 | VEH | STD |
| | 4 | VEH | vITI | | 4 | VEH | vITI |
| | 5 | VEH | STD | | 5 | VEH | STD |
| 7 | 1 | VEH | STD | 7 | 1 | VEH | STD |
| | 2 | VEH | vSD | | 2 | VEH | vSD |
| | 3 | VEH | STD | | 3 | VEH | STD |
| | 4 | VEH | vITI | | 4 | VEH | vITI |
| | 5 | VEH | STD | | 5 | VEH | STD |

### Statistical Analysis

All data were collated and entered into Microsoft Excel spreadsheets. The data were subsequently imported into SigmaPlot® 11.0 or JMVP® Pro version 16 for statistical analyses. Two and three factor analysis of variance (ANOVA) was used with repeated measures followed by Tukey's post-hoc tests. All results were expressed as the mean (±S.E.M.). Differences between means from experimental groups were considered significant at the p<0.05 level. The statistical F values and degrees of freedom associated with the ANOVA analyses are provided in the text of this report for the sustained attention and inhibitory outcome measures when the p values were statistically significant (p<0.05).

### Results

### Effects of Compound I-19 on Performance of the Standard (STD) Version of the 5C-SRTT

Sustained Attention—Applicant's first observation in this portion of the study was that the groups that were assembled and balanced based on their mean % Hit after baselines were re-established, maintained this level of consistent accuracy. The second observation was that Compound I-19 did not significantly affect either the % Hit or the % correct [i.e. the effects of group, treatment, and the group×treatment interactions were not statistically different (i.e., p>0.05) in any portion of the study].

Inhibitory Response Control—Three measures of inhibitory response control were taken: the number of premature, timeout, and perseverative responses, respectively. Surprisingly, even though Groups 1 and 2 were closely matched for % Hit, Group 2 was typically associated with modest impairments of inhibitory response control (statistically significant compared to group 1 during the post-drug period for premature responses, but also evident in the number of timeout and perseverative measures, see below). The following statistical results were obtained for premature responses: group effect [F(1,22)=4.8, p=0.039]; treatment effect [F(2,44)=28.0, p<0.001]; and group×treatment interaction [F(2,44)=3.8, p=0.030]. Post hoc analysis indicated that Compound I-19 treatment reduced the number of premature responses compared to pre- and post-drug conditions. For timeout responses, only the effect of treatment was statistically significant [F(2,44)=3.7, p=0.033], without a significant effect of group or group×treatment interaction. Interestingly, in Group 2, the number of timeout responses was significantly reduced in both the drug and post-drug period compared to the pre-drug period. For perseverative responses, there were increases across all treatment periods in Group 2; however, the effects of group, treatment, and the group×treatment interactions were not statistically different (p>0.05).

Effects of Compound I-19 on Performance of the Variable Stimulus Duration (vSD) Version of the 5C-SRTT

Sustained Attention—Two measures of sustained attention were taken: % Hit and the % correct, respectively in the vSD version of the task. The following statistical results were obtained for % Hit: effect of treatment [F(2,2)=19.6, p<0.001] and stimulus duration [F(2,2)=135.2, p<0.001]. All other effects of group and the interactions of group, treatment, and stimulus duration were not significant. Post hoc analysis indicated that both doses of Compound I-19 were associated with a modest decrease in accuracy that was limited to the 0.6 sec stimulus duration. For % correct, the only statistically significant finding was the expected effect of stimulus duration, [F(2,2)=178.4, p<0.001].

Inhibitory Response Control—Three measures of inhibitory response control were taken: the number of premature, timeout, and perseverative responses, respectively in the vSD version of the task. The following statistical results were obtained for premature responses: group effect [F(1,2)=7.9, p<0.01]; treatment effect [F(2,2)=61.8, p<0.001]; and group×treatment interaction [F(2,2)=5.4, p<0.01]. All other interactions were insignificant. Post hoc analysis indicated that Compound I-19 treatment reduced the number of premature responses compared to post-drug conditions. As observed in the STD version of the task, even though Groups 1 and 2 were closely matched for % Hit, Group 2 typically was associated with increases in premature responses, most notably in the post-drug period. For timeout responses, the following statistical results were obtained: effect of treatment [F(2,2)=6.4, p=0.002] and stimulus duration [F(2,2)=12.7, p<0.001]. The effects of group and all other interactions between group, treatment, and stimulus duration were not significant. In post hoc analyses, there were overall differences between the treatment groups (i.e., drug was statistically different than pre-drug and post drug), and the 0.3 sec SD was statistically different from the 0.6 and 0.9 sec SD, but all other pairwise comparisons were not statistically significant. For perseverative responses there was a significant effect of stimulus duration [F(2,2)=4.0, p=0.02], and while there were increases in perseverative responses across all treatment periods in Group 2, the effects of group, treatment, and all relevant interactions were not statistically different, p>0.05).

Effects of Compound I-19 on Performance of the Variable Intertrial Interval (vITI) version of the 5C-SRTT

Sustained Attention—Two measures of sustained attention were taken, % Hit and the % correct, respectively in the vITI version of the task. The following statistical results were obtained for % Hit: effect of treatment [F(2,2)=24.0, p<0.001]; ITI [F(2,2)=29.0, p<0.001]; and treatment×ITI [F(4,4)=3.4, p=0.01]. All other effects of group and the interactions of group, treatment, and ITI were not significant. Post hoc analysis indicated that both doses of Compound I-19 were associated with a modest decrease in accuracy that was limited to the 2.5 sec ITI. For % correct, the following statistical results were obtained: effect of group [F(1,2)=4.6, p=0.04] and treatment [F(2,2)=3.5, p=0.03]. Post hoc analysis indicated a significant overall difference between drug vs pre-drug conditions. All other effects and the interactions of group, treatment, and ITI were not significant.

Inhibitory Response Control—Three measures of inhibitory response control were taken: the number of premature, timeout, and perseverative responses, respectively in the vITI version of the task. The following statistical results were obtained for premature responses: treatment effect [F(2,2)=24.4, p<0.001]; ITI [F(2,2)=838.1, p<0.001]; group×ITI [F(2,2)=6.0, p=0.003]; and treatment×ITI [F(4,4)=20.5, p<0.001]. Post hoc analysis indicated that the number of premature responses was markedly elevated at the 10 sec ITI (p<0.001 vs the 2.5 and 5 sec ITI) at all treatment periods. In addition, both doses of Compound I-19 reduced the number of premature responses compared to both the pre- and post-drug conditions at the 10 sec ITI. The other statistically significant finding was the comparison of Group 1 and 2 during the post-drug period at the 10 sec ITI. For timeout responses, the following statistical results were obtained: group effect [F(1,2)=5.7, p=0.02]; treatment effect [F(2,2)=10.5, p<0.001]; ITI [F(2,2)=147.61, p<0.001]; group×ITI [F(2,2)=14.7, p<0.001]; and treatment×ITI [F(4,4)=8.6, p<0.001]. Post hoc analysis indicated that the number of timeout responses was elevated at the 10 sec ITI (p<0.001 vs the 2.5 and 5 sec ITI) at all treatment periods. In addition, both doses of Compound I-19 reduced the number of timeout responses compared to both the pre- and post-drug conditions at the 10 sec ITI. The other statistically significant findings were the comparison of Group 1 and 2 during the pre- and post-drug period at the 10 sec ITI. For perseverative responses, there were increases across all treatment periods in Group 2; however, the effects of group, treatment, stimulus duration, and all relevant interactions were not statistically different (p>0.05).

Additional Post Hoc Analyses of the vITI Data

Upon visual inspection of the data during the vITI sessions, there appeared to be differences in the response to Compound I-19, specifically decreases in the number of premature and timeout responses in week two versus week one in subjects administered the lower (0.1 mg/kg) dose. Accordingly, we performed additional analyses of the vITI data and compared the responses to Compound I-19 during week 1 of the drug exposure period to week 2 in both groups. For premature responses the following statistical results were obtained, group×session (week) effect [F(1,22)=14.1, p=0.001]. The effects of group and session were not statistically significant. Post hoc analyses indicated that in group 1 (0.1 mg/kg dose of Compound I-19) there were fewer premature responses during week 2 versus week 1. For timeout responses, the following statistical results were obtained, group×session (week) effect [F(1,22)=14.5, p=0.001]. The effects of group and session were not statistically significant. Post hoc analyses indicated that in group 1 (0.1 mg/kg dose of Compound I-19) there were fewer timeout responses during week 2 versus week 1. These significant group differences across session suggest that continued treatment with Compound I-19 (0.1 mg/kg) elicited stronger effects on inhibitory control measures than during the initial treatment period.

### Additional Outcome Measures Related to Compound I-19 in Different Versions of the 5C-SRTT.

Table 148 provides a variety of additional outcome measures that were assessed in each of the three versions of the 5C-SRTT. The values in the table represent the mean±SEM for all of the measured values irrespective of SD or ITI. In each case where a significant difference is identified (e.g., *+ϕ), there were group, treatment, or group×treatment interactions followed by a post hoc Tukey's test.

Omissions—The fourth column in Table 148 provides the mean total number of omissions associated with each group and treatment period in the three versions of the 5C-SRTT task. In all cases, the Treatment Period was associated with modest, but statistically significant, elevations in the number of omissions compared to either the pre-drug or post-drug period, or both the pre- and post-drug periods (p<0.05).

Magazine Head Entries—The fifth column in Table 148 provides the mean total number of magazine head entries. The only significant differences noted here were the within Group comparisons (drug vs post-drug) in the vITI version of the 5C-SRTT. Interestingly, in both groups 1 and 2, there were increases in magazine head entries in the post-drug (vehicle washout) period compared to the drug (exposure) period.

Latency to Incorrect Response—The seventh column in Table 148 provides the mean latencies to the incorrect responses. Again, the most notable findings were the within Group comparisons where the post-drug (washout) period was associated with a decrease in the incorrect response latencies compared to drug exposure conditions.

Reward Latencies—The eighth column in Table 148 provides the mean reward latencies. The most notable findings were the within Group comparisons where the post-drug (washout) period was associated with a modest decrease in reward latencies compared to drug exposure conditions. One exception was noted for Group 2 in the vSD task where drug exposure was associated with a modest elevation in the reward latencies compared to both pre-drug and post-drug conditions.

Trials Completed—The ninth column in Table 148 provides the mean trials completed. Here the only significant findings were a decrease in the number of trials completed in group 2 compared to group 1 under pre-drug conditions in the vITI task and an increase in trials completed in group 2 (in the within group comparison) for drug vs pre-drug conditions.

Latency to Correct Response—The sixth column in Table 148 provides the mean latencies to the correct responses. The most notable results in this analysis were the within Group comparisons where the post-drug (washout) period was associated with a decrease in the correct response latencies compared to drug and in a few cases the pre-drug conditions. An exception is the modest increase in response latencies under drug conditions compared to both pre- and post-drug conditions in the vITI task.

TABLE 148
Effects of Chronic Compound I-19 Treatment on 5C-SRTT Performance in Rats.

| | | | | Magazine | | | | |
|---|---|---|---|---|---|---|---|---|
| | Treatment | | | Head | Latency | Latency | Reward | Trials |
| Group | Period | Test | Omissions | Entries | (Correct) | (Incorrect) | Latency | Completed |
| 1 | Pre-Drug | STD | 5.29 ± 0.95 | 164.69 ± 9.98 | 0.91 ± 0.03 | 2.05 ± 0.11 | 3.00 ± | 98.42 ± 1.18 |
| | Exp | | | | | | 0.43 | |
| | Drug Exp | | 9.43 ± | 150.65 ± 9.46 | 0.94 ± | 2.15 ± | 3.53 ± | 96.99 ± 2.12 |
| | | | 1.19*⁺ | | 0.03⁺ | 0.11⁺ | 0.81 | |
| | Post-Drug | | 4.46 ± 0.81 | 168.06 ± 13.60 | 0.87 ± 0.03 | 1.86 ± 0.09 | 2.64 ± | 97.65 ± 2.23 |
| | Exp | | | | | | 0.40 | |
| 2 | Pre-Drug | STD | 6.08 ± 2.31 | 179.83 ± 19.71 | 0.89 ± 0.03 | 2.04 ± 0.12 | 2.95 ± | 100.00 ± |
| | Exp | | | | | | 0.49 | 0.00 |
| | Drug Exp | | 8.10 ± 2.32⁺ | 183.89 ± 32.19 | 0.92 ± | 2.28 ± | 4.08 ± | 98.44 ± 0.95 |
| | | | | | 0.03⁺ | 0.13⁺ | 1.03⁺ | |
| | Post-Drug | | 3.26 ± 0.95 | 211.72 ± 32.77 | 0.78 ± | 1.61 ± 0.07 | 2.27 ± | 100.00 ± |
| | Exp | | | | 0.02^{ϕ} | | 0.17 | 0.00 |
| 1 | Pre-Drug | vSD | 9.17 ± 1.54 | 184.08 ± 13.70 | 0.91 ± 0.04 | 1.95 ± 0.08 | 2.37 ± | 100.00 ± |
| | Exp | | | | | | 0.20 | 0.00 |
| | Drug Exp | | 15.79 ± | 168.21 ± 10.60 | 0.99 ± | 2.17 ± | 2.44 ± | 94.17 ± 2.75 |
| | | | 1.42*⁺ | | 0.04⁺ | 0.08⁺ | 0.19 | |
| | Post-Drug | | 8.12 ± 1.63 | 189.79 ± 14.47 | 0.86 ± 0.03 | 1.90 ± 0.09 | 2.13 ± | 97.79 ± 2.21 |
| | Exp | | | | | | 0.21 | |
| 2 | Pre-Drug | vSD | 8.63 ± 2.33 | 221.46 ± 35.34 | 0.89 ± 0.04 | 1.98 ± 0.09 | 2.48 ± | 100.00 ± |
| | Exp | | | | | | 0.29 | 0.00 |
| | Drug Exp | | 14.08 ± | 208.46 ± 28.88 | 0.91 ± | 2.21 ± | 2.99 ± | 98.29 ± 0.84 |
| | | | 2.19*⁺ | | 0.03⁺ | 0.08⁺ | 0.19*⁺ | |
| | Post-Drug | | 5.79 ± 1.35 | 245.17 ± 33.79 | 0.76 ± | 1.65 ± 0.08 | 2.11 ± | 100.00 ± |
| | Exp | | | | 0.02^{ϕ} | | 0.18 | 0.00 |
| 1 | Pre-Drug | vITI | 10.96 ± 0.98 | 298.71 ± 26.52 | 0.90 ± 0.02 | 2.04 ± 0.14 | 2.22 ± | 93.38 ± 3.12 |
| | Exp | | | | | | 0.28 | |
| | Drug Exp | | 17.04 ± | 236.13 ± | 0.99 ± | 2.27 ± 0.12 | 2.77 ± | 91.83 ± 4.15 |
| | | | 1.45*⁺ | 22.19⁺ | 0.03*⁺ | | 0.31 | |
| | Post-Drug | | 9.63 ± 1.15 | 318.71 ± 34.88 | 0.90 ± 0.03 | 2.03 ± 0.09 | 2.13 ± | 85.62 ± 4.17 |
| | Exp | | | | | | 0.19 | |
| 2 | Pre-Drug | vITI | 8.75 ± 1.46 | 323.08 ± 49.42 | 0.86 ± 0.03 | 2.00 ± 0.10 | 3.74 ± | 77.33 ± |
| | Exp | | | | | | 1.32 | 5.95^{#} |
| | Drug Exp | | 13.42 ± | 268.17 ± | 0.97 ± | 2.26 ± | 3.21 ± | 87.50 ± |
| | | | 2.96*⁺ | 39.13⁺ | 0.05*⁺ | 0.12⁺ | 0.32⁺ | 4.75* |
| | Post-Drug | | 6.75 ± 1.44 | 415.04 ± 70.50 | 0.81 ± 0.03 | 1.82 ± 0.10 | 2.06 ± | 78.46 ± 4.47 |
| | Exp | | | | | | 0.20 | |

Group 1 = Compound I-19 0.1 mg/kg treatment group;
Group 2 = Compound I-19 0.3 mg/kg treatment group. Table values represent the mean ± SEM. Significant differences are indicated by *p <0.05 drug vs pre-drug; ⁺p <0.05 drug vs post-drug; ^{ϕ}p <0.05 post-drug vs pre-drug; ^{#}p <0.05 group 1 vs 2.

### Summary and Conclusions

Neither dose of Compound I-19 was associated with improvements in sustained attention (% hit or % correct) in any version of the task. In a couple of cases, both doses of Compound I-19 were associated with a modest decrease in accuracy (i.e., in the vSD task % Hit at the 0.6 sec SD, and in the vITI task % Hit at the 2.5 sec ITI).

Both doses of Compound I-19 were associated with significant improvements of inhibitory response control (i.e., a decrease in the number of premature responses and timeout responses), primarily in the vITI version of the 5C-SRTT, which is indicative of decreased impulsivity and compulsivity-like behavior.

Both doses of Compound I-19 were associated with modest increases in the number of omissions, and in a few cases, the compounds were associated with very slight increases in response and reward latencies. Given these very modest effects, the doses of Compound I-19 that we evaluated do not appear to be associated with significant impairments in appetitive motivation, a decrease in information processing speed, sedation, or locomotor impairment.

Surprisingly, although Groups 1 and 2 were carefully assembled and balanced based on the % Hit measure, which takes into account both accuracy (# of correct responses) and all incorrect responses, which includes the number of omissions, Group 2 demonstrated some impairments of inhibitory response control (i.e., increases in premature, timeout, and perseverative responses compared to group 1) across the study. In some cases, these impairments were attenuated by Compound I-19 (e.g., in the STD version of the task in the premature and timeout response measurements).

Collectively, the results of this study indicate that the doses Compound I-19 that were evaluated do not appear to improve sustained attention in rats, but they were associated with significant improvements of inhibitory response control. Given the modest increase in omissions and a few cases where response and reward latencies were slightly increased, the latter conclusion should be viewed with caution, however.

### Example 17. Evaluation of the Effects of Compound I-4 ON Sleep/Wake and Eeg Parameters During the Active Period in the Orexin/tTA; Tet-O/Diphtheria Toxin a Mouse Model of Narcolepsy

This report describes the results of a study of the effects of Compound I-4 in a novel mouse model of narcolepsy. Using a repeated-measures, counter-balanced design, Compound I-4 (0.3, 1 and 3 mg/kg, p.o.) and desipramine (Des; 5 mg/kg, p.o.) were tested for their effects on cataplexy, sleep/wake parameters, core body temperature (Tb), and locomotor activity (LMA) compared to a vehicle control (Veh; 4% DMSO, 30% PEG400, 66% HPßCD [30% in H₂O]) in orexin tTA; Tet-O diphtheria toxin A mice (“DTA mice”). DTA mice are a conditional model of hypocretin/orexin neuron ablation and, as such, a novel mouse model of narcolepsy. EEG, EMG, Tb, and LMA were recorded via telemetry along with video recordings. Latency to sleep onset, hourly and cumulative sleep/wake amounts, and sleep/wake consolidation measures (bout duration and number of bouts per hour) were assessed for 6 h after injections that occurred just before onset of the dark period (at the start of Zeitgeber Hour [ZT]12). The EEG and EMG recordings were scored in 10 s epochs for waking (W), rapid eye movement sleep (REM), non-rapid eye movement sleep (NREM) and cataplexy (C). The EEG power spectrum (0.3-100 Hz, normalized) was calculated within state (W, NREM, REM and C).

Administration of Compound I-4 at the start of the active phase was followed by a suppression of REM and a strong, dose-related suppression of C. At the highest concentration, Compound I-4 significantly increased the consolidation of W without affecting time spent in W or NREM. Very few other effects were observed following Compound I-4 including on the EEG power spectra.

Strong suppression of C together with increased W consolidation following administration at the start of the active phase is an encouraging result for a possible narcolepsy therapeutic.

Further studies are required to determine if these positive effects of Compound I-4 administration continue during the second half of the active phase or if a “rebound” increase in C occurs.

As expected, Desipramine significantly increased the latency to REM, thereby validating the EEG biobehavioral assay used here. Desipramine also transiently decreased C for the first 2 h post administration; few other effects were observed on the parameters evaluated in this study.

### Introduction

The aim of this study was to investigate the dose-related effects of the Compound I-4 in a novel inducible mouse model of narcolepsy. Telemetry-based electroencephalography (EEG) was employed to determine whether Compound I-4 had a therapeutic effect on symptoms following the induction of the narcolepsy phenotype. EEG patterns, electromyograph (EMG), core body temperature (Tb), and gross locomotor activity (LMA) were collected and analyzed.

### Materials and Methods

### General Procedures

Animals were housed in a temperature-controlled recording room under a 12/12 light/dark cycle and had food and water available ad libitum. Room temperature (24±2° C.), humidity (50±20% relative humidity), and lighting conditions were monitored and recorded daily. Animals were inspected daily in accordance with AAALAC and SRI guidelines. All experimental procedures involving animals were approved by SRI International's Institutional Animal Care and Use Committee (IACUC Protocol 01026) and were in accordance with National Institutes of Health (NIH) guidelines.

Breeding of Orexin tTA; Tet-O Diphtheria Toxin a (“DTA”) Mice

A conditional model of hypocretin neuron ablation (orexin tTA; Tet-O diphtheria toxin A or “DTA mice”) was used in this study. In this model of narcolepsy, degeneration of hypocretin/orexin neurons occurs when the neurotoxic diphtheria toxin subunit A (DTA) protein is synthesized in these cells. Expression of the DTA transgene is controlled through the tetracycline transactivator (Tet-off) system. When doxycycline (Dox) is in the diet, it binds the tetracycline transactivator (tTA) which prevents tTA from binding to the Tet-O regulatory site upstream of the prepro-hypocretin DTA transgene. Removal of Dox from the diet enables tTA to bind Tet-O, thereby initiating transgene transcription. Because the Tet-O binding site is located exclusively in hypocretin/orexin (Hcrt) neurons, removal of dietary Dox (Dox(−)) results in accumulation of the neurotoxic DTA protein within these cells and degeneration of the Hcrt neurons occurs. After 6 weeks of Dox(−), >97% of Hcrt cells have degenerated and key features of narcolepsy, including wakefulness fragmentation and cataplexy, are readily evident.

Male DTA mice used in this study were bred at SRI and confirmed via genotyping. Mice were maintained on Dox+chow to approximately 14 weeks of age before entering a 6 week period of degeneration by removal of dietary Dox. Therefore, mice were approximately 20 weeks of age at the start of the experimental period. SRI staff was responsible for colony management, including daily monitoring, pairing, weaning, culling, and genotyping.

### Surgical Procedures

For this study, 8 male DTA mice were implanted with chronic recording devices for continuous recordings of EEG, EMG, Tb, and LMA via telemetry. Under isoflurane anesthesia (1-4%), the fur was shaved from the top of the head and from the midabdominal region. After the skin had been disinfected with chlorhexidine and sterile water, a ˜2.5 cm dorsal midline incision on top of the head was made. A subcutaneous pocket was blunt dissected along the left dorsal flank, and then irrigated with 1.5-3.0 ml of sterile saline. A sterile miniature transmitter (HD-X02, Data Sciences Inc., St Paul, MN) was then inserted through the incision and placed into the subcutaneous pocket. The temporalis muscle was then retracted, and the skull was cauterized and thoroughly cleaned with a 3% hydrogen peroxide solution. Holes were drilled through the skull at the coordinates −2.0 mm AP from bregma and 2.0 mm ML and at −1 mm AP from lambda on the midline. The two biopotential leads that were used as EEG electrodes were inserted into the holes and affixed to the skull with dental acrylic. The two biopotential leads that were used as EMG electrodes were sutured into the neck musculature. The incision was closed with absorbable suture.

Animals were administered an anti-inflammatory (NSAID, e.g., meloxicam), an analgesic (opioid, e.g., buprenorphine), and saline during anesthetic recovery as recommended by LAMD veterinary staff. Animals were closely monitored until they were ambulatory. Subsequently, they were carefully observed daily (˜5 min/day) until the incision was healed and the sutures were removed (1-2 wk post-surgery). NSAIDs were then administered once per day for 72 h and opioids once per day for 24 hours following surgery, or as needed for signs of pain. Signs of pain included decreased activity, decreased food/water consumption, weight loss, hunched posture, abnormal respiratory rate or character, chattering/grinding teeth, piloerection, changes in facial expression (e.g., position/status of ears, eyes, whiskers), failure to groom, or overgrooming in a study.

Note: The data reported in this report were collected as part of a larger study in which another test compound was investigated. Three concentrations of the second compound were tested along with three concentrations of Compound I-4, desipramine and vehicle (a total of 8 treatment conditions, of which 5 conditions are reported here).

### Experimental Design

Using a repeated-measures, counter-balanced design Compound I-4 (0.3, 1 and 3 mg/kg, p.o.) and desipramine (Des; 5 mg/kg, p.o.) were administered at 10 ml/kg and tested for their effects on cataplexy, sleep/wake parameters, Tb, and LMA compared to a vehicle control (Veh; 4% DMSO, 30% PEG400, 66% HPBCD [30% in H₂O]) in DTA mice. Injections occurred just prior to the start of the dark period (before the start of Zeitgeber Hour [ZT]12). EEG, EMG, Tb, and LMA were recorded via telemetry along with video recordings using Ponemah 6.41 software (Data Sciences Inc., St Paul, MN). A minimum of 3 days elapsed between treatments and the 8 dosings per animal were completed over a 4-week period (only 5 treatments reported here, see Note above). Animals were acclimated to the handling procedures and were administered multiple 0.2 ml water dosing (p.o.) during the week before the first experimental day.

### Data Analysis

Following completion of the data collection, expert scorers (blinded to experimental condition) determined states of sleep and wakefulness for eight mice per group (N=8) by examining the recordings visually using NeuroScore software (Data Sciences Inc., St Paul, MN). The EEG and EMG recordings for 6 h following dosing were scored in 10 s epochs for waking (W), rapid eye movement sleep (REM), non-rapid eye movement sleep (NREM) and cataplexy (C). Scored data were analyzed and expressed as time spent in each state per time bin. To determine whether any of the treatments affected behavioral state consolidation, the duration and number of bouts for each state were calculated in hourly bins. For W, NREM and REM, a “bout” consisted of a minimum of two consecutive 10 s epochs of a given state and ended with any single state change epoch. Latency to sleep onset was calculated from the time of each dosing to the first consecutive 60 s of sleep. Latency to REM onset was calculated from the time of each dosing to the first consecutive 30 s of REM. Cataplexy was scored according to the consensus criteria published by the International Working Group on Rodent Models of Narcolepsy (Scammell et al., 2009, SLEEP; 32(1):111-116.). The EEG power spectrum (0.3-100 Hz) during W, NREM, REM, and C were obtained offline with a fast Fourier transform algorithm on epochs without artifact. The EEG spectra were analyzed in 1 Hz bins and in standard frequency bands (delta: 0.5-4 Hz, theta: 4-9 Hz, alpha: 9-12 Hz, beta: 12-30 Hz, low gamma: 30-60 Hz and high gamma: 60-100 Hz). For each individual animal, power was normalized to the average power per bin during the 6 h vehicle recording period. Hourly averages of Tb and LMA data were also analyzed.

Latency to NREM and REM, REM:NR ratios, and cumulative state data were analyzed using one-way repeated-measures analysis of variance (ANOVA). The remaining data analyses were by two-way repeated-measures ANOVA. For two-way repeated-measures ANOVA, we expected both a treatment effect and an effect that changed over time. Therefore, we analyzed for both a treatment effect (factor A) and a time (factor B) by treatment effect within each animal. When ANOVA indicated statistical significance, paired two-tailed t-tests were performed to determine specific differences. All statistics were performed in MATLAB using functions from mathworks.com/matlabcentral/fileexchange. For the EEG frequency data, statistical comparisons were performed only on the standard frequency bands.

### Results

Compound I-4: Administration of Compound I-4 was followed by dose-related decreases in REM and C. Compound I-4 at 3 mg/kg significantly (p<0.05) increased the latency to REM and decreased REM:NR ratios. Total REM and C time significantly (p<0.05) decreased following Compound I-4 at the two highest doses (1 and 3 mg/kg) without any significant effects on total W or NREM.

REM and C decreased overall following Compound I-4 at 1 and 3 mg/kg (FIG. 3). C also decreased during ZT12-ZT14 and ZT16-ZT17 following Compound I-4 at 3 mg/kg and during ZT12-ZT14 and ZT16 following Compound I-4 at 1 mg/kg. No significant effects on hourly W or NREM were observed.

Cumulative REM significantly(p<0.05) decreased overall and during every hour of the recording following PV—03396 at 3 mg/kg. Cumulative REM also significantly(p<0.05) decreased during ZT16-ZT17 following PV—03396 at 1 mg/kg. Cumulative C significantly (p<0.05) decreased overall and during every hour of the recording following Compound I-4 at 1 and 3 mg/kg. No significant effects on cumulative W or NREM were observed.

Although no effects on cumulative time in W were observed (see above), Compound I-4 at 3 mg/kg consolidated W. W bout duration significantly (p<0.05) increased overall and the number of W bouts decreased overall following Compound I-4 at 3 mg/kg. REM bout duration significantly (p<0.05) decreased overall following Compound I-4 at 1 and 3 mg/kg and the number of REM bouts significantly (p<0.05) decreased overall following Compound I-4 at 3 mg/kg. The number of C bouts significantly (p<0.05) decreased overall following Compound I-4 at 1 and 3 mg/kg. NREM bout duration significantly (p<0.05) decreased during ZT14 following Compound I-4 at 0.3 mg/kg.

Only a small number of significant changes in EEG spectra were observed. For example: NREM alpha and beta decreased overall following Compound I-4 at 1 mg/kg. NREM alpha decreased during ZT13-ZT15 following Compound I-4 at 1 and 3 mg/kg. NREM low gamma decreased during ZT13-ZT15 following Compound I-4 at 1 mg/kg and during ZT15 following PV—03396 at 3 mg/kg. NREM high gamma decreased during ZT15 following Compound I-4 at 3 mg/kg and during ZT17 following Compound I-4 at 0.3. No significant effects on W EEG spectra were found. Too little REM and C occurred during some hours of some conditions to perform statistical analyses of the EEG spectra during these states.

LMA increased significantly overall following Compound I-4 at 3 mg/kg. No significant effects on Tb were found.

Desipramine: As expected for the positive control in this assay, Des increased the latency to REM onset, although the overall reduction in REM sleep did not reach significance (p=0.12). C decreased significantly for the first 2 h post administration but was not significant overall. NREM bout duration significantly (p<0.05) decreased during ZT14 (FIG. 5). No other significant effects were observed with Des treatment. All data collected and statistical analyses have been submitted in Excel format together with this report.

### Conclusions

Administration of Compound I-4 at the start of the active phase was followed by a suppression of REM and a strong, dose-related suppression of C. At the highest concentration, Compound I-4 significantly increased the consolidation of W without affecting time spent in W or NREM. Very few other effects were observed following Compound I-4 administration, including on the EEG power spectra.

Strong suppression of C together with increased W consolidation following Compound I-4 is an encouraging result for a possible narcolepsy therapeutic. Further studies are required to determine if these positive effects of Compound I-4 administration continue during the second half of the active phase or whether a “rebound” increase in C occurs.

### REFERENCES

Bittencourt, A. L., Takahashi, R. N. (1997). Eur J Pharmacol, 330 (2-3): 109-13. doi: 10.1016/s0014-2999(97)00182-9.
Bruera, E., et al. (1986). Cancer Treat Rep, 70 (2), 295-298.
Inoue, S. (1995). ObesRes, 3 Suppl 4, 549S-552S. doi: 10.1002/j.1550-8528.1995.tb00226.x.
Itoh, M., et al. (1995). Pediatr Neurol, 13 (4), 349-51. doi: 10.1016/0887-8994(95)00216-2.
Kanodia J., et al. (2021). Clin Pharmacokinet, 60 (1):121-131. doi: 10.1007/s40262-020-00918-7.
Korteling, D., et al. (2023). Am J Med Genet B Neuropsychiatr Genet, e32956
Lopez Quiñones A. J., et al. (2022). Drug Metab Dispos, 50 (9), 1218-27. doi: 10.1124/dmd.121.000707.
Nittur, N., et al. (2013). Sleep med, 14 (1), 30-36. doi: 10.1016/j.sleep.2012.07.008.
Robledo-Gonzalez, L. E., et al. (2017). JPain Res, 10, 1777-1786. doi: 10.2147/JPR.S136581.
Rothwell, N.J., et al. (1981). Br J Pharmacol, 74 (3), 539-546. doi: 10.1111/j.1476-5381.1981.tb10462.x.
Sagvolden, T. (2000). Neurosci Biobehav Rev, 24 (1), 31-39. doi: 10.1016/s0149-7634(99)00058-5.
Shah, F., Gupta, V. (2023). Kleine-Levin Syndrome (KLS). Treasure Island (FL): StatPearls Publishing; 2024 January
Suplicy, H., et al. (2013). Int J Obes (Lond), 38 (8), 1097-1103. doi: 10.1038/ijo.2013.225.
Sun, Y., et al. (2022). Sleep, 45 (12). doi: 10.1093/sleep/zsac039.
Tabuchi, S., et al. (2014). J Neurosci, 34 (19), 6495-6509. doi: 10.1523/JNEUROSCI.0073-14.2014.
Yoshida, T., et al. (1996). Clin Exp Pharmacol Physiol, 36 (6-7), 476-482. doi: 10.1111/j.1440-1681.1996.tb02764.x.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A method for treating a central nervous system disorder in a subject in need thereof, the method comprising administering to said subject in need thereof a therapeutically effective amount of a compound of Formula I, II, or III, or a stereoisomer thereof, or a pharmaceutically acceptable salt thereof: 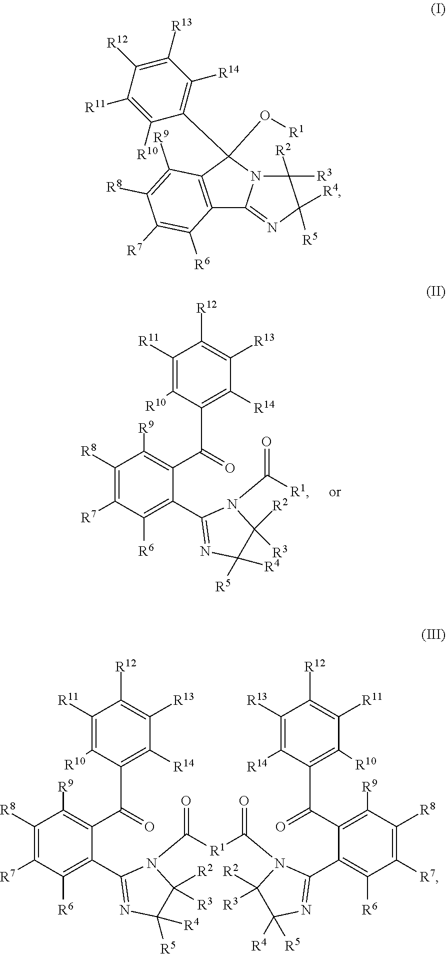
wherein:
for Formula I, R¹ is an alkyl group, an alkenyl group, an aralkyl group, an alkoxyalkyl group, a carboxyalkyl ester group, a cinnamyl group, a heteroalkyl group or a heterocycloalkyl group; R²-R⁵ are each independently H or alkyl; and R⁶-R¹⁴ are each independently H, alkyl, alkoxy, F, Cl, Br, CF₃ or I;
for Formula II, R¹ is a piperazinyl group, an alkyl group, an alkenyl group, a benzyl group, a phenyl group, or an amino acid residue derivative; and R²-R¹⁴ are each independently H, F, Cl, Br, or I; and
for Formula III, R¹ is an alkyl group, an alkenyl group, an aryl group, or a heteroaryl group; and R²-R¹⁴ are each independently H, F, Cl, Br, or I.

2. The method of claim 1, wherein the compound of Formula I is selected from the group consisting of:
5-(benzyloxy)-5-(4-chlorophenyl)-2,5-dihydro-3H-imidazo[2,1-a]isoindole (I-1),
5-(4-chlorophenyl)-5-((4-methoxybenzyl)oxy)-2,5-dihydro-3H-imidazo[2,1-a]isoindole (I-2),
5-(allyloxy)-5-(4-chlorophenyl)-2,5-dihydro-3H-imidazo[2,1-a]isoindole (I-3),
5-(4-chlorophenyl)-5-(2-methoxyethoxy)-2,5-dihydro-3H-imidazo[2,1-a]isoindole (I-4),
methyl 2-((5-(4-chlorophenyl)-2,5-dihydro-3H-imidazo[2,1-a]isoindol-5-yl)oxy)acetate (I-5),
5-(benzo[d][1,3]dioxol-5-ylmethoxy)-5-(4-chlorophenyl)-2,5-dihydro-3H-imidazo[2,1-a]isoindole (I-7),
5-(4-chlorophenyl)-5-ethoxy-2,5-dihydro-3H-imidazo[2,1-a]isoindole (I-8),
5-(4-chlorophenyl)-5-((3-methylbut-2-en-1-yl)oxy)-2,5-dihydro-3H-imidazo[2,1-a]isoindole (I-9),
5-(4-chlorophenyl)-5-isobutoxy-2,5-dihydro-3H-imidazo[2,1-a]isoindole (I-10),
(E)-5-(4-chlorophenyl)-5-((3,7-dimethylocta-2,6-dien-1-yl)oxy)-2,5-dihydro-3H-imidazo[2,1-a]isoindole (I-11),
5-(4-chlorophenyl)-5-(2-(2-(2-methoxyethoxy)ethoxy)ethoxy)-2,5-dihydro-3H-imidazo[2,1-a]isoindole (I-12),
5-(4-chlorophenyl)-5-(((2E,6E)-3,7,11-trimethyldodeca-2,6,10-trien-1-yl)oxy)-2,5-dihydro-3H-imidazo[2,1-a]isoindole (I-13),
5-(4-chlorophenyl)-5-((4-(trifluoromethyl)benzyl)oxy)-2,5-dihydro-3H-imidazo[2,1-a]isoindole (I-14),
5-(4-chlorophenyl)-5-((3,4-dimethoxybenzyl)oxy)-2,5-dihydro-3H-imidazo[2,1-a]isoindole (I-15),
5-((4-chlorobenzyl)oxy)-5-(4-chlorophenyl)-2,5-dihydro-3H-imidazo[2,1-a]isoindole (I-16),
(E)-5-(4-chlorophenyl)-5-((4-methylpent-2-en-1-yl)oxy)-2,5-dihydro-3H-imidazo[2,1-a]isoindole (I-17),
(E)-5-(4-chlorophenyl)-5-((3-(4-(trifluoromethyl)phenyl)allyl)oxy)-2,5-dihydro-3H-imidazo[2,1-a]isoindole (I-18),
5-((1,3-dioxolan-2-yl)methoxy)-5-(4-chlorophenyl)-2,5-dihydro-3H-imidazo[2,1-a]isoindole (I-19),
(E)-5-((3-(benzo[d][1,3]dioxol-5-yl)allyl)oxy)-5-(4-chlorophenyl)-2,5-dihydro-3H-imidazo[2,1-a]isoindole (I-20),
5-(4-chlorophenyl)-5-(cinnamyloxy)-2,5-dihydro-3H-imidazo[2,1-a]isoindole (I-21),
(E)-5-(4-chlorophenyl)-5-((3-(4-fluorophenyl)allyl)oxy)-2,5-dihydro-3H-imidazo[2,1-a]isoindole (I-22), and
(E)-5-(4-chlorophenyl)-5-((3-(4-chlorophenyl)allyl)oxy)-2,5-dihydro-3H-imidazo[2,1-a]isoindole (I-23).

3. The method of claim 1, wherein the compound of Formula II is selected from the group consisting of:
(2-(1-benzoyl-4,5-dihydro-1H-imidazol-2-yl)phenyl)(4-chlorophenyl)methanone (II-1),
(2-(2-(4-chlorobenzoyl)phenyl)-4,5-dihydro-1H-imidazol-1-yl)(4-methoxyphenyl)methanone (II-2),
(2-(2-(4-chlorobenzoyl)phenyl)-4,5-dihydro-1H-imidazol-1-yl)(p-tolyl)methanone (II-3),
benzo[d][1,3]dioxol-5-yl(2-(2-(4-chlorobenzoyl)phenyl)-4,5-dihydro-1H-imidazol-1-yl)methanone (II-4),
tert-butyl (1-(2-(2-(4-chlorobenzoyl)phenyl)-4,5-dihydro-1H-imidazol-1-yl)-3-methyl-1-oxobutan-2-yl)carbamate (II-5),
(2-(2-(4-chlorobenzoyl)phenyl)-4,5-dihydro-1H-imidazol-1-yl)(4-methylpiperazin-1-yl)methanone (II-6),
1-(2-(2-(4-chlorobenzoyl)phenyl)-4,5-dihydro-1H-imidazol-1-yl)-3-methylbut-2-en-1-one (II-7),
(E)-1-(2-(2-(4-chlorobenzoyl)phenyl)-4,5-dihydro-1H-imidazol-1-yl)-3-(4-methoxyphenyl)prop-2-en-1-one (II-8), and
(E)-1-(2-(2-(4-chlorobenzoyl)phenyl)-4,5-dihydro-1H-imidazol-1-yl)-3,7-dimethylocta-2,6-dien-1-one (II-9).

4. The method of claim 1, wherein the compound of Formula III is 1,3-bis(2-(2-(4-chlorobenzoyl)phenyl)-4,5-dihydro-1H-imidazol-1-yl)-2,2-dimethylpropane-1,3-dione (III-1).

5. The method of claim 1, wherein the central nervous system disorder is selected from the group consisting of an autism spectrum disorder, attention deficit hyperactivity disorder, an anxiety disorder, a movement disorder, an impulse control disorder, a personality disorder, a reward deficiency disorder, a somatoform disorder, a dementia disorder and an obsessive compulsive spectrum disorder, or a symptom of such a disorder.

6. The method of claim 1, wherein the central nervous system disorder is attention deficit hyperactivity disorder.

7. The method of claim 1, wherein administration is oral, anal, nasal, ocular, parenteral, intraperitoneal, intramuscular, or intravenous.

8. The method of claim 1, wherein the administration is oral.

9. The method of claim 1, wherein the subject is a human.

10. The method of claim 9, wherein the human is an infant, child, adolescent, or adult.

11. The method of claim 1, wherein the central nervous system disorder comprises at least disorder selected from the group consisting of Attention-Deficit/Hyperactivity Disorder (ADHD), Binge Eating Disorder, Chemotherapy-induced Pain, Chronic Fatigue Syndrome, Dopamine Beta-Hydrolase Deficiency, Fibromyalgia, Idiopathic Hypersomnia, Kleine-Levin Syndrome, Major Depressive Disorder, Narcolepsy Type I, Neuroendocrine Carcinoma, Neuropathic Pain, Obesity, Obsessive Compulsive Disorder, Orthostatic Hypotension and/or Orthostatic Intolerance, non-motor symptoms of Parkinson's Disease, Prader Willi Syndrome, Progressive Supranuclear Palsy, Smith Magenis Syndrome, and Spinocerebellar Ataxia.

12. The method of claim 1, wherein said administering reduces an impulsivity like behavior and/or a compulsivity-like behavior in the subject.

13. The method of claim 12, wherein said administering reduces at least one of premature responses, perseverative responses, and food magazine entries.

14. The method of claim 12, wherein said administering reduces an impulsivity-like and/or a compulsivity-like behavior caused by and/or associated with a disease or disorder selected from the group consisting of Prader Willi Syndrome, Smith Magenis Syndrome, Obsessive Compulsive Disorder, Binge Eating Disorder, Obesity, Attention-Deficit/Hyperactivity Disorder (ADHD), Kleine-Levin Syndrome, Substance Use Disorder, Parkinson's Disease, Spinocerebellar Ataxia, and Progressive Supranuclear Palsy.

15. The method of claim 1, wherein said administering reduces at least one symptom selected from the group consisting of hyperactivity, inattention, impulsivity, and challenges with concentration.

16. The method of claim 15, wherein the at least one symptom is caused by and/or associated with a disease or disorder selected from the group consisting of ADHD, Kleine-Levin Syndrome, and Smith Magenis Syndrome.

17. The method of claim 1, wherein said administering results in at least effect selected from the group consisting of: decreasing an excessive food consumption, altering mitochondria activity in a brown adipose tissue, decreasing white adipose tissue, increasing resting metabolic rate, decreasing blood glucose level and decreasing serum insulin levels.

18. The method of claim 17, wherein the subject has a disease or disorder selected from the group consisting of Prader Willi Syndrome, Smith Magenis Syndrome, Kleine-Levin Syndrome, Binge Eating Disorder, and Obesity.

19. The method of claim 1, wherein said administering results in at least one effect selected from the group consisting of: improving a food consumption level, improving weight management, and improving metabolic function.

20. The method of claim 19, wherein the subject has a disease or disorder selected from the group consisting of Prader Willi Syndrome, Smith Magenis Syndrome, Kleine-Levin Syndrome, Binge Eating Disorder, and Obesity.

21. The method of claim 1, wherein said administering results in reducing depression symptoms and/or anxiety symptoms.

22. The method of claim 21, wherein the subject has a disease or disorder selected from the group consisting of Major Depressive Disorder, Spinocerebellar Ataxia, Progressive Supranuclear Palsy, and Parkison's disease.

23. The method of claim 21, wherein said administering reduces depressive and/or apathetic symptoms in the subject with a disease or disorder selected from the group consisting of Spinocerebellar Ataxia, Progressive Supranuclear Palsy, and Parkison's disease.

24. The method of claim 1, wherein said administering results in at least one effect selected from the group consisting of modulating a sleeping parameter, treating a sleep disturbance, reducing a sleep disturbance, treating excessive daytime sleepiness and reducing excessive daytime sleepiness.

25. The method of claim 24, wherein the subject has a disease or disorder selected from the group consisting of ADHD, Prader Willi Syndrome, Smith Magenis Syndrome, Parkinson's disease and Kleine-Levin Syndrome.

26. The method of claim 1, wherein said administering results in treating and/or reducing at least one symptom selected from the group consisting of excessive daytime sleepiness, cataplexy, hypocretin deficiency and altered nocturnal sleep polysomnography characterized by altered rapid eye movement sleep phase transitions and onset.

27. The method of claim 26, wherein the at least one symptom is caused by and/or associated with Narcolepsy.

28. The method of claim 1, wherein said administering results in treating and/or reducing excessive daytime sleepiness.

29. The method of claim 28, wherein the excessive daytime sleepiness is caused by and/or associated with hypersomnia.

30. The method of claim 1, wherein said administering results in at least one effect selected from mitigating cataplexy and modifying a sleep parameter.

31. The method of claim 30, wherein the subject has narcolepsy.

32. The method of claim 1, wherein said administering improves at least one of wakefulness and cataplexy measures.

33. The method of claim 32, wherein the subject has a disease or disorder selected from the group consisting of narcolepsy and hypersomnia.

34. The method of claim 1, wherein said administering results in at least one effect selected from the group consisting of alleviating pain and/or pain symptoms, and regulating a mood of the subject.

35. The method of claim 34, wherein the subject has a disease or condition selected from the group consisting of a chronic pain disorder, arthritis, and cancer.

36. The method of claim 1, wherein said administering provides an anti-nociceptive effect.

37. The method of claim 1, wherein said administering inhibits norepinephrine and dopamine transporter.

38. The method of claim 1, wherein said administering results in noradrenergic modulation, thereby treating a disease or disorder, for which noradrenergic modulation is beneficial.

39. The method of claim 38, wherein the disease or disorder is selected from the group consisting of Dopamine Beta-Hydrolase Deficiency, orthostatic hypotension and orthostatic intolerance.

40. The method of claim 1, wherein said administering alleviates fatigue symptoms.

41. The method of claim 40, wherein said administering treats a chronic fatigue syndrome.

42. The method of claim 1, wherein said administering is used for treating and/or diagnosing neuroendocrine carcinoma or related disease.
